(19) United States
(12) Patent Application Publication
(10) Pub. No.: US 2023/0302015 A1
(43) Pub. Date: Sep. 28, 2023

(54) NATURAL COMBINATION HORMONE REPLACEMENT FORMULATIONS AND THERAPIES

(71) Applicant: TherapeuticsMD, Inc., Boca Raton, FL (US)

(72) Inventors: Julia M. Amadio, Boca Raton, FL (US); Brian A. Bernick, Boca Raton, FL (US); Sebastian Mirkin, Boca Raton, FL (US)

(21) Appl. No.: 18/077,212

(22) Filed: Dec. 7, 2022

Related U.S. Application Data
(63) Continuation of application No. 15/832,757, filed on Dec. 5, 2017, now abandoned.
(63) Continuation-in-part of application No. 18/053,120, filed on Nov. 7, 2022.
Application No. 18/053,120 is a continuation of application No. 16/520,167, filed on Jul. 23, 2019, now Pat. No. 11,529,360.
Application No. 16/520,167 is a continuation of application No. 15/999,040, filed on Aug. 16, 2018, now Pat. No. 11,166,963.
Application No. 15/999,040 is a continuation of application No. 14/690,955, filed on Apr. 20, 2015, now abandoned.
Application No. 14/690,955 is a division of application No. 14/099,582, filed on Dec. 6, 2013, now Pat. No. 9,012,434.
Application No. 14/099,582 is a continuation of application No. 13/843,428, filed on Mar. 15, 2013, now Pat. No. 9,301,920.
Application No. 13/843,428 is a continuation-in-part of application No. 13/684,002, filed on Nov. 21, 2012, now Pat. No. 8,633,178.
(60) Provisional application No. 62/430,339, filed on Dec. 5, 2016.

(51) Int. Cl. A61K 31/57; A61K 47/12; A61K 31/565; A61P 5/30; A61K 9/48

(52) U.S. Cl. CPC A61K 31/57; A61K 9/4858; A61K 31/565; A61K 47/12; A61P 5/30

(57) ABSTRACT

Pharmaceutical compositions for co-administering estradiol and progesterone to a human subject in need thereof are provided. In some embodiments, the pharmaceutical composition comprises solubilized estradiol, suspended progesterone, and a solubilizing agent comprising a medium chain (C6-C12) oil.

### CROSS-REFERENCE TO RELATED APPLICATIONS

This application is a continuation of U.S. Pat. Application No. 15/832,757, filed Dec. 5, 2017, which claims priority to U.S. Provisional Pat. Application No. 62/430,339, filed Dec. 5, 2016, and this application is a continuation-in-part of U.S. Pat. Application No. 18/053,120, filed Nov. 7, 2022, which is a continuation of U.S. Pat. Application No. 16/520,167, filed Jul. 23, 2019, now U.S. Pat. No. 11,529,360, issued Dec. 20, 2022, which is a continuation of U.S. Pat. Application No. 15/999,040, filed Aug. 16, 2018, now U.S. Pat. No. 11,166,963, issued Nov. 9, 2021, which is a continuation of U.S. Pat. Application No. 14/690,955, filed Apr. 20, 2015, which is a division of U.S. Pat. Application No. 14/099,582, filed Dec. 6, 2013, now U.S. Pat. No. 9,012,434, issued Apr. 21, 2015, which is a continuation of U.S. Pat. Application No. 13/843,428, filed Mar. 15, 2013, now U.S. Pat. No. 9,301,920, issued Apr. 5, 2016, which is a continuation-in-part of U.S. Pat. Application No. 13/684,002, filed Nov. 21, 2012, now U.S. Pat. No. 8,633,178, issued Jan. 21, 2014, the disclosures of which are incorporated herein by reference in their entirety.

### FIELD OF THE INVENTION

This application relates to pharmaceutical compositions and methods for hormone replacement therapy.

### BACKGROUND OF THE INVENTION

A decrease in estrogen at the time of menopause is associated with vasomotor instability (hot flushes and sweating), agitation, sleep disturbances, nervousness, mood changes, and urogenital atrophy.¹ The predominant estrogen produced by the ovaries is 17β-estradiol, the most active of the naturally occurring human estrogens. It is the principal intracellular human estrogen and is substantially more potent than its metabolites, estrone and estriol, at the receptor level. 17β-estradiol is used in menopausal hormone therapy.

The administration of 17β-estradiol to postmenopausal women significantly improves menopausal symptoms. In addition, estradiol may relieve or prevent many of the short-term physical and psychological consequences of estrogen deficiency. Estrogens can be considered the treatment of choice for most women with postmenopausal symptoms. Studies have shown that estrogen offers protection against osteoporosis.^{2,3}

Large prospective studies such as the Heart and Estrogen-Progestin Replacement Study (HERS) and the Women’s Health Initiative (WHI) have assessed the risks associated with the use of hormone therapy.⁴ The long-term effects of HT have been under close scrutiny since the results of these large randomized controlled trials, especially regarding the risk of breast cancer, coronary heart disease and venous thromboembolism. Following the publication of results of the WHI, the role of 17β-estradiol and progesterone for menopausal hormone therapy for non-hysterectomized postmenopausal women has continued.

Menopausal estrogen therapy is administered in a continuous daily dosage regimen or, alternatively, in a cyclic regimen. When estrogens are administered cyclically, the drugs are usually given once daily for 3 weeks followed by a 1 week hormone-free washout period, or once daily for 25 days followed by 5 hormone-free days, repeated as necessary.

¹ See, Balfour JA and Heel RC. Transdermal estradiol: A review of its pharmacodynamic and pharmacokinetic properties and therapeutic efficacy in the treatment of menopausal complaints. Drugs. 1990, 40(4):561-582.

² See, e.g., Rossouw JE, et al., Writing Group for the Women’s Health Initiative Investigators. Risks and benefits of estrogen plus progestin in healthy postmenopausal women: principal results From the Women’s Health Initiative randomized controlled trial. JAMA. 2002 Jul 17;288(3):321-33.

³ See, e.g., Anderson GL, et al. Women’s Health Initiative Steering Committee. Effects of conjugated equine estrogen in postmenopausal women with hysterectomy: the Women’s Health Initiative randomized controlled trial. JAMA. 2004 Apr 14;291 (14):1701-12.

⁴ See, e.g., Haas JS, et al., Changes in the use of postmenopausal hormone therapy after the publication of clinical trial results. Ann Intern Med. 2004 Feb 3; 140(3):184-8.

It has been known for more than 30 years that long-term use of unopposed estrogen is associated with an increased incidence of endometrial hyperplasia and endometrial cancer in postmenopausal women with a uterus. The addition of progestin to estrogen therapy reduces that risk.

As such, what is needed in the art is a combination product of 17β-estradiol and progesterone for menopausal hormone therapy that effectively treats menopausal symptoms without an increased incidence of endometrial hyperplasia. The present invention fulfills this need as well as other needs.

### BRIEF SUMMARY OF THE INVENTION

The invention provides a combination product consisting of a softgel formulation containing solubilized estradiol with micronized progesterone that can be used for the treatment of moderate to severe vasomotor symptoms associated with menopause. The combination product is comprised of active ingredients that are chemically and biologically identical to endogenous estradiol and progesterone in a softgel capsule form. The combination product provides a continuous combined hormone therapy regimen for menopausal women with an intact uterus who suffer from vasomotor symptoms associated with estrogen deficiency and who wish to avoid endometrial changes associated with unopposed estradiol therapy.

In one aspect, pharmaceutical compositions for co-administering estradiol and progesterone to a subject in need of natural hormone replacement therapies are provided. In some embodiments, the pharmaceutical composition comprises: solubilized estradiol, suspended progesterone, and a solubilizing agent, wherein the solubilizing agent is a medium chain (C6-C12) oil, and wherein the pharmaceutical composition, when administered to a subject, produces in a plasma sample from the subject one or more pharmacokinetic parameters as described herein (e.g., an area under the curve (AUC)₍₀₋ₜ₎ or a Cₘₐₓ for estradiol, progesterone, estrone, or total estrone as described herein, e.g., in Tables 18-21).

In some embodiments, the pharmaceutical composition comprises a solubilizing agent that comprises a glyceride of at least one C6-C12 fatty acid. In some embodiments, the glyceride ester is a mixture of mono- and diglycerides (e.g., glyceryl caprylate/caprate). In some embodiments, the fatty acid is predominantly a C8 to C10 fatty acid. In some embodiments, the pharmaceutical composition further comprises a surfactant (e.g., lauroyl polyoxyglyceride). In some embodiments, the pharmaceutical composition comprises estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg, and comprises progesterone at a dosage of about 25, 50, 75, 100, 125, 150, 175, 200, 250, 300, 350, or 400 mg.

In some embodiments, the pharmaceutical composition comprises about 0.25 mg estradiol and about 50 mg progesterone, and administration of the composition to the subject produces, in a plasma sample from the subject, one or more parameters selected from:
(i) an area under the curve (AUC)₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or both parameters selected from: an AUC₍₀₋ₜ₎ for estrone that is from 909.6091 pg·hr/ml to 1421.2642 pg·hr/ml; and a Cₘₐₓ for estrone that is from 42.6549 pg/ml to 66.6483 pg/ml.

In some embodiments, administration of the composition to subject further produces, in a plasma sample from the subject, one or both parameters selected from: an AUC₍₀₋ₜ₎ for total estrone that is from 20.1752 ng·hr/ml to 31.5238 ng·hr/ml; and a Cₘₐₓ for total estrone that is from 3.5429 ng/ml to 5.5358 ng/ml.

In some embodiments, the pharmaceutical composition comprises about 0.25 mg estradiol and about 50 mg progesterone, and administration of the composition to a subject produces, in a plasma sample from the subject, the following parameters:
(i) one or both of (a) an AUC₍₀₋ₜ₎ for estradiol that is from 140.3733 pg·hr/ml to 219.3333 pg·hr/ml and (b) a Cₘₐₓ for estradiol that is from 6.4790 pg/ml to 10.1235 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 909.6091 pg·hr/ml to 1421.2642 pg·hr/ml and (b) a Cₘₐₓ for estrone that is from 42.6549 pg/ml to 66.6483 pg/ml; and optionally
(iv) one or both of (a) an AUC₍₀₋ₜ₎ for total estrone that is from 20.1752 ng·hr/ml to 31.5238 ng·hr/ml and (b) a Cₘₐₓ for total estrone that is from 3.5429 ng/ml to 5.5358 ng/ml.

In some embodiments, a pharmaceutical composition for co-administering estradiol and progesterone to a human subject in need thereof comprises about 0.50 mg estradiol and about 50 mg progesterone, and administration of the composition to the subject produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or both parameters selected from: an AUC₍₀₋ₜ₎ for estrone that is from 1819.2181 pg·hr/ml to 2842.5283 pg·hr/ml, and a Cₘₐₓ for estrone that is from 85.3098 pg/ml to 133.2966 pg/ml.

In some embodiments, administration of the composition to the subject further produces, in a plasma sample from the subject, one or both parameters selected from: an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the pharmaceutical composition comprises about 0.50 mg estradiol and about 50 mg progesterone, and administration of the composition to a subject produces, in a plasma sample from the subject, the following parameters:
(i) one or both of (a) an UC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, a pharmaceutical composition for co-administering estradiol and progesterone to a human subject in need thereof comprises about 0.50 mg estradiol and about 100 mg progesterone, and administration of the composition to the subject produces, in a plasma sample from the subject, one or more parameters selected from:
(i) an area under the curve (AUC)₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or both parameters selected from: an AUC₍₀₋ₜ₎ for estrone that is from 1819.2181 pg·hr/ml to 2842.5283 pg·hr/ml, and a Cₘₐₓ for estrone that is from 85.3098 pg/ml to 133.2966 pg/ml.

In some embodiments, administration of the composition to the subject further produces, in a plasma sample from the subject, one or both parameters selected from: an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the pharmaceutical composition comprises about 0.50 mg estradiol and about 100 mg progesterone, and administration of the composition to a subject produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, a pharmaceutical composition for co-administering estradiol and progesterone to a human subject in need thereof comprises about 1 mg estradiol and about 100 mg progesterone, and administration of the composition to the subject produces, in a plasma sample from the subject, one or more parameters selected from:
(i) an area under the curve (AUC)₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or both parameters selected from: an AUC₍₀₋ₜ₎ for estrone that is from 3638.4363 pg·hr/ml to 5685.0567 pg·hr/ml, and a Cₘₐₓ for estrone that is from 170.6197 pg/ml to 266.5933 pg/ml.

In some embodiments, administration of the composition to the subject further produces, in a plasma sample from the subject, one or both parameters selected from: an AUC₍₀₋ₜ₎ for total estrone that is from 80.7010 ng·hr/ml to 126.0953 ng·hr/ml, and a Cₘₐₓ for total estrone that is from 14.1716 ng/ml to 22.1431 ng/ml.

In some embodiments, the pharmaceutical composition comprises about 0.50 mg estradiol and about 100 mg progesterone, and administration of the composition to a subject produces, in a plasma sample from the subject, the following parameters:
(i) one or both of (a) an AUC₍₀₋ₜ₎ for estradiol that is from 561.4933 pg·hr/ml to 877.3333 pg·hr/ml and (b) a Cₘₐₓ for estradiol that is from 25.9161 pg/ml to 40.4939 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 3638.4363 pg·hr/ml to 5685.0567 pg·hr/ml and (b) a Cₘₐₓ for estrone that is from 170.6197 pg/ml to 266.5933 pg/ml; and optionally
(iv) one or both of (a) an AUC₍₀₋ₜ₎ for total estrone that is from 80.7010 ng·hr/ml to 126.0953 ng·hr/ml and (b) a Cₘₐₓ for total estrone that is from 14.1716 ng/ml to 22.1431 ng/ml.

In some embodiments, the pharmaceutical composition has the blood plasma estradiol concentration profile of FIG. 1. In some embodiments, the pharmaceutical composition has the blood plasma progesterone concentration profile of FIG. 2. In some embodiments, the pharmaceutical composition has the blood plasma estrone concentration profile of FIG. 3. In some embodiments, the pharmaceutical composition has the blood plasma total estrone concentration profile of FIG. 4.

In some embodiments, the one or more parameters as described herein (e.g., the AUC₍₀₋ₜ₎ or Cₘₐₓ for progesterone, estradiol, estrone, or total estrone) are measured at regular intervals (e.g., about every 30 minutes, about every 60 minutes, or about every 90 minutes) or at irregular intervals over a period of time such as 24 hours or 48 hours. In some embodiments, the one or more parameters as described herein (e.g., the AUC₍₀₋ₜ₎ or Cₘₐₓ for progesterone, estradiol, estrone, or total estrone) are measured at about 0.25 hr, 0.5 hr, 0.67 hr, 0.83 hr, 1 hr, 1.33 hr, 1.67 hr, 2 hr, 2.5 hr, 3 hr, 4 hr, 5 hr, 6 hr, 7 hr, 8 hr, 10 hr, 12 hr, 18 hr, 24 hr, 36 hr, or 48 hr after administering the pharmaceutical composition to the subject. In some embodiments, the one or more parameters as described herein are measured at regular or irregular intervals following the administration of a single dose or of a first dose of the pharmaceutical composition to the subject.

In another aspect, methods of treating a subject are provided. In some embodiments, the subject has a condition that is caused at least in part by an estrogen deficiency (e.g., one or more symptoms of menopause, such as vasomotor symptoms). In some embodiments, the method comprises administering to the subject a pharmaceutical composition comprising solubilized estradiol, suspended progesterone, and a solubilizing agent that comprises a medium chain (C6-C12) oil as described herein, wherein administration of the pharmaceutical composition produces, in a plasma sample from the subject, one or more pharmacokinetic parameters as described herein. In some embodiments, the method comprises administering a pharmaceutical composition comprising estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg, and comprising progesterone at a dosage of about 25, 50, 75, 100, 125, 150, 175, 200, 250, 300, 350, or 400 mg. In some embodiments, the method comprises administering a pharmaceutical composition comprising: estradiol at a dosage of about 0.25 mg and progesterone at a dosage of about 50 mg; estradiol at a dosage of about 0.50 mg and progesterone at a dosage of about 50 mg; estradiol at a dosage of about 0.50 mg and progesterone at a dosage of about 100 mg; estradiol at a dosage of about 1 mg and progesterone at a dosage of about 100 mg; or estradiol at a dosage of about 2 mg and progesterone at a dosage of about 200 mg.

In some embodiments, the method comprises administering to the subject a pharmaceutical composition comprising about 0.25 mg estradiol and about 50 mg progesterone, wherein administration of the pharmaceutical composition produces, in a plasma sample from the subject, one or more parameters selected from:
(i) an area under the curve (AUC)₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml, and
(iv) a Cₘₐₓ for progesterone that is from 17.8444 ng/ml to 27.8819 ng/ml.

In some embodiments, administration of the pharmaceutical composition further produces, in a plasma sample from the subject, one or more parameters selected from: an AUC₍₀₋ₜ₎ for estrone that is from 909.6091 pg·hr/ml to 1421.2642 pg·hr/ml; a Cₘₐₓ for estrone that is from 42.6549 pg/ml to 66.6483 pg/ml; an AUC₍₀₋ₜ₎ for total estrone that is from 20.1752 ng·hr/ml to 31.5238 ng·hr/ml; and a Cₘₐₓ for total estrone that is from 3.5429 ng/ml to 5.5358 ng/ml.

In some embodiments, the method comprises administering to the subject a pharmaceutical composition comprising about 0.25 mg estradiol and about 50 mg progesterone, wherein administration of the pharmaceutical composition produces, in a plasma sample from the subject, the following parameters:
(i) one or both of (a) an AUC₍₀₋ₜ₎ for estradiol that is from 140.3733 pg·hr/ml to 219.3333 pg·hr/ml and (b) a Cₘₐₓ for estradiol that is from 6.4790 pg/ml to 10.1235 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 909.6091 pg·hr/ml to 1421.2642 pg·hr/ml and (b) a Cₘₐₓ for estrone that is from 42.6549 pg/ml to 66.6483 pg/ml; and optionally
(iv) one or both of (a) an an AUC₍₀₋ₜ₎ for total estrone that is from 20.1752 ng·hr/ml to 31.5238 ng·hr/ml and (b) a Cₘₐₓ for total estrone that is from 3.5429 ng/ml to 5.5358 ng/ml.

In some embodiments, the method comprises administering to the subject a pharmaceutical composition comprising about 0.50 mg estradiol and about 50 mg progesterone, wherein administration of the pharmaceutical composition produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or more parameters selected from: an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml; and a Cₘₐₓ for total estrone that is from 7.0858 ng/ml to 11.0715 ng/ml.

In some embodiments, the method comprises administering to the subject a pharmaceutical composition comprising about 0.50 mg estradiol and about 50 mg progesterone, wherein administration of the pharmaceutical composition produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, the method comprises administering to the subject a pharmaceutical composition comprising about 0.50 mg estradiol and about 100 mg progesterone, wherein administration of the pharmaceutical composition produces, in a plasma sample from the subject, one or more parameters selected from:
(i) an area under the curve (AUC)₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or more parameters selected from: an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the method comprises administering to the subject a pharmaceutical composition comprising about 0.50 mg estradiol and about 100 mg progesterone, wherein administration of the pharmaceutical composition produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, the method comprises administering to the subject a pharmaceutical composition comprising about 1 mg estradiol and about 100 mg progesterone, wherein administration of the pharmaceutical composition produces, in a plasma sample from the subject, one or more parameters selected from:
(i) an area under the curve (AUC)₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or more parameters selected from: an AUC₍₀₋ₜ₎ for estrone that is from 3638.4363 pg·hr/ml to 5685.0567 pg·hr/ml; a Cₘₐₓ for estrone that is from 170.6197 pg/ml to 266.5933 pg/ml; an AUC₍₀₋ₜ₎ for total estrone that is from 80.7010 ng·hr/ml to 126.0953 ng·hr/ml; and a Cₘₐₓ for total estrone that is from 14.1716 ng/ml to 22.1431 ng/ml.

In some embodiments, the method comprises administering to the subject a pharmaceutical composition comprising about 1 mg estradiol and about 100 mg progesterone, wherein administration of the pharmaceutical composition produces, in a plasma sample from the subject, the following parameters:
(i) one or both of (a) an AUC₍₀₋ₜ₎ for estradiol that is from 561.4933 pg·hr/ml to 877.3333 pg·hr/ml and (b) a Cₘₐₓ for estradiol that is from 25.9161 pg/ml to 40.4939 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 3638.4363 pg·hr/ml to 5685.0567 pg·hr/ml and (b) a Cₘₐₓ for estrone that is from 170.6197 pg/ml to 266.5933 pg/ml; and optionally
(iv) one or both of (a) an AUC₍₀₋ₜ₎ for total estrone that is from 80.7010 ng·hr/ml to 126.0953 ng·hr/ml and (b) a Cₘₐₓ for total estrone that is from 14.1716 ng/ml to 22.1431 ng/ml.

In still another aspect, pharmaceutical compositions for use in a method of treating a disease or condition that is caused at least in part by an estrogen deficiency are provided. In some embodiments, the pharmaceutical composition comprises solubilized estradiol, suspended progesterone, and a solubilizing agent that comprises a medium chain (C6-C12) oil, wherein the treatment produces, in a plasma sample from the subject, one or more pharmacokinetic parameters as described herein (e.g., an AUC₍₀₋ₜ₎ or Cₘₐₓ for estradiol, progesterone, estrone, or total estrone as described herein, e.g., as described in any of Tables 18-21). In some embodiments, the pharmaceutical compositions for use in a method of treating a disease or condition that is caused at least in part by an estrogen deficiency comprise estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg, and comprise progesterone at a dosage of about 25, 50, 75, 100, 125, 150, 175, 200, 250, 300, 350, or 400 mg.

In some embodiments, a pharmaceutical composition for use in a method of treating a disease or condition that is caused at least in part by an estrogen deficiency (e.g., one or more symptoms of menopause) comprises estradiol at a dosage of about 0.25 mg and progesterone at a dosage of about 50 mg, and produces one or more pharmacokinetic values disclosed in Table 18 following administration of a single dose of the pharmaceutical composition to a subject (e.g., about 24 hours or about 48 hours after administration).

In some embodiments, a pharmaceutical composition for use in a method of treating a disease or condition that is caused at least in part by an estrogen deficiency (e.g., one or more symptoms of menopause) comprises estradiol at a dosage of about 0.50 mg and progesterone at a dosage of about 50 mg, and produces one or more pharmacokinetic values disclosed in Table 19 following administration of a single dose of the pharmaceutical composition to a subject (e.g., about 24 hours or about 48 hours after administration).

In some embodiments, a pharmaceutical composition for use in a method of treating a disease or condition that is caused at least in part by an estrogen deficiency (e.g., one or more symptoms of menopause) comprises estradiol at a dosage of about 0.50 mg and progesterone at a dosage of about 100 mg, and produces one or more pharmacokinetic values disclosed in Table 20 following administration of a single dose of the pharmaceutical composition to a subject (e.g., about 24 hours or about 48 hours after administration).

In some embodiments, a pharmaceutical composition for use in a method of treating a disease or condition that is caused at least in part by an estrogen deficiency (e.g., one or more symptoms of menopause) comprises estradiol at a dosage of about 1 mg and progesterone at a dosage of about 100 mg, and produces one or more pharmacokinetic values disclosed in Table 21 following administration of a single dose of the pharmaceutical composition to a subject (e.g., about 24 hours or about 48 hours after administration).

### BRIEF DESCRIPTION OF THE DRAWINGS

FIG. 1 illustrates a semilogarithmic plot of mean plasma concentration (pg/ml) over time (hrs) for estradiol.

FIG. 2 illustrates a semilogarithmic plot of mean plasma concentration (ng/ml) over time (hrs) for progesterone.

FIG. 3 illustrates a semilogarithmic plot of mean plasma concentration (pg/ml) over time (hrs) for estrone.

FIG. 4 illustrates a semilogarithmic plot of mean plasma concentration (ng/ml) over time (hrs) for total estrone.

FIG. 5 illustrates mean change from baseline in weekly frequency of moderate to severse hot flashes for weeks 1 to 12.

FIG. 6 illustrates mean change from baseline in weekly severity of moderate to severse hot flashes for weeks 1 to 12.

FIG. 7 illustrates mean reduction in number of weekly moderate and severe VMS from week 1 through week 12 (MITT-VMS Population).

FIG. 8 illustrates mean reduction in severity of weekly moderate and severe VMS from week 1 through week 12 (MITT-VMS Population).

FIG. 9 illustrates mean reduction in number of weekly mild, moderate and severe VMS from week 1 through week 12 (MITT-VMS Population).

FIG. 10 illustrates mean reduction in severity of weekly mild, moderate, and severe VMS from week 1 through week 12 (MITT-VMS Population).

FIG. 11 illustrates percentage of subjects with ≥ 75% decrease in the mean number of moderate and severe vasomotor symptoms (MITT-VMS Population).

FIG. 12 illustrates the study design.

FIG. 13 illustrates Mean (±SD) Baseline-Adjusted Plasma Estradiol Concentration (pg/mL) versus Nominal Time (Linear Scale) - Sensitivity Analysis (PK Population).

FIG. 14 illustrates Mean Baseline-Adjusted Plasma Estradiol Concentration (pg/mL) versus Nominal Time (Semi-log Scale) - Sensitivity Analysis (PK Population).

FIG. 15 illustrates Mean (±SD) Unadjusted Plasma Estradiol Concentration (pg/mL) versus Nominal Time (Linear Scale) - Sensitivity Analysis (PK Population).

FIG. 16 illustrates Mean Unadjusted Plasma Estradiol Concentration (pg/mL) versus Nominal Time (Semi-log Scale) - Sensitivity Analysis (PK Population).

FIG. 17 illustrates Mean (±SD) Baseline-Adjusted Plasma Estrone Concentration (pg/mL) versus Nominal Time (Linear Scale) - Sensitivity Analysis (PK Population).

FIG. 18 illustrates Mean Baseline-Adjusted Plasma Estrone Concentration (pg/mL) versus Nominal Time (Semi-log Scale) - Sensitivity Analysis (PK Population).

FIG. 19 illustrates Mean (±SD) Unadjusted Plasma Estrone Concentration (pg/mL) versus Nominal Time (Linear Scale) - Sensitivity Analysis (PK Population).

FIG. 20 illustrates Mean Unadjusted Plasma Estrone Concentration (pg/mL) versus Nominal Time (Semi-log Scale) - Sensitivity Analysis (PK Population).

FIG. 21 illustrates Mean (±SD) Baseline-Adjusted Plasma Progesterone Concentration (ng/mL) versus Nominal Time (Linear Scale) - Sensitivity Analysis (PK Population).

FIG. 22 illustrates Mean Baseline-Adjusted Plasma Progesterone Concentration (ng/mL) versus Nominal Time (Semi-log Scale) - Sensitivity Analysis (PK Population).

FIG. 23 illustrates Mean (±SD) Unadjusted Plasma Progesterone Concentration (ng/mL) versus Nominal Time (Linear Scale) - Sensitivity Analysis (PK Population).

FIG. 24 illustrates Mean Unadjusted Plasma Progesterone Concentration (ng/mL) versus Nominal Time (Semi-log Scale) - Sensitivity Analysis (PK Population).

### DETAILED DESCRIPTION OF THE INVENTION

In the following detailed description of embodiments of this disclosure, reference is made to the accompanying drawings in which like references indicate similar elements, and in which is shown, by way of illustration, specific embodiments in which this disclosure may be practiced. These embodiments are described in sufficient detail to enable those skilled in the art to practice this disclosure, and it is to be understood that other embodiments may be utilized and that other changes may be made without departing from the scope of this disclosure. The following detailed description is, therefore, not to be taken in a limiting sense, and the scope of this disclosure is defined only by the appended claims. As used in this disclosure, the term “or” shall be understood to be defined as a logical disjunction (i.e., and/or) and shall not indicate an exclusive disjunction unless expressly indicated as such with the term “either,” “unless,” “alternatively,” and words of similar effect.

### I. Definitions

The singular forms “a,” “an,” and “the” include plural referents unless the context clearly dictates otherwise.

As used herein, the term “or” is a logical disjunction (i.e., and/or) and does not indicate an exclusive disjunction unless expressly indicated as such with the terms “either,” “unless,” “alternatively,” and words of similar effect.

The term “area under the curve” (“AUC”) refers to the area under the curve defined by changes in the blood concentration of an active pharmaceutical ingredient (e.g., estradiol or progesterone), or a metabolite of the active pharmaceutical ingredient, over time following the administration of a dose of the active pharmaceutical ingredient. “AUC_{0-∞}” is the area under the concentration-time curve extrapolated to infinity following the administration of a dose. “AUC₀₋ₜ” is the area under the concentration-time curve from time zero to time t following the administration of a dose, wherein t is the last time point with a measurable concentration.

The term “Cₘₐₓ” refers to the maximum value of blood concentration shown on the curve that represents changes in blood concentrations of an active pharmaceutical ingredient (e.g., progesterone or estradiol), or a metabolite of the active pharmaceutical ingredient, over time.

The term “tₘₐₓ” refers to the earliest time at which the blood concentration of an active pharmaceutical ingredient (e.g., estradiol or progesterone), or a metabolite of the active pharmaceutical ingredient is at its maximum value.

Collectively, AUC, Cₘₐₓ, and, optionally, Tₘₐₓ are the principal pharmacokinetic parameters that can characterize the pharmacokinetic response of a particular drug product, such as progesterone or estradiol, in an animal, especially a mammal, including human, subject.

The term “bioavailability,” which has the meaning defined in 21 C.F.R. § 320.1(a), refers to the rate and extent to which an active ingredient or active moiety is absorbed from a drug product and becomes available at the site of action. For drug products that are not intended to be absorbed into the bloodstream, bioavailability may be assessed by measurements intended to reflect the rate and extent to which the active ingredient or active moiety becomes available at the site of action. For example, bioavailability can be measured as the amount of active ingredient in the blood (serum or plasma) as a function of time. Pharmacokinetic (PK) parameters such as AUC, Cₘₐₓ, or tₘₐₓ may be used to measure and assess bioavailability.

The term “bioequivalent,” has the meaning defined in 21 C.F.R. § 320.1(e) and refers to the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Where there is an intentional difference in rate (e.g., in certain extended release dosage forms), certain pharmaceutical equivalents or alternatives may be considered bioequivalent if there is no significant difference in the extent to which the active ingredient or moiety from each product becomes available at the site of drug action. This applies only if the difference in the rate at which the active ingredient or moiety becomes available at the site of drug action is intentional and is reflected in the proposed labeling, is not essential to the attainment of effective body drug concentrations on chronic use, and is considered medically insignificant for the drug. In practice, two products are considered bioequivalent if the 90% confidence interval of the AUC or Cₘₐₓ is within 80.00% to 125.00%.

The terms “bio-identical hormone” and “body-identical” refer to an active pharmaceutical ingredient that is structurally identical to a hormone naturally or endogenously found in the human body (e.g., estradiol or progesterone).

As used herein, the term “about” refers to ±10% of the noted value, unless otherwise specified, and unless the upper bound of the range would exceed 100% of the pharmaceutical composition, in which case the upper limit of the range is limited to 99.9%. Thus, and by way of example only, a pharmaceutical composition including about 10 weight percent of a given compound could have from 9 to 11 weight percent of the compound. Similarly, a pharmaceutical composition including about 95 weight percent of a given compound could have from 85.5 to 99.9 weight percent of the compound in the pharmaceutical composition.

An “active pharmaceutical ingredient” (API), as used herein, means the active compound or compounds used in formulating a drug product, such as 17β-estradiol and progesterone. APIs are generally safe for administering to animals, especially mammals, including humans, according to established governmental standards, including those promulgated by the United States Food and Drug Administration.

The term “estradiol” refers to (17β)-estra-1,3,5(10)-triene-3,17-diol. Estradiol is also interchangeably called 17β-estradiol, oestradiol, or E2, and is found endogenously in the human body. As used herein, estradiol refers to the bio-identical or body-identical form of estradiol found in the human body having the structure:

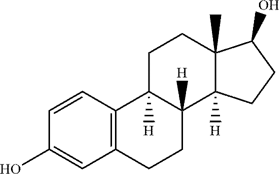

Estradiol is supplied in an anhydrous or hemi-hydrate form. For the purposes of this disclosure, the anhydrous form or the hemihydrate form can be substituted for the other by accounting for the water or lack of water according to well-known and understood techniques.

The term “solubilized estradiol” means that the estradiol or a portion thereof is solubilized or dissolved in the solubilizing agent(s) or the formulations disclosed herein. Solubilized estradiol may include estradiol that is about 80% solubilized, about 85% solubilized, about 90% solubilized, about 95% solubilized, about 96% solubilized, about 97% solubilized, about 98% solubilized, about 99% solubilized or about 100% solubilized. In some embodiments, the estradiol is “fully solubilized” with all or substantially all of the estradiol being solubilized or dissolved in the solubilizing agent. Fully solubilized estradiol may include estradiol that is about 97% solubilized, about 98% solubilized, about 99% solubilized or about 100% solubilized. Solubility can be expressed as a mass fraction (% w/w, which is also referred to as weight percent (wt%)).

The term “estrogen” refers to a group of several female sex hormones produced primarily by the ovaries, including estradiol, estrone, and estriol. As used herein, unless otherwise specified, estrogen refers to estradiol.

As used herein, the term “progesterone” refers to pregn-4-ene-3,20-dione. Progesterone is also interchangeably called P4 and is found endogenously in the human body. As used herein, progesterone refers to the bioidentical or body-identical form of progesterone found in the human body having the structure:

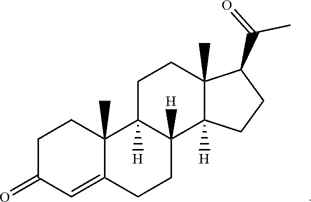

The term “solubilized progesterone” means that the progesterone or a portion thereof is solubilized or dissolved in the solubilizing agents or the formulations disclosed herein disclosed herein. In some embodiments, the progesterone is “partially solubilized” with a portion of the progesterone being solubilized or dissolved in the solubilizing agent and a portion of the progesterone being suspended in the solubilizing agent. Partially solubilized progesterone may include progesterone that is about 1% solubilized, about 5% solubilized, about 10% solubilized, about 15% solubilized, about 20% solubilized, about 30% solubilized, about 40% solubilized, about 50% solubilized, about 60% solubilized, about 70% solubilized, about 80% solubilized, about 85% solubilized, about 90% solubilized or about 95% solubilized. In other embodiments, the progesterone is “fully solubilized” with all or substantially all of the progesterone being solubilized or dissolved in the solubilizing agent. Fully solubilized progesterone may include progesterone that is about 97% solubilized, about 98% solubilized, about 99% solubilized or about 100% solubilized. Solubility can be expressed as a mass fraction (% w/w, which is also referred to as wt %).

The terms “micronized progesterone” and “micronized estradiol,” as used herein, include micronized progesterone and micronized estradiol having an X50 particle size value below about 15 microns or having an X90 particle size value below about 25 microns. The term “X50” means that one-half of the particles in a sample are smaller in diameter than a given number. For example, micronized progesterone having an X50 of 5 microns means that, for a given sample of micronized progesterone, one-half of the particles have a diameter of less than 5 microns. Similarly, the term “X90” means that ninety percent (90%) of the particles in a sample are smaller in diameter than a given number.

The solubility of a given steroid hormone can be measured using standard techniques by weighing a piece of filter paper, placing the weighed filter paper in a buchner funnel (porcelain or glass with a glass frit), and drawing a known quantity of pharmaceutical composition through the filter paper using vacuum (such as with a side-arm flask fitted with a neoprene collar). After drying for an appropriate period of time (either at room temperature or at elevated temperature), the filter paper is reweighed. The amount of steroid hormone on the filter paper is calculated and the amount of solubilized and insoluble steroid hormone is calculated.

The term “glyceride” refers to an ester of glycerol (1,2,3-propanetriol) with acyl radicals of fatty acids and is also known as an acylglycerol. If only one position of the glycerol molecule is esterified with a fatty acid, a “monoglyceride” or “monoacylglycerol” is produced; if two positions are esterified, a “diglyceride” or “diacylglycerol” is produced; and if all three positions of the glycerol are esterified with fatty acids, a “triglyceride” or “triacylglycerol” is produced. A glyceride is “simple” if all esterified positions contain the same fatty acid; whereas a glyceride is “mixed” if the esterified positions are substituted with different fatty acids. A glyceride is “complex” if it contains a combination of simple and mixed glycerides. The carbons of the glycerol backbone are designated sn-1, sn-2 and sn-3, with sn-2 being the middle carbon and sn-1 and sn-3 being the end carbons of the glycerol backbone.

As used herein, the term “hormone deficiency” refers to a low level of one or more steroid hormones in a subject. Normal hormone levels will vary from subject to subject and can be determined via known methods. Low hormone levels may or may not be associated with symptoms including, but not limited to, fatigue, irregular bleeding, lowered libido, and depression. Conditions that can be treated with estrogen and progesterone therapy to address estrogen and progesterone deficiencies include menopause-related symptoms including vasomotor symptoms (e.g., hot flashes and night sweats). Other hypoestrogenism related conditions and symptoms can also be treated with estrogen and progesterone therapy, including, for example and without limitation, vasomotor symptoms, sleep disturbances, mood changes, and vulvo-vaginal atrophy; and osteoporosis and other non-menopausal disease states or conditions that can be treated with supplemental estradiol and progesterone.

As used herein, the terms “host,” “subject,” and “patient” refer to any animal, including humans, especially female animals, including female humans.

The term “solubilizing agent” refers to an agent or combination of agents that solubilize an active pharmaceutical ingredient (e.g., estradiol or progesterone). For example and without limitation, suitable solubilizing agents include medium chain oils and other solvents and cosolvents that solubilize or dissolve an active pharmaceutical ingredient to a desirable extent. Solubilizing agents suitable for use in the pharmaceutical compositions disclosed herein are pharmaceutical grade solubilizing agents (e.g., pharmaceutical grade medium chain oils). It will be understood by those of skill in the art that other excipients or components can be added to or mixed with the solubilizing agent to enhance the properties or performance of the solubilizing agent or resulting pharmaceutical composition. Examples of such excipients include, but are not limited to, surfactants, emulsifiers, thickeners, colorants, flavoring agents, terpenes, etc. In some embodiments, the solubilizing agent is a medium chain oil and, in some other embodiments, the medium chain oil is combined with a co-solvent(s) or other excipient(s).

The term “medium chain” is used to describe the aliphatic chain length of fatty acid containing molecules. “Medium chain” specifically refers to fatty acids, fatty acid esters, or fatty acid derivatives that contain fatty acid aliphatic tails or carbon chains that contain, for example, 6 to 14 carbon atoms, 8 to 12 carbon atoms, or 8 to 10 carbon atoms.

The terms “medium chain fatty acid” and “medium chain fatty acid derivative” are used to describe fatty acids or fatty acid derivatives with aliphatic tails (i.e., carbon chains) having 6 to 14 carbon atoms. Fatty acids consist of an unbranched or branched aliphatic tail attached to a carboxylic acid functional group. Fatty acid derivatives include, for example, fatty acid esters and fatty acid containing molecules, including, without limitation, mono-, di- and triglycerides that include components derived from fatty acids. Fatty acid derivatives also include fatty acid esters of ethylene or propylene glycol. The aliphatic tails can be saturated or unsaturated (i.e., the latter having one or more double bonds between carbon atoms). In some embodiments, the aliphatic tails are saturated (i.e., no double bonds between carbon atoms). Medium chain fatty acids or medium chain fatty acid derivatives include those with aliphatic tails having 6-14 carbons, including those that are C6-C14, C6-C12, C8-C14, C8-C12, C6-C10, C8-C10, or others. Examples of medium chain fatty acids include, without limitation, caproic acid, caprylic acid, capric acid, lauric acid, myristic acid, and derivatives thereof. In certain embodiments, the medium chain fatty acids used to prepare the various medium chain oils described herein are C8, C10, or a combination thereof.

The term “oil,” as used herein, refers to any pharmaceutically acceptable oil, especially medium chain oils, and specifically excluding peanut oil, that can suspend or solubilize bioidentical progesterone or estradiol, including starting materials or precursors thereof, including micronized progesterone and/or micronized estradiol as described herein.

The term “medium chain oil” refers to an oil wherein the composition of the fatty acid fraction of the oil is substantially medium chain (i.e., C6 to C14) fatty acids, i.e., the composition profile of fatty acids in the oil is substantially medium chain. As used herein, “substantially” means that between 20% and 100% (inclusive of the upper and lower limits) of the fatty acid fraction of the oil is made up of medium chain fatty acids, i.e., fatty acids with aliphatic tails (i.e., carbon chains) having 6 to 14 carbons. In some embodiments, about 25%, about 30%, about 35%, about 40%, about 45%, about 50%, about 55%, about 60%, about 65%, about 70%, about 75%, about 85%, about 90% or about 95% of the fatty acid fraction of the oil is made up of medium chain fatty acids. Those of skill in the art will readily appreciate that the terms “alkyl content” or “alkyl distribution” of an oil can be used in place of the term “fatty acid fraction” of an oil in characterizing a given oil or solubilizing agent, and these terms are used interchangeably herein. As such, medium chain oils suitable for use in the pharmaceutical compositions disclosed herein include medium chain oils wherein the fatty acid fraction of the oil is substantially medium chain fatty acids, or medium chain oils wherein the alkyl content or alkyl distribution of the oil is substantially medium chain alkyls, e.g., C6-C14 alkyls, but also including, for example, C6-C12 alkyls, C8-C12 alkyls, and C8-C10 alkyls. It will be understood by those of skill in the art that the medium chain oils suitable for use in the pharmaceutical compositions disclosed herein are pharmaceutical grade (e.g., pharmaceutical grade medium chain oils). Examples of medium chain oils include, for example and without limitation, medium chain fatty acids, medium chain fatty acid esters of glycerol (e.g., for example, mono-, di-, and triglycerides), medium chain fatty acid esters of propylene glycol, medium chain fatty acid derivatives of polyethylene glycol, and combinations thereof.

The term “ECN” or “equivalent carbon number” means the sum of the number of carbon atoms in the fatty acid chains of an oil, and can be used to characterize an oil as, for example, a medium chain oil or a long-chain oil. For example, tripalmitin (tripalmitic glycerol), which is a simple triglyceride containing three fatty acid chains of 16 carbon atoms, has an ECN of 3 × 16=48. Conversely, a triglyceride with an ECN=40 may have “mixed” fatty acid chain lengths of 8, 16, and 16; 10, 14, and 16; 8, 14, and 18; etc. Naturally occurring oils are frequently “mixed” with respect to specific fatty acids, but tend not to contain both long chain fatty acids and medium chain fatty acids in the same glycerol backbone. Thus, triglycerides with ECN’s of 21-42 typically contain predominately medium chain fatty acids; while triglycerides with ECN’s of greater than 43 typically contain predominantly long chain fatty acids. For example, the ECN of corn oil triglyceride in the USP would be in the range of 51-54. Medium chain diglycerides with ECN’s of 12-28 will often contain predominately medium chain fatty chains, while diglycerides with ECN’s of 32 or greater will typically contain predominately long chain fatty acid tails. Monoglycerides will have an ECN that matches the chain length of the sole fatty acid chain. Thus, monoglyceride ECN’s in the range of 6-14 contain mainly medium chain fatty acids, and monoglycerides with ECN’s 16 or greater will contain mainly long chain fatty acids.

The average ECN of a medium chain triglyceride oil is typically 21-42. For example, as listed in the US Pharmacopeia (USP), medium chain triglycerides have the following composition as the exemplary oil set forth in the table below:

| Fatty-acid Tail Length | % of oil | Exemplary Oil |
|---|---|---|
| 6 | ≤2.0 | 2.0 |
| 8 | 50.0-80.0 | 70.0 |
| 10 | 20.0-50.0 | 25.0 |
| 12 | ≤3.0 | 2.0 |
| 14 | ≤1.0 | 1.0 |

and would have an average ECN of 3*[(6*0.02) + (8*0.70) + (10*0.25) + (12*0.02) + (14*0.01)] = 25.8. The ECN of the exemplary medium chain triglycerides oil can also be expressed as a range (per the ranges set forth in the USP) of 24.9 - 27.0. For oils that have mixed mono-, di-, and triglycerides, or single and double fatty acid glycols, the ECN of the entire oil can be determined by calculating the ECN of each individual component (e.g., C8 monoglycerides, C8 diglycerides, C10 monoglycerides, and C10 diglycerides) and taking the sum of the relative percentage of the component multiplied by the ECN normalized to a monoglyceride for each component. For example, an oil having C8 and C10 mono- and diglycerides shown in the table below has an ECN of 8.3, and is thus a medium chain oil.

| Fatty-acid ChainLength | % of oil | ECN as % of oil (chain length) × (% in oil) | ECN as % of oil normalized to monoglyceride |
|---|---|---|---|
| C8 monoglyceride | 47 | 8 × 0.47 = 3.76 | 3.76 |
| C10 monoglyceride | 8 | 10 × 0.08 = 0.8 | 0.8 |
| C8 diglyceride | 38 | 2 × (8 × 0.38) = 6.08 | 6.08/2 = 3.04 |
| C10 diglyceride | 7 | 2 × (10 × 0.07) = 1.4 | 1.4/2 = 0.7 |
| OIL ECN (normalized to monoglycerides) | | | 8.3 |

Expressed differently, ECN can be calculated as each chain length in the composition multiplied by its relative percentage in the oil: (8 * 0.85) + (10 ∗ 0.15) = 8.3.

The term “excipients,” as used herein, refers to non-API ingredients such as solubilizing agents, anti-oxidants, oils, lubricants, dissolution aids, terpenes, and others used in formulating pharmaceutical products.

The terms “treat,” “treating,” “treatment,” and the like refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient’s physical or mental well-being. The treatment or amelioration of symptoms can be based on objective or subjective parameters, including the results of a physical examination, neuropsychiatric examinations, or psychiatric evaluation.

As used herein, the term “prevent” refers to the prophylactic treatment of a subject who is at risk of developing a condition (e.g., steroid hormone deficiency) resulting in a decrease in the probability that the subject will develop the condition.

The phrase “therapeutically effective amount” refers to an amount of a pharmaceutical composition or of a given steroid hormone suitable to treat a particular symptom, disorder, or disease.

As used herein, the phrase “substantially pure” means that an identified component is at least about 90% pure by weight, in certain embodiments, at least about 95% pure by weight, and in still further embodiments, at least about 98% pure by weight.

As used herein, the phrase “steroid hormone” refers to progesterone, 17-hydroxyprogesterone, 5α-dihydroprogesterone, and estradiol.

As used herein, the phrase “reference product” refers to PROMETRIUM for progesterone and ESTRACE tablets for estradiol, unless otherwise specified.

The term “excipients,” as used herein, refers to non-active pharmaceutical ingredients such as solubilizing agents, anti-oxidants, oils, lubricants, and others used in formulating pharmaceutical products.

The terms “treat,” “treating,” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient’s physical or mental well-being. The treatment or amelioration of symptoms can be based on objective or subject parameters, including the results of a physical examination, neuropsychiatric examinations, or psychiatric evaluation.

### II. Pharmaceutical Compositions

In one aspect, this disclosure relates to pharmaceutical compositions for co-administering estradiol and progesterone to a human subject in need thereof. In some embodiments, the composition comprises estradiol, progesterone, and a solubilizing agent (e.g., a medium chain oil, e.g., a C6-C12 oil). In some embodiments, a pharmaceutical composition comprising estradiol, progesterone, and a solubilizing agent as described herein, when administered to a subject or a population of subjects, produces one or more AUC, Cₘₐₓ, or Tₘₐₓ parameters for estradiol, progesterone, estrone, or total estrone as described below.

### Formulations of Estradiol and Progesterone Compositions

In some embodiments, a pharmaceutical composition for use as described herein comprises solubilized estradiol with suspended progesterone; solubilized estradiol with both partially solubilized progesterone and partially suspended progesterone; or solubilized estradiol with fully solubilized progesterone. In some embodiments, the composition comprises solubilized estradiol and suspended progesterone. The underlying formulation concepts provided herein may be used with other natural or synthetic forms of estradiol and progesterone, although the natural or bio-identical forms of estradiol and progesterone are preferred.

In some embodiments, the composition comprises estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg. In some embodiments, the composition comprises progesterone at a dosage of about 25, 50, 75, 100, 125, 150, 175, 200, 250, 300, 350, or 400 mg.

In some embodiments, estradiol is solubilized. Solubilized estradiol may include estradiol that is approximately 80% to 100% soluble in a solubilizing agent, including specifically embodiments that are: 80%, 81%, 82%, 83%, 84%, 85%, 86%, 87%, 88%, 89%, 90%, 91%, 92%, 93%, 94%, 95%, 96%, 97%, 98%, 99%, or 100% soluble in a solubilizing agent. Solubility may be expressed as a mass fraction (% w/w, also referred to as wt%). In some embodiments, estradiol is micronized or partially micronized. In some embodiments, micronized estradiol has an X50 particle size value of less than about 15 microns, less than about 10 microns, less than about 5 microns or less than about 3 microns. In some embodiments, micronized estradiol has an X90 particle size value of less than about 25 microns, less than about 20 microns, or less than about 15 microns. In some embodiments, the composition comprises micronized and partially solubilized estradiol.

In some embodiments, the composition comprises micronized progesterone. The progesterone (or other active pharmaceutical ingredient, such as estradiol) may be micronized via any one of the multiple methods typically utilized by the ordinarily skilled artisan. In various embodiments, micronized progesterone has an X50 particle size value of less than about 15 microns, less than about 10 microns, less than about 5 microns or less than about 3 microns. In various embodiments, micronized progesterone has an X90 particle size value of less than about 25 microns, less than about 20 microns, or less than about 15 microns. Particle size may be determined in any suitable manner. For example, a Beckman Coulter LS 13 320 Laser Diffraction Particle Size Analyzer (the “Beckman Device”) may be used to determine particle size.

Estradiol and progesterone compositions and methods of preparing such compositions are described in U.S. Pat. No. 8,633,178; U.S. Publication No. 2013/0129818; U.S. Publication No. 2013/0338123; International Publication No. WO 2013/078422; and International Publication No. WO 2013/192251; each of which is incorporated by reference in its entirety.

### Solubilizing Agents

Estradiol and progesterone compositions of the present disclosure are prepared via blending with a solubilizing agent. In some embodiments, the solubilizing agent is a pharmaceutically acceptable oil that comprises a medium chain oil. In some embodiments, the solubilizing agent is a medium chain oil comprised substantially of C6-C12 medium chains, e.g., at least 20%, at least 30%, at least 40%, at least 50%, at least 60%, at least 70%, at least 80%, or at least 90% of the chains present in the oil are C6-C12. In some embodiments, the oil comprises at least one medium chain fatty acid such as medium chain fatty acids having at least one mono-, di-, or triglyceride, or derivatives thereof, or combinations thereof. In some embodiments, the medium chain oil comprises at least one medium chain fatty acid or propylene glycol, polyethylene glycol, or glyceride having esters of medium chain fatty acids. In some embodiments, the solubilizing agent is not peanut oil.

In some embodiments, oils used to solubilize estradiol and to suspend, partially suspend and partially solubilize, or fully solubilize progesterone include medium chain fatty acid esters, (e.g., esters of glycerol, polyethylene glycol, or propylene glycol) and mixtures thereof. In some embodiments, the medium chain fatty acids are C6, C8, C10, C12, C6-C12, C8-C12, C6-C10, C8-C10, or C10-C12 fatty acids. In some embodiments, the medium chain fatty acids are saturated, or predominantly saturated, e.g., greater than about 50% saturated, greater than about 60% saturated, or greater than about 75% saturated. In some embodiments, a solubilizing agent comprises predominantly medium chain length, saturated fatty acids or derivatives thereof, specifically predominantly C8 to C12 saturated fatty acids or derivatives thereof.

In some embodiments, medium chain solubilizing agents include, for example and without limitation, saturated medium chain fatty acids or derivatives of saturated medium chain fatty acids: caproic acid (C6), enanthic acid (C7), caprylic acid (C8), pelargonic acid (C9), capric acid (C10), undecylic acid (C11), lauric acid (C12), tridecylic acid (C13), or myristic acid (C14). In some embodiments, the solubilizing agent comprises oils made of these free medium chain fatty acids, oils of medium chain fatty acid esters of glycerin, propylene glycol, or ethylene glycol, or combinations thereof. These examples comprise predominantly saturated medium chain fatty acids (i.e., greater than 50% of the fatty acids are medium chain saturated fatty acids). In some embodiments, the solubilizing agent comprises predominantly C6 to C12 saturated fatty acids or derivatives of fatty acids.

In some embodiments, the solubilizing agent comprises one or more mono-, di-, or triglycerides or combinations thereof. Exemplary glycerin based solubilizing agents include MIGLYOLs®, which are caprylic/capric triglycerides (SASOL Germany GMBH, Hamburg). MIGLYOLsⓇincludes MIGLYOL® 810 (caprylic/capric triglyceride), MIGLYOL® 812 (caprylic/capric triglyceride), MIGLYOL® 816 (caprylic/capric triglyceride), and MIGLYOL® 829 (caprylic/capric/succinic triglyceride). Other caprylic/capric triglyceride solubilizing agents are likewise contemplated, including, for example: caproic/caprylic/capric/lauric triglycerides; caprylic/capric/linoleic triglycerides; or caprylic/capric/succinic triglycerides. Other exemplary caprylic/capric mono-, di-, or triiglyceride solubilizing agents include CAPMULs® (ABITEC, Columbus, Ohio), including, but are not limited to, CAPMULRO MCM, CAPMULRO MCM C10, CAPMUL® MCM C8, CAPMUL® MCM C8 EP, and CAPMUL® 708 G . Other mono-, di-, and triglycerides of fractionated vegetable fatty acids, and combinations or derivatives thereof can be the solubilizing agent, according to embodiments. For example, the solubilizing agent can be 1,2,3-propanetriol (glycerol, glycerin, glycerine) esters of saturated coconut and palm kernel oil and derivatives thereof.

In some embodiments, the solubilizing agent comprises one or more esters of propylene glycol, polyethylene glycol, or combinations thereof. Exemplary propylene and polyethylene glycol based solubilizing agents include glyceryl mono- and di-caprylates; propylene glycol monocaprylate (e.g., CAPMUL® PG-8 or CAPMUL® PG-8 NF); propylene glycol monocaprate (e.g., CAPMUL® PG-10); propylene glycol monolaurate (e.g., CAPMUL® PG-12 EP/NF); propylene glycol mono- and dicaprylates; propylene glycol mono- and dicaprate; propylene glycol dicaprylate/dicaprate (e.g., MIGLYOL® 840); propylene glycol dilaurate (e.g., CAPMUL® PG-2L EP/NF); diethylene glycol mono ester (e.g., TRANSCUTOL®,2-(2-Ethoxyethoxy)ethanol, GATTEFOSSÉ SAS, Saint-Priest, France); and diethylene glycol monoethyl ether.

In some embodiments, commercially available fatty acid glycerol and glycol ester solubilizing agents are prepared from natural oils and therefore may comprise components in addition to the fatty acid esters that predominantly comprise and characterize the solubilizing agent. Such other components may be, e.g., other fatty acid mono-, di-, and triglycerides, fatty acid mono- and diester ethylene or propylene glycols, free glycerols or glycols, or free fatty acids. For example, the Technical Data Sheet by ABITEC for CAPMUL® MCM C8 describes CAPMUL® MCM C8 as being composed of mono- and diglycerides of medium chain fatty acids (mainly caprylic) and describes the alkyl content as ≤ 1% C6, ≥ 95% C8, ≤ 5% C10, and ≤ 1.5% C12 and higher. By way of further example, MIGLYOL® 812 is generally described as a C8-C10 triglyceride because the fatty acid composition is at least about 80% caprylic (C8) acid and capric (C10) acid. However, it can also comprise small amounts of other fatty acids, e.g., less than about 5% of caproic (C6) acid, lauric (C12) acid, and myristic (C14) acid.

Any suitable amount of medium-chain oil can be used in the compositions disclosed herein. In general, the transdermal pharmaceutical compositions contain from about 10% (w/w) to about 30% (w/w). The compositions can contain, for example, from about 14% (w/w) to about 26% (w/w) medium-chain oil, or from about 18% (w/w) to about 22% (w/w) medium-chain oil, or from about 10% (w/w) to about 25% (w/w) medium-chain oil, or from about 10% (w/w) to about 20% (w/w) medium-chain oil, or from about 10% (w/w) to about 15% (w/w) medium-chain oil, or from about 15% (w/w) to about 20% (w/w) medium-chain oil, or from about 20% (w/w) to about 25% (w/w) medium-chain oil, or from about 25% (w/w) to about 30% (w/w) medium-chain oil. The compositions can contain about 10, 11, 12, 13, 14, 15, 16, 17, 18, 19, 20, 21, 22, 23, 24, 25, 26, 27, 28, 29 or 30% (w/w) medium-chain oil. In some embodiments, the transdermal pharmaceutical composition includes a medium-chain oil in an amount ranging from about 10% (w/w) to about 30% (w/w). In some embodiments, the transdermal pharmaceutical composition includes a medium-chain oil in an amount ranging from about 15% (w/w) to about 25% (w/w).

### Surfactants

In some embodiments, the pharmaceutical composition further comprises one or more non-ionic or ionic surfactants. In some embodiments, the non-ionic surfactant is selected from one or more of glycerol and polyethylene glycol esters of medium chain fatty acids or long chain fatty acids, for example, lauroyl macrogol-32 glycerides or lauroyl polyoxyl-32 glycerides, commercially available as GELUCIRE®, including, for example, GELUCIRE® 39/01 (glycerol esters of saturated C12-C18 fatty acids); GELUCIRE® 43/01 (hard fat NF/JPE); GELUCIRE® 44/14 (lauroyl macrogol-32 glycerides EP, lauroyl polyoxyl-32 glycerides NF, lauroyl polyoxylglycerides (USA FDA IIG)); and GELUCIRE® 50/13 (stearoyl macrogol-32 glycerides EP, stearoyl polyoxyl-32 glycerides NF, stearoyl polyoxylglycerides (USA FDA IIG)).

In some embodiments, non-ionic surfactants comprise combinations of mono- and dipropylene and ethylene glycols and mono-, di-, and triglyceride combinations. For example, in some embodiments, polyethylene glycol glyceride (GELUCIRE®, GATTEFOSSE SAS, Saint-Priest, France) can be used herein as the surfactant. For example, GELUCIRE® 44/14 (PEG-32 glyceryl laurate EP), a medium chain fatty acid esters of polyethylene glycol, is a polyethylene glycol glyceride composed of mono-, di- and triglycerides and mono- and diesters of polyethylene glycol.

In some embodiments, non-ionic surfactants include, for example and without limitation: one or more of oleic acid, linoleic acid, palmitic acid, and stearic acid. In some embodiments, non-ionic surfactants comprise polyethylene sorbitol esters, including polysorbate 80, which is commercially available under the trademark TWEEN 80® (Sigma Aldrich, St. Louis, MO). Polysorbate 80 comprises approximately 60%-70% oleic acid with the remainder comprising primarily linoleic acids, palmitic acids, and stearic acids.

In some embodiments, non-ionic surfactants include PEG-6 palmitostearate and ethylene glycol palmitostearate, which are available commercially as TEFOSE® 63 (GATTEFOSSE SAS, Saint-Priest, France), which can be used with, for example, CAPMUL® MCM having ratios of MCM to TEFOSE® 63 of, for example, 8:2 or 9:1. Other exemplary solubilizing agents/non-ionic surfactants combinations include, without limitation: MIGLYOL® 812:GELUCIRE 50/13 or MIGLYOL® 812:TEFOSE® 63.

A non-ionic or ionic surfactant may be used at concentrations greater than about 0.01%, for example at a concentration of about 0.01%-30.0%, about 0.1% to 10.0%, or about 1% to 10.0%, from 10% to 30%. In some embodiments, the pharmaceutical composition comprises about 10.0% surfactant by weight. In some embodiments, the pharmaceutical composition comprises about 15.0% surfactant by weight. In some embodiments, the pharmaceutical composition comprises about 0.1% to about 5.0% surfactant by weight, e.g., about 1.0 wt %. In some embodiments, the pharmaceutical composition comprises about 5.0% to about 15.0% surfactant by weight. In some embodiments, the pharmaceutical composition comprises about 10.0% to about 20.0% surfactant by weight. In some embodiments, the pharmaceutical composition comprises less than 30.0%, less than 29.0%, less than 28.0%, less than 27.0%, less than 26.0%, less than 25.0%, less than 24.0%, less than 23.0%, less than 22%, less than 21.0%, less than 20.0%, less than 19.0%, less than 18.0%, less than 17.0%, less than 16.0%, less than 15.0%, less than 14.0%, less than 13.0%, less than 12.0%, less than 11.0%, less than 10.0%, less than 9.0%, less than 8.0%, less than 7.0%, less than 6.0%, less than 5.0%, less than 4.0%, less than 3.0%, less than 2.0%, or less than 1.0% surfactant by weight.

### Other Excipients

In some embodiments, the pharmaceutical composition further comprises one more other excipients, such as but not limited to colorants, flavoring agents, preservatives, and taste-masking agents. The choice of excipients will, to a large extent, depend on factors such as the particular mode of administration, the effect of the excipients on solubility and stability, and the nature of the dosage form. Colorants, for example, may comprise about 0.1% to about 2% by weight. Preservatives may comprise methyl and propyl paraben, for example, in a ratio of about 10:1, and at a proportion of about 0.005% and 0.05% by weight.

Generally, the solubilizing agents, surfactants, and other excipients used in the pharmaceutical compositions described herein are non-toxic, pharmaceutically acceptable, compatible with each other, and maintain stability of the pharmaceutical composition and the various components with respect to each other. Additionally, the combination of various components that comprise the pharmaceutical compositions will result in the desired therapeutic effect when administered to a subject.

### Formulation

In some embodiments, combinations of solubilizing agents (e.g., two or more oils) or combinations of one or more solubilizing agents and one or more surfactants are used to form estradiol and progesterone compositions. Various ratios of these solubilizing agents or solubilizing agents and surfactants can be used. For example, CAPMUL® MCM and a non-ionic surfactant, e.g., GELUCIRE® 44/14 (lauroyl macrogol-32 glycerides EP; lauroyl polyoxyl-32 glycerides NF; lauroyl polyoxylglycerides (USA FDA IIG)), can be used at ratios of about 99:1 to about 2:1, including, for example and without limitation: 60:40, 65:35, 70:30, 75:25, 80:10, 80:15, 85:20, 90:10, and 98:1. As another example, CAPMUL® MCM and a non-ionic surfactant, e.g., TEFOSE® 63, can be used as rations of about 8:2 or 9:1. Other exemplary solubilizing agent/surfactant combinations include, without limitation: MIGLYOL® 812:GELUCIRE® 50/13 or MIGLYOL® 812:TEFOSE® 63. The ratios of oil (e.g., medium chain fatty acid esters of monoglycerides and diglycerides) to non-ionic surfactant can be significantly higher. For example, CAPMUL® MCM and GELUCIRE® can be used in ratios of up to about 65:1, e.g., 8:1, 22:1, 49:1, 65:1 and 66:1. Thus, useful ratios can be 8:1 or greater, e.g., 60 to 70:1.

In some embodiments, estradiol or progesterone is soluble in the solubilizing agent at room temperature, although it may be desirable to warm certain solubilizing agents. For example, when the formulation comprises medium chain fatty acid mono- and diglycerides (e.g., CAPMUL® MCM) and polyethylene glycol glycerides (e.g., GELUCIRE®) as a surfactant, the oil or the surfactant can be warmed up, e.g., to about 65° C. for the surfactant and less for the oil, to facilitate mixing of the oil and surfactant. The estradiol can be added at this temperature, or at lower temperatures as the mixture cools, e.g., about 40° C. or about 30° C., or even after the mixture has cooled to room temperature. The progesterone can also be added as the mixture cools, e.g., to below about 40° C. or to below about 30° C., or after the mixture has cooled to room temperature.

As a non-limiting example, a composition of this disclosure comprises solubilized estradiol; progesterone, at least 30% (e.g., at least about 30%, about 40%, about 50%, about 60%, about 70%, about 75%, about 80%, about 85%, or more) of the progesterone being solubilized (the balance being micronized as discussed elsewhere herein); and a solubilizing agent that is an oil, wherein the oil comprises medium chain fatty acid mono-, di-, or triglycerides, with or without a surfactant. In certain embodiments, a specification for progesterone is set at >80% solubilized, <20% micronized or >85% solubilized, <15% micronized. Specific examples of such illustrative embodiments, with CAPMUL® MCM NF (glyceryl caprylate/caprate) as a solubilizing agent and GELUCIRE® 44/14 (lauroyl polyoxyglyceride) as a surfactant, in which at least about 85% of the progesterone can be solubilized, include, e.g., the following five formulations A-E:

TABLE 1

Pharmaceutical Composition A - progesterone 50 mg / estradiol 0.25 mg

| Ingredient | Amount (% w/w) | Qty/Capsule (mg) |
|---|---|---|
| Progesterone, USP, micronized | 33.33 | 50.00 |
| Estradiol Hemihydrate | 0.17 | 0.26 |
| CAPMUL® MCM, NF | 65.49 | 98.24 |
| GELUCIRE® 44/14, NF | 1.00 | 1.50 |
| Total | 100.00 | 150.00 |

TABLE 2

Pharmaceutical Composition B - progesterone 50 mg / estradiol 0.5 mg

| Ingredient | Amount (% w/w) | Qty/Capsule (mg) |
|---|---|---|
| Progesterone, USP, micronized | 33.33 | 50.00 |
| Estradiol Hemihydrate | 0.35 | 0.52 |
| CAPMUL® MCM, NF | 65.32 | 97.98 |
| GELUCIRE® 44/14, NF | 1.00 | 1.50 |
| Total | 100.00 | 150.00 |

TABLE 3

Pharmaceutical Composition C - progesterone 100 mg / estradiol 0.5 mg

| Ingredient | Amount (% w/w) | Qty/Capsule (mg) |
|---|---|---|
| Progesterone, USP, micronized | 33.33 | 100.00 |
| Estradiol Hemihydrate | 0.17 | 0.52 |
| CAPMUL® MCM, NF | 65.49 | 196.48 |
| GELUCIRE® 44/14, NF | 1.00 | 3.00 |
| Total | 100.00 | 300.00 |

TABLE 4

Pharmaceutical Composition D - progesterone 100 mg / estradiol 1 mg

| Ingredient | Amount (% w/w) | Qty/Capsule (mg) |
|---|---|---|
| Progesterone, USP, micronized | 33.33 | 100.00 |
| Estradiol Hemihydrate | 0.34 | 1.03 |
| CAPMUL® MCM, NF | 65.32 | 195.97 |
| GELUCIRE® 44/14, NF | 1.00 | 3.00 |
| Total | 100.00 | 300.00 |

TABLE 5

Pharmaceutical Composition E - progesterone 200 mg / estradiol 2 mg

| Ingredient | Amount (% w/w) | Qty/Capsule (mg) |
|---|---|---|
| Progesterone, USP, micronized | 33.33 | 200.00 |
| Estradiol Hemihydrate | 0.34 | 2.06 |
| CAPMUL® MCM, NF | 65.32 | 391.94 |
| GELUCIRE® 44/14, NF | 1.00 | 6.00 |
| Total | 100.00 | 600.00 |

* Note: For all of the formulations disclosed herein, 1.00 mg Estradiol is equivalent to 1.03 mg Estradiol Hemihydrate

In general terms, the above formulations comprise 30 to 35 wt % progesterone, 0.1 to 0.4 wt % estradiol (or estradiol hemihydrate), 55 to 75 wt % of an oil that is predominantly medium chain fatty acid mono-, di-, or triglycerides, such as CAPMUL® MCM, and 0.5 to 10 wt % of a non-ionic surfactant, such as GELUCIRE® 44/14. The above formulations may be modified to comprise excipients, e.g., gelatin such as Gelatin 200 Bloom, glycerin, coloring agents such as Opatint red and white, and, optionally, MIGLYOL® 812.

Estradiol solubilization helps ensure high content uniformity and enhanced stability. Fully solubilized progesterone formulations or partially solubilized progesterone formulations in which at least about 50% of the progesterone, e.g., at least about 50%, 60%, 70%, 75%, 80%, 85%, 90%, 95% or more, is solubilized appear to provide improved PK-related properties.

### Pharmacokinetic Parameters of Estradiol and Progesterone Compositions

The pharmaceutical compositions of this disclosure are formulated to provide desirable pharmacokinetic parameters in a subject (e.g., a female subject) to whom the composition is administered. In some embodiments, a pharmaceutical composition as described herein produces desirable pharmacokinetic parameters for progesterone in the subject. In some embodiments, a pharmaceutical composition as described herein produces desirable pharmacokinetic parameters for estradiol in the subject. In some embodiments, a pharmaceutical composition as described herein produces desirable pharmacokinetic parameters for one or more metabolites of progesterone or estradiol in the subject, for example, estrone or total estrone.

Following the administration of a composition comprising progesterone and estradiol to a subject, the concentration and metabolism of progesterone or estradiol can be measured in a sample (e.g., a blood, serum, or plasma sample) from the subject. Progesterone is metabolized to pregnanediols and pregnanolones, which are then conjugated to glucuronide and sulfate metabolites that are excreted or further recycled. Estradiol is converted reversibly to estrone, and both estradiol and estrone can be converted to the metabolite estriol. In postmenopausal women, a significant proportion of circulating estrogens exist as sulfate conjugates, especially estrone sulfate. Thus, estrone can be measured with respect to “estrone” amounts (excluding conjugates such as estrone sulfate) and “total estrone” amounts (including both free, or unconjugated, estrone and conjugated estrone such as estrone sulfate).

The pharmaceutical compositions of this disclosure can be characterized by one or more pharmacokinetic parameters of progesterone, estradiol, or a metabolite thereof following administration of the composition to a subject or to a population of subjects. These pharmacokinetic parameters include AUC, Cₘₐₓ, and Tₘₐₓ. AUC is a determination of the area under the curve (AUC) plotting the blood, serum, or plasma concentration of drug along the ordinate (Y-axis) against time along the abscissa (X-axis). AUCs are well understood, frequently used tools in the pharmaceutical arts and have been extensively described. Cₘₐₓ is well understood in the art as an abbreviation for the maximum drug concentration in blood, serum, or plasma of a subject. Tₘₐₓ is well understood in the art as an abbreviation for the time to maximum drug concentration in blood, serum, or plasma of a subject.

In some embodiments, one or more pharmacokinetic parameters, e.g., AUC, Cₘₐₓ, or Tₘₐₓ, is measured for estradiol. In some embodiments, one or more pharmacokinetic parameters, e.g., AUC, Cₘₐₓ, or Tₘₐₓ, is measured for progesterone. In some embodiments, one or more pharmacokinetic parameters, e.g., AUC, Cₘₐₓ, or Tₘₐₓ, is measured for estrone. In some embodiments, one or more pharmacokinetic parameters, e.g., AUC, Cₘₐₓ, or Tₘₐₓ, is measured for total estrone.

Any of a variety of methods can be used for measuring the levels of progesterone, estradiol, estrone, or total estrone in a sample, including immunoassays, mass spectrometry (MS), high performance liquid chromatography (HPLC) with ultraviolet fluorescent detection, liquid chromatography in conjunction with mass spectrometry (LC-MS), tandem mass spectrometry (MS/MS), and liquid chromatography-tandem mass spectrometry (LC-MS/MS). In some embodiments, the levels of progesterone, estradiol, estrone, or total estrone are measured using a validated LC-MS/MS method. Methods of measuring hormone levels are well described in the literature.

The levels of progesterone, estradiol, estrone, or total estrone can be measured in any biological sample, e.g. a tissue or fluid such as blood, serum, plasma, or urine. In some embodiments, the sample is blood or plasma. In some embodiments, the levels of progesterone, estradiol, estrone, or total estrone are measured about 0.0, 0.10, 0.20, 0.05, 0.30, 0.35, 0.40, 0.45, 0.5, 1, 2, 3, 4, 5, 6, 7, 8, 9, 10, 12, 15, 18, 21, 24, 27, 30, 33, 36, 39, 42, 45, or 48 hours after dosing, or any other appropriate time period that is common or useful in determining the levels of each of the hormones. In some embodiments, the levels of progesterone, estradiol, estrone, or total estrone are measured about 18 hours, about 24 hours, about 18-36 hours, about 20-30 hours, about 22-26 hours, about 24-36 hours, about 36 hours, about 36-48 hours, about 40-48 hours, or about 48 hours after administration of a single dose or a first dose. Generally, assays to determine the levels of progesterone, estradiol, estrone, or total estrone are measured one or more times every 5, 10, 15, 20, 30, 60, 120, 360, 480, 720, or 1440 minutes after administration, or combinations thereof (e.g., the first measurements are taken every 15 minutes for the first hour, followed by every 120 minutes thereafter). In embodiments, the timing of such measurements are designed to accurately measure Cₘₐₓ, Tₘₐₓ, or AUC. Timing can be adjusted based on the given circumstances (i.e., one formulation may cause a more rapid Cₘₐₓ, in which case the initial times would be clustered closer together, closer to time zero, or both to ensure accurate measurement of Cₘₐₓ, Tₘₐₓ, and AUC). In some embodiments, the Cₘₐₓ, Tₘₐₓ, or AUC values for progesterone, estradiol, estrone, or total estrone are measured following administration of a single dose of a pharmaceutical composition as described herein.

In some embodiments, the values for Cₘₐₓ, Tₘₐₓ, or AUC represent a number of values taken from all the subjects in a patient population and are, therefore, mean values (e.g., arithmetic or geometric means) averaged over the entire population.

In some embodiments, oral administration of a pharmaceutical composition comprising estradiol, progesterone, and a medium chain solubilizing agent as described herein to a subject, or to a population of subjects, produces one or more AUC, Cₘₐₓ, or Tₘₐₓ parameters, or one or more mean AUC, mean Cₘₐₓ, or mean Tₘₐₓ parameters, respectively, for estradiol, progesterone, estrone, or total estrone as described below.

### AUC, Cₘₐₓ, and Tₘₐₓ Parameters for Formulation A

In some embodiments, a pharmaceutical composition of this disclosure comprises estradiol at a dosage of about 0.25 mg and progesterone at a dosage of about 50 mg. In some embodiments, the pharmaceutical composition comprises the formulation of Formulation A in Table 1 above.

In some embodiments, administration of a composition comprising about 0.25 mg estradiol and about 50 mg progesterone to a subject produces, in a plasma sample from the subject, one or both parameters selected from:
(i) an AUC₍₀₋ₜ₎ for estradiol that is from 140.3733 pg·hr/ml to 219.3333 pg·hr/ml; or
(ii) a Cₘₐₓ for estradiol that is from 6.4790 pg/ml to 10.1235 pg/ml.

In some embodiments, administration of the composition to the subject produces both an AUC₍₀₋ₜ₎ for estradiol that is from 140.3733 pg·hr/ml to 219.3333 pg·hr/ml, and a Cₘₐₓ for estradiol that is from 6.4790 pg/ml to 10.1235 pg/ml.

In some embodiments, administration of the composition to the subject further produces, in a plasma sample from the subject, one or both parameters selected from:
(i) an AUC₍₀₋ₜ₎ for progesterone that is from 24.0174 ng·hr/ml to 37.5272 ng·hr/ml; or
(ii) a Cₘₐₓ for progesterone that is from 17.8444 ng/ml to 27.8819 ng/ml.

In some embodiments, administration of the composition to the subject produces both an AUC₍₀₋ₜ₎ for progesterone that is from 24.0174 ng·hr/ml to 37.5272 ng·hr/ml, and a Cₘₐₓ for progesterone that is from 17.8444 ng/ml to 27.8819 ng/ml.

In some embodiments, administration of the composition to the subject produces, in a plasma sample from the subject,
(i) an AUC₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 909.6091 pg·hr/ml to 1421.2642 pg·hr/ml;
(ii) a Cₘₐₓ for estrone that is from 42.6549 pg/ml to 66.6483 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 20.1752 ng·hr/ml to 31.5238 ng·hr/ml;
(ii) a Cₘₐₓ for total estrone that is from 3.5429 ng/ml to 5.5358 ng/ml; or
(iii) a Tₘₐₓ for total estrone that is from 2 hr to 3.1 hr.

In some embodiments, a pharmaceutical composition comprising about 0.25 mg estradiol and about 50 mg progesterone is administered to a population of subjects in need thereof, and mean parameters are determined for samples (e.g., blood or plasma samples) from the subjects administered the composition. Thus, in some embodiments, administration of the composition to a population of subject produces, in plasma samples from the subjects, one or more of a mean AUC₍₀₋ₜ₎ for estradiol that is from 140.3733 pg·hr/ml to 219.3333 pg·hr/ml, a mean Cₘₐₓ for estradiol that is from 6.4790 pg/ml to 10.1235 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 24.0174 ng·hr/ml to 37.5272 ng·hr/ml, a mean Cₘₐₓ for progesterone that is from 17.8444 ng/ml to 27.8819 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 909.6091 pg·hr/ml to 1421.2642 pg·hr/ml, a mean Cₘₐₓ for estrone that is from 42.6549 pg/ml to 66.6483 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 20.1752 ng·hr/ml to 31.5238 ng·hr/ml, a mean Cₘₐₓ for total estrone that is from 3.5429 ng/ml to 5.5358 ng/ml, and a mean Tₘₐₓ for total estrone that is from 2 hr to 3.1 hr.

In some embodiments, methods of treating a subject with a pharmaceutical composition comprising estradiol and progesterone are provided. In some embodiments, the method comprises administering to the subject a pharmaceutical composition comprising about 0.25 mg estradiol and about 50 mg progesterone as described herein (e.g., a pharmaceutical composition having the formulation of Formulation A in Table 1 above), wherein administration of the pharmaceutical composition produces, in a plasma sample from the subject, one or more parameters selected from: an AUC₍₀₋ₜ₎ for estradiol that is from 140.3733 pg·hr/ml to 219.3333 pg·hr/ml; a Cₘₐₓ for estradiol that is from 6.4790 pg/ml to 10.1235 pg/ml; a Tₘₐₓ for estradiol that is from 7.2 hr to 11.3 hr; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; a Tₘₐₓ for progesterone that is from 2.4 hr to 3.8 hr; an AUC₍₀₋ₜ₎ for estrone that is from 909.6091 pg·hr/ml to 1421.2642 pg·hr/ml; a Cₘₐₓ for estrone that is from 42.6549 pg/ml to 66.6483 pg/ml; a Tₘₐₓ for estrone that is from 4.4 hr to 6.9 hr; an AUC₍₀₋ₜ₎ for total estrone that is from 20.1752 ng·hr/ml to 31.5238 ng·hr/ml; a Cₘₐₓ for total estrone that is from 3.5429 ng/ml to 5.5358 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation A in Table 1 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 140.3733 pg·hr/ml to 219.3333 pg·hr/ml; a Cₘₐₓ for estradiol that is from 6.4790 pg/ml to 10.1235 pg/ml; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; an AUC₍₀₋ₜ₎ for estrone that is from 909.6091 pg·hr/ml to 1421.2642 pg·hr/ml; a Cₘₐₓ for estrone that is from 42.6549 pg/ml to 66.6483 pg/ml; an AUC₍₀₋ₜ₎ for total estrone that is from 20.1752 ng·hr/ml to 31.5238 ng·hr/ml; or a Cₘₐₓ for total estrone that is from 3.5429 ng/ml to 5.5358 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

### AUC, Cₘₐₓ,and Tₘₐₓ Parameters (B)

In some embodiments, a pharmaceutical composition of this disclosure comprises estradiol at a dosage of about 0.50 mg and progesterone at a dosage of about 50 mg. In some embodiments, the pharmaceutical composition comprises the formulation of Formulation B in Table 2 above.

In some embodiments, administration of a composition comprising about 0.50 mg estradiol and about 50 mg progesterone to a subject produces, in a plasma sample from the subject, one or both parameters selected from:
(i) an AUC₍₀₋ₜ₎ for estradiol that is from 280.7467 pg·hr/ml to 438.6667 pg·hr/ml; or
(ii) a Cₘₐₓ for estradiol that is from 12.9580 pg/ml to 20.2469 pg/ml.

In some embodiments, administration of the composition to the subject produces both an AUC₍₀₋ₜ₎ for estradiol that is from 280.7467 pg·hr/ml to 438.6667 pg·hr/ml, and a Cₘₐₓ for estradiol that is from 12.9580 pg/ml to 20.2469 pg/ml.

In some embodiments, administration of the composition to the subject further produces, in a plasma sample from the subject, one or both parameters selected from:
(i) an AUC₍₀₋ₜ₎ for progesterone that is from 24.0174 ng·hr/ml to 37.5272 ng·hr/ml; or
(ii) a Cₘₐₓ for progesterone that is from 17.8444 ng/ml to 27.8819 ng/ml.

In some embodiments, administration of the composition to the subject produces both an (AUC)₍₀₋ₜ₎ for progesterone that is from 24.0174 ng·hr/ml to 37.5272 ng-hr/ml, and a Cₘₐₓ for progesterone that is from 17.8444 ng/ml to 27.8819 ng/ml.

In some embodiments, administration of the composition to the subject produces, in a plasma sample from the subject,
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; or
(iv) a Cₘₐₓ for progesterone that is from 17.8444 ng/ml to 27.8819 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 1819.2181 pg·hr/ml to 2842.5283 pg·hr/ml;
(ii) a Cₘₐₓ for estrone that is from 85.3098 pg/ml to 133.2966 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 40.3505 ng·hr/ml to 63.0476 ng·hr/ml;
(ii) a Cₘₐₓ for total estrone that is from 7.0858 ng/ml to 11.0715 ng/ml; or
(iii) a Tₘₐₓ for total estrone that is from 2 hr to 3.1 hr.

In some embodiments, a pharmaceutical composition comprising about 0.50 mg estradiol and about 50 mg progesterone is administered to a population of subjects in need thereof, and mean parameters are determined for samples (e.g., blood or plasma samples) from the subjects administered the composition. Thus, in some embodiments, administration of the composition to a population of subject produces, in plasma samples from the subjects, one or more of a mean AUC₍₀₋ₜ₎ for estradiol that is from 280.7467 pg·hr/ml to 438.6667 pg·hr/ml, a mean Cₘₐₓ for estradiol that is from 12.9580 pg/ml to 20.2469 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 24.0174 ng·hr/ml to 37.5272 ng·hr/ml, a mean Cₘₐₓ for progesterone that is from 17.8444 ng/ml to 27.8819 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 1819.2181 pg·hr/ml to 2842.5283 pg·hr/ml, a mean Cₘₐₓ for estrone that is from 85.3098 pg/ml to 133.2966 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 40.3505 ng·hr/ml to 63.0476 ng·hr/ml, a mean Cₘₐₓ for total estrone that is from 7.0858 ng/ml to 11.0715 ng/ml, and a mean Tₘₐₓ for total estrone that is from 2 hr to 3.1 hr.

In some embodiments, methods of treating a subject with a pharmaceutical composition comprising estradiol and progesterone are provided. In some embodiments, the method comprises administering to the subject a pharmaceutical composition comprising about 0.50 mg estradiol and about 50 mg progesterone as described herein (e.g., a pharmaceutical composition having the formulation of Formulation B in Table 2 above), wherein administration of the pharmaceutical composition produces, in a plasma sample from the subject, one or more parameters selected from: an AUC₍₀₋ₜ₎ for estradiol that is from 280.7467 pg·hr/ml to 438.6667 pg ·hr/ml; a Cₘₐₓ for estradiol that is from 12.9580 pg/ml to 20.2469 pg/ml; a Tₘₐₓ for estradiol that is from 7.2 hr to 11.3 hr; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; a Tₘₐₓ for progesterone that is from 2.4 hr to 3.8 hr; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; a Tₘₐₓ for estrone that is from 4.4 hr to 6.9 hr; an AUC₍₀₋ₜ₎ for total estrone that is from 40.3505 ng·hr/ml to 63.0476 ng·hr/ml; a Cₘₐₓ for total estrone that is from 7.0858 ng/ml to 11.0715 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation B in Table 2 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 280.7467 pg·hr/ml to 438.6667 pg·hr/ml; a Cₘₐₓ for estradiol that is from 12.9580 pg/ml to 20.2469 pg/ml; an AUC₍₀₋ₜ₎ for progesterone that is from 24.0174 ng·hr/ml to 37.5272 ng·hr/ml; a Cₘₐₓ for progesterone that is from 17.8444 ng/ml to 27.8819 ng/ml; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml; and a Cₘₐₓ for total estrone that is from 7.0858 ng/ml to 11.0715 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

### AUC, Cₘₐₓ, and Tₘₐₓ Parameters (C)

In some embodiments, a pharmaceutical composition of this disclosure comprises estradiol at a dosage of about 0.50 mg and progesterone at a dosage of about 100 mg. In some embodiments, the pharmaceutical composition comprises the formulation of Formulation C in Table 3 above.

In some embodiments, administration of a composition comprising about 0.50 mg estradiol and about 100 mg progesterone to a subject produces, in a plasma sample from the subject, one or both parameters selected from:
(i) an AUC₍₀₋ₜ₎ for estradiol that is from 280.7467 pg·hr/ml to 438.6667 pg·hr/ml; or
(ii) a Cₘₐₓ for estradiol that is from 12.9580 pg/ml to 20.2469 pg/ml.

In some embodiments, administration of the composition to the subject produces both an AUC₍₀₋ₜ₎ for estradiol that is from 280.7467 pg·hr/ml to 438.6667 pg·hr/ml, and a Cₘₐₓ for estradiol that is from 12.9580 pg/ml to 20.2469 pg/ml.

In some embodiments, administration of the composition to the subject further produces, in a plasma sample from the subject, one or both parameters selected from:
(i) an AUC₍₀₋ₜ₎ for progesterone that is from 48.0348 ng·hr/ml to 75.0543 ng·hr/ml; or
(ii) a Cₘₐₓ for progesterone that is from 35.6889 ng/ml to 55.7639 ng/ml.

In some embodiments, administration of the composition to the subject produces both an AUC₍₀₋ₜ₎ for progesterone that is from 48.0348 ng·hr/ml to 75.0543 ng·hr/ml, and a Cₘₐₓ for progesterone that is from 35.6889 ng/ml to 55.7639 ng/ml.

In some embodiments, administration of the composition to the subject produces, in a plasma sample from the subject,
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one, two, three or more parameters selected from:
(i) an AUC₍₀₋ₜ₎ for estrone that is from 1819.2181 pg·hr/ml to 2842.5283 pg·hr/ml;
(ii) a Cₘₐₓ for estrone that is from 85.3098 pg/ml to 133.2966 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 40.3505 ng·hr/ml to 63.0476 ng·hr/ml;
(ii) a Cₘₐₓ for total estrone that is from 7.0858 ng/ml to 11.0715 ng/ml; or
(iii) a Tₘₐₓ for total estrone that is from 2 hr to 3.1 hr.

In some embodiments, a pharmaceutical composition comprising about 0.50 mg estradiol and about 100 mg progesterone is administered to a population of subjects in need thereof, and mean parameters are determined for samples (e.g., blood and plasma samples) from the subjects administered the composition. Thus, in some embodiments, administration of the composition to a population of subject produces, in plasma samples from the subjects, one or more of a mean AUC₍₀₋ₜ₎ for estradiol that is from 280.7467 pg·hr/ml to 438.6667 pg·hr/ml, a mean Cₘₐₓ for estradiol that is from 12.9580 pg/ml to 20.2469 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 48.0348 ng·hr/ml to 75.0543 ng·hr/ml, a mean Cₘₐₓ for progesterone that is from 35.6889 ng/ml to 55.7639 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 1819.2181 pg·hr/ml to 2842.5283 pg·hr/ml, a mean Cₘₐₓ for estrone that is from 85.3098 pg/ml to 133.2966 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 40.3505 ng·hr/ml to 63.0476 ng·hr/ml, a mean Cₘₐₓ for total estrone that is from 7.0858 ng/ml to 11.0715 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 0.50 mg estradiol and about 100 mg progesterone as described herein (e.g., a pharmaceutical composition having the formulation of Formulation C in Table 3 above), wherein administration of the pharmaceutical composition produces, in a plasma sample from the subject, one or more parameters selected from: an AUC₍₀₋ₜ₎ for estradiol that is from 280.7467 pg·hr/ml to 438.6667 pg·hr/ml; a Cₘₐₓ for estradiol that is from 12.9580 pg/ml to 20.2469 pg/ml; a Tₘₐₓ for estradiol that is from 7.2 hr to 11.3 hr; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; a Tₘₐₓ for progesterone that is from 2.4 hr to 3.8 hr; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; a Tₘₐₓ for estrone that is from 4.4 hr to 6.9 hr; an AUC₍₀₋ₜ₎ for total estrone that is from 40.3505 ng·hr/ml to 63.0476 ng·hr/ml; a Cₘₐₓ for total estrone that is from 7.0858 ng/ml to 11.0715 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation C in Table 3 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 280.7467 pg·hr/ml to 438.6667 pg·hr/ml; a Cₘₐₓ for estradiol that is from 12.9580 pg/ml to 20.2469 pg/ml; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml; and a Cₘₐₓ for total estrone that is from 7.0858 ng/ml to 11.0715 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

### AUC, Cₘₐₓ, and Tₘₐₓ Parameters (D)

In some embodiments, a pharmaceutical composition of this disclosure comprises estradiol at a dosage of about 1 mg and progesterone at a dosage of about 100 mg. In some embodiments, the pharmaceutical composition comprises the formulation of Formulation D in Table 4 above.

In some embodiments, administration of a composition comprising about 1 mg estradiol and about 100 mg progesterone to a subject produces, in a plasma sample from the subject, one or both parameters selected from:
(i) an AUC₍₀₋ₜ₎ for estradiol that is from 561.4933 pg·hr/ml to 877.3333 pg·hr/ml; or
(ii) a Cₘₐₓ for estradiol that is from 25.9161 pg/ml to 40.4939 pg/ml.

In some embodiments, administration of the composition to the subject produces both an AUC₍₀₋ₜ₎ for estradiol that is from 561.4933 pg·hr/ml to 877.3333 pg·hr/ml, and a Cₘₐₓ for estradiol that is from 25.9161 pg/ml to 40.4939 pg/ml.

In some embodiments, administration of the composition to the subject further produces, in a plasma sample from the subject, one or both parameters selected from:
(i) an AUC₍₀₋ₜ₎ for progesterone that is from 48.0348 ng·hr/ml to 75.0543 ng·hr/ml; or
(ii) a Cₘₐₓ for progesterone that is from 35.6889 ng/ml to 55.7639 ng/ml.

In some embodiments, administration of the composition to the subject produces both an AUC₍₀₋ₜ₎ for progesterone that is from 48.0348 ng·hr/ml to 75.0543 ng·hr/ml, and a Cₘₐₓ for progesterone that is from 35.6889 ng/ml to 55.7639 ng/ml.

In some embodiments, administration of the composition to the subject produces, in a plasma sample from the subject,
(i) an AUC₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the composition to the subject produces, in a plasma sample from the subject, one, two, three or more parameters selected from:
(i) an AUC₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; or
(iv) a Cₘₐₓ for progesterone that is from 35.6889 ng/ml to 55.7639 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 3638.4363 pg·hr/ml to 5685.0567 pg·hr/ml;
(ii) a Cₘₐₓ for estrone that is from 170.6197 pg/ml to 266.5933 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 80.7010 ng·hr/ml to 126.0953 ng·hr/ml;
(ii) a Cₘₐₓ for total estrone that is from 14.1716 ng/ml to 22/1431 ng/ml; or
(iii) a Tₘₐₓ for total estrone that is from 2 hr to 3.1 hr.

In some embodiments, a pharmaceutical composition comprising about 1 mg estradiol and about 100 mg progesterone is administered to a population of subjects in need thereof, and mean parameters are determined for samples (e.g., blood or plasma samples) from the subjects administered the composition. Thus, in some embodiments, administration of the composition to a population of subject produces, in plasma samples from the subjects, one or more of a mean AUC₍₀₋ₜ₎ for estradiol that is from 561.4933 pg·hr/ml to 877.3333 pg·hr/ml, a mean Cₘₐₓ for estradiol that is from 25.9161 pg/ml to 40.4939 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 48.0348 ng·hr/ml to 75.0543 ng·hr/ml, a mean Cₘₐₓ for progesterone that is from 35.6889 ng/ml to 55.7639 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 3638.4363 pg·hr/ml to 5685.0567 pg·hr/ml, a mean Cₘₐₓ for estrone that is from 170.6197 pg/ml to 266.5933 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 80.7010 ng·hr/ml to 126.0953 ng·hr/ml, a mean Cₘₐₓ for total estrone that is from 14.1716 ng/ml to 22/1431 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 1 mg estradiol and about 100 mg progesterone as described herein (e.g., a pharmaceutical composition having the formulation of Formulation D in Table 4 above), wherein administration of the pharmaceutical composition produces, in a plasma sample from the subject, one or more parameters selected from: an AUC₍₀₋ₜ₎ for estradiol that is from 561.4933 pg·hr/ml to 877.3333 pg·hr/m; a Cₘₐₓ for estradiol that is from 25.9161 pg/ml to 40.4939 pg/ml; a Tₘₐₓ for estradiol that is from 7.2 hr to 11.3 hr; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; a Tₘₐₓ for progesterone that is from 2.4 hr to 3.8 hr; an AUC₍₀₋ₜ₎ for estrone that is from 3638.4363 pg·hr/ml to 5685.0567 pg·hr/ml; a Cₘₐₓ for estrone that is from 170.6197 pg/ml to 266.5933 pg/ml; a Tₘₐₓ for estrone that is from 4.4 hr to 6.9 hr; an AUC₍₀₋ₜ₎ for total estrone that is from 80.7010 ng·hr/ml to 126.0953 ng·hr/ml; a Cₘₐₓ for total estrone that is from 14.1716 ng/ml to 22/1431 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation D in Table 4 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 561.4933 pg·hr/ml to 877.3333 pg·hr/m; a Cₘₐₓ for estradiol that is from 25.9161 pg/ml to 40.4939 pg/ml; an AUC₍₀₋ₜ₎ for progesterone that is from 48.0348 ng·hr/ml to 75.0543 ng·hr/ml; a Cₘₐₓ for progesterone that is from 35.6889 ng/ml to 55.7639 ng/ml; an AUC₍₀₋ₜ₎ for estrone that is from 3638.4363 pg·hr/ml to 5685.0567 pg·hr/ml; a Cₘₐₓ for estrone that is from 170.6197 pg/ml to 266.5933 pg/ml; an AUC₍₀₋ₜ₎ for total estrone that is from 80.7010 ng·hr/ml to 126.0953 ng·hr/ml; and a Cₘₐₓ for total estrone that is from 14.1716 ng/ml to 22/1431 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

### AUC, Cₘₐₓ, and Tₘₐₓ Parameters (E)

In some embodiments, a pharmaceutical composition of this disclosure comprises estradiol at a dosage of about 2 mg and progesterone at a dosage of about 200 mg. In some embodiments, the pharmaceutical composition comprises the formulation of Formulation E in Table 5 above.

In some embodiments, administration of a pharmaceutical composition comprising about 2 mg estradiol and about 200 mg progesterone to a subject produces, in a plasma sample from the subject, one or both parameters selected from:
(i) an AUC₍₀₋ₜ₎ for estradiol that is from 1123 pg·h/ml to 1755 pg·h/ml; or
(ii) a Cₘₐₓ for estradiol that is from 52 pg/ml to 81 pg/ml.

In some embodiments, administration of the composition to the subject produces both an AUC₍₀₋ₜ₎ for estradiol that is from 1123 pg·h/ml to 1755 pg·h/ml, and a Cₘₐₓ for estradiol that is from 52 pg/ml to 81 pg/ml.

In some embodiments, administration of the composition to the subject further produces, in a plasma sample from the subject, one or both parameters selected from:
(i) an AUC₍₀₋ₜ₎ for progesterone that is from 96 ng·hr/ml to 150 ng·hr/ml; or
(ii) a Cₘₐₓ for progesterone that is from 71 ng/ml to 112 ng/ml.

In some embodiments, administration of the composition to the subject produces both an AUC₍₀₋ₜ₎ for progesterone that is from 96 ng·hr/ml to 150 ng·hr/ml, and a Cₘₐₓ for progesterone that is from 71 ng/ml to 112 ng/ml.

In some embodiments, administration of the composition to the subject produces, in a plasma sample from the subject,
(i) an AUC₍₀₋ₜ₎ for estradiol that is from 1123 pg·h/ml to 1755 pg·h/ml;
(ii) a Cₘₐₓ for estradiol that is from 52 pg/ml to 81 pg/ml;
(iii) an AUC₍₀₋ₜ₎ for progesterone that is from 96 ng·hr/ml to 150 ng·hr/ml; or
(iv) a Cₘₐₓ for progesterone that is from 71 ng/ml to 112 ng/ml.

In some embodiments, administration of the composition to the subject further produces, in a plasma sample from the subject, a Tₘₐₓ for estradiol that is from 7.2 hr to 11.3 hr. In some embodiments, administration of the composition to the subject further produces, in a plasma sample from the subject, a Tₘₐₓ for progesterone that is from 2.4 hr to 3.8 hr.

In some embodiments, administration of the pharmaceutical composition to the subject produces, in a plasma sample from the subject, one, two, three or more parameters selected from:
(i) an AUC₍₀₋ₜ₎ for estradiol that is from 1123 pg·h/ml to 1755 pg·h/ml;
(ii) a Cₘₐₓ for estradiol that is from 52 pg/ml to 81 pg/ml;
(iii) an AUC₍₀₋ₜ₎ for progesterone that is from 96 ng·hr/ml to 150 ng·hr/ml; or
(iv) a Cₘₐₓ for progesterone that is from 71 ng/ml to 112 ng/ml.

In some embodiments, administration of the pharmaceutical composition to the subject produces both parameters (i) and (ii). In some embodiments, administration of the composition to the subject produces both parameters (i) and (iii). In some embodiments, administration of the composition to the subject produces both parameters (i) and (iv). In some embodiments, administration of the composition to the subject produces both parameters (ii) and (iii). In some embodiments, administration of the composition to the subject produces both parameters (ii) and (iv). In some embodiments, administration of the composition to the subject produces both parameters (iii) and (iv). In some embodiments, administration of the composition to the subject produces all of parameters (i), (ii), and (iii). In some embodiments, administration of the composition to the subject produces both parameters (i), (iii), and (iv). In some embodiments, administration of the composition to the subject produces both parameters (ii), (iii), and (iv). In some embodiments, administration of the composition to the subject produces all of parameters (i), (ii), (iii), and (iv).

In some embodiments, administration of the pharmaceutical composition to the subject further produces, in a plasma sample from the subject, one or more parameters selected from:
(i) an AUC₍₀₋ₜ₎ for estrone that is from 7277 pg·hr/ml to 11370 pg·hr/ml;
(ii) a Cₘₐₓ for estrone that is from 341 pg/ml to 533 pg/ml; or
(iii) a Tₘₐₓ for estrone that is from 4.4 hr to 6.9 hr.

In some embodiments, administration of the pharmaceutical composition to the subject further produces, in a plasma sample from the subject, one or more parameters selected from:
(i) an AUC₍₀₋ₜ₎ for total estrone that is from 161 ng·h/ml to 252 ng·h/ml
(ii) a Cₘₐₓ for total estrone that is from 28 ng/ml to 44 ng/ml; or
(iii) a Tₘₐₓ for total estrone that is from 2 hr to 3.1 hr.

In some embodiments, a pharmaceutical composition comprising about 2 mg estradiol and about 200 mg progesterone is administered to a population of subjects in need thereof, and mean parameters are determined for samples (e.g., blood or plasma samples) from the subjects administered the composition. Thus, in some embodiments, administration of the composition to a population of subject produces, in plasma samples from the subjects, one or more of a mean AUC₍₀₋ₜ₎ for estradiol that is from 1123 pg·h/ml to 1755 pg·h/ml, a mean Cₘₐₓ for estradiol that is from 52 pg/ml to 81 pg/ml, and a mean Tₘₐₓ for estradiol that is from 7.2 hr to 11.3 hr. In some embodiments, administration of the composition to a population of subject produces, in plasma samples from the subjects, one or more of a mean AUC₍₀₋ₜ₎ for progesterone that is from 96 ng·hr/ml to 150 ng·hr/ml, a mean Cₘₐₓ for progesterone that is from 71 ng/ml to 112 ng/ml, and a mean Tₘₐₓ for progesterone that is from 2.4 hr to 3.8 hr. In some embodiments, administration of the composition to a population of subject produces, in plasma samples from the subjects, one or more of a mean AUC₍₀₋ₜ₎ for estrone that is from 7277 pg·hr/ml to 11370 pg·hr/ml, a mean Cₘₐₓ for estrone that is from 341 pg/ml to 533 pg/ml, and a mean Tₘₐₓ for estrone that is from 4.4 hr to 6.9 hr. In some embodiments, administration of the composition to a population of subject produces, in plasma samples from the subjects, one or more of a mean AUC₍₀₋ₜ₎ for total estrone that is from 161 ng·h/ml to 252 ng·h/ml, a mean Cₘₐₓ for total estrone that is from 28 ng/ml to 44 ng/ml, and a mean Tₘₐₓ for total estrone that is from 2 hr to 3.1 hr.

In some embodiments, method of treating a subject with a pharmaceutical composition comprising estradiol and progesterone are provided. In some embodiments, the method comprises administering to the subject a pharmaceutical composition comprising about 2 mg estradiol and about 200 mg progesterone as described herein (e.g., a pharmaceutical composition having the formulation of Formulation E in Table 5 above), wherein administration of the pharmaceutical composition produces, in a plasma sample from the subject, one or more parameters selected from: an AUC₍₀₋ₜ₎ for estradiol that is from 1123 pg·h/ml to 1755 pg·h/ml; a Cₘₐₓ for estradiol that is from 52 pg/ml to 81 pg/ml; a Tₘₐₓ for estradiol that is from 7.2 hr to 11.3 hr; an AUC₍₀₋ₜ₎ for progesterone that is from 96 ng·hr/ml to 150 ng·hr/ml; a Cₘₐₓ for progesterone that is from 71 ng/ml to 112 ng/ml; a Tₘₐₓ for progesterone that is from 2.4 hr to 3.8 hr; an AUC₍₀₋ₜ₎ for estrone that is from 7277 pg·hr/ml to 11370 pg·hr/ml; a Cₘₐₓ for estrone that is from 341 pg/ml to 533 pg/ml; a Tₘₐₓ for estrone that is from 4.4 hr to 6.9 hr; an AUC₍₀₋ₜ₎ for total estrone that is from 161 ng-h/ml to 252 ng·h/ml; a Cₘₐₓ for total estrone that is from 28 ng/ml to 44 ng/ml; and a Tₘₐₓ for total estrone that is from 2 hr to 3.1 hr.

In some embodiments, the method further comprises obtaining a sample from the subject (e.g., a blood or plasma sample) following administration of a single dose of the pharmaceutical composition (e.g., a pharmaceutical composition having the formulation of Formulation E in Table 5 above), and measuring one or more pharmacokinetic parameters selected from an AUC₍₀₋ₜ₎ for estradiol, a Cₘₐₓ for estradiol, an AUC₍₀₋ₜ₎ for progesterone, a Cₘₐₓ for progesterone, an AUC₍₀₋ₜ₎ for estrone, a Cₘₐₓ for estrone, an AUC₍₀₋ₜ₎ for total estrone, and a Cₘₐₓ for total estrone; wherein the presence of one or more of the following values is indicative of a therapeutically effective dose: an AUC₍₀₋ₜ₎ for estradiol that is from 1123 pg·h/ml to 1755 pg·h/ml; a Cₘₐₓ for estradiol that is from 52 pg/ml to 81 pg/ml; an AUC₍₀₋ₜ₎ for progesterone that is from 96 ng·hr/ml to 150 ng·hr/ml; a Cₘₐₓ for progesterone that is from 71 ng/ml to 112 ng/ml; an AUC₍₀₋ₜ₎ for estrone that is from 7277 pg·hr/ml to 11370 pg·hr/ml; a Cₘₐₓ for estrone that is from 341 pg/ml to 533 pg/ml; an AUC₍₀₋ₜ₎ for total estrone that is from 161 ng·h/ml to 252 ng·h/ml; and a Cₘₐₓ for total estrone that is from 28 ng/ml to 44 ng/ml. In some embodiments, the one or more pharmacokinetic parameters are measured about 18 hours, about 24 hours, about 18-36 hours, about 20-30 hours, about 22-26 hours, about 24-36 hours, about 36 hours, about 36-48 hours, about 40-48 hours, or about 48 hours after administration of the single dose.

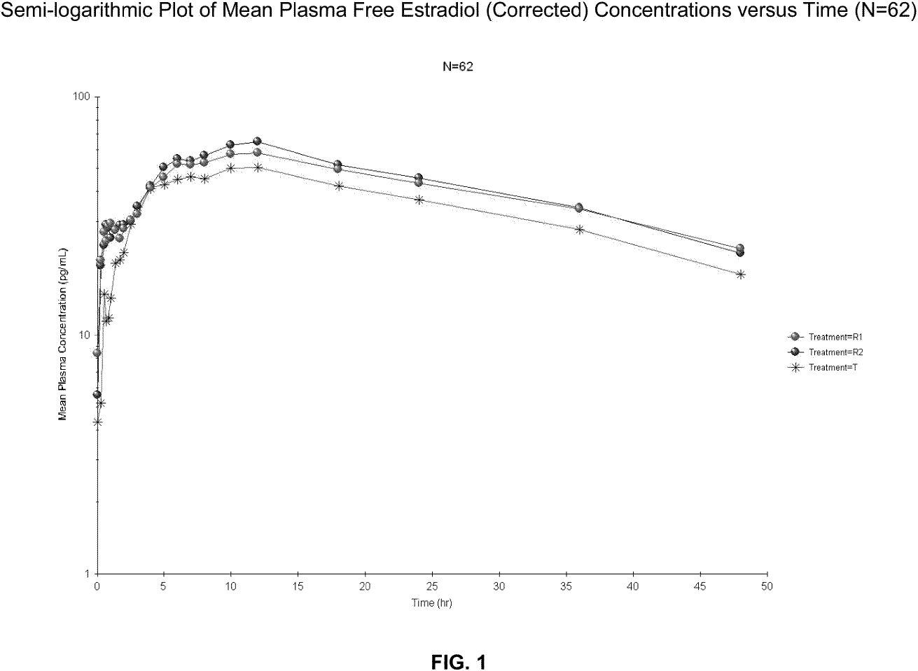

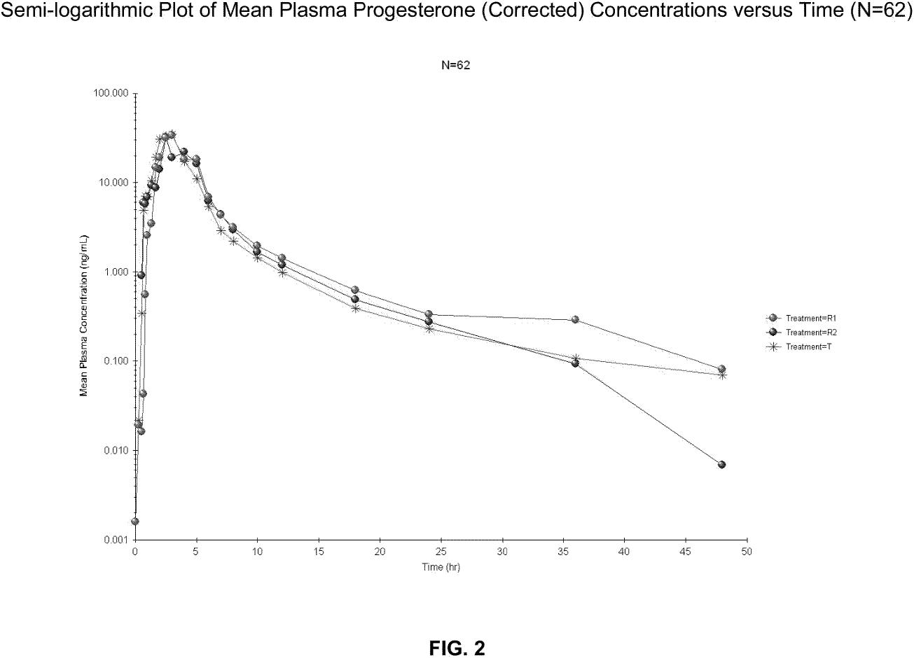

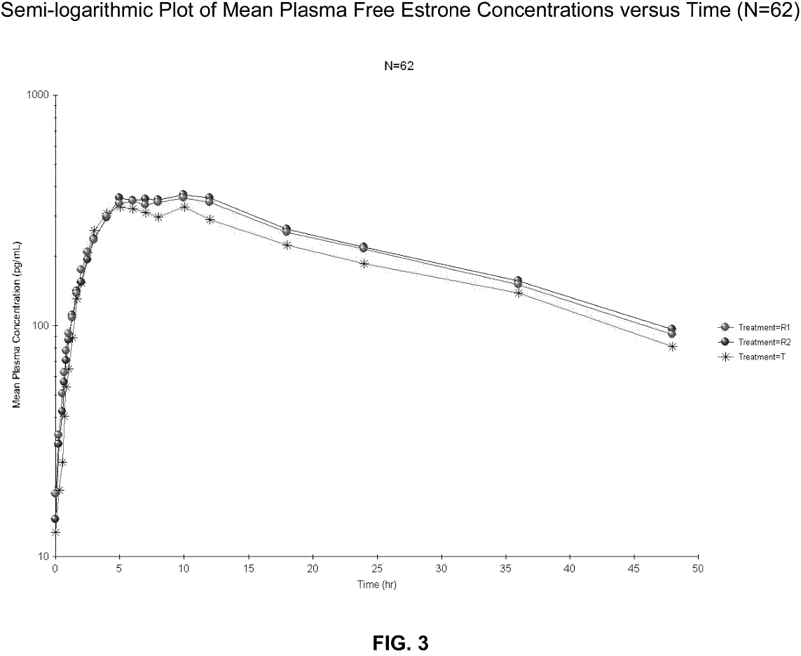

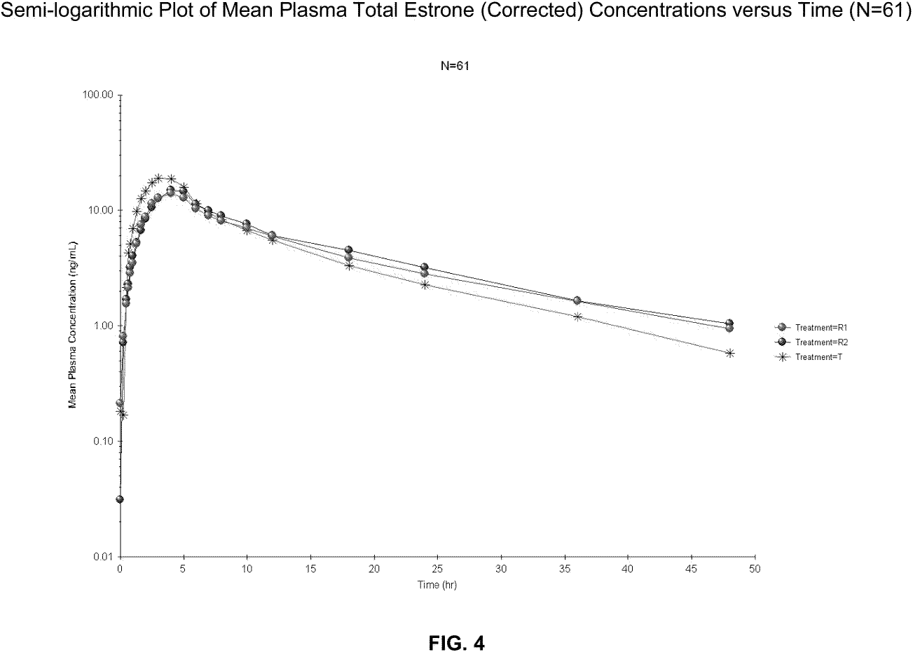

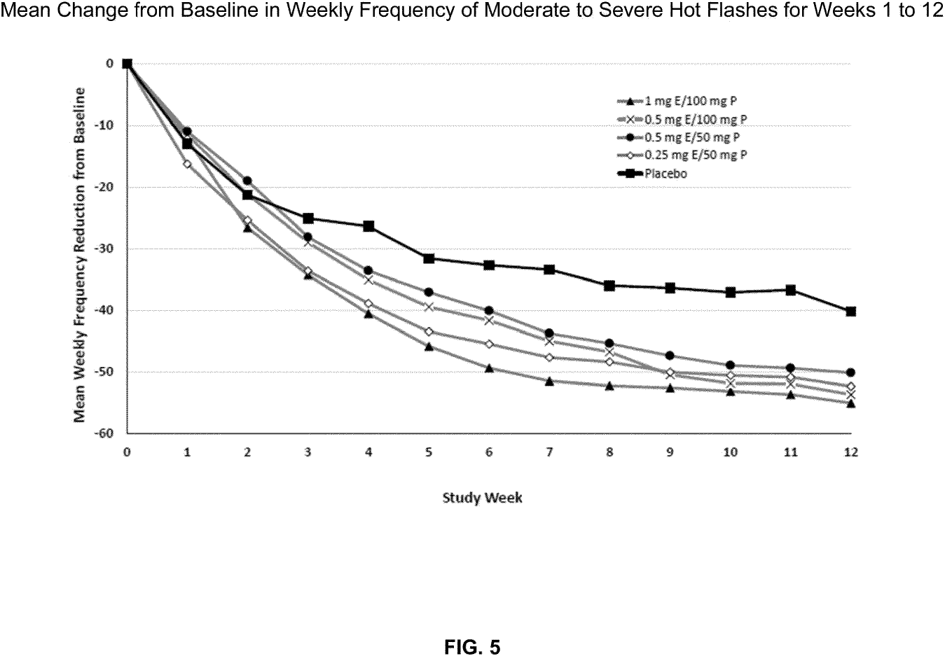

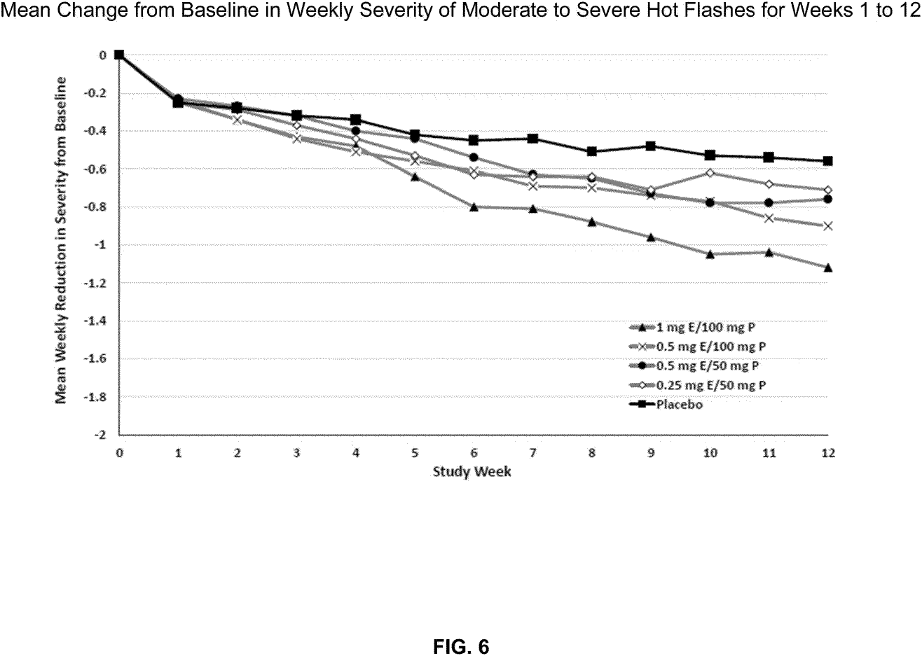

In some embodiments, administration of the pharmaceutical composition as described herein results in the blood plasma estradiol concentration profile of FIG. 1. In some embodiments, administration of the pharmaceutical composition results in the blood plasma progesterone concentration profile of FIG. 2. In some embodiments, administration of the pharmaceutical composition results in the blood plasma estrone concentration profile of FIG. 3. In some embodiments, administration of the pharmaceutical composition results in the blood plasma total estrone concentration profile of FIG. 4. In some embodiments, administration of the pharmaceutical compositions as dscribed herein provide mean change from baseline in weekly frequency of moderate to severe hot flashes/flushes for weeks 1 to 12 as shown in FIG. 5. In some embodiments, administration of the pharmaceutical compositions as dscribed herein provide mean change from baseline in weekly severity of moderate to severe hot flashes/flushes for weeks 1 to 12 as shown in FIG. 6.

### Administration and Treatment

Pharmaceutical compositions comprising estradiol and progesterone as described herein (e.g., compositions comprising solubilized estradiol, suspended progesterone, and a medium chain solubilizing agent) can be prepared and administered in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. Pharmaceutical compositions can be formulated for any appropriate manner of administration, including, for example, topical, oral, nasal, intrathecal, rectal, vaginal, sublingual or parenteral administration, including subcutaneous, intravenous, intramuscular, intrasternal, intracavernous, intrameatal, or intraurethral injection or infusion. In some embodiments, administration is by injection, that is, intravenously, intramuscularly, intracutaneously, subcutaneously, intraduodenally, or intraperitoneally.

For preparing pharmaceutical compositions from the compounds of this disclosure, the pharmaceutically acceptable compositions can be either solid or liquid. Solid form preparations include powders, tablets, pills, capsules, cachets, suppositories, and dispersible granules. A solid preparation can comprise one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington’s Pharmaceutical Sciences, Mack Publishing Co, Easton PA (“Remington’s”).

In general, the type of composition is selected based on the mode of administration. A pharmaceutical composition (e.g., for oral administration or delivery by injection) can be in the form of a liquid (e.g., an elixir, syrup, solution, emulsion or suspension). Alternatively, a pharmaceutical composition as described herein can take the form of a pill, tablet, or capsule containing the liquid oil, and thus, the composition can contain any of the following: a diluent such as lactose, sucrose, dicalcium phosphate, and the like; a disintegrant such as starch or derivatives thereof; a lubricant such as magnesium stearate and the like; and a binder such a starch, gum acacia, polyvinylpyrrolidone, gelatin, cellulose and derivatives thereof. The composition can also be formulated into a suppository disposed, for example, in a polyethylene glycol (PEG) solubilizing agent.

Administration of the compositions of this disclosure can be carried out via any of the accepted modes of administration. Thus, administration can be, for example, intravenous, topical, subcutaneous, transcutaneous, transdermal, intramuscular, oral, intra-joint, parenteral, intra-arteriole, intradermal, intraventricular, intracranial, intraperitoneal, intralesional, intranasal, rectal, vaginal, or by inhalation. In some embodiments, a composition as described herein is administered orally. For example, a pharmaceutical composition as described herein can be administered via capsules such as soft capsules.

In some embodiments, a pharmaceutical composition as described herein is administered once daily for a period of 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 days or more. In some embodiments, a pharmaceutical composition as described herein is administered daily for at least one week, at least two weeks, at least three weeks, at least four weeks, at least one month, at least two months, at least three months, at least four months, at least five months, at least six months, at least seven months, at least eight months, at least nine months, at least ten months, at least eleven months, at least twelve months, or more. In some embodiments, a pharmaceutical composition as described herein is administered as a continuous-combined therapy regimen.

In some embodiments, a 28-day or monthly regimen of daily doses is packaged in a single kit (e.g., a blister pack) having administration days identified to improve compliance and reduce associated symptoms, among others. In some embodiments, each daily dose contains both estradiol and progesterone. In some embodiments, one or more of the daily doses contains no estradiol or no progesterone. Daily doses that comprise no estradiol or progesterone API may be referred to as placebos. A blister pack can have a plurality of scores or perforations separating the blister pack into 28 days. Each day may further comprise a single blister or a plurality of blisters. In various embodiments, each unit dose may contain micronized or partially solubilized, or fully solubilized progesterone or solubilized estradiol in amounts as set forth herein, although other dose ranges may be contemplated. In addition, kits having other configurations are also contemplated herein. For example, without limitation, kits having such blister packs may contain any number of daily doses.

In some embodiments, the pharmaceutical compositions disclosed herein are useful in treating conditions in subjects caused, at least in part, by estrogen deficiency, particularly for women with a uterus. For example, in embodiments, the pharmaceutical compositions disclosed herein are useful for the treatment of one or more of the following conditions: endometrial hyperplasia; secondary amenorrhea; prevention of preterm birth, when the subject has a shortened cervix; menopause-related symptoms including, for example, vasomotor symptoms; in relation to treatment of hypoestrogenism related symptoms including, for example and without limitation, hot flashes and night sweats (vasomotor symptoms), sleep disturbances, mood changes and vulvo-vaginal atrophy; and osteoporosis and other non-menopausal disease states or conditions treated with supplemental progesterone or estrogen. In some embodiments, the pharmaceutical compositions disclosed herein are useful in treating vasomotor symptoms, including but not limited to, hot flashes and night sweats. In some embodiments, the pharmaceutical compositions disclosed herein are useful in treating hot flashes and night sweats. In some embodiments, the pharmaceutical compositions disclosed herein are useful in treating hot flashes. Thus, in some embodiments, this disclosure provides methods of treating such a condition by administering to the subject a composition comprising estradiol and progesterone as described herein.

### III. Examples

The following examples are offered to illustrate, but not to limit, the claimed subject matter.

### Example 1

In an exemplary embodiment, a soft gelatin capsule contains a pharmaceutical composition comprising suspended progesterone and solubilized estradiol:

TABLE 6

| Ingredient | Mass (mg) | % w/w | Qty/Capsule (mg) |
|---|---|---|---|
| Progesterone, USP, micronized | 50.00 | 7.14 | 50.00 |
| Estradiol Hemihydrate, USP | 2.03 | 0.29 | 2.03 |
| CAPMUL® MCM, NF | | 82.57 | 577.97 |
| GELUCIRE® 44/14, NF | | 10.0 | 70.00 |
| TOTAL | | 100.00 | 700.00 |

The encapsulated pharmaceutical composition of Table 6 may be manufactured in any suitable manner. For the purposes of this Example, mixing may be facilitated by an impellor, agitator, or other suitable means. Also for the purposes of this Example, heating or mixing may be performed under an inert or relatively inert gas atmosphere, such as nitrogen gas (N₂). Mixing or heating for the purposes of this Example may be performed in any suitable vessel, such as a stainless steel vessel.

For example, CAPMUL® MCM may be heated to between 30° C. to 50° C., more preferably from 35° C. to 45° C., and more preferably to 40° C. ± 2° C. GELUCIRE® 44/14 may be added to the CAPMUL® MCM and mixed until dissolved (to increase the solubility of progesterone in the final solution, GELUCIRE® 44/14 was added at about 10% w/w). The addition may occur all at once or may occur gradually over a period of time. Heat may continue to be applied during the mixing of the GELUCIRE® 44/14 and the CAPMUL® MCM.

Heat may be removed from the GELUCIRE® 44/14 and CAPMUL® MCM mixture. Estradiol Hemihydrate may be added to the mixture. The addition may occur all at once or may occur gradually over a period of time. Micronized progesterone may then be added to the GELUCIRE® 44/14, CAPMUL® MCM and Estradiol Hemihydrate mixture until dissolved. The addition may occur all at once or may occur gradually over a period of time.

### Example 2

An example of the final scale-up formulation is provided in Table 7. To manufacture, CAPMUL® MCM is heated to 40° C. GELUCIRE® 44/14 is heated to 65° C. and added and mixed until dissolved. Heat is removed. Estradiol is added and mixed until dissolved. Micronized progesterone is then added and mixed until fully suspended.

TABLE 7

Quantitative Formula: Batch Size 10,000 capsules

| Item No. | Ingredient | Label Claim (mg) | % w/w | Qty/Capsule (mg) | Amount/Batch (kg) |
|---|---|---|---|---|---|
| 1. | Progesterone, USP, micronized | 50.00 | 7.14 | 50.00 | 0.50 |
| 2. | Estradiol Hemihydrate, USP | 2.03 | 0.29 | 2.03 | 0.02 |
| 3. | CAPMUL® MCM, NF | | 82.57 | 577.97 | 5.78 |
| 4. | GELUCIRE® 44/14, NF | | 10.0 | 70.00 | 0.70 |
| | | Total: | 100.00 | 700.00 | 7.00 |

### Example 3

In an exemplary embodiment, a soft gelatin capsule contains a pharmaceutical composition having fully solubilized estradiol and partially solubilized progesterone comprising:

TABLE 8

| Item No. | Ingredient | Label Claim (mg) | % w/w | Qty/Capsule (mg) | Amount/Batch (g) |
|---|---|---|---|---|---|
| 1. | Progesterone, USP, micronized | 50.00 | 25.000 | 50.00 | 500.00 |
| 2. | Estradiol Hemihydrate | 0.25 | 0.129 | 0.26 | 2.58 |
| 3. | CAPMUL® MCM, NF | | 73.371 | 146.74 | 1467.42 |
| 4. | GELUCIRE® 44/14, NF | | 1.500 | 3.00 | 30.00 |
| | | Total: | 100.000 | 200.00 mg | 2000.00 |

To manufacture, CAPMUL® MCM is heated to 65° C. GELUCIRE® 44/14 is added and mixed until dissolved. Heat is removed. Estradiol is added and mixed until dissolved. Micronized progesterone is then added and dispersed. The mixture is then passed through a colloid mill. The resultant fill mass can be used for encapsulation.

### Example 4

In an exemplary embodiment, a soft gelatin capsule contains a pharmaceutical composition having fully solubilized estradiol and partially solubilized progesterone comprising:

TABLE 9

| Item No. | Ingredient | Label Claim (mg) | % w/w | Qty/Capsule (mg) | Amount/Batch (g) |
|---|---|---|---|---|---|
| 1. | Progesterone, USP, micronized | 200.00 | 33.33 | 200.0 | 2000.0 |
| 2. | Estradiol Hemihydrate | 2.00 | 0.35 | 2.07 | 20.7 |
| 3. | CAPMUL® MCM, NF | | 65.32 | 391.93 | 3919.3 |
| 4. | GELUCIRE® 44/14, NF | | 1.00 | 6.0 | 60.0 |
| | | Total: | 100.00 | 600.0 mg | 6000.0 |

To manufacture, CAPMUL® MCM is heated to 65° C. GELUCIRE® 44/14 is added and mixed until dissolved. Heat is removed. Estradiol is added and mixed until dissolved. Micronized progesterone is then added and dispersed. The mixture is then passed through a colloid mill. The resulting pharmaceutical composition is encapsulated in soft gelatin capsules. Alternatively, GELUCIRE® 44/14 is heated to 65° C. and CAPMUL® MCM is heated to 40° C. ± 5° C. to achieve mixing of the oil and the surfactant before heat is removed; estradiol is added while the mixture is cooling; progesterone is added when the mixture has dropped below about 40° C.; the mixture is then passed through a colloid mill one or more times, e.g., three times.

### Example 5

Pharmacokinetics of the First Combination 17β-Estradiol/Progesterone Capsule in Clinical Development for Hormone Therapy

The objective of this study was to evaluate the pharmacokinetic and oral bioavailability of a combination capsule of 17β-estradiol/progesterone in comparison to co-administration of the individual products ESTRACE® and PROMETRIUM®.

Subjects and Study Design: An open label, balanced, randomized, single-dose, 2-treatment, 3-period, 3-sequence, crossover, partial-replicate, reference-scaled, oral, relative bioavailability study compared the bioavailability of an investigational 2-mg 17β-estradiol/200-mg progesterone combination capsule, without peanut oil (formulated in a manner similar to that set forth in Table 9), with that of co-administered 200-mg PROMETRIUM® (progesterone) and 2-mg ESTRACE® (17β-estradiol) tablets in healthy postmenopausal women aged 40-65 years (N=66). Key inclusion criteria for subjects included a BMI 18.50 to 29.99 kg/m² who were nonsmokers or ex-smokers (no smoking in the last 3 months). Key exclusion criteria for subjects included consuming grapefruit juice or poppy-containing foods within 48 hours before and throughout the study, use of any hormonal agent within 14 days before the study, and use of menopausal hormone therapy within 6 months before dosing.

Patients were randomly assigned sequentially to 1 of 3 dosing sequences of the same dose of the combination capsule (Test, T) and reference products (Reference, R): TRR, RTR, or RRT. 66 subjects were randomized and 62 (94.0%) completed the study. Subjects had a mean age of 49.5 ± 5.6 years (range 40 to 64) and a mean BMI of 24.8 ± 3.1 kg/m² (range 18.7-29.9).

After consuming a high-fat, high-calorie breakfast, each woman received a single dose of the combination (Test) capsule in 1 period of the study and single doses of the co-administered products (Reference) in each of the 2 remaining periods. Blood samples were collected within 75 minutes before dosing and post-dose at 0.25, 0.5, 0.67, 0.83, 1, 1.33, 1.67, 2, 2.5, 3, 4, 5, 6, 7, 8, 10, 12, 18, 24, 36, and 48 hours after dosing to determine progesterone, free (unconjugated) estradiol, and free and total (conjugated+free, including estrone sulfates) estrone concentrations. After collection of blood samples at each time point, the blood samples were centrifuged at 4000 RPM for 10 minutes at 4° C. to separate the plasma. The plasma from samples was separated into two aliquots. 1.5 mL from the plasma sample was transferred into aliquot I, and the remaining plasma sample was transferred into aliquot II. These aliquots were stored at -30° C. for interim storage, then at -70° C. until completion of the analysis.

Progesterone, estradiol, estrone, and total estrone in human plasma was determined using the LC-MS/MS method. The primary (Cₘₐₓ, AUC₀₋ₜ, and AUC_{0-∞}) and secondary (Tₘₐₓ, t_{½}, and Kₑ) PK parameters for each analyte were determined for each subject during each period by non-compartment analyses using baseline-adjusted concentrations. Statistical analyses were conducted using the SAS® statistical software.

Results: The mean, standard deviation (SD), geometric mean, coefficient of variation (CV %), minimum, median, and maximum were calculated for Cₘₐₓ, AUC₀₋ₜ, AUC_{0-∞,}, Tₘₐₓ, t_{½}, Kₑ₁, K_{e1_lower}, Kₑ₁_{_}_{Upper}, and AUC_{%Extrap_obs} for progesterone, estradiol, estrone, and total estrone. The results are presented in Tables 10, 11, 12, and 13 below. For each of Tables 10-13, “Test Product (T)” refers to the progesterone + estradiol pharmaceutical composition, while “Reference product (R1)” and “Reference product (R2)” refers to co-administered PROMETRIUM® (progesterone) and ESTRACE® (estradiol). Blood plasma concentrations of progesterone, estradiol, estrone, and total estrone over time are also shown in FIGS. 1-4.

TABLE 10

Summary of Pharmacokinetic Parameters of Test Product (T) versus Reference Product (R₁, R₂) for Progesterone
Untransformed Data (Mean ± SD)

| PK Parameter | N | Test Product (T) | N | Reference product (R1) | N | Reference product (R2) |
|---|---|---|---|---|---|---|
| Cₘₐₓ (ng/mL) | 62 | 89.2222 ± 149.7309 | 62 | 72.7228 ± 101.8885 | 62 | 69.7590 ± 87.0777 |
| AUC₀₋ₜ (ng.hr/mL) | 62 | 120.0869 ± 164.1385 | 62 | 125.9406 ± 152.3483 | 62 | 111.5867 ± 113.3200 |
| AUC_{0-∞} (ng.hr/mL) | 57 | 131.3817 ± 172.4806 | 57 | 142.1332 ± 160.4853 | 56 | 126.6006 ± 117.2665 |
| Tₘₐₓ (hr) | 62 | 3.00(0.83-10.00) | 62 | 3.00(1.00-12.00) | 62 | 4.00(0.67-18.00) |
| Kₑₗ(hr⁻¹) | 57 | 0.3064 ± 0.2427 | 57 | 0.2684 ± 0.1912 | 56 | 0.2795 ± 0.2475 |
| t_{½} (hr) | 57 | 4.6445 ± 4.5366 | 57 | 5.1555 ± 4.9794 | 56 | 5.0389 ± 4.5887 |
| K_{el_Lower}(hr⁻¹) | 57 | 7.6667 ± 4.6047 | 57 | 7.4123 ± 4.2164 | 56 | 7.9018 ± 3.9120 |
| K_{el_Upper} (hr⁻¹) | 57 | 16.2218 ± 11.0051 | 57 | 19.1728 ± 12.3801 | 56 | 18.1975 ± 10.0858 |
| AUC_Extra (%) | 57 | 4.3374 ± 2.5528 | 57 | 4.8416 ± 3.7526 | 56 | 5.1868 ± 4.1434 |

*Expressed in terms of median (range)

TABLE 11

Summary of Pharmacokinetic Parameters of Test Product (T) versus Reference Product (R₁, R₂) for Estradiol

| PK Parameter | Untransformed Data (Mean ± SD) | | |
|---|---|---|---|
| | Test Product (T) | Reference product (R1) | Reference product (R2) |
| Cmax (pg/mL) | 64.7902 ± 50.9833 | 69.1286 ± 33.0484 | 73.4236 ± 43.4077 |
| AUC₀₋ₜ (pg.hr/mL) | 1403.7333 ± 763.8136 | 1508.2206 ± 876.7390 | 1658.2502 ± 976.5556 |
| AUC_{0-∞}(pg.hr/mL) | 2459.4394 ± 4498.2737 | 2842.8805 ± 4582.6502 | 2110.9591 ± 1175.3995 |
| Tₘₐₓ (hr) | 9.00(0.50-36.00) | 10.0(0.50-35.12) | 10.00(0.25-36.60) |
| Kₑₗ (hr⁻¹) | 0.0438 ± 0.0197 | 0.0457 ± 0.0358 | 0.0464 ± 0.0338 |
| t_{½} (hr) | 31.9104 ± 95.9769 | 25.0908 ± 28.8346 | 20.8774 ± 12.0825 |
| K_{el_Lower}(hr⁻¹) | 14.9472 ± 7.2715 | 14.9667 ± 7.0150 | 14.7953 ± 5.8774 |
| K_{el_Upper} (hr⁻¹) | 45.3602 ± 6.3668 | 44.3277 ± 7.4003 | 43.8330 ± 7.6449 |
| AUC_Extra (%) | 22.8106 ± 16.6498 | 25.4773 ± 20.2911 | 24.9566 ± 16.4713 |

*Expressed in terms of median (range)

TABLE 12

Summary of Pharmacokinetic Parameters of Test Product (T) versus Reference Product (R₁, R₂) for Free Estrone

| PK Parameter | Untransformed Data (Mean ± SD) | | |
|---|---|---|---|
| | Test Product (T) | Reference product (R1) | Reference product (R2) |
| Cₘₐₓ (pg/mL) | 426.5492 ± 179.3303 | 455.5107 ± 189.448 | 467.2302 ± 207.4373 |
| AUC₀₋ₜ (pg.hr/mL) | 9096.0907 ± 4377.2730 | 10156.0282 ± 5140.5831 | 10507.3557 ± 5183.1289 |
| AUC_{0-∞}(pg.hr/mL) | 11994.9695 ± 6678.5468 | 13445.9048 ± 8699.4068 | 14066.2362 ± 7563.2370 |
| Tₘₐₓ (hr) | 5.50(0.83-36.00) | 8.00(1.67- 18.00) | 10.00(1.67-18.00) |
| Kₑₗ (hr⁻¹) | 0.0399 ± 0.0146 | 0.0424 ± 0.0172 | 0.0406 ± 0.0209 |
| t_{½} (hr) | 20.3172 ± 9.4052 | 19.4595 ±9.8711 | 20.7515 ± 9.3985 |
| K_{el_ Lower}(hr⁻¹) | 13.8443 ± 7.0649 | 14.8871 ± 6.6459 | 14.9194 ± 6.4485 |
| K_{el_Upper} (hr⁻¹) | 46.0238 ± 5.5080 | 46.2547 ± 5.3060 | 46.2244 ± 5.3126 |
| AUC_Extra (%) | 21.2980 ± 11.2283 | 20.3648 ± 11.1060 | 21.8900 ± 11.8537 |

*Expressed in terms of median (range)

TABLE 13

Summary of Pharmacokinetic Parameters of Test Product (T) versus Reference Product (Ri, R₂) for Total Estrone
Untransformed Data (Mean ± SD)

| PK Parameter | N | Test Product (T) | N | Reference product (R1) | N | Reference product (R2) |
|---|---|---|---|---|---|---|
| Cₘₐₓ (ng/mL) | 61 | 35.4289 ± 17.0856 | 61 | 19.8716 ± 7.4485 | 61 | 19.9048 ± 8.0288 |
| AUC₀₋ₜ(ng.hr/mL) | 61 | 201.7524 ± 94.2081 | 61 | 182.7729 ± 88.8386 | 61 | 199.8295 ± 94.9392 |
| AUC_{0-∞} (ng.hr/mL) | 61 | 213.2402 ± 104.6011 | 60 | 193.6387 ± 100.5831 | 56 | 203.0289 ± 81.4884 |
| Tₘₐₓ (hr) | 61 | 2.50(0.67-7.00) | 61 | 4.00(1.33-18.00) | 61 | 4.00(1.33-10.00) |
| Kₑₗ(hr⁻¹) | 61 | 0.0799 ± 0.0398 | 60 | 0.0803 ± 0.0399 | 56 | 0.0718 ± 0.0243 |
| t_{½} (hr) | 61 | 10.3619 ± 4.0023 | 60 | 9.8448 ± 3.0702 | 56 | 10.7830 ± 3.6624 |
| K_{el_Lower}(hr⁻¹) | 61 | 13.0492 ± 6.8585 | 60 | 13.5945 ± 8.0129 | 56 | 11.8870 ± 6.8696 |
| K_{el_Upper}(hr⁻¹) | 61 | 45.3979 ± 6.6589 | 60 | 46.3775 ± 5.2525 | 56 | 46.7054 ± 4.3888 |
| AUC_Extra (%) | 61 | 4.5030 ± 3.7366 | 60 | 4.5913 ± 3.4953 | 56 | 5.3450 ± 3.9831 |

*Expressed in terms of median (range)

### Example 6

Pharmacokinetic data (Cₘₐₓ, AUC₍₀₋ₜ₎, AUC_{(0-∞}), and Tₘₐₓ) for progesterone, estradiol, free estrone, and total estrone is presented in Tables 14-17. Pharmaceutical compositions A-E are disclosed in Tables 1-5. The pK values for pharmaceutical composition E were calculated as disclosed in Example 5. For pharmaceutical compositions A-D, expected pharmacokinetic data is calculated from the data disclosed for pharmaceutical composition E.

TABLE 14

Summary of Pharmacokinetic Parameters of the Pharmaceutical Compositions of Tables 1-5 for Progesterone

| Pharmaceutical Composition | Progesterone Content | Estradiol Content | Cmax (ng/mL) | AUC₍₀₋ₜ₎ (ng.hr/mL) | AUC_{(0-∞)} (ng.hr/mL) | Tmax (hr) |
|---|---|---|---|---|---|---|
| A | 50 mg | 0.25 mg | 22.30555 | 30.0217 | 32.8454 | 3.00 |
| B | 50 mg | 0.50 mg | 22.3055 | 30.0217 | 32.8454 | 3.00 |
| C | 100 mg | 0.50 mg | 44.6111 | 60.0435 | 65.6909 | 3.00 |
| D | 100 mg | 1 mg | 44.6111 | 60.0435 | 65.6909 | 3.00 |
| E | 200 mg | 2 mg | 89.2222 | 120.0869 | 131.3817 | 3.00 |

TABLE 15

Summary of Pharmacokinetic Parameters of the Pharmaceutical Compositions of Tables 1-5 for Estradiol

| Pharmaceutical Composition | Progesterone Content | Estradiol Content | Cₘₐₓ (pg/mL) | AUC₍₀₋ₜ₎ (pg.hr/mL) | AUC_{(0-∞)} (pg.hr/mL) | Tₘₐₓ (hr) |
|---|---|---|---|---|---|---|
| A | 50 mg | 0.25 mg | 8.0988 | 175.4667 | 307.4299 | 9.00 |
| B | 50 mg | 0.50 mg | 16.1976 | 350.9333 | 614.8599 | 9.00 |
| C | 100 mg | 0.50 mg | 16.1976 | 350.9333 | 614.8599 | 9.00 |
| D | 100 mg | 1 mg | 32.3951 | 701.8667 | 1229.7197 | 9.00 |
| E | 200 mg | 2 mg | 64.7902 | 1403.7333 | 2459.4394 | 9.00 |

TABLE 16

Summary of Pharmacokinetic Parameters of the Pharmaceutical Compositions of Tables 1-5 for Free Estrone

| Pharmaceutical Composition | Progesterone Content | Estradiol Content | Cₘₐₓ (pg/mL) | AUC₍₀₋ₜ₎ (pg.hr/mL) | AUC_{(0-∞)} (pg.hr/mL) | Tₘₐₓ (hr) |
|---|---|---|---|---|---|---|
| A | 50 mg | 0.25 mg | 53.3187 | 1137.0113 | 1499.3712 | 5.50 |
| B | 50 mg | 0.50 mg | 106.6373 | 2274.0227 | 2998.7424 | 5.50 |
| C | 100 mg | 0.50 mg | 106.6373 | 2274.0227 | 2998.7424 | 5.50 |
| D | 100 mg | 1 mg | 213.2746 | 4548.0454 | 5997.4848 | 5.50 |
| E | 200 mg | 2 mg | 426.5492 | 9096.0907 | 11994.9695 | 5.50 |

TABLE 17

Summary of Pharmacokinetic Parameters of the Pharmaceutical Compositions of Tables 1-5 for Total Estrone

| Pharmaceutical Composition | Progesterone Content | Estradiol Content | Cₘₐₓ (ng/mL) | AUC₍₀₋ₜ₎ (ng.hr/mL) | AUC_{(0-∞)} (ng.hr/mL) | Tₘₐₓ (hr) |
|---|---|---|---|---|---|---|
| A | 50 mg | 0.25 mg | 4.4286 | 25.2191 | 26.6550 | 2.50 |
| B | 50 mg | 0.50 mg | 8.8572 | 50.4381 | 53.3101 | 2.50 |
| C | 100 mg | 0.50 mg | 8.8572 | 50.4381 | 53.3101 | 2.50 |
| D | 100 mg | 1 mg | 17.7145 | 100.8762 | 106.6201 | 2.50 |
| E | 200 mg | 2 mg | 35.4289 | 201.7524 | 213.2402 | 2.50 |

The ranges of expected pK values for each of the pharmaceutical compositions of Tables 1-4 are disclosed in Tables 18-21, respectively.

TABLE 18

pK Ranges for the Pharmaceutical Composition of Table 1 (Pharmaceutical Composition A)

| | | | |
|---|---|---|---|
| | Cₘₐₓ | AUC₍₀₋ₜ₎ | AUC_{(0-∞)} |
| Progesterone | 17.8444 ng/mL to 27.8819 ng/mL | 24.0174 ng.hr/mL to 37.5272 ng.hr/mL | 26.2763 ng.hr/mL to 41.0568 ng.hr/mL |
| Estradiol | 6.4790 pg/mL to 10.1235 pg/mL | 140.3733 pg.hr/mL to 219.3333 pg.hr/mL | 245.9439 pg.hr/mL to 384.2874 pg.hr/mL |
| Free estrone | 42.6549 pg/mL to 66.6483 pg/mL | 909.6091 pg.hr/mL to 1421.2642 pg.hr/mL | 1199.4970 pg.hr/mL to 1874.2140 pg.hr/mL |
| Total estrone | 3.5429 ng/mL to 5.5358 ng/mL | 20.1752 ng.hr/mL to 31.5238 ng.hr/mL | 21.3240 ng.hr/mL to 33.3188 ng.hr/mL |

TABLE 19

pK Ranges for the Pharmaceutical Composition of Table 2 (Pharmaceutical Composition B)

| | | | |
|---|---|---|---|
| | Cₘₐₓ | AUC₍₀₋ₜ₎ | AUC_{(0-∞)} |
| Progesterone | 17.8444 ng/mL to 27.8819 ng/mL | 24.0174 ng.hr/mL to 37.5272 ng.hr/mL | 26.2763 ng.hr/mL to 41.0568 ng.hr/mL |
| Estradiol | 12.9580 pg/mL to 20.2469 pg/mL | 280.7467 pg.hr/mL to 438.6667 pg.hr/mL | 491.8879 pg.hr/mL to 768.5748 pg.hr/mL |
| Free estrone | 85.3098 pg/mL to 133.2966 pg/mL | 1819.2181 pg.hr/mL to 2842.5283 pg.hr/mL | 2398.9939 pg.hr/mL to 3748.4280 pg.hr/mL |
| Total estrone | 7.0858 ng/mL to 11.0715 ng/mL | 40.3505 ng.hr/mL to 63.0476 ng.hr/mL | 42.6480 ng.hr/mL to 66.6376 ng.hr/mL |

TABLE 20

pK Ranges for the Pharmaceutical Composition of Table 3 (Pharmaceutical Composition C)

| | | | |
|---|---|---|---|
| | Cₘₐₓ | AUC₍₀₋ₜ₎ | AUC_{(0-∞)} |
| Progesterone | 35.6889 ng/mL to 55.7639 ng/mL | 48.0348 ng.hr/mL to 75.0543 ng.hr/mL | 52.5527 ng.hr/mL to 82.1136 ng.hr/mL |
| Estradiol | 12.9580 pg/mL to 20.2469 pg/mL | 280.7467 pg.hr/mL to 438.6667 pg.hr/mL | 491.8879 pg.hr/mL to 768.5748 pg.hr/mL |
| Free estrone | 85.3098 pg/mL to 133.2966 pg/mL | 1819.2181 pg.hr/mL to 2842.5283 pg.hr/mL | 2398.9939 pg.hr/mL to 3748.4280 pg.hr/mL |
| Total estrone | 7.0858 ng/mL to 11.0715 ng/mL | 40.3505 ng.hr/mL to 63.0476 ng.hr/mL | 42.6480 ng.hr/mL to 66.6376 ng.hr/mL |

TABLE 21

PK Ranges for the Pharmaceutical Composition of Table 4 (Pharmaceutical Composition D)

| | | | |
|---|---|---|---|
| | Cₘₐₓ | AUC₍₀₋ₜ₎ | AUC_{(0-∞)} |
| Progesterone | 35.6889 ng/mL to 55.7639 ng/mL | 48.0348 ng.hr/mL to 75.0543 ng.hr/mL | 52.5527 ng.hr/mL to 82.1136 ng.hr/mL |
| Estradiol | 25.9161 pg/mL to 40.4939 pg/mL | 561.4933 pg.hr/mL to 877.3333 pg.hr/mL | 983.7758 pg.hr/mL to 1537.1496 pg.hr/mL |
| Free estrone | 170.6197 pg/mL to 266.5933 pg/mL | 3638.4363 pg.hr/mL to 5685.0567 pg.hr/mL | 4797.9878 pg.hr/mL to 7496.8559 pg.hr/mL |
| Total estrone | 14.1716 ng/mL to 22.1431 ng/mL | 80.7010 ng.hr/mL to 126.0953 ng.hr/mL | 85.2961 ng.hr/mL to 133.2751 ng.hr/mL |

As such, in some embodiments, the pharmaceutical composition comprises about 0.25 mg estradiol and about 50 mg progesterone, and administration of the composition to the subject produces, in a plasma sample from the subject, one or more parameters selected from:
(i) an area under the curve (AUC)₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or both parameters selected from: an AUC₍₀₋ₜ₎ for estrone that is from 909.6091 pg·hr/ml to 1421.2642 pg·hr/ml; and a Cₘₐₓ for estrone that is from 42.6549 pg/ml to 66.6483 pg/ml.

In some embodiments, administration of the composition to subject further produces, in a plasma sample from the subject, one or both parameters selected from: an AUC₍₀₋ₜ₎ for total estrone that is from 20.1752 ng·hr/ml to 31.5238 ng·hr/ml; and a Cₘₐₓ for total estrone that is from 3.5429 ng/ml to 5.5358 ng/ml.

In some embodiments, the pharmaceutical composition comprises about 0.25 mg estradiol and about 50 mg progesterone, and administration of the composition to a subject produces, in a plasma sample from the subject, the following parameters:
(i) one or both of (a) an AUC₍₀₋ₜ₎ for estradiol that is from 140.3733 pg·hr/ml to 219.3333 pg·hr/ml and (b) a Cₘₐₓ for estradiol that is from 6.4790 pg/ml to 10.1235 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 909.6091 pg·hr/ml to 1421.2642 pg·hr/ml and (b) a Cₘₐₓ for estrone that is from 42.6549 pg/ml to 66.6483 pg/ml; and optionally
(iv) one or both of (a) an AUC₍₀₋ₜ₎ for total estrone that is from 20.1752 ng·hr/ml to 31.5238 ng·hr/ml and (b) a Cₘₐₓ for total estrone that is from 3.5429 ng/ml to 5.5358 ng/ml.

In some embodiments, a pharmaceutical composition for co-administering estradiol and progesterone to a human subject in need thereof comprises about 0.50 mg estradiol and about 50 mg progesterone, and administration of the composition to the subject produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or both parameters selected from: an AUC₍₀₋ₜ₎ for estrone that is from 1819.2181 pg·hr/ml to 2842.5283 pg·hr/ml, and a Cₘₐₓ for estrone that is from 85.3098 pg/ml to 133.2966 pg/ml.

In some embodiments, administration of the composition to the subject further produces, in a plasma sample from the subject, one or both parameters selected from: an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the pharmaceutical composition comprises about 0.50 mg estradiol and about 50 mg progesterone, and administration of the composition to a subject produces, in a plasma sample from the subject, the following parameters:
(i) one or both of (a) an UC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, a pharmaceutical composition for co-administering estradiol and progesterone to a human subject in need thereof comprises about 0.50 mg estradiol and about 100 mg progesterone, and administration of the composition to the subject produces, in a plasma sample from the subject, one or more parameters selected from:
(i) an area under the curve (AUC)₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or both parameters selected from: an AUC₍₀₋ₜ₎ for estrone that is from 1819.2181 pg·hr/ml to 2842.5283 pg·hr/ml, and a Cₘₐₓ for estrone that is from 85.3098 pg/ml to 133.2966 pg/ml.

In some embodiments, administration of the composition to the subject further produces, in a plasma sample from the subject, one or both parameters selected from: an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the pharmaceutical composition comprises about 0.50 mg estradiol and about 100 mg progesterone, and administration of the composition to a subject produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, a pharmaceutical composition for co-administering estradiol and progesterone to a human subject in need thereof comprises about 1 mg estradiol and about 100 mg progesterone, and administration of the composition to the subject produces, in a plasma sample from the subject, one or more parameters selected from:
(i) an area under the curve (AUC)₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or both parameters selected from: an AUC₍₀₋ₜ₎ for estrone that is from 3638.4363 pg·hr/ml to 5685.0567 pg·hr/ml, and a Cₘₐₓ for estrone that is from 170.6197 pg/ml to 266.5933 pg/ml.

In some embodiments, administration of the composition to the subject further produces, in a plasma sample from the subject, one or both parameters selected from: an AUC₍₀₋ₜ₎ for total estrone that is from 80.7010 ng·hr/ml to 126.0953 ng·hr/ml, and a Cₘₐₓ for total estrone that is from 14.1716 ng/ml to 22.1431 ng/ml.

In some embodiments, the pharmaceutical composition comprises about 0.50 mg estradiol and about 100 mg progesterone, and administration of the composition to a subject produces, in a plasma sample from the subject, the following parameters:
(i) one or both of (a) an AUC₍₀₋ₜ₎ for estradiol that is from 561.4933 pg·hr/ml to 877.3333 pg·hr/ml and (b) a Cₘₐₓ for estradiol that is from 25.9161 pg/ml to 40.4939 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 3638.4363 pg·hr/ml to 5685.0567 pg·hr/ml and (b) a Cₘₐₓ for estrone that is from 170.6197 pg/ml to 266.5933 pg/ml; and optionally
(iv) one or both of (a) an AUC₍₀₋ₜ₎ for total estrone that is from 80.7010 ng·hr/ml to 126.0953 ng·hr/ml and (b) a Cₘₐₓ for total estrone that is from 14.1716 ng/ml to 22.1431 ng/ml.

In some embodiments, the pharmaceutical composition has the blood plasma estradiol concentration profile of FIG. 1. In some embodiments, the pharmaceutical composition has the blood plasma progesterone concentration profile of FIG. 2. In some embodiments, the pharmaceutical composition has the blood plasma estrone concentration profile of FIG. 3. In some embodiments, the pharmaceutical composition has the blood plasma total estrone concentration profile of FIG. 4.

In some embodiments, the one or more parameters as described herein (e.g., the AUG₍₀₋ₜ₎ or Cₘₐₓ for progesterone, estradiol, estrone, or total estrone) are measured at regular intervals (e.g., about every 30 minutes, about every 60 minutes, or about every 90 minutes) or at irregular intervals over a period of time such as 24 hours or 48 hours. In some embodiments, the one or more parameters as described herein (e.g., the AUC₍₀₋ₜ₎ or Cₘₐₓ for progesterone, estradiol, estrone, or total estrone) are measured at about 0.25 hr, 0.5 hr, 0.67 hr, 0.83 hr, 1 hr, 1.33 hr, 1.67 hr, 2 hr, 2.5 hr, 3 hr, 4 hr, 5 hr, 6 hr, 7 hr, 8 hr, 10 hr, 12 hr, 18 hr, 24 hr, 36 hr, or 48 hr after administering the pharmaceutical composition to the subject. In some embodiments, the one or more parameters as described herein are measured at regular or irregular intervals following the administration of a single dose or of a first dose of the pharmaceutical composition to the subject.

In another aspect, methods of treating a subject are provided. In some embodiments, the subject has a condition that is caused at least in part by an estrogen deficiency (e.g., one or more symptoms of menopause, such as vasomotor symptoms). In some embodiments, the method comprises administering to the subject a pharmaceutical composition comprising solubilized estradiol, suspended progesterone, and a solubilizing agent that comprises a medium chain (C6-C12) oil as described herein, wherein administration of the pharmaceutical composition produces, in a plasma sample from the subject, one or more pharmacokinetic parameters as described herein. In some embodiments, the method comprises administering a pharmaceutical composition comprising estradiol at a dosage of about 0.05, 0.1, 0.125, 0.15, 0.20, 0.25, 0.30, 0.35, 0.375, 0.40, 0.45, 0.50, 0.55, 0.60, 0.625, 0.65, 0.70, 0.75, 0.80, 0.85, 0.90, 0.95, 1.00, 1.125, 1.25, 1.375, 1.50, 1.625, 1.75, or 2.00 mg, and comprising progesterone at a dosage of about 25, 50, 75, 100, 125, 150, 175, 200, 250, 300, 350, or 400 mg. In some embodiments, the method comprises administering a pharmaceutical composition comprising: estradiol at a dosage of about 0.25 mg and progesterone at a dosage of about 50 mg; estradiol at a dosage of about 0.50 mg and progesterone at a dosage of about 50 mg; estradiol at a dosage of about 0.50 mg and progesterone at a dosage of about 100 mg; estradiol at a dosage of about 1 mg and progesterone at a dosage of about 100 mg; or estradiol at a dosage of about 2 mg and progesterone at a dosage of about 200 mg.

In some embodiments, the method comprises administering to the subject a pharmaceutical composition comprising about 0.25 mg estradiol and about 50 mg progesterone, wherein administration of the pharmaceutical composition produces, in a plasma sample from the subject, one or more parameters selected from:
(i) an area under the curve (AUC)₍₀₋ₜ₎ for estradiol that is from 140.3733 pg·hr/ml to 219.3333 pg·hr/ml;
(ii) a Cₘₐₓ for estradiol that is from 6.4790 pg/ml to 10.1235 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the pharmaceutical composition further produces, in a plasma sample from the subject, one or more parameters selected from: an AUC₍₀₋ₜ₎ for estrone that is from 909.6091 pg·hr/ml to 1421.2642 pg·hr/ml; a Cₘₐₓ for estrone that is from 42.6549 pg/ml to 66.6483 pg/ml; an AUC₍₀₋ₜ₎ for total estrone that is from 20.1752 ng·hr/ml to 31.5238 ng·hr/ml; and a Cₘₐₓ for total estrone that is from 3.5429 ng/ml to 5.5358 ng/ml.

In some embodiments, the method comprises administering to the subject a pharmaceutical composition comprising about 0.25 mg estradiol and about 50 mg progesterone, wherein administration of the pharmaceutical composition produces, in a plasma sample from the subject, the following parameters:
(i) one or both of (a) an AUC₍₀₋ₜ₎ for estradiol that is from 140.3733 pg·hr/ml to 219.3333 pg·hr/ml and (b) a Cₘₐₓ for estradiol that is from 6.4790 pg/ml to 10.1235 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 909.6091 pg·hr/ml to 1421.2642 pg·hr/ml and (b) a Cₘₐₓ for estrone that is from 42.6549 pg/ml to 66.6483 pg/ml; and optionally
(iv) one or both of (a) an an AUC₍₀₋ₜ₎ for total estrone that is from 20.1752 ng·hr/ml to 31.5238 ng·hr/ml and (b) a Cₘₐₓ for total estrone that is from 3.5429 ng/ml to 5.5358 ng/ml.

In some embodiments, the method comprises administering to the subject a pharmaceutical composition comprising about 0.50 mg estradiol and about 50 mg progesterone, wherein administration of the pharmaceutical composition produces, in a plasma sample from the subject, one or more parameters selected from:
(i) an AUC₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 24.0174 ng·hr/ml to 37.5272 ng·hr/ml; and
(iv) a Cₘₐₓ for progesterone that is from 17.8444 ng/ml to 27.8819 ng/ml.

In some embodiments, administration of the composition to the subject further produces, in a plasma sample from the subject, one or more parameters selected from: an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml; and a Cₘₐₓ for total estrone that is from 7.0858 ng/ml to 11.0715 ng/ml.

In some embodiments, the method comprises administering to the subject a pharmaceutical composition comprising about 0.50 mg estradiol and about 50 mg progesterone, wherein administration of the pharmaceutical composition produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 24.0174 ng·hr/ml to 37.5272 ng·hr/ml and (b) a Cₘₐₓ for progesterone that is from 17.8444 ng/ml to 27.8819 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) an an AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, the method comprises administering to the subject a pharmaceutical composition comprising about 0.50 mg estradiol and about 100 mg progesterone, wherein administration of the pharmaceutical composition produces, in a plasma sample from the subject, one or more parameters selected from:
(i) an area under the curve (AUC)₍₀₋ₜ₎ for estradiol that is from 280.7467 pg·hr/ml to 438.6667 pg·hr/ml;
(ii) a Cₘₐₓ for estradiol that is from 12.9580 pg/ml to 20.2469 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or more parameters selected from: an AUC₍₀₋ₜ₎ for estrone that is from 1819.2181 pg·hr/ml to 2842.5283 pg·hr/ml; a Cₘₐₓ for estrone that is from 85.3098 pg/ml to 133.2966 pg/ml; an AUC₍₀₋ₜ₎ for total estrone that is from 40.3505 ng·hr/ml to 63.0476 ng·hr/ml, and a Cₘₐₓ for total estrone that is from 7.0858 ng/ml to 11.0715 ng/ml.

In some embodiments, the method comprises administering to the subject a pharmaceutical composition comprising about 0.50 mg estradiol and about 100 mg progesterone, wherein administration of the pharmaceutical composition produces, in a plasma sample from the subject, the following parameters:
(i) one or both of (a) an AUC₍₀₋ₜ₎ for estradiol that is from 280.7467 pg·hr/ml to 438.6667 pg·hr/ml and (b) a Cₘₐₓ for estradiol that is from 12.9580 pg/ml to 20.2469 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 1819.2181 pg·hr/ml to 2842.5283 pg·hr/ml and (b) a Cₘₐₓ for estrone that is from 85.3098 pg/ml to 133.2966 pg/ml; and optionally
(iv) one or both of (a) AUC₍₀₋ₜ₎ for total estrone that is from 40.3505 ng·hr/ml to 63.0476 ng·hr/ml and (b) a Cₘₐₓ for total estrone that is from 7.0858 ng/ml to 11.0715 ng/ml.

In some embodiments, the method comprises administering to the subject a pharmaceutical composition comprising about 1 mg estradiol and about 100 mg progesterone, wherein administration of the pharmaceutical composition produces, in a plasma sample from the subject, one or more parameters selected from:
(i) an area under the curve (AUC)₍₀₋ₜ₎ for estradiol that is from 561.4933 pg·hr/ml to 877.3333 pg·hr/ml;
(ii) a Cₘₐₓ for estradiol that is from 25.9161 pg/ml to 40.4939 pg/ml;
(iii) an AUC₍₀₋ₜ₎ for progesterone that is from 48.0348 ng·hr/ml to 75.0543 ng·hr/ml; and
(iv) a Cₘₐₓ for progesterone that is from 35.6889 ng/ml to 55.7639 ng/ml.

In some embodiments, administration of the composition to the subject further produces, in a plasma sample from the subject, one or more parameters selected from: an AUC₍₀₋ₜ₎ for estrone that is from 3638.4363 pg·hr/ml to 5685.0567 pg·hr/ml; a Cₘₐₓ for estrone that is from 170.6197 pg/ml to 266.5933 pg/ml; an AUC₍₀₋ₜ₎ for total estrone that is from 80.7010 ng ·hr/ml to 126.0953 ng·hr/ml; and a Cₘₐₓ for total estrone that is from 14.1716 ng/ml to 22.1431 ng/ml.

In some embodiments, the method comprises administering to the subject a pharmaceutical composition comprising about 1 mg estradiol and about 100 mg progesterone, wherein administration of the pharmaceutical composition produces, in a plasma sample from the subject, the following parameters:
(i) one or both of (a) an AUC₍₀₋ₜ₎ for estradiol that is from 561.4933 pg·hr/ml to 877.3333 pg·hr/ml and (b) a Cₘₐₓ for estradiol that is from 25.9161 pg/ml to 40.4939 pg/ml; and
(ii) one or both of (a) an AUC₍₀₋ₜ₎ for progesterone that is from 48.0348 ng·hr/ml to 75.0543 ng·hr/ml and (b) a Cₘₐₓ for progesterone that is from 35.6889 ng/ml to 55.7639 ng/ml; and optionally
(iii) one or both of (a) an AUC₍₀₋ₜ₎ for estrone that is from 3638.4363 pg·hr/ml to 5685.0567 pg·hr/ml and (b) a Cₘₐₓ for estrone that is from 170.6197 pg/ml to 266.5933 pg/ml; and optionally
(iv) one or both of (a) an AUC₍₀₋ₜ₎ for total estrone that is from 80.7010 ng·hr/ml to 126.0953 ng·hr/ml and (b) a Cₘₐₓ for total estrone that is from 14.1716 ng/ml to 22.1431 ng/ml.

### Example 7

A Phase 3, Double-Blind, Placebo-Controlled, Randomized, Multi-Center Study to Evaluate the Safety and Efficacy of Estradiol in Combination with Progesterone in Postmenopausal Women with an Intact Uterus.

This study had the following two main objectives:
Vasomotor symptoms (VMS): To determine whether the TX-001HR given in a continuous fashion is effective at reducing the frequency and severity of moderate to severe vasomotor symptoms associated with menopause when compared with placebo treatment at weeks 4 and 12.
Endometrial hyperplasia: To determine whether TX-001HR given in a continuous fashion is effective at achieving a ≤1% incidence rate of endometrial hyperplasia following 12 months of therapy.

### A. Study Design/Description

### A.1 Study Design Overview

This study was a prospective, randomized, double-blind, placebo-controlled, parallel group, multicenter trial to determine whether estradiol/progesterone combinations (TX-001HR) given in a continuous fashion are effective at reducing the frequency and severity of vasomotor symptoms associated with menopause as well as establishing an appropriate progesterone dose by demonstrating an acceptably low incidence of endometrial hyperplasia.

Postmenopausal subjects with an intact uterus who met the study entry criteria were randomized to one of five treatment arms (four active and one placebo) and received blinded study medication for 12 months. During the Screening period, all subjects were provided with a diary to self-assess the frequency and severity of their vasomotor symptoms. Subjects experiencing a minimum daily frequency of≥7 (or ≥50 per week) moderate to severe hot flushes participated in a VMS Substudy during the first 12 weeks of treatment. The Substudy subjects were stratified by treatment arm within the sites, and only Substudy subjects had the possibility of being randomized to placebo. Subjects who qualified for the study except for experiencing a minimum daily frequency of ≥7 (or ≥50 per week) moderate to severe hot flushes were stratified by treatment arm within sites to one of the four active treatment arms and received blinded study medication for 12 months, but did not participate in the VMS Substudy. (However, VMS information was collected from all subjects during the first 12 weeks of treatment.)

Postmenopausal women with an intact uterus who sought relief from hot flushes and met all other inclusion/exclusion criteria were eligible for 12 months of study treatment. A subset of All Study Subjects who had ≥7 per day or ≥50 moderate to severe hot flushes per week (as determined during Screening) were eligible for the 12-week VMS Substudy and for a total of 12 months of study treatment.

Clinical evaluations were performed at the following time points:
Screening Period/Baseline (-approximately 60 Days)
Visit 1 Randomization (Week 0) (Day 1)
Visit 2 Interim (Week 4, Day 28 [±3d])
Visit 3 Interim (Week 8, Day 56 [±3d])
Visit 4 Interim (Week 12, Day 84 [±3d])
Visit 5 Interim (Month 6, Day 180 [±4d])
Visit 6 Interim (Month 9, Day 270 [±4d])
Visit 7 End of Treatment (Month 12, Day 360 [±4d])
Post Treatment Phone Contact Safety follow up visit (approximately 15 days after the last dose of study medication)

Unscheduled visits were allowed by investigator discretion for safety reasons, administrative reasons (e.g., re-supply of study medication or diaries), or to address subject concerns or questions about the study.

### A.2 Study Sequence and Duration of Subject Participation

The study involved a Screening period of approximately 60 days before randomization, approximately 12 months (13 cycles) of treatment, and a 15 day follow up period for a total duration of up to approximately 14.5 months of participation (see, Table 22, below).

Table 22: Study Sequence

[image]

### B. Study Population

Approximately 120 investigative sites in the United States recruited subjects for participation in this study. A sufficient number of subjects were screened in order for approximately 1750 subjects to be randomized (400/active treatment group; 150 placebo group). For the VMS Substudy, approximately 750 subjects were randomized (150 per treatment group). Enrollment among treatment centers was competitive, and discontinued subjects were not replaced.

### B.1 Inclusion Criteria

To participate in the study, a subject MUST: (1) be a female between the ages of 40 and 65 years (at the time of randomization) who is willing to participate in the study, as documented by signing the informed consent form; (2) be a postmenopausal woman with an intact uterus and a Screening serum estradiol level of ≤50 pg/mL. Postmenopausal is defined herein as: (a) ≥ 12 months of spontaneous amenorrhea, or (b) at least 6 months of spontaneous amenorrhea with a Screening serum FSH level of >40 mIU/ml, or (c) ≥ 6 weeks postsurgical bilateral oophorectomy; (3) be seeking treatment or relief for vasomotor symptoms associated with menopause; (4) to participate in the VMS Substudy, a subject must also report ≥7 moderate to severe hot flushes per day, or ≥50 per week, at the baseline assessment during Screening; subjects whose hot flushes are less frequent may still participate as non-Substudy subjects. [Note: A minimum of 14 consecutive days of complete hot flush diary data are required during the baseline assessment at Screening, and these consecutive days must occur within the last 14 days prior to the Randomization visit (not counting the Randomization visit day itself). The most recent 7 consecutive days of data prior to randomization (Day -7 to Day -1) will be used to determine the baseline number of mild, moderate and severe hot flushes for each subject.]; (5) have a Body Mass Index (BMI) less than or equal to 34 kg/m² (BMI values should be rounded to the nearest integer [e.g., 34.4 rounds down to 34, while 26.5 rounds up to 27]); (6) be willing to abstain from using products (other than study medication) that contain estrogen, progestin, or progesterone throughout study participation; (7) be judged by the principal or sub-investigator physician as being in otherwise generally good health based on a medical evaluation performed during the Screening period prior to the initial dose of study medication. The medical evaluation findings must include: (a) a normal or non-clinically significant physical examination, including vital signs (sitting blood pressure, heart rate, respiratory rate and temperature). Sitting systolic blood pressure must be ≤140 mmHg and diastolic blood pressure must be ≤90 mmHg at Screening. A subject may be taking up to two antihypertensive medications; (b) a normal or non-clinically significant pelvic examination; (c) a mammogram that shows no sign of significant disease (can be performed within previous 6 months prior to initial dose of study medication). Subjects must have a BI-RADS 1 or 2 to enroll in the study. An incomplete mammogram result, i.e. BI-RADS 0, is not acceptable. The site must obtain a copy of the official report for the subject’s study file, and it must be verified that the mammogram itself is available if needed for additional assessment; (d) a normal or non-clinically significant clinical breast examination. An acceptable breast examination is defined as no masses or other findings identified that are suspicious of malignancy; (e) a normal Screening Papanicolaou (“Pap”) smear. (Subjects with findings of atypical glandular cells [AGC], AGUS, ASCUS with high risk HPV type upon reflex testing, LSIL, ASC-H, HSIL, dysplastic cells, or malignant cells must be excluded from randomization.); (f) an acceptable result from an evaluable Screening endometrial biopsy. The endometrial biopsy reports by the two central pathologists at Screening must each specify one of the following: proliferative endometrium; weakly proliferative endometrium; disordered proliferative pattern; secretory endometrium; endometrial tissue other (including benign, inactive or atrophic fragments of endometrial epithelium, glands, stroma, etc.); endometrial tissue insufficient for diagnosis; no endometrium identified; or no tissue identified. However, at least one pathologist must identify sufficient tissue to evaluate the biopsy. Additionally, the endometrial biopsy reports by the two central pathologists of Other Findings at Screening must each specify one of the following: endometrial polyp not present; benign endometrial polyp; or polyp other. See Exclusion criteria #27 and #28; (g) a normal or non-clinically significant 12-lead ECG.

### B.2 Exclusion Criteria

To participate in the study, a subject must NOT: (1) be currently hospitalized; (2) have a history of thrombosis of deep veins or arteries or a thromboembolic disorder; (3) have a history of coronary artery or cerebrovascular disease (e.g., myocardial infarction, angina, stroke, TIA); (4) have a history of a chronic liver or kidney dysfunction/disorder (e.g., Hepatitis C or chronic renal failure); (5) have a history of a malabsorption disorder (e.g., gastric bypass, Crohn’s disease); (6) have a history of gallbladder dysfunction/disorders (e.g., cholangitis, cholecystitis), unless gallbladder has been removed; (7) have a history of diabetes, thyroid disease or any other endocrinological disease. (Subjects with diet-controlled diabetes or controlled hypothyroid disease at Screening are not excluded.); (8) have a history of estrogen-dependent neoplasia; (9) have a history of atypical ductal hyperplasia of the breast; (10) have a finding of clinically significant uterine fibroids at Screening; (11) have had a uterine ablation; (12) have a history of undiagnosed vaginal bleeding; (13) have any history of endometrial hyperplasia, melanoma, or uterine/endometrial, breast or ovarian cancer; (14) have any history of other malignancy within the last 5 years, with the exception of basal cell (excluded if within 1 year) or non-invasive squamous cell (excluded if within 1 year) carcinoma of the skin; (15) have a history of any other cardiovascular, hepatic, renal, pulmonary, hematologic, gastrointestinal, endocrine, immunologic, dermatologic, neurologic, psychological (e.g., bipolar disorder, schizophrenia, major depressive disorder), or musculoskeletal disease or disorder that is clinically significant in the opinion of the Principal Investigator or Medical Sub-Investigator; (16) have any of the following clinical laboratory values at Screening: (a) fasting triglyceride of ≥300 mg/dL and/or total cholesterol of ≥300 mg/dL; (b) positive laboratory finding for Factor V Leiden mutation; (c) AST or ALT ≥1.5 times the upper limit of normal (ULN); (d) fasting glucose >125 mg/dL; (17) be known to be pregnant or have a positive urine pregnancy test. (Note: A pregnancy test is not required for subjects who have had bilateral tubal ligation, bilateral oophorectomy, or are 55 years old or greater and have experienced cessation of menses for at least 1 year.); (18) have contraindication to estrogen and/or progestin therapy or allergy to the use of estradiol and/or progesterone or any components of the investigational drugs; (19) use 15 or more cigarettes per day or currently use any electronic cigarettes; (20) have a history of drug and/or alcohol abuse within one year of start of study; (21) have used, within 28 days prior to the initial dose of study medication at Visit 1, any medication known to induce or inhibit CYP3A4 enzyme activity that may affect estrogen and/or progestin drug metabolism; (22) have used, within 28 days prior to Screening, or plan to use during the study, any prescription or over-the-counter (OTC) medication (including herbal products, such as St. John’s Wort) that would be expected to alter progesterone or estrogen activity or is being used to treat vasomotor symptoms; (23) have used estrogen alone or estrogen/progestin, SERM (selective estrogen receptor modulator), testosterone, or estrogen/testosterone for any of the following time periods: (a) vaginal nonsystemic hormonal products (rings, creams, gels) within 7 days prior to Screening, or vaginal systemic products (e.g., FemRing) within 28 days prior to Screening; (b) transdermal estrogen alone or estrogen/progestin products within 8 weeks prior to Screening; (c) Oral estrogen and/or progestin therapy and/or SERM within 8 weeks prior to Screening; (d) Progestational implants, estrogen or estrogen/progestational injectable drug therapy within 3 months prior to Screening; (e) estrogen pellet therapy or progestational injectable drug therapy within 6 months prior to Screening; (f) percutaneous estrogen lotions/gels within 8 weeks prior to Screening; (g) oral, topical, vaginal, patch, implantable or injectable androgen therapy within 8 weeks prior to Screening; (24) have used an intrauterine device (IUD) within the 12 weeks prior to Screening; (25) For subjects in the VMS Substudy only: use of medication that may affect the outcome of the vasomotor symptom endpoints within 28 days prior to Screening (e.g. SSRIs [selective serotonin reuptake inhibitors], SNRIs [serotonin and norepinephrine reuptake inhibitors], aldomet, dopaminergic or antidopaminergic drugs, gabapentin, clonidine, or bellergal); (26) have any reason which, in the opinion of the Principal Investigator or Medical Sub-Investigator, would prevent the subject from safely participating in the study or complying with protocol requirements; (27) have a Screening endometrial biopsy sample that is found by both primary pathologists to have endometrial tissue insufficient for diagnosis, no endometrium identified, or no tissue identified. (With the approval of the Medical Monitor, the Screening endometrial biopsy may be repeated once.); (28) endometrial polyps with atypical nuclei reported by at least 1 central pathologist; (29) have contraindication to any planned study assessments (e.g., endometrial biopsy); (30) have participated in another clinical trial within 30 days prior to Screening, have received an investigational drug within the three months prior to the initial dose of study medication, or be likely to participate in a clinical trial or receive another investigational medication during the study; (31) currently use marijuana.

### B.3 Concomitant and Prohibited Medications

Study participants were instructed not to take estrogen, SERMs, progestin or progesterone other than study medication in the specified timeframes prior to Screening outlined in Section B.2 (Exclusion Criteria) nor during the study.

The use of any medication known to induce or inhibit CYP3A4 enzyme activity that may affect estrogen/progestin drug metabolism was prohibited within 28 days prior to Visit 1 and throughout the study.

The use of any medication, herbal products or nutritional supplements known or suspected to interact with hormone therapy was prohibited within 28 days prior to Screening and throughout the study. Testosterone will be prohibited within 8 weeks prior to Screening and during the study.

For subjects in the VMS Substudy only: use of medication that may affect the outcome of the vasomotor symptom endpoints within 28 days prior to Screening and during participation in the VMS Substudy (through the first 12 weeks of treatment) was prohibited (e.g., SSRIs, SNRIs, aldomet, dopaminergic or antidopaminergic drugs, gabapentin, clonidine, or bellergal).

Subjects were instructed to report all concomitant medications, including over the counter (OTC) products and herbal or nutritional supplements/medications. Subject were also instructed to report any changes in concomitant medications; they are to be questioned by site personnel regarding concomitant medications at each site visit and, when appropriate, during contacts between visits.

### C. Study Procedures and Evaluations

### C.1 Study Procedures

The Study Flow Chart with the schedule of activities can be found in Table 23, below.

TABLE 23

Study Flow Chart

| Activity | Screening Period | Visit 1: Randomization | Visit 2: Interim | Visit 3: Interim | Visit 4: Interim | Visit 5: Interim |
|---|---|---|---|---|---|---|
| | Days -60 - 0* | Week 0 Day 1 | Week 4 Day 28 (±3 d) | Week 8 Day 56 (±3 d) | Week 12 Day 84 (±3 d) | Month 6 Day 180 (±4 d) |
| Informed Consent | X | | | | | |
| Demographics | X | | | | | |
| Medical History and Prior Medications | X | X | | | | |
| Physical Examination | X | | | | | |
| Body Weight^{A} | X | | | | X | X |
| Height | X | | | | | |
| Vital Signs | X | X | X | X | X | X |
| Urine Pregnancy test P^{B} | X | | | | | |
| Pelvic and Breast Examination | X | | | | | X |
| Pap Smear | X | | | | | |
| Mammography | XP ^{C} | | | | | |
| ECG, 12-lead | X | | | | | |
| Hematology, Chemistry, Urinalysis, and Coagulation | X | | | | X | X |
| Serum FSH (if applicable), Thyroid Panel | X | | | | | |
| Endometrial BiopsyP^{E} | X | | | | | |
| Estradiol, Estrone | X | | X | | X | X |
| Progesterone | X | | | | X | |
| Hot Flush Diary DispensedP^{F} | X | X | X | X | | |
| Bleeding/Spotting and Dosing Diary DispensedP^{F} | | X | X | X | X | X |
| Diary Collection/Review | | X | X | X | X | X |
| Eligibility Review/Randomization | | XP ^{G} | | | | |
| Study Medication Dispensed | | X | X | X | X | X |
| Treatment Instruction and Compliance | | X | X | X | X | X |
| Collect Unused Drug/Used Containers | | | X | X | X | X |
| Concomitant Medication | X | X | X | X | X | X |
| Assessment of Adverse Events | | X | X | X | X | X |
| MENQOL Questionnaire | | X | | | X | |
| MOS-Sleep Questionnaire | | X | | | X | |
| CGI (VMS Substudy Only) | | | X | X | X | |

TABLE 23

Study Flow Chart (continued)

| Activity | Visit 6: Interim | Visit 7: End of Treatment | Early Discontinuation Requirements | Post Treatment Follow up Phone Contact Approximately 15 days after last dose of study medication) |
|---|---|---|---|---|
| | Month 9 Day 270 (±4 d) | Month 12 Day 360 (±4 d) | | |
| Informed Consent | | | | |
| Demographics | | | | |
| Medical History and Prior Medications | | | | |
| Physical Examination | | X | X | |
| Body Weight^{A} | | X | X | |
| Height | | | | |
| Vital Signs | X | X | X | |
| Urine Pregnancy test P^{B} | | | | |
| Pelvic and Breast Examination | | X | X | |
| Pap Smear | | X | XP ^{H} | |
| Mammography | | X | XP^{J} | |
| ECG, 12-lead | | X | X | |
| Hematology, Chemistry, Urinalysis, and Coagulation^{D} | X | X | X | |
| Serum FSH (if applicable), Thyroid Panel | | | | |
| Endometrial Biopsy^{E} | | X | X P^{I} | |
| Estradiol, Estrone | X | X | X | |
| Progesterone | | X | X | |
| Hot Flush Diary DispensedP^{F} | | | | |
| Bleeding/Spotting and Dosing Diary DispensedP^{F} | X | | | |
| Diary Collection/Review | X | X | X | |
| Eligibility Review/Randomization | | | | |
| Study Medication Dispensed | X | | | |
| Treatment Instruction and Compliance | X | X | X | |
| Collect Unused Drug/Used Containers | X | X | X | |
| Concomitant Medication | X | X | X | X |
| Assessment of Adverse Events | X | X | X | X |
| MENQOL Questionnaire | | X | X | |
| MOS-Sleep Questionnaire | | X | X | |
| CGI (VMS Substudy Only) | | | | |

* - Screening period will occur within 60 days prior to study medication administration at Visit 1. If approved by the Medical Monitor, the screening period may be extended.
A - Body weight will be obtained with the subject’s shoes off, jacket or coat removed.
B - A pregnancy test is not required for subjects who had a bilateral tubal ligation, bilateral oophorectomy, or are ≥55 years old and have experienced cessation of menses for at least 1 year. One may be performed at any time during the study if a pregnancy is suspected.
C - Mammography can be performed within previous 6 months: mammogram should be available for review.
D - Factor V Leiden at Screening only.
E - The screening endometrial biopsy can be performed at any time during the screening period. It is suggested that is be performed last to avoid performing unnecessary invasive procedures on subjects who screen fail for other reasons.
F - Hot flush, dosing, and bleeding/spotting diaries will be combined as appropriate for each collection period of the study.
G - Subjects with ≥7 moderate to severe hot flushes per day (or ≥50 per week) will be randomized into VMS Substudy. All other eligible subjects will be randomized into non-Substudy.
H - A Pap smear is not required if one has been done <5 months prior to early discontinuation.
I - Subjects who discontinue ≥12 weeks of study medication will be required to have an endometrial biopsy. End of Treatment or Early Discontinuation endometrial biopsies that need to be repeated per protocol must be performed within 30 days of the final dose of study medication.
J - Subjects who discontinue ≥6 months of study medication will be required to have a mammogram.

### C.1.1 Informed Consent

The Postmenopausal women 40 to 65 years of age signed a written informed consent that was approved by an Institutional Review Board (IRB). No study-related procedures or activities were performed until each subject was fully informed about the study and the consent form was properly signed and dated. The Investigator, or a qualified person designated by the Investigator, explained the purpose and procedures of the study as well as potential benefits and risks. All subjects were given a copy of the signed and dated consent form.

### C.1.2 Demographics, Medical/Gynecological History, and Concomitant Medications

At Screening, a complete medical history, including demographic data (age, sex, race and ethnicity), surgical and gynecological history (date of last menstrual period, date of bilateral oophorectomy, if applicable), and use of tobacco and alcohol history was recorded. The medical history included a review of all past and current diseases. It also included the history of hot flushes. Any hormonal therapy taken within 6 months prior to this visit was recorded (using generic names, if known) with the corresponding indication. The other medications to be recorded included prescription and OTC medications, dietary supplements, and all products taken within 30 days prior to the Screening visit.

### C.1.3 Physical Examination, Including Height, Weight, and BMI

A complete physical examination was conducted at Screening and Visit 7/End of Treatment. The physical examination included, at a minimum, examination of the subject’s general appearance, HEENT (head, eyes, ears, nose and throat), heart, lungs, musculoskeletal system, gastrointestinal (GI) system, neurological system, lymph nodes, abdomen and extremities. The subject’s height was measured at Screening only and body weight (while the subject is lightly clothed) was measured at Screening, Week 12, Month 6, and the End of Treatment. BMI was calculated.

### C.1.4 Pregnancy Test

A urine pregnancy test was performed at the very start of Screening. If the pregnancy test were positive, the subject was excluded from study participation. A pregnancy test was not required for subjects who had a bilateral tubal ligation, bilateral oophorectomy, or who are 55 years old or greater and have experienced cessation of menses for at least 1 year.

### C.1.5 Vital Signs

Vital signs (body temperature, heart rate [HR], respiration rate [RR], and sitting blood pressure [BP]) were measured after the subject has been sitting for > 10 minutes.

### C.1.6 Pelvic and Breast Examination

Each subject was required to have normal or non-clinically significant pelvic and breast examinations performed prior to initial dose of study medication. The pelvic and breast exam was repeated at Visits 5 and 7/End of Treatment.

### C.1.7 Laboratory Measurements

### C.1.7.1 Clinical Laboratory Tests

Blood samples for blood chemistry, hematology, coagulation tests, and hormone levels were collected. The schedule associated with laboratory measurements can be found in Table 24, Laboratory Tests Schedule, below.

TABLE 24

Laboratory Tests Schedule

| Laboratory Tests^{1,2} | Screening | VISIT 1 | VISIT 2 | VISIT 3 | VISIT 4 | VISIT 5 | VISIT 6 | VISIT 7 |
|---|---|---|---|---|---|---|---|---|
| Hematology | | | | | | | | |
| Hemoglobin | X | - | - | - | X | X | X | X |
| Hematocrit | X | - | - | - | X | X | X | X |
| Red Blood Cell Count | X | - | - | - | X | X | X | X |
| White Blood Cell Count with differential | X | - | - | - | X | X | X | X |
| Platelet Count | X | - | - | - | X | X | X | X |
| Serum Chemistry | | | | | | | | |
| Sodium | X | - | - | - | X | X | X | X |
| Potassium | X | - | - | - | X | X | X | X |
| Chloride | X | - | - | - | X | X | X | X |
| Bicarbonate | X | - | - | - | X | X | X | X |
| Blood urea nitrogen | X | - | - | - | X | X | X | X |
| Iron | X | - | - | - | X | X | X | X |
| Albumin | X | - | - | - | X | X | X | X |
| Total Protein | X | - | - | - | X | X | X | X |
| Aspartate Aminotransferase | X | - | - | - | X | X | X | X |
| Alanine Aminotransferase | X | - | - | - | X | X | X | X |
| Alkaline Phosphatase | X | - | - | - | X | X | X | X |
| Amylase | X | - | - | - | X | X | X | X |
| Creatinine | X | - | - | - | X | X | X | X |
| Calcium | X | - | - | - | X | X | X | X |
| Phosphate | X | - | - | - | X | X | X | X |
| Uric Acid | X | - | - | - | X | X | X | X |
| Total Bilirubin | X | - | - | - | X | X | X | X |
| Glucose³ | X | - | - | - | X | X | X | X |
| Coagulogram | | | | | | | | |
| Prothrombin time, Activated partial thromboplastin time, Fibrinogen, Antithrombin III, Protein C and Protein S | X | - | - | - | X | X | X | X |
| Factor V Leiden | X | - | - | - | - | - | - | - |
| Hormone Levels | | | | | | | | |
| Follicle-stimulating hormone (FSH)⁴ | X | - | - | - | - | - | - | - |
| Thyroid-stimulating hormone | X | - | - | - | - | - | - | - |
| Estradiol, estrone | X | - | X | - | X | X | X | X |
| Progesterone | X | | | | X | | | X |
| Fasting LDL, HDL, Triglycerides, Total Cholesterol | X | - | - | - | X | X | X | X |
| Appearance, pH, Specific Gravity, Protein | X | - | - | - | X | X | X | X |
| Urine pregnancy test⁵ | X | - | - | - | - | - | - | - |

¹Additional tests may be performed, if necessary, based on:
• Standard lab panels utilized by the clinical site;
• Country regulatory requirements
• Based on clinical judgment and necessity
² Normal laboratory values will be provided in study-specific laboratory manual
³ Must be fasting
⁴ Subjects with ≥12 months of spontaneous amenorrhea or bilateral oophorectomy excluded from FSH test
⁵ A pregnancy test is not required for subjects who have had a hysterectomy, bilateral oophorectomy, or bilateral tubal ligation or are ≥55 years old and has have experienced cessation of menses for at least 1 year

The following parameters were monitored:
Blood Chemistry: Sodium, potassium, chloride, bicarbonate, blood urea nitrogen (BUN), iron, albumin, total protein, aspartate aminotransferase (AST), alanine aminotransferase (ALT), amylase, alkaline phosphatase, serum creatinine, calcium, phosphate, uric acid, total bilirubin, glucose, triglycerides, total cholesterol, HDL, LDL (must be fasting a minimum of 8 hours).
Hematology: Complete blood count (CBC) including white blood cell count (WBC) and differential, red blood cell (RBC) count, hemoglobin, hematocrit and platelet count.
Coagulation Tests: Prothrombin time (PT/INR), activated partial thromboplastin time (APTT), fibrinogen, Protein C and Protein S, antithrombin III, Factor V Leiden (Screening only).
Hormone Levels: Follicle-stimulating hormone (FSH) (not required for subjects with ≥12 months of spontaneous amenorrhea or bilateral oophorectomy), estradiol, estrone, progesterone, thyroid-stimulating hormone (TSH). If TSH is abnormal as per lab range, reflex testing of free triiodothyronine (T3) and free thyroxine (T4) will be performed.
Urine analysis: Appearance, specific gravity, protein, pH.
Urine Pregnancy Test: A pregnancy test was not required for subjects who had a bilateral tubal ligation, bilateral oophorectomy or are ≥55 years old and have experienced cessation of menses for at least 1 year. A test could have been performed at any time during the study if a pregnancy was suspected.

Clinical laboratory tests could be repeated with prior approval of the Sponsor or designee only. The Principal Investigator or a qualified and delegated Medical Sub-Investigator were responsible for interpreting the laboratory findings (i.e., determining the clinical significance of any abnormal values indicated) and for signing and dating the laboratory report. Any clinically relevant changes requiring treatment, interruption or discontinuation of study medication occurring during the trial were reported as an adverse event.

Any authorized and qualified person was allowed to collect biological samples from the subject. The date and time of sample collection was recorded. A central laboratory was designated for this study to perform the analyses of blood and urine samples and to provide applicable kits, supplies, and instructions for the collection and handling of samples. Estradiol evaluations for screening inclusion criteria were conducted by a validated rapid chromatographic assay. All baseline and post-treatment estradiol, estrone, and progesterone were done by a validated bioanalytical assay. Sample collection and handling procedures for laboratory assessments were performed according to the procedures designated by the central laboratory. Contact information and relevant documentation regarding the central laboratory were provided separately.

### C.1.8 12-Lead Electrocardiography (ECG)

A standard 12-lead ECG was obtained at Screening and Visit 7/End of Treatment and read locally. The investigator was responsible for reviewing the interpretation of the ECG and for retaining hardcopies.

### C.1.9 Pap Smear and Mammography

Each subject was required to have the following examinations at Screening:
Screening Pap smear for subjects with an intact cervix. (All subjects must have had a Pap smear done during Screening, regardless of any recent prior assessment.)
Mammogram (may have been performed within previous 6 months of first dose of study medication; the site must obtain a copy of the official report for the subject’s study file, and it must be verified that the mammogram itself is available if needed for additional assessment. If the subject had not had a mammogram within the previous 6 months prior to the first dose of study medication or relevant documentation cannot be obtained, one was performed before the subject can be randomized).

A Pap smear and mammogram were repeated at Visit 7/End of Treatment.

### C.1.10 Endometrial Biopsy

Endometrial biopsies were performed by a board-certified gynecologist and the procedure, including instrument used, will be documented in the subject’s source file.

An endometrial biopsy was performed at Screening and at Visit 7/End of Treatment. Subjects who discontinue study participation after receiving ≥12 weeks of study medication were also required to have an endometrial biopsy. Unscheduled endometrial biopsies could be performed during the study, if indicated for medical reasons.

The Screening endometrial biopsy was performed at various times during the screening period; however, it was suggested that it be performed last, after other screening assessments have indicated that the subject is otherwise an eligible candidate for the study to avoid performing unnecessary invasive procedures on subjects who screen fail for other reasons (preferably it was carried out around the middle of the Screening period, allowing for sufficient time to receive the pathologists’ reports during the Screening window).

Biopsy specimens were processed by a central laboratory. To ensure uniformity in interpretation, a chartered Pathology Committee consisting of four independent pathologists who are experts in the field of endometrial pathology assessed endometrial biopsy samples in a blinded fashion. Instructions and other additional information regarding performing the endometrial biopsies, submission of samples and reporting were provided separately in the study Pathology Committee Charter.

All screening endometrial biopsies were read centrally by two pathologists. At Screening, at least one pathologist was required to identify sufficient tissue to evaluate the biopsy for study eligibility. With the approval of the medical monitor, the Screening endometrial biopsy could be repeated once when an initial endometrial biopsy was performed and both of the primary pathologists report endometrial tissue insufficient for diagnosis, no endometrium identified, or no tissue identified, and if the subject has met all other protocol-specified eligibility criteria to date.

During screening, if either pathologist assessed the endometrial biopsy as hyperplasia or cancer, or if either pathologist identified an endometrial polyp with either hyperplasia, glandular atypia of any degree (e.g., atypical nuclei) or cancer, the subject was excluded from the study.

Visit 7/End of Treatment, Early Termination and on-treatment unscheduled biopsies were centrally read by three pathologists.

The End of Treatment or Early Termination biopsy was repeated once when all three of the pathologists report endometrial tissue insufficient for diagnosis, no endometrium identified, or no tissue identified. End of Treatment or Early Discontinuation endometrial biopsies that needed to be repeated per protocol were performed within 30 days of the final dose of study medication.

For unscheduled biopsies, the histological diagnosis of endometrial polyp did not require withdrawal, unless hyperplasia or atypical nuclei was present.

### C.1.11 Evaluation of Vasomotor Symptoms and Bleeding/Spotting Episodes

### C.1.11.1 Screening Period

Upon completion of the initial screening procedures, all subjects who were determined to be eligible to continue screening were provided with a Hot Flush diary that was completed for the remainder of the Screening period. Subjects were instructed to complete the diary on a daily basis by recording the number and severity of vasomotor symptoms (hot flushes) in their diaries.

The severity of vasomotor symptoms is defined clinically in Table C.1.11.1, below.

TABLE C.1.11.1

Severity of Hot Flushes

| | |
|---|---|
| Severity | Description |
| Mild | Sensation of heat without sweating |
| Moderate | Sensation of heat with sweating, able to continue activity |
| Severe | Sensation of heat with sweating, causing cessation of activity |

A minimum of 14 consecutive days of complete hot flush diary data was required during the baseline assessment at Screening, and these consecutive days had to occur within the last 14 days prior to the Randomization visit (however, not counting the Randomization visit day itself). The most recent 7 consecutive days of data prior to randomization was used to determine the baseline number of mild, moderate and severe hot flushes for each subject.

At Visit 1, subjects who continued to meet the eligibility criteria with a minimum daily frequency of ≥7 (or ≥50 per week) moderate to severe hot flushes in the 7 days prior to Visit 1 were randomized into the VMS Substudy. All other eligible subjects not meeting the VMS Substudy hot flush requirements were randomized into the non-Substudy portion of the trial until enrollment was completed.

### C.1.11.2 Treatment Period - Day 1-84

Upon All subjects (both VMS Substudy and non-Substudy) will complete a Hot Flush/Bleeding and Spotting diary through Week 12. In addition to collecting the hot flush frequency and severity, the subject diary will also collect the date and time of study medication administration, the time of food intake closest to dosing and the intensity of bleeding/spotting episodes. Bleeding/Spotting definitions are presented in Table C.1.11.2, below.

TABLE C.1.11.2

Bleeding/Spotting Definitions

| | |
|---|---|
| Term | Definition |
| None | No bleeding or spotting |
| Spotting | Not requiring sanitary protection |
| Bleeding | Requiring sanitary protection |

Subjects were instructed to return their diary at each study visit. Study staff reviewed the returned subject diaries to ensure proper completion; subjects were re-instructed as needed on correctly completing the diary.

Any changes made to the subject’s diary entries were documented in accordance with Good Clinical Practice standards. Any change or correction to source documents were dated, initialed, explained (if necessary) and did not obscure the original entry. As appropriate, corrections were preferably documented during review of the diary while the subject was present at the site clinic. The Investigator or his/her designee were able to make self-evident corrections to subject initials, subject number, and dates when proper entries could be determined unambiguously. Corrections could be made to the diaries when appropriate to document the subject’s intended meaning, but missing diary data was not to be completed by recall more than one week after the date of the event. Corrections for study drug administration could be made based on study drug reconciliation when appropriate.

The study staff entered the subject diary data into the CRF promptly in order for the Sponsor to assess subject compliance with completion of the subject diaries. Subjects who did not complete the diary correctly or did not submit the diary despite repeated instructions could be discontinued for non-compliance after discussion with the Medical Monitor. If a subject forgot to return the diary to the site, the study staff made every reasonable attempt to collect the diary or any missing diary pages.

### C.1.11.3 Treatment Period - Day 85-360

Upon completion of the initial 12 weeks of the Treatment Period, all subjects were requested to continue their participation and complete 12 months of therapy. At the Week 12 visit, subjects were dispensed 3 months supply of a Bleeding and Spotting diary for completion daily. Subjects were instructed to return the diary at the next scheduled visit. Subjects continued to complete the Bleeding and Spotting diary and returned the diary at each clinic visit until the End of Treatment at Month 12. The study staff continued to monitor compliance with diary completion.

### C.1.12 Menopause-Specific Quality of Life Questionnaire (MENQOL)

The 1996 version of the Menopause-specific quality of life questionnaire (MENQOL), which was developed by Hilditch, et al., Maturitas, 1996;24(3):161-175), was utilized to assess changes in quality of life of study participants. The MENQOL was conducted at Visits 1, 4, 5 and 7.

### C.1.13 Medical Outcomes Study-Sleep Questionnaire (MOS-Sleep)

The 1992 version of the Medical Outcomes Study-Sleep Questionnaire (MOS-Sleep), which is described by Hays, RD and Stewart, AL, Sleep Measures, in A.L. Stewart and J.E. Ware (eds.), Measuring functioning and well-being: The Medical Outcomes Study approach (pp.235-259), Durham, NC: Duke University Press, 1992), was utilized to assess changes in sleep. The MOS-Sleep questionnaire was conducted at Visits 1, 4, 5 and 7.

### C.1.14 Clinical Global Impression (CGI)

At weeks 4, 8 and 12, subjects in the VMS Substudy were asked to provide a Clinical Global Impression (CGI) as described by Gerlinger et al., Menopause: The Journal of The North American Menopause Society, 2012:19(7):799-803. Subjects were instructed to answer the following question: “Rate the total improvement, whether or not in your judgment it is due entirely to drug treatment. Compared to your condition at admission to the study, how much has it changed?” The subjects were asked to answer this question using a symmetric seven-point scale, as follows:
Very much improved
Much improved
Minimally improved
No change
Minimally worse
Much worse
Very much worse

### C.2 Screening Procedures (Approximately 60 days)

The Screening Period began on the date that the subject signed the informed consent form. The prospective subjects visited the study center and were assessed by qualified and properly delegated study staff to verify eligibility and exclude any co-morbid conditions.

The screening evaluation period was completed within 60 days; however, the period may have been longer with the approval of the Medical Monitor. All Screening assessments were completed prior to randomization. Completion of Screening procedures typically required at least two clinic visits prior to randomization at Visit 1.

Subjects were instructed to return to the study site for additional visits as appropriate within the Screening period for completion of the required Screening assessments. The Investigator reviewed the results from all screening procedures, and determined if the subjects were eligible for enrollment into the study. The following procedures and evaluations were conducted at Screening: informed consent, medical and gynecologic history were taken; prior/concomitant medical information was collected; collection of vital signs (body temperature, heart rate (hereinafter, “HR”), respiratory rate (hereinafter, “RR”), and blood pressure (hereinafter, “BP”); height and body weight measurements were taken, and BMI was calculated; a physical exam was carried out; 12-lead ECG was carried out; pelvic and breast examination was carried out; a pap smear was done; blood and urine samples were collected for blood chemistry, hematology and coagulations tests; blood samples were collected for measurements of FSH (subjects with ≥ 12 months of spontaneous amenorrhea or bilateral oophorectomy were excluded), estradiol, estrone, progesterone, and TSH levels (note: if TSH was abnormal as per lab range, reflex testing of T3 and T4 was performed); a urine pregnancy test (subjects with history of tubal ligation, bilateral oophorectomy, or ≥55 years of age and amenorrheic for at least 1 year were excluded); mammogram was performed (if not completed within 6 months of Visit 1 or if a report was not available); endometrial biopsy was performed; dispensed Issue Treatment Hot Flush/Bleeding and Spotting diaries (minimum of 14 days prior to Visit 1); and instructions were given regarding the importance of the diary to the study, methods of completion and methods for returning the diary to the study site.

### C.3 Double-Blind Treatment Phase

All on-therapy visits had a visit window to allow for slight variations in subject schedules and weekends; however, every effort was made to have the subject return on the designated study day. The visit window could be longer, but only with the approval of the Medical Monitor. When planning visits, the overall treatment period was maintained; with subsequent visits based on the Randomization day.

Study visits were typically conducted so as to include the activities outlined in Table 23, above, and as set forth below.

### C.1.1 Visit 1 (Week 0/Day 1)

Subjects who met the eligibility criteria were randomized into one of the treatment arms in the VMS Substudy or non-Substudy. Study medication were dispensed once all Screening Procedures had been completed and eligibility had been verified. The following procedures and evaluations were conducted at this visit: review of study entry criteria; update of medical and gynecological history (to record any new conditions or information that emerged during Screening); collection of vital signs (body temperature, HR, RR, and BP); distribution and collection of subject completed Menopause-Specific Quality of Life Questionnaire (hereinafter, “MENQOL form”)⁵; distribution and collection of subject completed the Medical Outcomes Study-Sleep Questionnaire (hereinafter, “MOS-Sleep form”)⁶; recording and documentation of adverse events (AEs) since the last visit; collection of prior/concomitant medication information since the last visit; collection of Screening diaries and assignment to VMS Substudy⁷ and non-Substudy⁸; subjects were randomized to study medication; subjects were instructed on study medication self-administration and on completion of subject diary; ⁹ study medication was dispensed for the subsequent 4 weeks of treatment (with allowances for the visit window), and subjects were instructed to take it at bedtime with food; and subjects were further instructed to return to return to the study site in approximately 4 weeks (Day 28 ± 3d).

⁵ The 1996 version of the Menopause-specific quality of life questionnaire (MENQOL) developed by Hilditch, et al., Maturitas, 1996;24(3): 161-175), was utilized to assess changes in quality of life of study participants. The MENQOL was conducted at Visits 1, 4, 5 and 7.

⁶ The 1992 version of the Medical Outcomes Study-Sleep Questionnaire (MOS-Sleep), which is described by Hays, RD and Stewart, AL, Sleep Measures, in A.L. Stewart and J.E. Ware (eds.), Measuring functioning and well-being: The Medical Outcomes Study approach (pp.23>5-259), Durham, NC: Duke University Press, 1992), was utilized to assess changes in sleep. The MOS-Sleep questionnaire was conducted at Visits 1, 4, 5 and 7.

⁷ A minimum of 14 consecutive days of complete hot flush diary data was required during the baseline assessment at Screening, and these consecutive days must have occurred within the 14 days prior to the Randomization visit (not counting the Randomization visit day itself). The most recent 7 consecutive days of data prior to randomization (Day -7 to Day -1) was used to determine the baseline number of mild, moderate and severe hot flushes for each subject.

⁸ To participate in the VMS Substudy, a subject must have reported ≥7 moderate to severe hot flushes per day, or ≥50 per week, at the baseline assessment during Screening; subjects whose hot flushes were less frequent may still have participated as non-Substudy subjects until enrollment of the non-VMS Substudy was reached.

⁹ Issue Treatment Hot Flush/Bleeding and Spotting diaries were handed out for the first 4 weeks of treatment (with allowances for the visit window).

### C.1.2 Visit 2 (Week 4/Day 28 ±3, Interim)

Subjects returned to the study site at Week 4 (Day 28 ± 3 days) of treatment initiation. The following procedures and evaluations were conducted at this visit: collection of vital signs (body temperature, HR, RR, and BP); blood collection for the monitoring of serum levels of estradiol and estrone; collection and review of completed subject diaries for the previous 4 weeks of treatment by site personnel; Issue Treatment Hot Flush/Bleeding and Spotting diaries were dispersed for the subsequent 4 weeks of treatment (with allowances for the visit window) and instructions for completion were reviewed, if necessary by site personnel; study medication was dispensed for the subsequent 4 weeks of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; for VMS Substudy subjects only, the Clinical Global Impression assessment (hereinafter, the “CGI assessment”)¹⁰ was completed; and subjects were instructed to return to the study site in approximately 4 weeks (Day 56 ± 3 days).

¹⁰ At weeks 4, 8 and 12, subjects in the VMS Substudy were asked to provide a Clinical Global Impression (CGI) as described by Gerlinger et al., Menopause: The Journal of The North American Menopause Society, 2012:19(7):799-803.

### C.1.3 Visit 3 (Week 8/Day 56 ±3, Interim)

Subjects returned to the study site at Week 8 (Day 56 ± 3 days) of treatment initiation. The following procedures and evaluations were conducted at this visit: collection of vital signs (body temperature, HR, RR, and BP); collection and review of completed subject diaries for the previous 4 weeks of treatment by site personnel; Issue Treatment Hot Flush/Bleeding and Spotting diaries were dispersed for the subsequent 4 weeks of treatment (with allowances for the visit window) and instructions for completion were reviewed, if necessary by site personnel; study medication was dispensed for the subsequent 4 weeks of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; for VMS Substudy subjects only, the CGI assessment was completed; and subjects were instructed to return to the study site in approximately 4 weeks (Week 12, Day 84 ± 3 days).

### C.1.4 Visit 4 (Week 12/Day 84 ± 3, Interim)

Subjects returned to the study site at Week 12 (Day 84 ± 3 days) of treatment initiation. The following procedures and evaluations were conducted at this visit: collection of vital signs (body temperature, HR, RR, and BP); body weight measurements were taken; blood sample was collected for blood chemistry, hematology and coagulations tests; blood sample was collected for measurements of estradiol, estrone, and progesterone levels; a urine analysis was done; collection and review of completed subject diaries for the previous 4 weeks of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; distribution and collection of subject completed MENQOL; distribution and collection of subject completed MOS-Sleep form; and for VMS Substudy subjects only, the CGI assessment was completed.

Subjects were instructed to return to the study site in approximately 3 months (Month 6, Day 180 ± 4 days). In addition, study subjects were notified that they may be contacted via telephone (or other means as appropriate) between visits. If contacted, subjects would be queried for treatment compliance, adverse events, and concomitant medications. If necessary, the subject will be re-instructed by site personnel on study medication self-administration and compliance with other study requirements (e.g., diary completion). All contacts with the subjects should have been documented in their source files. NOTE: Subjects enrolled in the VMS Substudy will continue their participation along with non-Substudy subjects after 12 weeks of evaluation.

### C.1.5 Visit 5 (Month 6/Day 180 ± 3, Interim)

Subjects returned to the study site at Month 6 (Day 180 ± 3 days) of treatment initiation. The following procedures and evaluations were conducted at this visit: collection of vital signs (body temperature, HR, RR, and BP); body weight measurements were taken; pelvic and breast examination was carried out; blood sample was collected for blood chemistry, hematology and coagulations tests; blood sample was collected for measurements of estradiol and estrone levels; a urine analysis was done; collection and review of completed subject diaries for the previous 6 months of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; concomitant medication information since the last visit were collected; distribution and collection of subject completed MENQOL form); and distribution and collection of subject completed MOS-Sleep form.

Subjects were instructed to return to the study site in approximately 3 months (Month 9, Day 270 ± 4 days). In addition, study subjects were notified that they may be contacted via telephone (or other means as appropriate) between visits. If contacted, subjects would be queried for treatment compliance, adverse events, and concomitant medications. If necessary, the subject will be re-instructed by site personnel on study medication self-administration and compliance with other study requirements (e.g., diary completion). All contacts with the subjects should have been documented in their source files.

### C.1.6 Visit 6 (Month 9/Day 270 ± 3, Interim)

Subjects returned to the study site at Month 9 (Day 270 ± 3 days) of treatment initiation. The following procedures and evaluations were conducted at this visit: collection of vital signs (body temperature, HR, RR, and BP); blood sample was collected for blood chemistry, hematology and coagulations tests; blood sample was collected for measurements of estradiol and estrone levels; a urine analysis was done; collection and review of completed subject diaries for the previous 3 months of treatment by site personnel; Issue Treatment Hot Flush/Bleeding and Spotting diaries were dispersed for the subsequent 3 months of treatment (with allowances for the visit window) and instructions for completion were reviewed, if necessary by site personnel; study medication was dispensed for the subsequent 3 months of treatment (with allowances for the visit window) and instructions for self-administration with food were reviewed, by study personnel; all study medication containers and unused study medication from the previous period of treatment were collected by study personnel; treatment instructions and compliance were reviewed with the subject; all AEs since the last visit were recorded and documented; and concomitant medication information since the last visit were collected;

Subjects were instructed to return to the study site in approximately 3 months (Month 12, Day 360 ± 4 days). In addition, study subjects were notified that they may be contacted via telephone (or other means as appropriate) between visits. If contacted, subjects would be queried for treatment compliance, adverse events, and concomitant medications. If necessary, the subject will be re-instructed by site personnel on study medication self-administration and compliance with other study requirements (e.g., diary completion). All contacts with the subjects should have been documented in their source files.

### C.1.7 Visit 7 (Month 12/Day 360 ± 4, End of Treatment)

Subjects returned to the study site at Moth 12 (Day 360 ± 4 days) of treatment initiation. The following procedures and evaluations were conducted at this visit: a physical exam was carried out; collection of vital signs (body temperature, HR, RR, and BP); body weight measurements were taken; pelvic and breast examination was carried out; a pap smear was done; mammography was done; 12-lead ECG was carried out; endometrial biopsy was done;¹¹ blood sample was collected for blood chemistry, hematology and coagulations tests; blood sample was collected for measurements of estradiol, estrone, and progesterone levels; a urine analysis was done; collection and review of completed Issue Treatment Hot Flush/Bleeding and Spotting diaries for the previous 3 months of treatment by site personnel; all study medication containers and unused study medication from the previous period of treatment were collected by study personnel; treatment compliance was reviewed with the subject; all AEs since the last visit were recorded and documented; concomitant medication information since the last visit were collected; distribution and collection of subject completed MENQOL form; distribution and collection of subject completed MOS-Sleep form; and instructions were given for reporting of serious adverse events that occur within 30 days after the last dose of the study medication.

¹¹ End of Treatment endometrial biopsies were repeated per protocol and were performed within 30 days of the final dose of study medication.

### C.1.8 Post Treatment Phone Contact Safety Follow Up (Approximately 15 Days After the Last Dose of Study Medication)

Each subject who received study medication received a follow-up phone call, regardless of the duration of therapy, approximately 15 days following the last dose of study medication. The follow-up generally took place after receipt of all safety assessments (e.g., endometrial biopsy and mammography results). The follow-up phone call included: review of ongoing adverse events and any new adverse events that occurred during the 15 days following the last dose of study medication; review of ongoing concomitant medications and any new concomitant medications that occurred during the 15 days following the last dose of study medication; discussion of all end of study safety assessments and determination if further follow up or clinic visit is required; and instructions were again provided for reporting of serious adverse events that occur within 30 days after the last dose of study medication.

### D. Subject Identification and Randomization Procedures

Each subject was given a unique subject number at the start of Screening that was used to identify their clinical site and sequential number. In addition to the assigned subject number, subject initials were also used for identification.

Eligible subjects were randomized at Visit 1 (Week 0/Day 1). The randomization code was created using a computer-generated randomization schedule prepared by a statistician prior to the start of the study.

### E. Test Product, Dose and Mode of Administration

### E.1 Study Medication Description and Packaging

TX-001HR is an oval, opaque, pink, soft gelatin formulation of a combination product comprising of 17β-estradiol hemihydrate and micronized progesterone.

Since two different sizes of capsules were necessary to accommodate the different doses, a double-dummy technique was used. The two sizes of placebo capsules were an identical match to the active study medication, but without the estrogen/progesterone.

Study medication was packaged in blister packs, labeled and sent to the each site. The carton and packaging labels did not contain information that unblinds the identity of the medication.

### E.2 Study Treatment

Subjects that were randomized to active treatment self-administered orally one of the following four arms of active TX-001HR treatment daily at bedtime with food for 12 months.
Treatment 1: Combined Estradiol 1 mg / Progesterone 100 mg formulation [large active; small placebo]
Treatment 2: Combined Estradiol 0.5 mg / Progesterone 100 mg formulation [large active; small placebo]
Treatment 3: Combined Estradiol 0.5 mg / Progesterone 50 mg formulation [large placebo; small active]
Treatment 4: Combined Estradiol 0.25 mg / Progesterone 50 mg formulation [large placebo; small active]

Two placebo gel capsules matching the test product were taken orally by subjects participating in the VMS Substudy that were randomized to placebo. In order to maintain the study blind, the study had a double blind, double dummy treatment. Subjects randomized to active treatment took a placebo gel capsule matching the alternate capsule size from their active treatment. All subjects took 1 large and 1 small capsule.
Treatment 5: Placebo [large placebo; small placebo]

### E.3 Study Medication Administration

All subjects self-administered orally two capsules daily at bedtime with food for 12 months. Each subject was dispensed enough study medication to last until the next scheduled visit, with allowances for visit windows. The subjects were instructed to return the used and unused containers of study medication in the original packaging to the study site at Visits 2, 3, 4, 5, 6 and 7. The sites verified and documented compliance based on counts of dispensed/returned study medication and any additional information reported by the subjects (e.g., regarding lost capsules).

### E.4 Dispensing and Return of Study Medication

Study medication were dispensed to all eligible subjects at Visits 1 to 6. At Visits 2 to 7 subjects were instructed to return all used study medication containers and any unused study medication to study personnel in the original packaging, and were dispensed with the new medication for the subsequent period.

If a subject discontinued study participation or was terminated from the study, the subject was instructed to return all study medication containers and any unused study medication at the time of discontinuation/terminations.

### E.4 Study Medication Accountability/Compliance

The first day that the study medication was administered by the subject was considered Day 1 and all subsequent visits were based on this day. Compliance was determined from the subject diary and pill count through week 12; during this time, the subject was instructed to record all administrations taken and missed. After the week 12 visit, study medication compliance was monitored through counts of capsules dispensed and returned as well as explanatory information reported by the subject. Each study subject was required to be at least 80% compliant with study medication, based on capsule count over each study visit interval to be considered compliant. If a subject was less than 80% compliant, the investigator was instructed to discuss withdrawing the subject with the Medical Monitor.

Subjects were instructed to return completed study diaries to study personnel at their visits. Study personnel reviewed the subject diary with the subject to ensure proper documentation of study medication dosing and other information recorded on the diary. Upon return of the study medication containers, study personnel were responsible for recording the amount of study medication returned, the amount of study medication used by the subject, and the amount of study medication unused by the subject on a drug accountability log.

### F. Endpoints

### F.1 Key Endpoints

### F.1.1 Primary Efficacy Endpoints: Vasomotor Symptoms (VMS Substudy)

A minimum of 14 consecutive days of complete hot flush diary data was required during the baseline assessment at Screening, and these consecutive days had to occur within the last 14 days prior to the Randomization visit (not counting the Randomization visit day itself). The most recent 7 consecutive days of data prior to Randomization (Day-7 to Day-1) was used to determine the baseline number of mild, moderate and severe hot flushes for each subject. The number of moderate to severe hot flushes from these 7 days was also used to determine eligibility for the VMS Substudy.
Mean change in frequency of moderate to severe vasomotor symptoms from baseline to week 4 in an active treatment group compared with placebo.
Mean change in frequency of moderate to severe vasomotor symptoms from baseline to week 12 in an active treatment group compared with placebo.
Mean change in severity of moderate to severe vasomotor symptoms at baseline to mild, moderate to severe vasomotor symptoms at week 4 in an active treatment group compared with placebo.
Mean change in severity of moderate to severe vasomotor symptoms at baseline to mild, moderate to severe vasomotor symptoms at week 12 in an active treatment group compared with placebo.

### F.1.2 Primary Safety Endpoint: Endometrial Hyperplasia

The primary safety endpoint was the incidence rate of endometrial hyperplasia at 12 months (to demonstrate a hyperplasia rate that is ≤1 percent with an upper bound of the one-sided 95 percent CI for that rate that does not exceed 4 percent) based on an a priori plan in which a consensus among two out of three pathologists is the final endometrial pathology diagnosis. When the two primary pathologists disagree on the presence of hyperplasia, the read of the third pathologist will be utilized.

For the primary endpoint, all endometrial biopsies were centrally read by three pathologists. Two pathologists designated by the sponsor, were considered to be the primary pathologists (the pathologists were blinded to this designation).

Each pathologist classified the biopsies into one of the following three categories:
Category 1: Non-endometrial malignancy/non-hyperplasia- includes proliferative endometrium, weakly proliferative endometrium, disordered proliferative pattern, secretory endometrium, endometrial tissue (other) [i.e., benign, inactive or atrophic fragments of endometrial epithelium, glands, stroma, etc.], endometrial tissue insufficient for diagnosis, no endometrium identified, no tissue identified, other.
Category 2: Endometrial hyperplasia- includes simple hyperplasia with or without atypia and complex hyperplasia with or without atypia.
Category 3: Endometrial malignancy.

The reads of the two primary pathologists were utilized. Consensus was reached when the two primary pathologist readers agreed on any of the above categories. For example, any 2 subcategories of “Non-endometrial malignancy/non-hyperplasia” will be classified as “Category 1: Non-endometrial malignancy/non-hyperplasia,” if the primary pathologists disagreed on the presence of hyperplasia, the result of the third pathologist was utilized and the final decision regarding the presence of hyperplasia was based on the diagnosis of the majority.

If all three readings are disparate (i.e., each falls into a different category- Category 1, 2, or 3), the final diagnosis was based on the most severe of the three readings.

A confidence interval approach was used to determine if the hyperplasia incidence rate was acceptable. For each active treatment group, the incidence rate of hyperplasia at year 1 and the associated upper 95% 1-sided confidence limit was be calculated. An observed incidence rate of 1% or less with an upper 1-sided 95% confidence limit of 4% or less was considered acceptably low.

### F.2 Secondary VMS SubStudy Endpoints

Secondary VMS SubStudy Endpoints include the following:
Mean change in frequency of moderate to severe vasomotor symptoms from baseline to each week up to week 12.
Mean change in severity of moderate to severe vasomotor symptoms from baseline to mild, moderate to severe vasomotor symptoms each week up to week 12.
Mean change in frequency and severity of mild, moderate and severe vasomotor symptoms from baseline to each week up to week 12.
Percent of subjects with 50% and, separately, 75% reduction in moderate to severe vasomotor symptoms from baseline at each week up to week 12.
Percent treatment responders at weeks 4, 8 and 12 based on subject satisfaction with treatment (Clinical Global Impression [CGI]) compared to changes in frequency of moderate to severe vasomotor symptoms from baseline.

### F.3 Secondary Endometrial Hyperplasia Endpoint

A supplemental secondary analysis was performed. The secondary endpoint was the incidence rate of endometrial hyperplasia at 12 months based on agreement of two of the three pathologists’ reads. All biopsies were read by three blinded pathologists, and the results from the three pathologists were utilized. In this supplemental analysis, the final diagnosis was based on agreement of two of the three pathologist reads. Consensus was reached when two of the three pathologist readers agreed on any of the above categories. For example, any 2 subcategories of “Non-endometrial malignancy/non-hyperplasia” was classified as “Category 1: Non-endometrial malignancy/non-hyperplasia.” If all three readings were disparate (i.e., each falls into a different category- Category 1, 2, or 3), the final diagnosis was based on the most severe of the three readings.

### F.4 Other Secondary Endpoints

Other secondary endpoints included the following:
Proportion of subjects with cumulative amenorrhea from day 1 to day 364
No bleeding: % by cycle and cumulative for consecutive cycles.
Number of days with bleeding/spotting.
Evaluation of frequency and severity of hot flushes over 12 weeks in the overall study subjects.
MENQOL evaluation parameters.
MOS-Sleep evaluation parameters.
Trough hormone assessment for serum estradiol and estrone.

The severity of vasomotor symptoms is defined clinically as follows:

| | |
|---|---|
| Severity | Description |
| Mild | Sensation of heat without sweating |
| Moderate | Sensation of heat with sweating, able to continue activity |
| Severe | Sensation of heat with sweating, causing cessation of activity |

### F.5 Safety Endpoints

Vital signs, weight, changes in clinical laboratory measurements (including hematology, clinical chemistry, urinalysis, and Pap smear), and adverse events were evaluated as part of the safety endpoints. Changes in physical exam, ECG, pelvic exam, and mammogram were evaluated. The incidence of hyperplastic polyps and polyps associated atypia were considered in the safety review. The comparability of the active treatment groups with regard to the frequency and severity distribution of any adverse event reported by at least 5% of the subjects in either the treatment groups would be evaluated by comparing the frequency distributions of the 5 treatment groups.

### G. Statistical Considerations

### G.1 Randomizationand Stratification

Subjects in the VMS Substudy were randomized within each study site to one of the treatment groups below in a 1:1:1:1:1 allocation ratio. Subjects not in the VMS Substudy were randomized to one of the active treatment groups in a 1:1:1:1 allocation ratio. Subjects were randomized to study medication within each site using a reproducible, computer-generated block randomization schedule. Randomization codes were generated and held with restricted access to decrease the chance of unblinding and to minimize bias.
Treatment 1: Combined Estradiol 1 mg / Progesterone 100 mg formulation
Treatment 2: Combined Estradiol 0.5 mg / Progesterone 100 mg formulation
Treatment 3: Combined Estradiol 0.5 mg / Progesterone 50 mg formulation
Treatment 4: Combined Estradiol 0.25 mg / Progesterone 50 mg formulation
Treatment 5: Placebo

### G.2 Sample Size Rationale

The overall study sample size was based on the target that the combination therapy is effective at achieving a ≤ 1% incidence rate of endometrial hyperplasia following 12 months of therapy and that the upper bound of the 95% confidence interval of the estimated incidence rate is ≤ 4%. The VMS sub-study sample size was based on the expected changes in average weekly frequency and severity of vasomotor symptoms from baseline to weeks 4 and 12.

Overall Study: With 250 subjects in each active treatment group completing 12 months of treatment and being successfully evaluated for endometrial hyperplasia at baseline and 12 months, two or fewer reports of endometrial hyperplasia would result in an annual incidence rate of ≤ 1% and an upper bound on a one-sided 95% confidence interval of ≤ 2.5% (exact binomial).

VMS Sub-study: The primary method of analysis for both frequency counts and severity index was to account for missing information using imputation by last observation carried forward and a linear mixed effects covariance pattern model that treated subjects as a random effect and accounted for the repeated frequency and severity measures at baseline, week 4 and week 12. Each of the four active treatment groups and the 4 co-primary outcomes were compared to the placebo group in a hierarchical order to preserve the test level of significance for each comparison at 5% (two-sided). A two-group t-test was used to estimate sample size requirements.

Change in frequency: The mean changes from baseline in weekly frequency of moderate to severe hot flushes was assumed to be at least -56 for any given active treatment group and -35 for the placebo group at both weeks 4 and 12. A common, between subject standard deviation of 35 across treatment groups and weeks was further assumed.

Change in severity index: The mean changes in the severity score from baseline for mild, moderate and severe hot flushes was assumed to be at least -0.7 for any given active treatment group and -0.4 for the placebo group at both weeks 4 and 12. A common, between subject standard deviation of 0.6 across treatment groups and weeks was further assumed.

Enrolling 150 subjects in each treatment group and allowing for up to 20% of the subjects in each group being ineligible for the primary analyses provides at least 90% power to test the primary VMS hypotheses.

### G.3 Datasets to Be Analyzed

Population datasets for analyses are defined below for the estimation of endometrial hyperplasia in the active treatment groups, for the comparison between active treatment groups and the placebo group of the change from baseline in the weekly frequency and severity of moderate to severe hot flushes in the VMS Sub-study, and for the overall safety evaluation.
All Randomized: All subjects that are randomized into the study.
All Treated/Safety: All randomized subjects that took at least one application of study treatment.
Endometrial Hyperplasia (EH): All Treated subjects randomized to an active treatment group who remain on study treatment for 12 months, no major protocol violations, and have a biopsy at baseline and month 12 that can be evaluated for the presence of endometrial hyperplasia. In addition, if endometrial hyperplasia is diagnosed at a mid-study visit in a subject who had a definitive biopsy at baseline, the subject with the endometrial hyperplasia event will be counted in calculating the annual incidence.
VMS Sub-study Modified Intent-to-Treat (MITT): All Treated subjects who qualify for the VMS Sub-study, had baseline measurement of frequency and severity of moderate to severe hot flushes, and had at least one week of reporting of frequency and severity of hot flushes following initiation of study treatment;
VMS Sub-study Efficacy Evaluable (EE): MITT subjects who were correctly included at randomization for efficacy reasons and who had all 4-week and 12-week efficacy evaluation data.

Efficacy analyses for endometrial hyperplasia will be performed using the EH population, efficacy analyses for VMS will be performed using the MITT and EE populations, and safety analyses will be performed using the All Treated/Safety population.

Other analyses of spotting and bleeding, flushing experience in non-Substudy subjects, MENQOL and MOS-Sleep responses will rely on available data.

Subjects with major deviations from the protocol as specified in the Statistical Analysis Plan will be excluded from the analyses.

### G.4 Analyses

### G.4.1 Disposition of Subjects

The number of subjects who are randomized, treated, attended the various assessments, and who complete the clinical trial were tabulated.

### G.4.2 Demographic and Other Subject Characteristics

Subject demographics and pre-treatment characteristics were summarized descriptively. No statistical tests were performed to compare demographics between treatment assignments.

### G.4.3 Pre-Trial and Concomitant Medications

Concomitant Medications included any medication or health product taken during the active study treatment period. Pre-trial medications included any medications taken within 30 days of randomization. The number of subjects and percent using medications were tabulated according to the medication’s World Health Organization (WHO)Therapeutic Drug Class and Generic Term (2010 March dictionary or later) by treatment assignment for the All Treated population. There are separate tables for Pre-trial and Concomitant Medications. Subjects taking a medication more than once were only counted once for that medication.

### G.5 Interim Analysis

No interim analysis was planned.

### G.6 Final Statistical Analysis Plan

A final Statistical Analysis Plan was signed off by the Sponsor prior to database lock. The Statistical Analysis Plan provided a detailed description of all intended analyses.

### G.6.1.1 Data Handling of Hot Flush Data

Frequency and severity of hot flushes were recorded from the Screening visit through the week twelve visit using a daily hot flush diary. The number of mild, moderate or severe hot flushes for the fourth and twelfth weeks was calculated by adding up the number of moderate and severe hot flushes from each of the seven days from the fourth and twelfth weeks.

A minimum of 14 consecutive days of complete hot flush diary data was required during the baseline assessment at Screening, and these consecutive days had to occur within the last 14 days prior to the Randomization visit (not counting the Randomization visit day itself). The most recent 7 consecutive days of data prior to randomization (Day -7 to Day -1) were used to determine the baseline number of mild, moderate and severe hot flushes for each subject. The number of moderate to severe hot flushes from these 7 days was also be used to determine eligibility for the VMS Substudy. (To participate in the VMS Substudy, a subject must have also reported ≥7 moderate to severe hot flushes per day, or ≥50 per week, at the baseline assessment during Screening; subjects whose hot flushes were less frequent still could participate as non-Substudy subjects until enrollment of the non-VMS Substudy was reached.)

The primary endpoints were summarized for the baseline visit, change from baseline to week 4, and change from baseline to week 12. For each visit, the sample size, mean, median, minimum, maximum, and an estimate of the pooled SD will be presented.

Diary data that is performed after the last day of dosing will be excluded from all analyses.

Missing Data: If there was missing data within a week of diary data, the following methods were used to estimate missing data.
For weeks with at least four days of evaluable data, the missing days of data within that week were estimated by the average of the non-missing days within that week. Once the missing values within the week hae been replaced with the average, the 7 days of values were used to calculate the score for that week.
For weeks with three or less days of data, the data for the week was excluded from the analysis unless the subject’s data only consists of weeks that had less than four days of data. This resulting dataset was considered the observed cases data.
Any weeks that are totally composed of missing values (due to having three or less days of data) were replaced using LOCF. In the LOCF analysis, weeks of missing data were replaced with the previous week of data unless the previous week of data is baseline.

### G.6.1.2 Data Handling of Hot Flush Data

### Primary Efficacy Analyses-VMS

The weekly number of moderate to severe hot flushes and the weekly severity score were assessed as mean changes from baseline to weeks 4 and 12 for each subject.

The weekly number of moderate to severe hot flushes for each assessment week (baseline, and weeks 4 and 12) were derived as:
Weekly Frequency = (total number of moderate and severe hot flushes for the 7 days of the subject week)
The weekly severity of hot flushes for each assessment week (baseline, and weeks 4 and 12) will be derived as:
   Weekly Severity Score = [(number of mild hot flushes for 7 days) x 1 +
   (number of moderate hot flushes for 7 days) x 2 +
   (number of severe hot flushes for 7 days) x 3] /-
   (total number of hot flushes over 7 days).

A weekly severity score of zero (0) will be assigned for subjects reporting no hot flushes for a given assessment week.

Absolute, changes from baseline and respective differences from placebo in frequency and severity of vasomotor symptoms were listed and summarized. Means, standard deviations (S.D.), minimum (MIN) and maximum (MAX) were provided for all 4 co-primary endpoints.

Mean changes from baseline in frequency and severity of vasomotor symptoms was assessed using a linear mixed effects covariance pattern model with baseline as a covariate, treatment (active vs. placebo), week (4 and 12) and treatment by week interaction. Subject was included in the model as a random effect and a compound symmetry model was assumed. A test for an overall treatment effect was assessed, as well as, weekly comparisons of active to placebo. A separate mixed model was run for each active dose comparing back to placebo. Statistical significance was declared if p<0.05 for each dose comparison of each of the 4 co-primary endpoints. The highest estradiol dose (1 mg) was tested first for each of the 4 co-primary endpoints and all endpoints should reach statistical significance compared to placebo prior to moving to the next test. The second test was a three-way comparison of the two 0.5 mg doses and placebo; if each of the 4 co-primary endpoints demonstrate statistical significance then separate pairwise comparisons would be made between each 0.5 mg doses and placebo. Finally, if all pairwise comparisons with placebo of the 0.5 mg doses were statistically significant then the 0.5 mg dose was compared to placebo. Ninety five percent (95%), two-sided confidence intervals were derived for changes from baseline and respective differences from placebo, and graphically displayed for each dose and week.

### Primary Safety Analysis-Endometrial Hyperplasia

The primary safety endpoint was the incidence rate of endometrial hyperplasia at 12 months (to demonstrate a hyperplasia rate that is ≤1 percent with an upper bound of the one-sided 95 percent CI for the rate that does not exceed 4 percent) based on an a priori plan in which a consensus among two out of three pathologists was the final endometrial pathology diagnosis. When the two primary pathologists disagreed on the presence of hyperplasia, the read of the third pathologist was utilized.

For the primary endpoint, all endometrial biopsies were centrally read by three pathologists. Two pathologists, designated by the sponsor, were considered to be the primary pathologists (the pathologists were blinded to this designation).

Each pathologist classified the biopsies into one of the following three categories:
Category 1: Non-endometrial malignancy/non-hyperplasia (includes proliferative endometrium, weakly proliferative endometrium, disordered proliferative pattern, secretory endometrium, endometrial tissue (other) [i.e., benign, inactive or atrophic fragments of endometrial epithelium, glands, stroma, etc.], endometrial tissue insufficient for diagnosis, no endometrium identified, no tissue identified, other.
Category 2: Endometrial hyperplasia- includes simple hyperplasia with or without atypia and complex hyperplasia with or without atypia.
Category 3: Endometrial malignancy.

For the primary analysis:

The reads of the two primary pathologists were utilized. Consensus was reached when both of the primary pathologist readers agreed on any of the above categories. For example, any 2 subcategories of “Non-endometrial malignancy/non-hyperplasia” were classified as “Category 1: Non-endometrial malignancy/non-hyperplasia”; if the primary pathologists disagreed on the presence of hyperplasia, the result of the third pathologist was utilized and the final decision regarding the presence of hyperplasia was based on the diagnosis of the majority.

If all three readings were disparate (i.e., each falls into a different category- Category 1, 2, or 3), the final diagnosis was based on the most severe of the three readings.

A confidence interval approach was used to determine if the hyperplasia incidence rate was acceptable. For each active treatment group, the incidence rate of hyperplasia at year 1 and the associated upper 95% 1-sided confidence limit was calculated. The primary analysis population for endometrium hyperplasia was endometrial hyperplasia (EH) population. A EH subject at year 1 is one who is randomly assigned and takes at least 1 dose of study medication, with no exclusionary protocol violation (as detailed at the Statistical Analysis Plan) and has a pretreatment endometrial biopsy and a biopsy at year 1, or who has developed endometrial hyperplasia at any time during the study. Any biopsy performed within the month 11 through month 13 (relative to the first day on study medication) will be considered a year 1 biopsy, and it will be included in the analysis as long as it was done within 30 days of the last dose of study medication.

The incidence rate of endometrial hyperplasia at year 1 was calculated as follows:
I = A/B
where I = incidence rate at 1 year evaluation,
A = all subjects with biopsies positive for endometrial hyperplasia during the study,
B = all subjects with biopsies during months 11 through 13 meeting the criteria specified above, plus all subjects with biopsies positive for endometrial hyperplasia by any of the pathologist before month 11.

An observed incidence rate of 1% or less with an upper 1-sided 95% confidence limit of 4% or less will be considered acceptably low.

In addition, 95% 2-sided confidence intervals were calculated for pairwise differences between groups in hyperplasia incidence rates.

### G.6.1.3 Secondary VMS Analyses

Similar to the co-primary endpoints for weeks 4 and 12, assessments were made for changes in frequency and severity of mild, moderate and severe vasomotor symptoms for each assessment week up to week 12. Two assessments were made with one including only moderate and severe vasomotor symptoms and one including mild, moderate and severe vasomotor symptoms. Severity scores were set to zero (0) for subjects reporting no moderate or severe flushes for the moderate and severe assessment, and set to zero (0) for subjects reporting no hot flushes for the mild moderate and severe assessments. Additionally, percent reductions from baseline and respective differences from placebo were reported.

The percent of treatment responders was identified within each treatment arm and compared (active treatments to placebo) at weeks 4, 8, and 12 by using the methodology described by Gerlinger et al., supra, that combines subject self-assessment of satisfaction with treatment (using the CGI) and changes in reported moderate to severe flushes. In particular, the Gerlinger approach first stratifies subjects’ change from baseline in the number of moderate to severe hot flushes by their CGI response (7 levels ranging from “very much worse” to “very much better”); then discriminant analysis is applied to identify minimally clinically important reductions in the number of hot flushes that study subjects perceive as beneficial and that are then used to define a responder.

A second approach to identifying treatment responders was to calculate the percent of subjects with 50% and, separately, 75% reduction from baseline in moderate to severe vasomotor symptoms at each week up to week 12 and compare between active and placebo treatments.

### G.6.1.4 Secondary Endometrial Hyperplasia Analysis

A supplemental secondary analysis was performed. The results from the three pathologists were utilized. In this supplemental analysis the final diagnosis was based on agreement of two of the three pathologist reads. Consensus was reached when two of the three pathologist readers agreed on any of the above categories. For example, any 2 subcategories of “Non-endometrial malignancy/non-hyperplasia” was classified as “Category 1: Non-endometrial malignancy/non-hyperplasia.” If all three readings were disparate (i.e., each fell into a different category - Category 1, 2, or 3), the final diagnosis was based on the most severe of the three readings.

### G.6.2 Other Analyses

Other analyses included the following:
Proportion of subjects with cumulative amenorrhea from day 1 to day 364.
No bleeding: % by cycle and cumulative for consecutive cycles.
Number of days with bleeding/spotting.

Percent amenorrhea: Amenorrhea is defined as absence of bleeding or spotting. Within each treatment arm, the portion of subjects with cumulative amenorrhea from day 1 to day 364 was calculated and compared between active and placebo treatments.

Percent no bleeding: No bleeding is defined as absence of bleeding. Within each treatment arm, the percent of subjects with no bleeding was calculated by cycle and for consecutive cycles and compared between active and placebo treatments.

Number of days with bleeding/spotting: The number of days with bleeding/spotting, as reported on subject diaries, was summarized by cycle and treatment group.

Changes in frequency and severity of hot flushes over 12 weeks in non-Substudy subjects: Hot flush experience in the non-Substudy group (all actively treated subjects) was evaluated in the manner described earlier for the secondary analysis of the VMS Substudy data.

MENQOL: The menopause-specific quality of life questionnaire assesses changes in quality of life of study subjects over a one month period. It was self-administered and was measured at baseline and at week 12, month 6 and month 12 during the study. It is composed of 29 questions distributed across 4 domains: vasomotor, psychosocial, physical and sexual. There is no total score so domain scores were analyzed separately. Change in monthly scores were summarized and described within each treatment group.

MOS-Sleep: The Medical Outcomes Study Sleep self-report questionnaire has 12 items that measure six dimensions of sleep over the past four weeks. It was self-administered and was measured at baseline and was measured at baseline and at week 12, month 6 and month 12 during the study. Change in scores over the past four weeks (total and subscales) was analyzed within each treatment.

### G.6.3 Serum Concentrations of Estradiol, Estrone, and Progesterone

Serum concentrations of estradiol obtained at Screening Visit 1 were summarized.

Serum concentrations of estradiol and estrone obtained at Screening and Visits 2, 4, 5, 6 and 7 and serum concentrations of progesterone obtained at Screening and Visits 4 and 7 were summarized by treatment.

### G.6.4 Safety and Tolerability Analyses

Safety and tolerability were assessed by summarizing the incidence, relatedness, severity, and type of adverse events and treatment-emergent changes in safety evaluation criteria. Safety evaluation results were listed for all subjects and summaries will be tabulated by treatment. All safety and tolerability analyses were descriptive and used the All Treated/Safety population.
Adverse Events: The number (percentage) of subjects with at least one treatment-emergent AE will be presented in a frequency table by MedDRA system-organ class and per MedDRA “preferred” term. A similar summary will be created for SAEs and AEs resulting in interruption of treatment or leading to discontinuation of study medication. Summaries will also be presented by severity and relationship to study medication.
Vital Signs, Weight, ECGs, Mammograms, and Clinical Laboratory Values: Change from baseline will be summarized over time and any abnormal values considered clinically significant will be tabulated. Shift tables will be generated.
Physical Examinations: Changes from baseline to final evaluation will be categorized as improved, no change, or worsened for each body system. The number and percentage of subjects in each category of change will be given at final evaluation for each treatment group and body system.

### G.6.5 Treatment Exposure and Compliance

Duration of exposure to study medication and compliance with administration of study medication were summarized by treatment group. Compliance was estimated as the percent of study medication actually used compared to the theoretical amount of drug that could have been used during each subject’s exposure to study medication. No adjustment were made for missed doses or interruption of study medication.

### H. Initial Efficacy Results and Analysis

The Replenish Trial studied TX-001HR, an investigational bio-identical hormone therapy combination of 17ß-estradiol and natural progesterone in a single, oral softgel, for the treatment of moderate to severe vasomotor symptoms (VMS) due to menopause in post-menopausal women with an intact uterus.

As explained above, the Replenish Trial evaluated four doses of TX-001HR and placebo in 1,835 post-menopausal women between 40 and 65 years old. The doses studied were as follows:
17ß-estradiol 1 mg/progesterone 100 mg (n = 416)
17ß-estradiol 0.5 mg/progesterone 100 mg (n = 423)
17ß-estradiol 0.5 mg/progesterone 50 mg (n = 421)
17ß-estradiol 0.25 mg/progesterone 50 mg (n = 424)
Placebo (n = 151)

The results of the Replenish Trial demonstrated:
TX-001 HR estradiol 1 mg/progesterone 100 mg and TX-001HR estradiol 0.5 mg/progesterone 100 mg both achieved all four of the co-primary efficacy endpoints and the primary safety endpoint (see, Tables 26-29 as well as Table 30, below).
TX-001HR of estradiol 1 mg/progesterone 100 mg and TX-001HR estradiol 0.5 mg/progesterone 100 mg both demonstrated a statistically significant and clinically meaningful reduction from baseline in both the frequency and severity of hot flashes compared to placebo (see, Tables 26-29, below).
TX-001HR estradiol 0.5 mg/progesterone 50 mg and TX-001HR estradiol 0.25 mg/progesterone 50 mg were not statistically significant at all of the co-primary efficacy endpoints (see, Tables 26-29, below). The estradiol 0.25 mg/progesterone 50 mg dose was included in the clinical trial as a non-effective dose to meet the requirement of the FDA guidance to identify the lowest effective dose.
The incidence of consensus endometrial hyperplasia or malignancy was 0% across all four TX-001HR doses, meeting the recommendations as established by the U.S. Food and Drug Agency’s (FDA)¹² draft guidance (see,Table 30, below).

¹² 2003 FDA Draft Guidance for Industry Estrogen and Estrogen/Progestin Drug Products to Treat Vasomotor Symptoms and Vulvar and Vaginal Atrophy Symptoms - Recommendations for Clinical Evaluation http://www.fda.gov/ucm/groups/fdagov-public/@fdagov-drugs-gen/docmnents/document/ucm071643.pdf.

As outlined in the FDA guidance, the co-primary efficacy endpoints in the Replenish Trial were the change from baseline in the number and severity of hot flashes at weeks 4 and 12 as compared to placebo.¹³ The primary safety endpoint was the incidence of endometrial hyperplasia with up to 12 months of treatment. General safety was also evaluated.

¹³ Id.

The results of the Replenish Trial (p-values of < 0.05 meet FDA guidance and support evidence of efficacy) are summarized, below, in Table 25 (as well as in Table 31) and included multimedia.

TABLE 25

Overview/Summary of Replenish Results
Replenish Trial Co-Primary Efficacy Endpoints: Mean Change in Frequency and Severity of Hot Flashes Per Week Versus Placebo at Weeks 4 and 12, VMS-MITT Population

| | | | | | |
|---|---|---|---|---|---|
| Estradiol/Progesterone | 1 mg/100 mg (n = 141) | 0.5 mg/100 mg (n = 149) | 0.5 mg/50 mg (n = 147) | 0.25 mg/50 mg (n = 154) | Placebo (n = 135) |

Frequency

| | | | | | |
|---|---|---|---|---|---|
| Week 4 P-value versus placebo | <0.001 | 0.013 | 0.141 | 0.001 | - |
| Week 12 P-value versus placebo | <0.001 | <0.001 | 0.002 | <0.001 | - |

Severity

| | | | | | |
|---|---|---|---|---|---|
| Week 4 P-value versus placebo | 0.031 | 0.005 | 0.401 | 0.100 | - |
| Week 12 P-value versus placebo | <0.001 | <0.001 | 0.018 | 0.096 | - |

Replenish Trial Primary Safety Endpoint: Incidence of Consensus Endometrial Hyperplasia or Malignancy up to 12 months, Endometrial Safety Population (Ŧ)

| | | | | | |
|---|---|---|---|---|---|
| Endometrial Hyperplasia | 0% (0/280) | 0% (0/303) | 0% (0/306) | 0% (0/274) | 0% (0/92) |

MITT = Modified intent to treat
^{Ŧ}Per FDA, consensus hyperplasia refers to the concurrence of two of the three pathologists be accepted as the final diagnosis’
P-value < 0.05 meets FDA guidance and supports evidence of efficacy

The Replenish study also demonstrated a dose response favoring the higher doses of estradiol in combination with progesterone. Importantly, multiple doses of TX-001HR allow for the ability to individualize therapy to meet the needs of a diverse population of women.

The most common adverse events (>5%) reported in the active treatment groups were breast tenderness, headache, nasopharyngitis, abdominal pain, upper respiratory infection, nausea, and back pain. Surprisingly, there was a very low reported incidence of adverse events of somnolence with TX-001HR, in contrast to commercially available oral progesterone (such as the reference listed drug Prometrium) where somnolence has been reported as a significant side effect. There were no unexpected safety signals.

TX-001HR estradiol 1 mg/progesterone 100 mg and estradiol 0.5 mg/progesterone 100 mg were also associated with clinically meaningful and statistically significant improvements in secondary endpoints including:
Menopause-Specific Quality of Life (MENQOL), a self-reported quality of life measure that had statistically significant improvements in the total score and vasomotor symptoms domain (see, Tables 32-35);
Clinical Global Impression (CGI) scale, a well-established patient reported outcome tool that showed a statistically significant and clinically meaningful improvement (see, Table 36); and
Responder analysis of the reduction of hot flashes of greater than 50% and 75% were statistically significant in the active treatment groups as compared to placebo (see, Table 37).

Based on the results, it is clear that multiple doses of doses of TX-001HR studied in the Replenish Trial provided positive results, demonstrating that that this drug product candidate is capable of addressing the significant demand for bio-identical hormone therapy. Over the past 14 years, women in the U.S. have moved from synthetic FDA-approved hormone therapy to unapproved bio-identical hormones mixed together or compounded at independent or community based pharmacies. The goal of the Replenish Trial was to provide a proven safe and effective bio-identical combination hormone therapy to meet the needs of women, physicians and pharmacies, that have largely been ignored by the pharmaceutical industry, and it has been found that TX-001HR at multiple different doses does just that. TX-001HR, quite importantly, provides the hormonal solution that women demand with proven safety and efficacy.

TX-001HR is the first bio-identical hormone therapy of estradiol in combination with natural progesterone to be evaluated in a large, well-controlled, randomized clinical trial. The Replenish Trial demonstrated for the first-time safety and robust efficacy for the treatment of hot flashes of multiple doses of estradiol in combination with natural progesterone with a consistency of effect noted on the primary and secondary efficacy endpoints. Importantly, TX-001HR establishes the FDA required endometrial safety for multiple doses of estradiol in combination with natural progesterone in continuous combined regimen.

TABLE 26

Change From Baseline to Weeks 4 and 12 in Frequency Of Moderate To Severe Vasomotor Symptoms - MMRM MITT-VMS Population

| | TRT1 (N=141) | | TRT2( N=149) | | TRT3 (N=147) | | TRT4 (N=154) | | Placebo (N=135) | |
|---|---|---|---|---|---|---|---|---|---|---|
| Study Week | Actual Value | Change From Baseline | Actual Value | Change From Baseline | Actual Value | Change From Baseline | Actual Value | Change From Baseline | Actual Value | Change From Baseline |
| Baseline | | | | | | | | | | |
| N | 141 | | 149 | | 147 | | 154 | | 135 | |
| Mean (SD) | 74.4 (35.26) | | 72.1 (27.76) | | 75.9 (28.04) | | 77.0 (30.42) | | 72.4 (23.26) | |
| Min, Max | 0, 331 | | 38, 215 | | 29, 190 | | 32, 208 | | 10, 155 | |
| Median | 66 | | 62 | | 66 | | 65 | | 66 | |

| Week 4 | | | | | | | | | | |
|---|---|---|---|---|---|---|---|---|---|---|
| N | 134 | 134 | 144 | 144 | 142 | 142 | 152 | 152 | 126 | 126 |
| Mean (SD) | 31.5 (29.45) | -40.6 (30.59) | 37.2 (26.68) | -35.1 (29.14) | 41.5 (33.85) | -33.6 (30.64) | 38.4 (32.79) | -38.9 (31.04) | 45.9 (27.52) | -26.4 (27.05) |
| Min, Max | 0, 161 | -140, 51 | 0, 115 | -177, 23 | 0, 233 | -140, 105 | 0, 207 | -25, 41 | 0, 164 | -122, 80 |
| Median | 23 | -41 | 35 | -32 | 34 | -35 | 30 | -36 | 41 | -24 |
| Difference from Placebo[1] | | | | | | | | | | |
| LS Mean | | -12.81 | | -8.07 | | -4.81 | | -10.40 | | |
| Standard Error | | 3.30 | | 3.25 | | 3.26 | | 3.22 | | |
| 95% C1 | | (-19.29, -6.32) | | (-14.46, -1.68) | | (-11.21, 1.59) | | (-16.73, -4.08) | | |
| P-value | | <0.001 | | 0.013 | | 0.141 | | 0.001 | | |
| Week 12 | | | | | | | | | | |
| N | 124 | 124 | 129 | 129 | 124 | 124 | 135 | 135 | 115 | 115 |
| Mean (SD) | 17.1 (20.65) | -55.1 (31.36) | 19.1 (21.87) | -53.7(31.93) | 25.2(27.45) | -50.2(31.35) | 24.1 (28.42) | -52.4(33.90) | 32.0(26.24) | -40.2(29.79) |
| Min, Max | 0, 84 | -207, 19 | 0, 92 | -177, 26 | 0, 129 | -148, 20 | 0, 178 | -159, 68 | 0, 131 | -155, 38 |
| Median | 9 | -53 | 14 | -51 | 16 | -51 | 16 | -51 | 30 | -40 |
| Difference from Placebo[1] | | | | | | | | | | |
| LS Mean | | -16.58 | | -15.07 | | -10.79 | | -11.71 | | |
| Standard Error | | 3.44 | | 3.39 | | 3.41 | | 3.36 | | |
| 95% CI | | (-23.33,-9.82) | | (-21.72, -8.42) | | (-17.48,-4.10) | | (-18.31,-5.11) | | |
| P-value | | <0.001 | | <0.001 | | 0.002 | | <0.001 | | |

[1] Derived from the MMRM model with Treatment, Week (1 -12), Treatment-by-Week interaction as factors, Baseline as covariate, and Subject as repeated measures unit. Difference is estimated from the simple contrast between the least squares means.

TABLE 27

Change From Baseline to Weeks 4 and 12 in Frequency Of Moderate To Severe Vasomotor Symptoms - LOCF ANCOVA MITT-VMS Population

| Study Week | TRT1 (N=141) | | TRT2 (N=149) | | TRT3 (N=147) | | TRT4 (N=154) | | Placebo (N=135) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Baseline | | | | | | | | | | |
| N | 141 | | 149 | | 147 | | 154 | | 135 | |
| Mean (SD) | 74.4 (35.26) | | 72.1 (27.76) | | 75.9 (28.04) | | 77.0 (30.42) | | 72.4 (23.26) | |
| Min, Max | 0, 331 | | 38, 215 | | 29, 190 | | 32, 208 | | 10, 155 | |
| Median | 66 | | 62 | | 66 | | 65 | | 66 | |
| Week 4 | | | | | | | | | | |
| N | 141 | 141 | 149 | 149 | 147 | 147 | 154 | 154 | 135 | 135 |
| Mean (SD) | 35.4 (37.79) | -39.0 (30.97) | 37.7 (26.45) | -34.4 (28.93) | 43.4 (35.76) | -32.5 (30.72) | 38.3 (32.59) | -38.7 (30.89) | 45.8 (27.23) | -26.67.31) |
| Min, Max | 0, 294 | -140, 51 | 0, 115 | -177, 23 | 0, 233 | -140, 105 | 0, 207 | -125, 41 | 0, 164 | -122, 80 |
| Median | 25 | -40 | 36 | -31 | 34 | -34 | 30 | -36 | 41 | -22 |
| Difference from Placebo [1] | | | | | | | | | | |
| LS Mean | | -11.60 | | -7.97 | | -4.50 | | -10.19 | | |
| Standard Error | | 3.28 | | 3.23 | | 3.25 | | 3.21 | | |
| 95% CI | | (-18.04, -5.17) | | (-14.32. -1.62) | | (-10.88, 1.87) | | (-16.50, -3.89) | | |
| P-value | | <0.001 | | 0.014 | | 0.166 | | 0.002 | | |
| Week 12 | | | | | | | | | | |
| N | 141 | 141 | 149 | 149 | 147 | 147 | 154 | 154 | 135 | 135 |
| Mean (SD) | 21.0 (34.96) | -53.4 (32.93) | 20.0 (22.61) | -52.0 (30.85) | 27.3 (31.01) | -48.6 (33.25) | 26.3 (30.67) | -50.7 (34.06) | 36.0 (30.41) | -36.4 (31.95) |
| Min, Max | 0, 294 | -207, 22 | 0, 92 | -177, 26 | 0, 171 | -148, 49 | 0, 178 | -159, 68 | 0, 164 | -155, 80 |
| Median | 9 | -53 | 14 | -51 | 18 | -50 | 18 | -51 | 33 | -33 |
| Difference from Placebo [1] | | | | | | | | | | |
| LS Mean | | -15.83 | | -15.84 | | -10.12 | | -11.60 | | |
| Standard Error | | 3.32 | | 3.28 | | 3.29 | | 3.26 | | |
| 95% CI | | (-22.35,-9.31) | | (-22.27,-9.41) | | (-16.58, -3.66) | | (-17.99, -5.21) | | |
| P-value | | <0.001 | | <0.001 | | 0.002 | | <0.001 | | |

[1] Derived from the ANCOVA model with Treatment as factor and Baseline as covariate. Difference is estimated from the simple contrast between the least squares means.

TABLE 28

Change From Baseline to Weeks 4 and 12 in Severity Of Moderate To Severe Vasomotor Symptoms - MMRM MITT-VMS Population

| Study Week | TRT1 (N=141) | | TRT2 (N=149) | | TRT3 (N=147) | | TRT4 (N=154) | | Placebo (N=135) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Baseline | | | | | | | | | | |
| N | 141 | | 149 | | 147 | | 154 | | 135 | |
| Mean (SD) | 2.54 (0.320) | | 2.51 (0.249) | | 2.50 (0.231) | | 2.51 (0.262) | | 2.52 (0.246) | |
| Min, Max | 0.00, 3.00 | | 2.00, 3.00 | | 2.00, 3.00 | | 2.00,3.00 | | 2.00, 3.00 | |
| Median | 2.55 | | 2.54 | | 2.50 | | 2.53 | | 2.55 | |
| Week 4 | | | | | | | | | | |
| N Mean (SD) | 134 2.05 (0.567) | 134 -0.48 (0.547) | 144 2.00 (0.597) | 144 -0.51 (0.563) | 142 2.10 (0.522) | 142 -0.40 (0.469) | 152 2.08 (0.580) | 152 -0.44 (0.514) | 126 2.17 (0.446) | 126 -0.34 (0.386) |
| Min, Max | 0.00, 3.00 | -2.39, 2.00 | 0.00, 3.00 | -3.00, 0.58 | 0.00, 3.00 | -2.54, 0.52 | 0.00, 3.00 | -2.55, 0.38 | 1.00. 3.00 | -1.42, 0.80 |
| Median | 2.09 | -0.40 | 2.03 | -0.46 | 2.08 | -0.36 | 2.12 | -0.32 | 2.17 | -0.32 |
| Difference from Placebo[1] | | | | | | | | | | |
| LS Mean | | -0.13 | | -0.17 | | -0.05 | | -0.10 | | |
| Standard Error | | 0.061 | | 0.060 | | 0.060 | | 0.059 | | |
| 95% Cl | | (-0.25, -0.01) | | (-0.28, -0.05) | | (-0.17, 0.07) | | (-0.21, 0.02) | | |
| P-value | | 0.031 | | 0.005 | | 0.401 | | 0.100 | | |
| Week 12 | | | | | | | | | | |
| N | 124 | 124 | 129 | 129 | 124 | 124 | 135 | 135 | 115 | 115 |
| Mean(SD) | 1.43 (0.977) | -1.12 (0.963) | 1.61 (0.817) | -0.90 (0.783) | 1.74 (0.756) | -0.76 (0.744) | 1.79 (0.814) | -0.71 (0.806) | 1.96 (0.618) | -0.56 (0.603) |
| Min, Max | 0.00. 3.00 | -3.00, 0.47 | 0.00, 3.00 | -3.00. 0.44 | 0.00, 3.00 | -2.78, 0.71 | 0.00, 3.00 | -2.69, 1.00 | 0.00. 3.00 | -2.60, 0.83 |
| Median | 1.63 | -0.86 | 1.82 | -0.72 | 1.93 | -0.58 | 1.96 | -0.58 | 2.00 | -0.48 |
| Difference from Placebo[1] | | | | | | | | | | |
| LS Mean | | | | | | | | | | |
| Standard Error | | -0.57 | | -0.39 | | -0.24 | | -0.16 | | |
| 95% CI | | 0.100 | | 0.099 | | 0.100 | | 0.098 | | |
| P-value | | (-0.77, -0.38) <0.001 | | (-0.59, -0.20) <0.001 | | (-0.43, -0.04) 0.018 | | (-0.36, 0.03) 0.096 | | |

[1] Derived from the MMRM model with Treatment, Week (1-12), Treatment-by-Week interaction as factors, Baseline as covariate, and Subject as repeated measures unit. Difference is estimated from the simple contrast between the least squares means

TABLE 29

Change From Baseline to Weeks 4 and 12 in Severity Of Moderate To Severe Vasomotor Symptoms - LOCF ANCOVA MITT-VMS Population

| Study Week | TRT1 (N=141) | | TRT2 (N=149) | | TRT3 (N=147) | | TRT4 (N=154) | | Placebo (N=135) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Baseline | | | | | | | | | | |
| N | 141 | | 149 | | 147 | | 154 | | 135 | |
| Mean (SD) | 2.54 (0.320) | | 2.51 (0.249) | | 2.50 (0.231) | | 2.51 (0.262) | | 2.52 (0.246) | |
| Min, Max | 0.00, 3.00 | | 2.00, 3.00 | | 2.00, 3.00 | | 2.00, 3.00 | | 2.00, 3.00 | |
| Median | 2.55 | | 2.54 | | 2.50 | | 2.53 | | 2.55 | |
| Week 4 | | | | | | | | | | |
| N | 141 | 141 | 149 | 149 | 147 | 147 | 154 | 154 | 135 | 135 |
| Mean (SD) | 2.07 (0.563) | -0.47 (0.538) | 2.01 (0.596) | -0.51 (0.556) | 2.11 (0.518) | -0.39 (0.465) | 2.07 (0.578) | -0.44 (0.512) | 2.18 (0.442) | -0.34 (0.377) |
| Min, Max | 0.00, 3.00 | -2.39, 2.00 | 0.00, 3.00 | -3.00, 0.58 | 0.00, 3.00 | -2.54, 0.52 | 0.00, 3.00 | -2.55, 0.38 | 1.00, 3.00 | -1.42, 0.80 |
| Median | 2.11 | -0.39 | 2.03 | -0.45 | 2.09 | -0.35 | 2.10 | -0.32 | 2.16 | -0.32 |
| Difference from Placebo[1] | | | | | | | | | | |
| LS Mean | | -0.12 0.059 | | -0.16 0.059 | | -0.05 0.059 | | -0.10 0.058 | | |
| Standard Error | | | | | | | | | | |
| 95% CI | | (-0.24, -0.00) | | (-0.28, -0.05) | | (-0.16, 0.07) | | (-0.21, 0.02) | | |
| P-value | | 0.042 | | 0.006 | | 0.401 | | 0.093 | | |
| Week 12 | | | | | | | | | | |
| N | 141 | 141 | 149 | 149 | 147 | 147 | 154 | 154 | 135 | 135 |
| Mean (SD) | 1.47 (0.984) | -1.07 (0.967) | 1.60 (0.836) | -0.91 (0.792) | 1.75 (0.758) | -0.75 (0.749) | 1.81 (0.794) | -0.70 (0.775) | 1.99 (0.609) | -0.53 (0.581) |
| Min, Max | 0.00, 3.00 | -3.00, 0.47 | 0.00, 3.00 | -3.00, 0.44 | 0.00, 3.00 | -2.78, 0.71 | 0.00, 3.00 | -2.69, 1.00 | 0.00, 3.00 | -2.60, 0.83 |
| Median | 1.67 | -0.81 | 1.81 | -0.74 | 1.92 | -0.56 | 1.95 | -0.57 | 2.07 | -0.44 |
| Difference from Placebo[1] | | | | | | | | | | |
| LS Mean | | -0.54 | | -0.39 | | -0.23 | | -0.17 | | |
| Standard Error | | 0.094 | | 0.093 | | 0.093 | | 0.092 | | |
| 95% CI | | (-0.73, -0.36) | | (-0.57, -0.20) | | (-0.41, -0.04) | | (-0.35, 0.01) | | |
| P-value | | <0.001 | | <0.001 | | 0.016 | | 0.061 | | |

[1] Derived from the ANCOVA model with Treatment as factor and Baseline as covariate. Difference is estimated from the simple contrast between the least squares means.

TABLE 30

Summary of 1 Year Incidence Rate Of Hyperplasia, Based on Primary Hyperplasia Endpoint ES Population

| | TRT1 (N=280) | TRT2 (N=303) | TRT3 (N=306) | TRT4 (N=274) | Placebo (N=92) |
|---|---|---|---|---|---|
| Month 12 | | | | | |
| Hyperplasia Incidence Rate | 0 / 280 (0.00%) | 0 / 303 (0.00%) | 0 / 306 (0.00%) | 0 / 274 (0.00%) | 0 / 92 (0.00%) |
| 1-sided Upper 95% Confidence Limit | 1.06% | 0.98% | 0.97% | 1.09% | 3.20% |

Note: No between group comparisons are performed because all treatment groups have 0 subject incidence rate.

TABLE 31

Replenish Trial Co-Primary Efficacy Endpoints: Mean Change in Frequency and Severity of Hot Flashes Per Week Versus Placebo at Weeks 4 and 12, VMS-MITT Population

| Estradiol/Progesterone | 1 mg/100 mg | 0.5 mg/100 mg | 0.5 mg/50 mg | 0.25 mg/50 mg | Placebo |
|---|---|---|---|---|---|
| Baseline | | | | | |
| | n=141 | n=149 | n=147 | n=154 | n=135 |
| Frequency (Mean) | 74.4 | 72.1 | 75.9 | 77.0 | 72.4 |
| Severity (Mean) | 2.54 | 2.51 | 2.50 | 2.51 | 2.52 |

Week 4

| | | | | | |
|---|---|---|---|---|---|
| Frequency (Mean) | 31.5 | 37.2 | 41.5 | 38.4 | 45.9 |
| Difference from placebo (LS Mean)* | -12.81 | -8.07 | -4.81 | -10.40 | - |
| P-value versus placebo | <0.001 | 0.013 | 0.141 | 0.001 | - |
| Severity (Mean) | 2.05 | 2.00 | 2.10 | 2.08 | 2.17 |
| P-value versus placebo | 0.031 | 0.005 | 0.401 | 0.1 | - |

Week 12

| | | | | | |
|---|---|---|---|---|---|
| Frequency (Mean) | 17.1 | 19.1 | 25.2 | 24.1 | 32.0 |
| Difference from placebo (LS Mean)* | -16.58 | -15.07 | -10.79 | -11.71 | - |
| P-value versus placebo | <0.001 | <0.001 | 0.002 | <0.001 | - |
| Severity (Mean) | 1.43 | 1.61 | 1.74 | 1.79 | 1.96 |
| P-value versus placebo | <0.001 | <0.001 | 0.018 | 0.096 | - |

Replenish Trial Primary Safety Endpoint: Incidence of Consensus Endometrial Hyperplasia or Malignancy up to 12 months, Endometrial Safety Population^{Ŧ}

| | | | | | |
|---|---|---|---|---|---|
| Endometrial Hyperplasia | 0% (0/280) | 0/303 (0%) | 0/306 (0%) | 0/274 (0%) | 0/92 (0%) |

MITT = Modified intent to treat
^{Ŧ}Per FDA, consensus hyperplasia refers to the concurrence of two of the three pathologists be accepted as the final diagnosis¹
^{∗}Least Squares Means derived from the MMRM model

TABLE 32

MENQOL, Changes In Overall Scores From Baseline To Each Visit (Week 12, Month 6 And Month 12) MITT-VMS Population

| | TRT1 (N=141) | | TRT2 (N=149) | | TRT3 (N=147) | | TRT4 N=154) | | Placebo (N=135) | |
|---|---|---|---|---|---|---|---|---|---|---|
| MENQOL Domain Study Week | Actual Value | Change From Baseline | Actual Value | Change From Baseline | Actual Value | Change From Baseline | Actual Value | Change From Baseline | Actual Value | Change From Baseline |
| Overall MENQOL Score | | | | | | | | | | |
| Baseline | | | | | | | | | | |
| N | 140 | | 149 | | 147 | | 154 | | 135 | |
| Mean (SD) | 4.5 (1.17) | | 4.3 (1.25) | | 4.7 (1.44) | | 4.5 (1.27) | | 4.6 (1.34) | |
| Min. Max | 2, 7 | | 2, 8 | | 2, 8 | | 2, 8 | | 2, 8 | |
| Median | 4 | | 4 | | 4 | | 4 | | 5 | |

Week 12

| N | 125 | 124 | 135 | 135 | 132 | 132 | 142 | 142 | 116 | 116 |
|---|---|---|---|---|---|---|---|---|---|---|
| Mean (SD) | 26(1.13) | -1.9(1.20) | 2.7(1.26) | 2.8(1.33) | 2.8(1.33) | -1.9(1.41) | 2.9(1.36) | -1.7(1.31) | 3.2(1.29) | -1.4(1.36) |
| Min, Max | 1,7 | -5, 1 | 1,7 | 1, 7 | -5,2 | -6, 1 | 1,7 | -6, 1 | 1, 7 | -5, 2 |
| Median | 2 | -2 | 2 | 2 | -1 | -2 | 3 | -1 | 3 | -1 |
| Difference from Placebo [1] | | | | | | | | | | |
| LS Mean | | -0.56 | | -0.34 | | -0.47 | | -0.32 | | |
| Standard Error | | 0.143 | | 0.141 | | 0.141 | | 0.139 | | |
| 95% CI | | (-0.84, -0.28) | | (-0.62, -0.06) | | (0.74, -0.19) | | (-0.59, -0.05) | | |
| P-value | | <0.001 | | 0.016 | | <001 | | 0.021 | | |

[1] Derived from the MMRM model with Treatment, Week. Treatment-by-Week interaction as factors, Baseline as covariate, and Subject as repeated measures unit. Difference is estimated from the simple contrast between the least squares means.

TABLE 32 CON’T

MENQOL Changes In Overall Scores From Baseline To Each Visit (Week 12, Month 6 And Month 12) MITT-VMS Population

| MENQOL Domain Study Week | TRT1 (N=141) | | TRT2 (N=149) | | TRT3 (N=147) | | TRT4 (N=154) | | Placebo (N=135) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Overall MENQOL Score | | | | | | | | | | |
| Month 6 | | | | | | | | | | |
| N | 117 | 116 | 130 | 130 | 118 | 118 | 126 | 126 | 104 | 104 |
| Mean (SD) | 2.4(1.09) | -2.0(1.22) | 2.4(1.20) | -1.8(1.22) | 2.6(1.36) | -2.1(1.50) | 2.7(1.30) | -1.7(1.24) | 3.0(1.29) | -1.6(1.31) |
| Min, Max | 1, 6 | -5, 1 | 1, 6 | -5, 1 | 1, 7 | -6, 1 | 1, 7 | -6, 1 | 1, 7 | -5, 1 |
| Median | 2 | -2 | 2 | -2 | 2 | -2 | 2 | -2 | 3 | -1 |
| Difference from Placebo[1] | | | | | | | | | | |
| LS Mean | | -0.56 | | -0.43 | | -0.43 | | -0.22 | | |
| Standard Error | | 0.144 | | 0.142 | | 0.143 | | 0.141 | | |
| 95% CI | | (-0.84, -0.27) | | (-0.71, -0.16) | | (-0.71,-0.15) | | (-0.49, 0.06) | | |
| P-value | | <0.001 | | 0.002 | | 0.003 | | 0.124 | | |
| Month 12 | | | | | | | | | | |
| N | 126 | 125 | 134 | 134 | 134 | 134 | 130 | 130 | 124 | 124 |
| Mean (SD) | 2.7(1.39) | -1.8 (1.41) | 2.3 (1.11) | 1.9(1.35) | 2.9 (1.25) | -1.8(1.51) | 2.9 (1.30 | -1.6(1.33) | 3.2(1.47) | -1.3 (1.50) |
| Min, Max | 1, 7 | -5, 2 | 1, 7 | -5, 3 | 1, 7 | -6, 1 | 1, 7 | -6, 2 | 1, 7 | -5, 2 |
| Median | 2 | -2 | 2 | -2 | 3 | -2 | 3 | -2 | 3 | -1 |
| Difference from Placebo[1] | | | | | | | | | | |
| LS Mean | | -0.48 | | -0.72 | | -0.38 | | -0.29 | | |
| Standard Error | | 0.152 | | 0.149 | | 0.149 | | 0.149 | | |
| 95% CI | | (-0.78. -0.19) | | (-1.01, -0.42) | | (-0.67. -0.08) | | (-0.58, 0.01) | | |
| P-value | | 0.001 | | <0.001 | | 0.012 | | 0.055 | | |

[1] Derived from the MMRM model with Treatment, Week, Treatment-by-Week interaction as factors, Baseline as covariate, and Subject as repeated measures unit. Difference is estimated from the simple contrast between the least squares means.

TABLE 33

MENQOL Changes In Vasomotor Domain Score From Baseline To Each Visit (Week 12, Month 6 And Month 12) MITT Population-VMS Population

| MENQOL Domain Study Week | TRT1 (N=416) | | TRT2 (N=422) | | TRT3 (N=421) | | TRT4 (N=423) | | Placebo (N=151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Vasomotor Domain | | | | | | | | | | |
| Baseline | | | | | | | | | | |
| N | 140 | | 149 | | 147 | | 154 | | 135 | |
| Mean (SD) | 7.1 (0.88) | | 6.9(1.06) | | 7.1 (1.00) | | 7.1 (0.96) | | 7.2(1.00) | |
| Min, Max | 4, 8 | | 3, 8 | | 3, 8 | | 3, 8 | | 3, 8 | |
| Median | 7 | | 7 | | 7 | | 7 | | 7 | |
| Week 12 | | | | | | | | | | |
| N | 125 | 124 | 135 | 135 | 132 | 132 | 142 | 142 | 116 | 116 |
| Mean (SD) | 3.3 (1.93) | -3.8 (1.98) | 3.6 (1.91) | -3.3 (2.04) | 3.8(1.89) | -3.3 (1.97) | 3.9(2.07) | -3.2(2.13) | 5.0(1.79) | -2.2 (1.83) |
| Min, Max | 1, 8 | -7, 2 | 1, 8 | -7, 3 | 1, 8 | -7, 2 | 1, 8 | -7, 1 | 1, 8 | -7, 3 |
| Median | 3 | -4 | 3 | -4 | 4 | -4 | 4 | -3 | 5 | -2 |
| Difference from Placebo[1] | | | | | | | | | | |
| LS Mean | | -1.68 | | -1.37 | | -1.19 | | -1.09 | | |
| Standard Error | | 0.243 | | 0.239 | | 0.239 | | 0.236 | | |
| 95% Cl | | (-2.16, -1.20) | | (-1.84, -0.90) | | (-1.66, -0.72) | | (-1.55, -0.62) | | |
| P-value | | <0.001 | | <0.001 | | <0.001 | | <0.001 | | |

TABLE 33 CON’T

MENQOL Changes In Vasomotor Domain Score From Baseline To Each Visit (Week 12, Month 6 And Month 12) MITT Population-VMS Population

| MENQOL Domain Study Week | TRT1 (N=416) | | TRT2 (N=422) | | TRT3 (N=421) | | TRT4 (N=423) | | Placebo (N=151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Vasomotor Domain | | | | | | | | | | |
| Month 6 | | | | | | | | | | |
| N | 117 | 116 | 130 | 130 | 118 | 118 | 126 | 126 | 104 | 104 |
| Mean (SD) | 2.8 (1.83) | -4.3 (1.94) | 2.9 (1.75) | -4.1 (1.99) | 3.1 (1.99) | -4.0 (2.12) | 3.5 (1.93) | -3.5 (1.95) | 4.2 (2.12) | -3.0 (2.26) |
| Min, Max | 1, 7 | -7, 1 | 1, 8 | -7, 1 | 1, 8 | -7, 1 | 1, 8 | -7, 1 | 1, 8 | -7, 2 |
| Median | 2 | -5 | 2 | -5 | 3 | -4 | 3 | -4 | 4 | -3 |
| Difference from Placebo[1] | | | | | | | | | | |
| LS Mean | | -1.48 | | -1.41 | | -1.16 | | -0.80 | | |
| Standard Error | | 0.251 | | 0.246 | | 0.249 | | 0.245 | | |
| 95% CI | | (-1.97, -0.95) | | (-1.89,-0.92) | | (-1.64,-0.67) | | (-1.28,-0.32) | | |
| P-value | | <0.001 | | <0.001 | | <0.001 | | 0.001 | | |

[1] Derived from the MMRM model with Treatment, Week, Treatment-by-Week interaction as factors, Baseline as covariate, and Subject as repeated measures unit. Difference is estimated from the simple contrast between the least squares means

TABLE 33 CON’T

MENQOL Changes In Vasomotor Domain Score From Baseline To Each Visit (Week 12, Month 6 And Month 12) MITT Population-VMS Population

| MENQOL Domain Study Week | TRT1 (N=141) | | TRT2 (N=149) | | TRT3 (N=147) | | TRT4 (N=154) | | | Placebo (N=135) |
|---|---|---|---|---|---|---|---|---|---|---|
| | Actual Value | Change From Baselin e | Actual Value | Change From Baselin e | Actual Value | Change From Baselin e | Actual Value | Change From Baselin e | Actual Value | Change From Baselin e |
| Month 12 | | | | | | | | | | |
| N | 126 | 125 | 134 | 134 | 134 | 134 | 130 | 130 | 123 | 123 |
| Mean (SD) | 3.2 (2.13) | -3.9(2.18) | 2.9 (1.67) | -4.0 (1.90) | 3.5 (2.14) | -3.6 (2.26) | 3.8 (1.99) | -3.2 (1.87) | 4.7(2.21) | -2.4 (2.31) |
| Min, Max | 1, 8 | -7, 2 | 1, 8 | -7, 2 | 1, 8 | -7, 2 | 1, 8 | -7.1 | 1, 8 | -7, 3 |
| Median | 3 | -4 | 3 | -4 | 3 | -3 | 4 | -3 | 5 | -2 |
| Difference from Placebo[1] | | | | | | | | | | |
| LS Mean | | | -1.53 | | -1.69 | | -1.19 | | -0.87 | |
| Standard Error | | | 0.252 | | 0.248 | | 0.248 | | 0.248 | |
| 95% CI | | | (-2.02, -1.03) | | (-2.18, -1.20) | | (-1.68, -0.70) | | (-1.36, -0.38) | |
| P-value | | | <0.001 | | <0.001 | | <0.001 | | <0.001 | |

[1] Derived from the MMRM model with Treatment, Week, Treatment-by-Week interaction as factors, Baseline as covariate, and Subject as repeated measures unit. Difference is estimated from the simple contrast between the least squares means

TABLE 34

MENQOL Changes In Overall Scores From Baseline To Each Visit (Week 12, Month 6 And Month 12) MITT Population

| MENQOL Domain Study Week | TRT1 (N=416) | | TRT2 (N=422) | | TRT3 (N=421) | | TRT4 (N=423) | | Placebo (N=151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Overall MENQOL Score | | | | | | | | | | |
| Baseline | | | | | | | | | | |
| N | 414 | | 422 | | 421 | | 422 | | 151 | |
| Mean (SD) | 4.3(1.28) | | 4.3 (1.30) | | 4.4 (1.40) | | 4.3 (1.33) | | 4.7(1.37) | |
| Min, Max | 2, 8 | | 2, 8 | | 2, 8 | | 2, 8 | | 2, 8 | |
| Median | 4 | | 4 | | 4 | | 4 | | 5 | |
| Week 12 | | | | | | | | | | |
| N | 355 | 354 | 364 | 364 | 375 | 375 | 377 | 376 | 117 | 117 |
| Mean (SD) | 2.5 (1.13) | -1.8 (1.31) | 2.6 (1.14) | -1.6 (1.38) | 2.6(1.22) | -1.7 (1.35) | 2.7(1.22) | -1.6(1.35) | 3.2 (1.30) | -1.4 (1.36) |
| Min, Max | 1, 7 | -6, 1 | 1, 7 | -6, 2 | 1, 8 | -6, 1 | 1, 7 | -6, 3 | 1, 7 | -5, 2 |
| Median | 2 | -2 | 2 | -1 | 2 | -2 | 3 | -1 | 3 | -1 |
| Change from Baseline[1] | | | | | | | | | | |
| 95% CI | | (-1.93, -1.65) | | (-1.77,-1.48) | | (-1.87,-1.60) | | (-1.76, -1.48) | | (-1.60.-1.10) |
| P-value | | <0.001 | | <0.001 | | <0.001 | | <0.001 | | <0.001 |

[1] Paired t-test for the mean change from baseline.

TABLE 34 CON’T

MENQOL Changes In Overall Scores From Baseline To Each Visit (Week 12, Month 6 And Month 12) MITT Population

| MENQOL Domain Study Week | TRT1 (N=416) | | TRT2 (N=422) | | TRT3 (N=421) | | TRT4 (N=423) | | Placebo (N=151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Overall MENQOL Score | | | | | | | | | | |
| Month 6 | | | | | | | | | | |
| N | 317 | 316 | 333 | 333 | 343 | 343 | 325 | 324 | 104 | 104 |
| Mean (SD) | 2.3 (1.01) | -2.0 (1.32) | 2.4 (1.10) | -1.8 (1.38) | 2.5 (1.25) | -1.8 (1.39) | 2.6(1.24) | -1.7 (1.29) | 3.0 (1.29) | -1.6 (1.31) |
| Min, Max | 1, 6 | -6, 1 | 1, 6 | -6, 3 | 1, 7 | -6, 2 | 1, 7 | -6, 2 | 1, 7 | -5, 1 |
| Median | 2 | -2 | 2 | -2 | 2 | -2 | 2 | -2 | 3 | -1 |
| Change from Baseline[1] | | | | | | | | | | |
| 95% Cl | | (-2.13, -1.84) | | (-1.96, -1.67) | | (-1.92, -1.62) | | (-1.79, -1.51) | | (-1.85, -1.34) |
| P-value | | <0.001 | | <0.001 | | <0.001 | | <0.001 | | <0.001 |
| Month 12 | | | | | | | | | | |
| N | 367 | 365 | 365 | 3 | 381 | 38 | 36 | 36 | 125 | 125 |
| Mean (SD) | 2.5 (1.25) | -1.8 (1.44) | 2.5 (1.15) | -1.7 (1.45) | 2.7 (1.25) | -1.6 (1.40) | 2.9 (1.38) | -1.5 (1.41) | 3.2 (1.47) | -1.3 (1.50) |
| Min, Max | 1, 7 | -6, 3 | 1, 7 | -6, | 1, 8 | -6, | 1, | -6, 2 | 1, 7 | -5, 2 |
| Median | 2 | -2 | 2 | - | 2 | - | 3 | - | 3 | -1 |
| Change from Baseline[1] | | | | | | | | | | |
| 95% Cl | | (-1.95, -1.66) | | (-1.89, -1.59) | | (-1.78, -1.50) | | (-1.65, -1.36) | | (-1.59, -1.06) |
| P-value | | <0.001 | | <0.001 | | <0.001 | | <0.001 | | <0.001 |

[1] Paired t-test for the mean change from baseline.

TABLE 35

MENQOL Changes In Vasomotor Domain Score From Baseline To Each Visit (Week 12, Month 6 And Month 12) MITT Population

| MENQOL Domain Study Week | TRT1 (N=416 | | TRT2 (N=422) | | TRT3 (N=421) | | TRT4 (N=423) | | Placebo (N-151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Vasomotor Domain | | | | | | | | | | |
| Baseline | | | | | | | | | | |
| N | 414 | | 422 | | 421 | | 422 | | 151 | |
| Mean (SD) | 6.3 (1.45) | | 6.4 (1.30) | | 6.4 (1.40) | | 6.4 (1.33) | | 7.1 (0.99) | |
| Min, Max | 1, 8 | | 2, 8 | | 2, 8 | | 2, 8 | | 3, 8 | |
| Median | 7 | | 7 | | 7 | | 7 | | 7 | |
| Week 12 | | | | | | | | | | |
| N | 355 | 354 | 364 | 364 | 375 | 375 | 377 | 376 | 117 | 117 |
| Mean (SD) | 2.8 (1.75 | -3.5 (2.02) | 3.3 (1.79) | -3.1 (2.00) | 3.3 (1.85) | -3.1 (2.01) | 3.5 (1.86) | -2.9 (1.94) | 5.0 (1.80) | -2.2 (1.84) |
| Min, Max | 1, 8 | -7, 2 | 1, 8 | -7, 5 | 1, 8 | -7, 2 | 1, 8 | -7, 3 | 1, 8 | -7, 3 |
| Median | 2 | -4 | 3 | -3 | 3 | -3 | 3 | -3 | 5 | -2 |
| Difference from Placebo[1] | | | | | | | | | | |
| LS Mean | | -1.96 | | -1.50 | | -1.49 | | -1.31 | | |
| Standard Error | | 0.188 | | 0.187 | | 0.187 | | 0.187 | | |
| 95% CI | | (-2.33, -1.59) | | (-1.87. -1.13) | | (-1.86, -1.13) | | (-1.67. -0.94) | | |
| P-value | | <0.001 | | <0.001 | | <0.001 | | <0.001 | | |

[1] Derived from the MMRM model with Treatment, Week, Treatment-by-Week interaction as factors, Baseline as covariate, and Subject as repeated measures unit. Difference is estimated from the simple contrast between the least squares means

TABLE 35 CON’T

MENQOL Changes In Vasomotor Domain Score From Baseline To Each Visit (Week 12, Month 6 And Month 12) MITT Population

| MENQOL Domain Study Week | TRT1 (N=416) | | TRT2 (N=422) | | TRT3 (N=421) | | TRT4 (N=423) | | Placebo (N=151) | |
|---|---|---|---|---|---|---|---|---|---|---|
| | Actual Value | Change From Baseline | Actual Value | Change From Baseline | Actual Value | Change From Baseline | Actual Value | Change From Baseline | Actual Value | Change From Baseline |
| Vasomotor Domain | | | | | | | | | | |
| Month 6 | | | | | | | | | | |
| N | 317 | 316 | 333 | 333 | 342 | 342 | 325 | 324 | 104 | 104 |
| Mean (SD) | 2.3 (1.53) | -4.0 (1.95) | 2.8 (1.69) | -3.7 (2.02) | 2.9(1.85) | -3.5 (2.15) | 3.1 (1.80) | -3.3 (1.94) | 4.2 (2.12) | -3.0 (2.26) |
| Min, Max | 1, 7 | -7, 3 | 1, 8 | -7, 3 | 1, 8 | -7, 2 | 1, 8 | -7, 2 | 1, 8 | -7, 2 |
| Median | 2 | -4 | 2 | -4 | 3 | -4 | 3 | -4 | 4 | -3 |
| Difference from Placebo[1] | | | | | | | | | | |
| LS Mean | | -1.70 | | -1.33 | | -1.20 | | -0.94 | | |
| Standard Error | | 0.193 | | 0.192 | | 0.192 | | 0.192 | | |
| 95% Cl | | (-2.08, -1.32) | | (-1.70, -0.95) | | (-1.58, -0.82) | | (-1.31, -0.56) | | |
| P-value | | <0.001 | | <0.001 | | <0.001 | | <0.001 | | |
| Month 12 | | | | | | | | | | |
| N | 367 | 365 | 365 | 365 | 381 | 381 | 360 | 359 | 124 | 124 |
| Mean (SD) | 2.7 (1.86) | -3.6 (2.15) | 2.9 (1.74) | -3.5 (2.00) | 3.1 (1.96) | -3.2 (2.14) | 3.5 (1.98) | -2.9 (1.98) | 4.7 (2.22) | -2.4 (2.31) |
| Min, Max | 1, 8 | -7, 3 | 1, 8 | -7, 2 | 1, 8 | -7, 3 | 1, 8 | -7, 3 | 1, 8 | -7, 3 |
| Median | 2 | -4 | 3 | -4 | 3 | -3 | 3 | -3 | 5 | -2 |
| Difference from Placebo[1] | | | | | | | | | | |
| LS Mean | | -1.80 | | -1.62 | | -1.42 | | -1.10 | | |
| Standard Error | | 0.194 | | 0.193 | | 0.193 | | 0.193 | | |
| 95% CI | | (-2.18, -1.42) | | (-2.00, -1.24) | | (-1.80, -1.04) | | (-1.48, -0.72) | | |
| P-value | | <0.001 | | <0.001 | | <0.001 | | <0.001 | | |

[1] Derived from the MMRM model with Treatment, Week, Treatment-by-Week interaction as factors, Baseline as covariate, and Subject as repeated measures unit. Difference is estimated from the simple contrast between the least squares means

TABLE 36

Summary of Percent Treatment Responders at Weeks 4, 8, And 12 Based On Subject Satisfaction with Treatment (Clinical Global Impression [CGI]) Compared to Changes in Frequency of Moderate to Severe Vasomotor Symptoms from Baseline MITT-VMS Population

| | TRT1 (N=141) | | TRT2 (N=149) | | TRT3 (N=147) | | TRT4 (N=154) | | Placebo (N=135) | |
|---|---|---|---|---|---|---|---|---|---|---|
| Clinical Global Impression | N (%) [1] | Mean Chg [2] | N (%) [1] | MeanChg [2] | N (%) [1] | Mean Chg [2] | N (%) [1] | MeanChg [2] | N (%) [1] | Mean Chg [2] |
| Week 4 | | | | | | | | | | |
| Very Much Improved | 42/136 (30.9) | -56.5 | 29/141 (20.6) | -53.5 | 23/144 (16.0) | -47.8 | 28/148 (18.9) | -63.2 | 8/125 (6.4) | -53.5 |
| Much Improved | 44/136 (32.4) | -46.9 | 42/141 (29.8) | -44.4 | 49/144 (34.0) | -44.5 | 47/148 (31.8) | -50.5 | 33/125 (26.4) | -46.8 |
| Minimally Improved | 37/136 (27.2) | -24.1 | 49/141 (34.8) | -25.9 | 49/144 (34.0) | -28.8 | 51/148 (34.5) | -28.9 | 49/125 (39.2) | -27.2 |
| No Change | 11/136 (8.1) | -14.9 | 21/141 (14.9) | -6.6 | 15/144 (10.4) | -6.3 | 18/148 (12.2) | -3.4 | 33/125 (26.4) | -5.6 |
| Minimally Worse | 2/136 (1.5) | 24.5 | 0 | | 4/144(2.8) | 1.2 | 3/148 (2.0) | -7.7 | 2/125 (1.6) | 1.0 |
| Much Worse | 0 | | 0 | | 2/144 (1.4) | -3.5 | 0 | | 0 | |
| Very Much Worse | 0 | | 0 | | 2/144 (1.4) | 14.5 | 1/148 (0.7) | -31.0 | 0 | |
| ^{∗∗∗} P-Value[3] ^{∗∗∗} | <0.001 | | 0.004 | | 0.005 | | 0.003 | | | |

Week 8

| Very Much Improved | 64/130 (49.2) | -65.5 | 55/139 (39.6) | -61.6 | 50/134 (37.3) | -62.3 | 38/141 (27.0) | -64.1 | 26/117 (22.2) | -62.1 |
|---|---|---|---|---|---|---|---|---|---|---|
| Much Improved | 37/130 (28.5) | -49.9 | 48/139 (34.5) | -46.7 | 48/134 (35.8) | -50.2 | 55/141 (39.0) | -53.5 | 36/117 (30.8) | -44.2 |
| Minimally Improved | 23/130 (17.7) | -28.7 | 24/139 (17.3) | -27.3 | 23/134 (17.2) | -27.2 | 35/141 (24.8) | -36.8 | 25/117 (21.4) | -28.5 |
| No Change | 6/130 (4.6) | -0.2 | 8/139 (5.8) | -12.0 | 8/134 (6.0) | 0.0 | 11/141 (7.8) | -11.9 | 24/117 (20.5) | -4.5 |
| Minimally Worse | 0 | | 4/139 (2.9) | -21.5 | 3/134 (2.2) | -1.7 | 1/141 (0.7) | 0.0 | 51/117 (4.3) | -21.9 |
| Much Worse | 0 | | 0 | | 1/134(0.7) | -29.0 | 0 | | 0 | |
| Very Much Worse | 0 | | 0 | | 1/134 (0.7) | 13.0 | 1/141 (0.7) | 35.0 | 1/117 (0.9) | 5.0 |
| ^{∗∗∗} P′-Value[3] ^{∗∗∗} | <0.001 | | <0.001 | | 0.001 | | 0.041 | | | |
| Week 12 | | | | | | | | | | |
| Very Much Improved | 54/133 (43.9) | -66.3 | 54/133 (40.6) | -66.3 | 47/131 (35.9) | -68.5 | 52/139 (37.4) | -67.0 | 29/116 (25.0) | -68.4 |
| Much Improved | 47/123 (38.2) | -51.8 | 43/133 (32.3) | -51.7 | 55/131 (42.0) | -48.6 | 49/139 (35.3) | -52.4 | 33/116 (28.4) | -43.1 |
| Minimally Improved | 17/123 (13.8) | -35.5 | 29/133 (21.8) | -34.7 | 22/131 (16.8) | -28.8 | 24/139 (17.3) | -39.1 | 26/116 (22.4) | -35.7 |
| No Change | 4/123 (3.3) | -26.6 | 6/133 (4.5) | -40.8 | 4/131 (3.1) | -7.3 | 10/139 (7.2) | -11.6 | 22/116 (19.0) | -9.9 |
| Minimally Worse | 1/123 (0.8) | -47.0 | 1/133 (0.8) | -27.0 | 2/131 (1.5) | -9.5 | 2/139 (1.4) | -25.0 | 5/116 (4.3) | -9.8 |
| Much Worse | 0 | | 0 | | 1/131 (0.8) | -12.0 | 2/139 (1.4) | 36.0 | 1/116 (0.9) | -13.0 |
| ^{∗∗∗} P-Value[3] ^{∗∗∗} | <0.001 | | 0.002 | | <0.001 | | 0.002 | | | |

[1] N (%) of subjects with CGI in the respective category.
[2] Change in weekly frequency of the moderate to severe vasomotor symptoms for subjects in the respective CGI category.
[3] P-value from the Fisher’s exact test comparing % Very Much Improved+Much Improved to Placebo.
Source: V:/replenish\production\programs\tlf\t_14_2_2_10_freqms_cgi.sas

TABLE 37

Summary of Percent of Subjects with >=50% and >=75% Reduction in Frequency of Moderate to Severe Vasomotor Symptoms from Baseline at Each Week Up to Week 12 MITT-VMS Population

| | TRT1 (N=141) | TRT2 (N=149) | TRT3 (N=147) | TRT4 (N=154) | Placebo (N=135) |
|---|---|---|---|---|---|
| Week 1 | | | | | |
| >=50% Reduction | 15/141 (10.6) | 171149 (11.4) | 15/147 (10.2) | 25/154 (61.2) | 16/115 (11.9) |
| >=75% Reduction | 3/141 (2.1) | 4/149(2.7) | 4/147 (2.7) | 5/154 (3.2) | 1/135 (0.7) |
| Week 2 | | | | | |
| >=50% Reduction | 44/141 (31.2) | 35/149 (23.5) | 34/147 (23.1) | 49/154 (31.8) | 35/135 (25.9) |
| >=75% Reduction | 21/141 (14.9) | 12/149 (8.1) | 15/147 (10.2) | 20/154 (13.0) | 6/135 (4.4) |
| Week 3 | | | | | |
| >=50% Reduction | 68/141 (48.2) | 59/149 (39.6) | 54/147 (36.7) | 71/154 (46.1) | 43/135 (31.9) |
| >=75% Reduction | 38/141 (27.0) | 24/149 (16.1) | 21/147 (14.3) | 29/154 (18.8) | 15/135 (11.1) |
| Week 4 | | | | | |
| >=50% Reduction | 82/141 (58.2) | 70/149 (47.0) | 74/147 (50.3) | 81/154 (52.6) | 44/135 (32.6) |
| >=75% Reduction | 55/141 (39.0) | 34/149 (22.8) | 32/147 (21.8) | 45/154 (29.2) | 17/135 (12.6) |
| Week 5 | | | | | |
| >=50% Reduction | 96/141 (68.1) | 81/149 (54.4) | 77/147 (52.4) | 93/154 (60.4) | 56/135 (41.5) |
| >=75% Reduction | 58/141 (41.1) | 47/149 (31.5) | 38/147(25.9) | 56/154 (36.4) | 27/135 (20.0) |
| Week 6 | | | | | |
| >=50% Reduction | 100/141 (70.9) | 87/149 (58.4) | 85/147 (57.8) | 97/154 (63.0) | 56/135 (41.5) |
| >=75% Reduction | 70/141 (49.6) | 52/149 (34.9) | 47/147 (32.0) | 57/154 (37.0) | 30/135 (22.2) |
| Week 7 | | | | | |
| >=50% Reduction | 99/141 (70.2) | 96/149 (64.4) | 91/147 (61.9) | 103/154 (66.9) | 61/135 (45.2) |
| >=75% Reduction | 74/141 (52.5) | 68/149 (45.6) | 56/147 (38.1) | 59/154 (38.3) | 34/135 (25.2) |
| Week 8 | | | | | |
| >=50% Reduction | 106/141 (75.2) | 103/149 (69.1) | 94/147 (63.9) | 102/154 (66.2) | 64/135 (47.4) |
| >=75% Reduction | 82/141 (58.2) | 68/149 (45.6) | 60/147 (40.8) | 63/154 (40.9) | 38/135 (28.1) |
| Week 9 | | | | | |
| >=50% Reduction | 106/141 (75.2) | 115/149 (77.2) | 100/147 (68.0) | 106/154 (68.8) | 67/135 (49.6) |

Source: V:/replenish\production\programs\tlf\t 14 2 2 9 freqms percent reduction.sas

TABLE 37 CONT

Summary of Percent of Subjects with >=50% and >=75% Reduction in Frequency of Moderate to Severe Vasomotor Symptoms from Baseline at Each Week Up to Week 12 MITT-VMS Population

| | TRT1 (N=141) | TRT2 (N=149) | TRT3 (N=147) | TRT4 (N=154) | Placebo (N=135) |
|---|---|---|---|---|---|
| >=75% Reduction | 79/141 (56.0) | 79/149 (53.0) | 65/147 (44.2) | 67/154 (43.5) | 37/135 (27.4)) |
| Week 10 | | | | | |
| >=50%Reduction | 102/141 (72.3) | 113/149 (75.8) | 110/147 (74.8) | 106/154 (68.8) | 73/135 (54.1) |
| >=75% Reduction | 85/141 (60.3) | 78/149 (52.3) | 65/147 (44.2) | 68/154 (44.2) | 40/135 (29.6) |
| Week 11 | | | | | |
| >=50% Reduction | 103/141 (73.0) | 115/149 (77.2) | 107/147 (72.8) | 112/154 (72.7) | 64/135 (47.4) |
| >=75% Reduction | 87/141 (61.7) | 82/149 (55.0) | 69/147 (46.9) | 74/154 (48.1) | 37/135 (27.4) |
| Week 12 | | | | | |
| >=50% Reduction | 107/141 (75.9) | 117/149 (78.5) | 109/147 (74.1) | 107/154 (69.5) | 72/135 (53.3) |
| >=75% Reduction | 93/141 (66.0) | 85/149 (57.0) | 74/147 (50.3) | 74/154 (48.1) | 39/135 (28.9) |

TABLE 38

Summary of Serum Concentrations (InVentiv Lab) of Estrone at Screening, and Visits 2, 4, 5, 6, and 7 Safety Population
Parameter = Estrone (pg/mL)

| Visit | | TRT1 (N=415) | TRT2 (N=424) | TRT3 (N=421) | TRT4 (N=424) | Placebo (N=151) |
|---|---|---|---|---|---|---|
| Screening | | 412 | 419 | 418 | 415 | 150 |
| | Mean (SD) | 23.46 (12.538) | 23.45 (11.995) | 22.87 (12.917) | 23.87 (10.866) | 23.38 (11.148) |
| | Min, Max | 5.2, 139.0 | 5.9,98.9 | 5.7, 128.0 | 5.6, 60.7 | 6.6, 71.7 |
| | Median | 21.2 | 20.6 | 20.4 | 21.4 | 20.8 |
| Visit 2 Week 4 | N | 382 | 393 | 405 | 401 | 127 |
| | Mean (SD) | 213.79 (158.984) | 113.86 (72.219) | 119.50 (83.787) | 69.18 (39.744) | 24.22 (18.463) |
| | Min, Max | 7.8, 1430.0 | 8.8, 485.0 | 5.4,951.0 | 7.4, 274.0 | 5.8, 159.0 |
| | Median | 188.5 | 103.0 | 111.0 | 62.8 | 19.4 |
| Visit 4 Week 12 | N | 352 | 364 | 373 | 372 | 115 |
| | Mean (SD) | 227.31 (168.122) | 128.27 (81.039) | 125.56 (94.267) | 69.93 (38.102) | 29.95 (17.104) |
| | Min, Max | 8.9, 1820.0 | 6.6, 582.0 | 5.6,981.0 | 8.0, 213.0 | 6.2, 115.0 |
| | Median | 211.0 | 114.5 | 108.0 | 63.9 | 21.4 |
| Visit 5 Month 6 | N | 315 | 332 | 338 | 321 | 102 |
| | Mean (SD) | 235.03 (176.290) | 129.30 (92.857) | 128.26 (77.033) | 73.86 (43.701) | 24.34 (14.065) |
| | Min, Max | 10.4, 1440.0 | 7.8, 876.0 | 9.3, 439.0 | 6.6,360.0 | 6.9, 124.0 |
| | Median | 209.0 | 117.0 | 120.0 | 67.3 | 22.8 |
| Visit 6 Month 9 | N | 293 | 318 | 319 | 296 | 92 |
| | Mean (SD) | 241.57 (185.724) | 126.05 (88.286) | 132.47 (83.365) | 72.92 (41.457) | 30.66 (45.175) |
| | Min, Max | 9.3, 1850.0 | 6.0, 800.0 | 8.1,490.0 | 6.5, 354.0 | 5.1,424.0 |
| | Median | 212.0 | 115.5 | 123.0 | 68.7 | 22.4 |
| Visit 7 Month 12 | N | 280 | 301 | 311 | 280 | 90 |
| | Mean (SD) | 230.19 (187.668) | 119.97 (77.990) | 127.60 (93.805) | 72.48 (46.474) | 28.32 (34.808) |
| | Min, Max | 10.1, 1360.0 | 5.4, 450.0 | 6.1, 586.0 | 8.4, 352.0 | 6.8, 322.0 |
| | Median | 197.0 | 115.0 | 112.0 | 66.2 | 22.0 |

### I. Detailed Efficacy Results and Tabulations of Individual Subject Data

### Analysis of Co-Primary Efficacy Endpoints (MITT-VMS Population)

### Change From Baseline and LS Mean Change From Placebo in Frequency of Moderate to Severe VMS

Baseline values, the mean changes from Baseline, and the LS mean change from placebo in the number of weekly moderate and severe VMS at Weeks 4 and 12 for the MITT-VMS population, analyzed by the MMRM method, are shown in Table 38 and full details can be found in Tables 26 and 27. Percentage change from Baseline and percent LS mean change from placebo are shown in Table 28. At Week 12, there was a high placebo response rate (55% decrease from Baseline in moderate to severe VMS).

At Week 4, all treatment arms demonstrated a statistically significant reduction in the number of moderate and severe VMS compared to placebo, except for 0.5 mg E2/50 mg P (p = 0.141). The mean change from Baseline for the active treatment groups ranged from -40.6 (1 mg E2/100 mg P) to -33.6 (0.5 mg E2/50 mg P) compared to -26.4 for placebo (Table 38). LS mean change from placebo for each treatment arm was: -12.81 for 1 mg E2/100 mg P; -8.07 for 0.5 mg E2/100 mg P; -4.81 for 0.5 mg E2/50 mg P; and -10.40 for 0.25 mg E2/50 mg P.

By Week 12, all doses were statistically significantly different from placebo in reducing the number of moderate to severe VMS (p < 0.002). The mean change from Baseline for the active treatment groups ranged from -55.1 (1 mg E2/100 mg P) to -50.2 (0.5 mg E2/50 mg P) compared to -40.2 for placebo (Table 38). LS mean change from placebo for each treatment arm was: -16.58 for 1 mg E2/100 mg P; -15.07 for 0.5 mg E2/100 mg P; -10.79 for 0.5 mg E2/50 mg P; and -11.71 for 0.25 mg E2/50 mg P.

TABLE 38

Change from Baseline and Placebo in the Mean Number of Weekly Moderate and Severe VMS at Week 4 and Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Week 4 (n) | 134 | 144 | 142 | 152 | 126 |
| Baseline | 72.1 (27.80) | 72.3 (28.06) | 75.2 (27.10) | 77.3 (30.51) | 72.3 (23.44) |
| Mean (SD) change from Baseline | -40.6 (30.59) | -35.1 (29.14) | -33.6 (30.64) | -38.9 (31.04) | -26.4 (27.05) |
| LS Mean (SE) change from placebo | -12.81 (3.30) | -8.07 (3.25) | -4.81 (3.26) | -10.40 (3.22) | - |
| MMRM P-value vs placebo | < 0.001 | 0.013 | 0.141 | 0.001 | - |
| Week 12 (n) | 124 | 129 | 124 | 135 | 115 |
| Baseline | 72.2 (25.04) | 72.8 (28.96) | 75.4 (27.08) | 76.5 (29.29) | 72.2 (22.66) |
| Mean (SD) change from Baseline | -55.1 (31.36) | -53.7 (31.93) | -50.2 (31.35) | -52.4 (33.90) | -40.2 (29.79) |
| LS Mean (SE) change from placebo | -16.58 (3.44) | -15.07 (3.39) | -10.79 (3.41) | -11.71 (3.36) | - |
| MMRM P-value vs placebo | < 0.001 | < 0.001 | 0.002 | < 0.001 | - |

Abbreviations: MITT-VMS - modified intent to treat -vasomotor symptom; E2 - 17β-estradiol; P - progesterone; SD - standard deviation; LS - least square; SE - standard error; MMRM - mixed model repeated measures

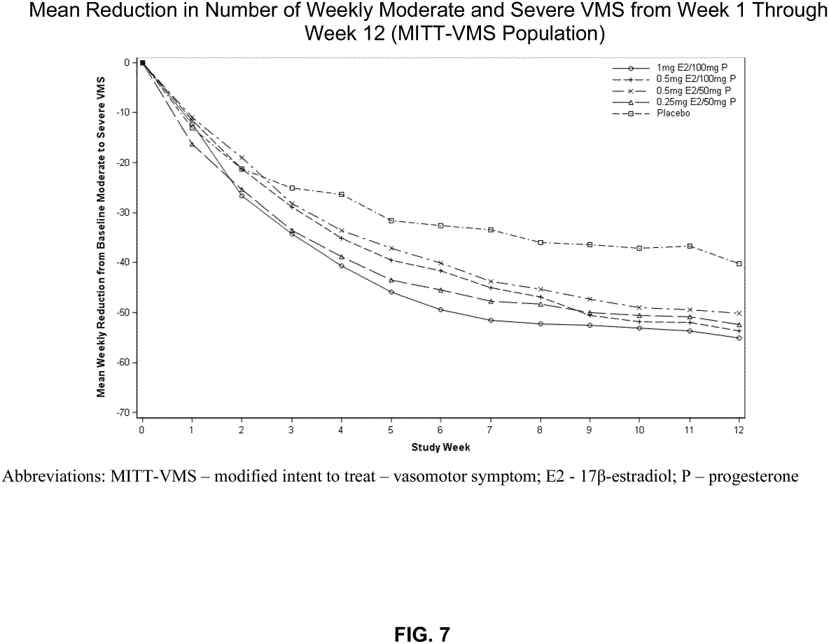

The results of the analysis for the mean number of weekly moderate and severe VMS for each week from Week 1 through Week 12, a secondary endpoint, are provided in Table 28 and presented graphically in FIG. 7.

Statistical significance, compared to placebo, was reached by Week 3 for the 1 mg E2/100 mg P and the 0.25 mg E2/50 mg P doses, by Week 4 for the 0.5 mg E2/100 mg P dose, and by Week 6 for the 0.5 mg E2/50 mg P. For the overall 12 weeks, all doses were statistically significantly different from placebo (p < 0.05).

Results utilizing an MMRM analysis of the change from Baseline to Weeks 4 and 12 in frequency of moderate to severe VMS for the EE-VMS population were obtained. Similar results were noted at Week 4 and Week 12 as compared to the MITT-VMS population, except that the 0.5 mg E2/100 mg P dose was not statistically different (p = 0.053) at Week 4.

A LOCF analysis was also performed on the MITT-VMS population and the EE-VMS population. The LOCF results were similar to those noted in the MMRM analyses for both populations.

### Change From Baseline and LS Mean Change From Placebo in Severity of Moderate to Severe VMS

Baseline values, mean changes from Baseline, and the LS mean change from placebo in the severity of weekly moderate and severe VMS at Weeks 4 and 12 for the MITT-VMS population are presented in Table 39 and full details can be found in Table 28. Percentage change from Baseline and percent LS mean change from placebo were also calculated.

At Week 4, the two highest doses (1 mg E2/100 mg P and 0.5 mg E2/100 mg P) demonstrated a statistically significant reduction in the severity of VMS compared to placebo (p = 0.031 and p = 0.005, respectively). The mean change from Baseline for the active treatment arms ranged from -0.51 (0.5 mg E2/100 mg P) to -0.40 (0.5 mg E2/50 mg P) compared to -0.34 for placebo. The LS mean change from placebo was: -0.13 for 1 mg E2/100 mg P, -0.17 for 0.5 mg E2/100 mg P, -0.05 for 0.5 mg E2/50 mg P, and -0.10 for 0.25 mg E2/50 mg (Table 39).

At Week 12, all doses were statistically significantly different from placebo in reducing the severity of moderate to severe VMS except for the lowest active dose (0.25 mg E2/50 mg P). The mean change from Baseline ranged from -1.12 (1 mg E2/100 mg P) to -0.71 (0.25 mg E2/50 mg P) compared to -0.56 for placebo. The LS mean change from placebo was: -0.57 for 1 mg E2/100 mg P, -0.39 for 0.5 mg E2/100 mg P, -0.24 for 0.5 mg E2/50 mg P, and -0.16 for 0.25 mg E2/50 mg (Table 39).

TABLE 39

Change from Baseline and Placebo in the Mean Weekly Severity Scores of VMS at Week 4 and Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Week 4 (n) | 134 | 144 | 142 | 152 | 126 |
| Baseline | 2.54 (0.325) | 2.51 (0.248) | 2.50 (0.230) | 2.51 (0.259) | 2.52 (0.249) |
| Mean (SD) change from Baseline | -0.48 (0.547) | -0.51 (0.563) | -0.40 (0.469) | -0.44 (0.514) | -0.34 (0.386) |
| LS Mean (SE) change from placebo | -0.13 (0.061) | -0.17 (0.060) | -0.05 (0.060) | -0.10 (0.059) | - |
| MMRM P-value vs placebo | 0.031 | 0.005 | 0.401 | 0.100 | - |
| Week 12 (n) | 124 | 129 | 124 | 135 | 115 |
| Baseline | 2.55 (0.235) | 2.51 (0.248) | 2.50 (0.235) | 2.50 (0.254) | 2.52 (0.245) |
| Mean (SD) change from Baseline | -1.12 (0.963) | -0.90 (0.783) | -0.76 (0.744) | -0.71 (0.806) | -0.56 (0.603) |
| LS Mean (SE) change from placebo | -0.57 (0.100) | -0.39 (0.099) | -0.24 (0.100) | -0.16 (0.098) | - |
| MMRM P-value vs placebo | < 0.001 | < 0.001 | 0.018 | 0.096 | |

Source: Table 28
Abbreviations: MITT-VIMS - modified intent to treat -vasomotor symptom; E2 - 17β-estradiol; P - progesterone; LS - least square; SE - standard error; MMRM - mixed model repeated measures

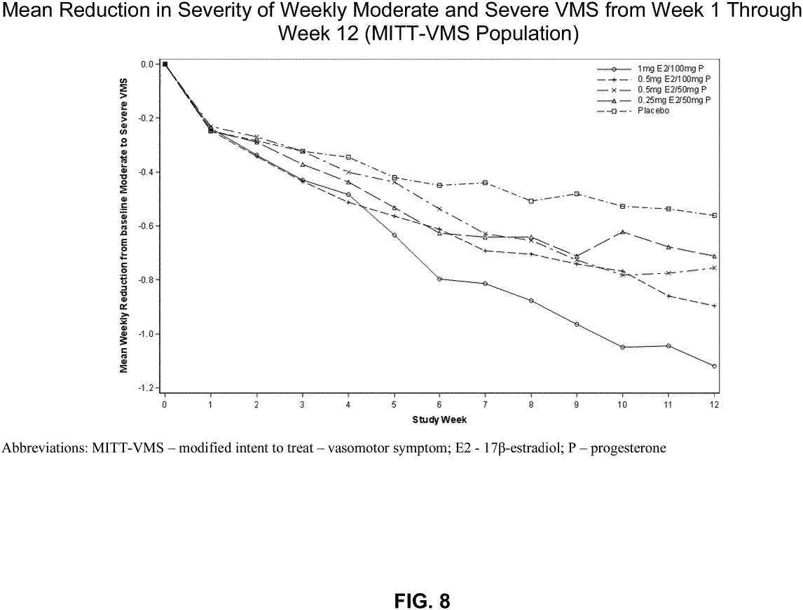

The results of the analysis for the mean severity of weekly moderate and severe VMS for each week from Week 1 through Week 12, a secondary endpoint, are presented graphically in FIG. 8.

The mean reduction in severity of moderate to severe VMS was statistically significantly different from placebo by Week 3 for the two highest doses, 1 mg E2/100 mg P and 0.5 mg E2/100 mg P. For the 0.5 mg E2/50 mg P group, statistically significant differences were noted at Week 7 and Weeks 9 to 12 and for the 0.25 mg E2/50 mg P dose at Weeks 6, 7, and 9.

For the overall 12 weeks, the mean change in severity was statistically significantly different from placebo for the 1 mg E2/100 mg P, 0.5 mg E2/100 mg P, and 0.25 mg E2/50 mg P treatment groups.

Results of the MMRM analysis of the change from Baseline to Weeks 4 and 12 in severity of moderate to severe VMS for the EE-VMS population and for Week 1 through Week 12 were also determined. At Week 4, the 0.5 mg E2/100 mg P dose was statistically different from placebo (p = 0.037). At Week 12, results were similar to the MITT-VMS population with the 1 mg E2/100 mg P, 0.5 mg E2/100 mg P, and 0.5 mg E2/50 mg P active treatment groups being statistically different from placebo.

LOCF analyses of change in the severity of VMS were also performed on both the MITT-VMS and EE-VMS populations. Results for the MITT-VMS population are presented in Table 29, and results were also obtained for the EE-VMS population. The LOCF results were similar to those noted in the MMRM analyses for the MITT-VMS and EE-VMS populations.

### Secondary Efficacy Endpoints From VMS Substudy

Secondary efficacy endpoints evaluated from the VMS Substudy participants included:
1) mean change in the reduction in the number and severity of mild, moderate, and severe VMS from Baseline to each week up to Week 12
2) percentage of subjects with 50% and, separately, 75% reduction in frequency of moderate and severe VMS from Baseline at each week up to Week 12
3) percentage of subjects with 50% and, separately, 75% reduction in frequency of mild, moderate and severe VMS from Baseline at each week up to Week 12
4) CGI distribution (number and percentage of subjects) at Week 4, Week 8, and Week 12, with mean change in the frequency of moderate and severe VMS from Baseline summarized within each CGI category at Weeks 4, 8, and 12
5) change from Baseline in MENQOL parameters
6) and change from Baseline in MOS - Sleep score

### LS Mean Change From Placebo in Frequency of Mild, Moderate, and Severe VMS from Baseline to Week 1 Through Week 12 (MITT-VMS)

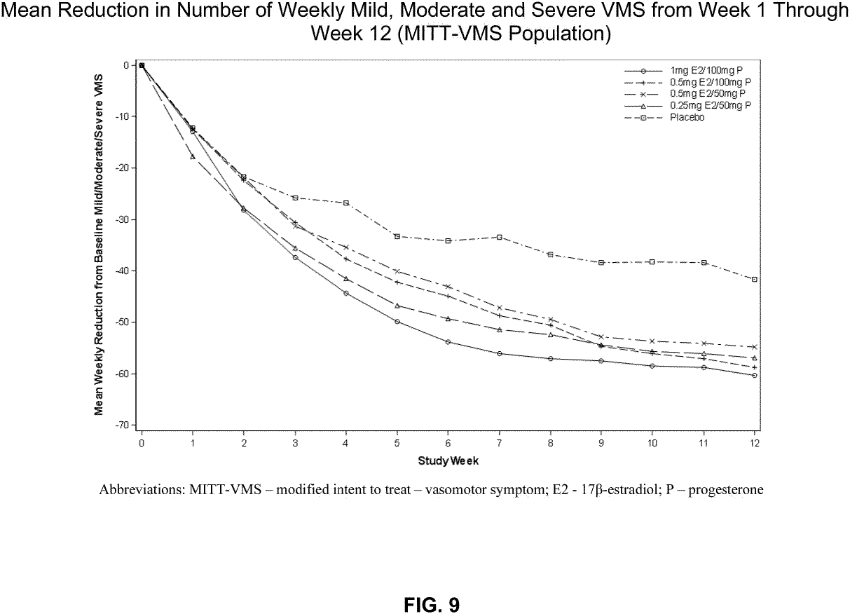

Baseline values, mean changes from Baseline, and LS mean change from placebo in the weekly frequency of mild, moderate, and severe VMS at Weeks 4 and 12 are shown in Table 40. Data for Weeks 1-12 were also collected. The mean change from Baseline for all groups are presented graphically in FIG. 9.

Statistically significant reductions from placebo in the number of mild, moderate, and severe VMS were observed by Week 3 for the 1 mg E2/100 mg P and 0.25 mg E2/50 mg P groups, by Week 4 for the 0.5 mg E2/100 mg P group, and by Week 6 for the 0.5 mg E2/50 mg P group. All doses demonstrated a statistically significant reduction in the number of mild, moderate, and severe VMS at Week 12.

TABLE 40

Change from Baseline and Placebo in the Mean Number of Weekly Mild, Moderate and Severe VMS for Week 1 Through Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Baseline | | | | | |
| Mean (SD) | 86.2 (40.61) | 85.1 (33.92) | 89.2 (30.19) | 88.6 (37.11) | 83.0 (26.47 ) |
| Week4(n) | 134 | 144 | 142 | 152 | 126 |
| Mean (SD) change from Baseline | -44.4 (34.53) | -37.7 (35.38) | -35.4 (34.58) | -41.5 (37.40) | -26.8 (30.52) |
| LS Mean (SE) change from placebo | -15.32 (3.78) | -8.92 (3.73) | -4.56 (3.74) | -11.32 (3.69) | - |
| MMRM P-value vs placebo | < 0.001 | 0.017 | 0.223 | 0.002 | - |
| Week 12 (n) | 124 | 129 | 124 | 135 | 115 |
| Mean (SD) change from Baseline | -60.3 (36.42) | -58.8 (39.59) | -54.8 (34.94) | -57.0 (41.71) | -41.7 (36.35) |
| LS Mean (SE) change from placebo | -20.61 (3.93) | -18.24 (3.87) | -12.62 (3.89) | -13.97 (3.84) | - |
| MMRM P-value vs placebo | < 0.001 | < 0.001 | 0.001 | < 0.001 | - |

Abbreviations: MITT-VMS - modified intent to treat -vasomotor symptom; E2 - 17β-estradiol; P - progesterone; LS - least square; SE - standard error; MMRM - mixed model repeated measures
Results for the EE-VMS population are not different than those for the MITT-VMS population.

### LS Mean Change From Placebo in Severity of Mild, Moderate, and Severe VMS from Baseline to Week 1 Through Week 12 (MITT-VMS)

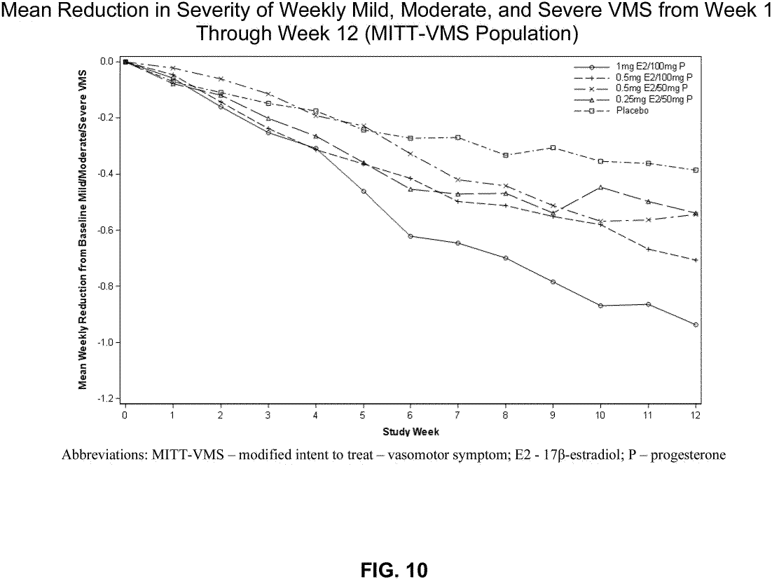

Baseline values, mean changes from Baseline, and LS mean change from placebo in the weekly frequency of mild, moderate, and severe VMS at Weeks 4 and 12 are shown in Table 41 and data for Weeks 1-12 was also collected. The mean change from Baseline for all groups are presented graphically in FIG. 10.

Statistically significant reductions from placebo in the severity of mild, moderate, and severe VMS were observed by Week 3 for the 1 mg E2/100 mg P group. For the remaining groups, statistically significant reductions in severity were noted at various timepoints but were not consistent across the 12 weeks. Given that this analysis includes mild, moderate, and severe VMS, small variations would be expected given a four-point severity scale. At Week 12, all doses, except 0.25 mg E2/50 mg P, demonstrated a statistically significant reduction in the severity of mild, moderate, and severe VMS.

Results for the EE-VMS population were not different than those for the MITT-VMS population.

TABLE 41

Change from Baseline and Placebo in the Mean Severity of Weekly Mild, Moderate and Severe VMS for Week 1 Through Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Baseline | | | | | |
| Mean (SD) | 2.36 (0.337) | 2.31 (0.333) | 2.29 (0.321) | 2.34 (0.325) | 2.34 (0.325) |
| Week4(n) | 134 | 144 | 142 | 152 | 126 |
| Mean (SD) change from Baseline | -0.31 (0.527) | -0.31 (0.540) | -0.19 (0.434) | -0.27 (0.507) | -0.17 (0.368) |
| LS Mean (SE) change from placebo | -0.13 (0.058) | -0.15 (0.057) | -0.02 (0.057) | -0.10 (0.057) | - |
| MMRM P-value vs placebo | 0.027 | 0.011 | 0.710 | 0.072 | - |
| Week 12 (n) | 124 | 129 | 124 | 135 | 115 |
| Mean (SD) change from Baseline | -0.94 (0.986) | -0.71 (0.784) | -0.54 (0.761) | -0.54 (0.824) | -0.39 (0.585) |
| LS Mean (SE) change from placebo | -0.57 (0.101) | -0.37 (0.100) | -0.21 (0.100) | -0.17 (0.099) | - |
| MMRM P-value vs placebo | < 0.001 | < 0.001 | 0.039 | 0.088 | - |

Abbreviations: MITT-VMS - modified intent to treat -vasomotor symptom; E2 - 17β-estradiol; P - progesterone; LS - least square; SE - standard error; MMRM - mixed model repeated measures

### Responder Analysis

A responder was defined as a subject with ≥ 50% reduction from Baseline in the number of moderate and severe VMS. An analysis of those with ≥ 75% reduction from Baseline in the number of moderate and severe VMS was also performed. The same analyses were performed for the reduction in the number of mild, moderate, and severe VMS. Assessment of responder rates was performed at Week 4 and Week 12.

### Subjects With ≥ 50% and ≥ 75% Reduction in Frequency of Moderate and Severe VMS from Baseline to Week 1 Through Week 12 (MITT-VMS Population)

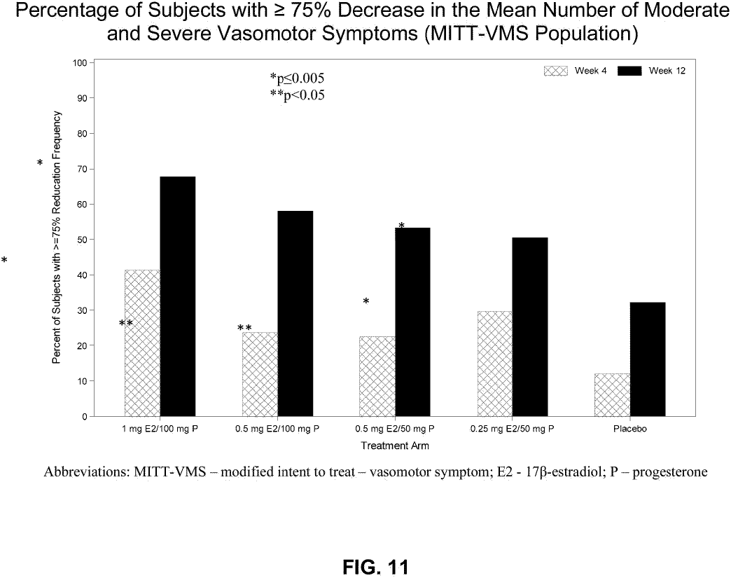

The number and percentage of subjects with a decrease from Baseline of ≥ 50% and, separately, ≥ 75% in the mean weekly number of moderate and severe VMS at Weeks 4 and 12 are shown in Table 42 and data for all 12 weeks was also collected. Those subjects with ≥ 75% reduction for Weeks 4 and 12 are also shown graphically in FIG. 11.

A statistically significant difference between all treatment groups compared to placebo was observed at Weeks 4 and 12.

TABLE 42

Number (%) of Subjects with ≥ 50% and ≥ 75% Reduction in Frequency of Moderate and Severe VMS from Baseline to Week 4 and Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Week 4 (n) | 133 | 144 | 142 | 152 | 126 |
| ≥ 50% Reduction | 82 (61.7) | 70 (48.6) | 74 (52.1) | 81 (53.3) | 41 (32.5) |
| p-value | <0.001 | 0.009 | 0.001 | <0.001 | - |
| ≥ 75% Reduction | 55 (41.4) | 34 (23.6) | 32 (22.5) | 45 (29.6) | 15 (11.9) |
| p-value | < 0.001 | 0.017 | 0.025 | < 0.001 | - |
| Week 12 (n) | 124 | 129 | 124 | 135 | 115 |
| ≥ 50% Reduction | 98 (79.0) | 104 (80.6) | 94 (75.8) | 99 (73.3) | 67 (58.3) |
| p-value | <0.001 | <0.001 | 0.006 | 0.015 | - |
| ≥ 75% Reduction | 84 (67.7) | 75 (58.1) | 66 (53.2) | 68 (50.4) | 37 (32.2) |
| p-value | < 0.001 | < 0.001 | 0.001 | 0.005 | - |

Abbreviations: VMS - vasomotor symptom; MITT-VMS - modified intent to treat - vasomotor symptom; E2 - 17β-estradiol: P - progesterone

### Subjects With ≥ 50% and ≥ 75% Reduction in Frequency of Mild, Moderate, and Severe VMS from Baseline to Week 1 Through Week 12 (MITT-VMS Population)

A responder analysis was also calculated for mild, moderate, and severe VMS. The number and percentage of subjects with a decrease from Baseline of ≥ 50% and, separately, ≥ 75% in the mean weekly number of mild, moderate and severe VMS for Weeks 1 to 12 were collected.

At Week 4, a statistically significantly difference vs placebo in the number of subjects who had a ≥ 50% and a ≥ 75% reduction in the number of mild, moderate, and severe VMS was observed for all treatment groups. Similar results were reported at Week 12 with the exception of the 0.5 mg E2/50 mg P group (p = 0.056).

### Clinical Global Impression for MITT-VMS Population

### Response to CGI Question

Subjects answered the following question: “Rate the total improvement, whether or not in your judgment it is due entirely to drug treatment. Compared to your condition at admission to the study, how much has it changed?” Potential responses included: very much improved, much improved, minimally improved, no change, minimally worse, much worse, or very much worse.

A summary of the number and percentage of responses at Week 4, Week 8, and Week 12, along with the mean change in the frequency of moderate and severe VMS from Baseline were reported.

Table 43 displays the number and percentage of subjects for each possible response to the CGI at Weeks 4, 8, and 12. The results for the top two responses for improvement (very much improved and much improved) and no change or worsening (minimally worse, much worse, or very much worse) were combined for each group and the active treatment groups were compared to placebo. At Week 4, the percentage of subjects who reported ‘very much improved’ or ‘much improved’ for the active treatment groups ranged from 50.0 to 63.2% compared to 32.8% for placebo. By Week 8, the percentage of subjects who reported ‘very much improved’ or ‘much improved’ increased to between 66.0 to 77.7% for active treatment groups and 53.0% for the placebo group. At the last assessment, Week 12, the range for active treatment groups was 72.7 to 82.1% compared to 53.4% for the placebo group. At all timepoints, a statistically significant improvement in the active treatment groups was observed compared to placebo. The highest percentage of subjects reporting ‘very much improved’ or ‘much improved’ was in the 10 mg E2/100 mg P treatment group. All groups increased from Week 4 to Week 8.

TABLE 43

Clinical Global Impression for Weeks 4, 8, and 12 (MITT-VMS Population)

| Response, n (%) | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Week 4 (n) | 136 | 141 | 144 | 148 | 125 |
| Very much improved/much improved | 86 (63.2) | 71 (50.4) | 72 (50.0) | 75 (50.7) | 41 (32.8) |
| Minimally improved | 37 (27.2) | 49 (34.8) | 49 (34.0) | 51 (34.5) | 49 (39.2) |
| No change and worsening | 13 (9.6) | 21 (14.9) | 23 (16.0) | 22 (14.9) | 35 (28.0) |
| p-value | < 0.001 | 0.005 | 0.007 | 0.004 | - |
| Week 8 (n) | 130 | 139 | 134 | 141 | 117 |
| Very much improved/much improved | 101 (77.7) | 103 (74.1) | 98 (73.1) | 93 (66.0) | 62 (53.0) |
| Minimally improved | 23 (17.7) | 24 (17.3) | 23 (17.2) | 35 (24.8) | 25 (21.4) |
| No change and worsening | 6 (4.6) | 12 (8.6) | 13 (9.7) | 13 (9.2) | 30 (25.6) |
| p-value | < 0.001 | < 0.001 | 0.001 | 0.002 | - |
| Week 12 (n) | 123 | 133 | 131 | 139 | 116 |
| Very much improved/much improved | 101 (82.1) | 97 (72.9) | 102 (77.9) | 101 (72.7) | 62 (53.4) |
| Minimally improved | 17 (13.8) | 29 (21.8) | 22 (16.8) | 24 (17.3) | 26 (22.4) |
| No change and worsening | 5 (4.1) | 7 (5.3) | 7 (5.3) | 14(10.1) | 28 (24.1) |
| p-value | < 0.001 | < 0.001 | < 0.001 | 0.002 | - |

Abbreviations: MITT-VMS - modified intent to treat - vasomotor symptom; E2 - 17β-estradiol; P - progesterone

### Clinical Meaningfulness Analysis

Based on the nonparametric discriminant analysis, the threshold for reporting a meaningful decrease in weekly moderate to severe VMS, based on the best discrimination between women who reported ‘minimally improved’ and those women who reported ‘much or very much improved’, was a decrease of 36 VMS at Week 4 and a decrease of 39 VMS at Week 12. Based on the CGI analyses, the responder definition should be based on criteria of a decrease of 36 to 39 moderate to severe VMS.

The number and percentage of subjects who were responders, based on the above definition, are shown in Table 44. Statistically significant differences were observed for all active treatment groups when compared to placebo at Weeks 4 and 12.

TABLE 44

Number (%) of Subjects with ≥ 36 and ≥ 39 Reduction in Frequency ofModerate and Severe VMS from Baseline to Week 4 and Week 12 (MITT-VMS Population)

| | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Week 4 (n) | 134 | 144 | 142 | 152 | 126 |
| ≥ 36 VMS Reduction | 79 (59.0) | 66 (45.8) | 70 (49.3) | 79 (52.0) | 41 (32.5) |
| p-value | < 0.001 | 0.034 | 0.006 | 0.002 | - |
| Week 12 (n) | 124 | 129 | 124 | 135 | 115 |
| ≥ 39 VMS Reduction | 91 (73.4) | 94 (72.9) | 84 (67.7) | 93 (68.9) | 60 (52.2) |
| p-value | < 0.001 | < 0.001 | 0.017 | 0.009 | - |

Abbreviations: MITT-VMS - modified intent to treat - vasomotor symptom; E2 - 17β-estradiol; P - progesterone

### Menopause-Specific Quality of Life Questionnaire for MITT-VMS Population

Baseline scores, mean change from Baseline, and LS mean change from placebo results to Week 12, Month 6, and Month 12 in the MENQOL total score and the vasomotor domain score are shown in Table 45.

At Week 12, statistically significant improvements in the MENQOL Total Score was observed for all active treatment groups compared to placebo. At Months 6 and 12, the Total Scores for the three highest doses (1 mg E2/100 mg P, 0.5 mg E2/100 mg P, and 0.5 mg E2/50 mg P) were statistically significantly improved over placebo.

Also, statistically significant improvements in the Vasomotor Domain were observed at Week 12 and continued through Month 12 for all treatment groups compared to placebo (p ≤ 0.008), indicating sustained efficacy over one year of treatment.

Generally, there were no statistically significant differences noted between groups for the Psychosocial, Physical, or Sexual Domain.

TABLE 45

Mean Change from Baseline and LS Mean Change from Placebo in the MENQOL Score at Week 12, Month 6, and Month 12 (MITT-VMS Population)

| Parameter | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Total Score | | | | | |
| Baseline (n) | 140 | 149 | 147 | 154 | 135 |
| Mean (SD) | 4.5 (1.17) | 4.3 (1.25) | 4.7 (1.44) | 4.5 (1.27) | 4.6(1.34) |
| Week 12 (n) | 124 | 135 | 132 | 142 | 116 |
| Mean change from Baseline (SD) | -1.9 (1.20) | -1.6 (1.23) | -1.9(1.41) | -1.7 (1.31) | -1.4 (1.36) |
| LS Mean change from placebo (SE) | -0.58 (0.145) | -0.34 (0.143) | -0.48 (0.143) | -0.32 (0.141) | - |
| MMRM p-value vs placebo | < 0.001 | 0.016 | < 0.001 | 0.023 | - |
| Month 6 (n) | 116 | 130 | 118 | 126 | 104 |
| Mean change from Baseline (SD) | -2.0 (1.22) | -1.8 (122) | -2.1 (1.50) | -1.7(1.24) | -1.6 (1.31) |
| LS Mean change from placebo (SE) | -0.55 (0.150) | -0.42 (0.146) | -0.44 (0.149) | -0.20 (0.147) | - |
| MMRM p-value vs placebo | < 0.001 | 0.004 | 0.003 | 0.179 | - |
| Month 12 (n) | 97 | 118 | 104 | 104 | 93 |
| Mean change from Baseline (SD) | -1.8 (1.45) | -2.0 (1.27) | -2.0 (1.50) | -1.7 (1.29) | -1.5 (1.50) |
| LS Mean change from placebo (SE) | -0.43 (0.169) | -0.73 (0.162) | -0.49 (0.166) | -0.30 (0.166) | - |
| MMRM p-value vs placebo | 0.012 | < 0.001 | 0.004 | 0.070 | - |
| Vasomotor Domain | | | | | |
| Baseline (n) | 140 | 149 | 147 | 154 | 135 |
| Mean (SD) | 7.1 (0.88) | 6.9 (1.06) | 7.1 (1.00) | 7.1 (0.96) | 7.2 (1.00) |
| Week 12 (n) | 124 | 135 | 132 | 142 | 116 |
| Mean change from Baseline (SD) | -3.8 (1.98) | -3.3 (2.04) | -3.3(1.97) | -3.2 (2.13) | -2.2(1.83) |
| LS Mean change from placebo (SE) | -1.65 (0.246) | -1.31 (0.242) | -1.17 (0.242) | -1.04 (0.238) | - |
| MMRM p-value vs placebo | < 0.001 | < 0.001 | < 0.001 | < 0.001 | - |
| Month 6 (n) | 116 | 130 | 118 | 126 | 104 |
| Mean change from Baseline (SD) | -4.3 (1.94) | -4.1 (1.99) | -4.0 (2.12) | -3.5 (1.95) | -3.0 (2.26) |
| LS Mean change from placebo (SE) | -1.40 (0.258) | -1.32 (0.253) | -1.12 (0.257) | -0.69 (0.254) | - |
| MMRM p-value vs placebo | < 0.001 | < 0.001 | < 0.001 | 0.007 | - |
| Month 12 (n) | 97 | 118 | 104 | 104 | 93 |
| Mean change from Baseline (SD) | -4.0 (2.15) | -4.1 (1.77) | -4.0 (2.11) | -3.4(1.75) | -2.8 (2.26) |
| LS Mean change from placebo (SE) | -1.20 (0.276) | -1.46 (0.265) | -1.23 (0.272) | -0.72 (0.272) | - |
| MMRM p-value vs placebo | < 0.001 | < 0.001 | < 0.001 | 0.008 | - |

Abbreviations: LS - least square: MENQOL - menopause-specific quality of life; MITT-VMS - modified intent to treat - vasomotor symptom; E2 - 17β-estradiol; P - progesterone; SD - standard deviation; SE - standard error; MMRM - mixed model repeated measures

### Medical Outcomes Study Sleep Scale for MITT-VMS Population

Baseline, mean change from Baseline, and LS mean change from placebo for Week 12, Month 6, and Month 12 in MOS Total Sleep Scores are shown in XVIII. The Total Score is the average of nine of the twelve questions.

At Months 6 and 12, statistically significant improvements were noted for all active treatment groups compared to placebo (p < 0.05), except for the 1 mg E2/100 mg P group at Month 12 (p = 0.058).

TABLE 46

Mean Change from Baseline and LS Mean Change from Placebo to Week 12, Month 6, and Month 12 in MOS Total Sleep Score (MITT-VMS Population)

| Parameter | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Baseline (n) | 140 | 148 | 146 | 152 | 134 |
| Mean (SD) | 48.0 (19.08) | 44.9 (17.43) | 49.8 (20.41) | 47.5 (19.25) | 47.3 (18.87) |
| Week 12 (n) | 122 | 134 | 131 | 136 | 111 |
| Mean change from Baseline (SD) | -16.7 (16.99) | -13.1 (16.22) | -18.5 (19.41) | -14.6 (18.80) | -11.5 (19.67) |
| LS Mean change from placebo (SE) | -4.39 (2.059) | -2.54 (2.015) | -4.60 (2.030) | -2.53 (2.007) | - |
| MMRM p-value vs placebo | 0.033 | 0.207 | 0.024 | 0.207 | - |
| Month 6 (n) | 113 | 124 | 118 | 123 | 101 |
| Mean change from Baseline (SD) | -17.8 (17.28) | -16.0 (16.60) | -19.8 (21.18) | -16.6 (19.01) | -11.7 (19.40) |
| LS Mean change from placebo (SE) | -5.48 (2.138) | -5.25 (2.093) | -5.58 (2.122) | -4.99 (2.096) | - |
| MMRM p-value vs placebo | 0.011 | 0.012 | 0.009 | 0.018 | - |
| Month 12 (n) | 96 | 117 | 102 | 100 | 92 |
| Mean change from Baseline (SD) | -14.9 (21.09) | -15.8 (17.72) | -20.6 (21.58) | -17.6 (18.81) | -10.3 (21.78) |
| LS Mean change from placebo (SE) | -4.61 (2.427) | -7.48 (2.322) | -7.96 (2.397) | -6.78 (2.404) | - |
| MMRM p-value vs placebo | 0.058 | 0.001 | < 0.001 | 0.005 | - |

Abbreviations: LS - least square; MOS - medical outcomes study; MITT-VMS - modified intent to treat - vasomotor symptom; E2 - 17β-estradiol; P - progesterone; SD - standard deviation; SE - standard error; MMRM - mixed model repeated measures

Individual scale scores (sleep disturbance, snoring, sleep short of breath or headache, sleep adequacy, sleep somnolence, sleep problems index I, and sleep problems index II) as well as sleep quantity and optimal sleep assessment, based on the average number of hours sleep each night during the past 4 weeks, was assessed. Results are presented in Table 47. Sleep disturbance has been identified as the chief complaint of postmenopausal women.

Sleep disturbance was statistically significantly reduced for the 1 mg E2/100 mg P and 0.5 mg E2/50 mg P groups at Week 12 and for all active treatment groups compared to the placebo at Months 6 and 12 (p < 0.05). At Month 6, there were also statistically significant improvements in the Sleep Problems Index I and Index II for all groups compared to placebo and for most groups at Month 12.

There were no statistically significant differences noted in somnolence between active treatment groups with natural progesterone and placebo, except at Month 12 for the 0.5 mg E2/100 mg P and 0.25 mg E2/50 mg P groups.

TABLE 47

Mean Change from Baseline and LS Mean Change from Placebo to Week 12, Month 6, and Month 12 in MOS - Sleep Scales (MITT-VMS Population)

| Parameter | 1 mg E2/ 100 mg P (N=141) | 0.5 mg E2/ 100 mg P (N=149) | 0.5 mg E2/ 50 mg P (N=147) | 0.25 mg E2/ 50 mg P (N=154) | Placebo (N=135) |
|---|---|---|---|---|---|
| Sleep Disturbance | | | | | |
| Baseline (n) | 140 | 149 | 147 | 154 | 135 |
| Mean (SD) | 53.4 (26.63) | 49.8 (24.12) | 55.6 (26.62) | 54.1 (25.12) | 52.3 (27.61) |
| Week 12 (n) | 124 | 135 | 132 | 142 | 116 |
| Mean change from Baseline (SD) | -22.3 (23.72) | -17.7 (24.75) | -23.6 (25.07) | -19.3 (26.31) | -15.1 (26.65) |
| LS Mean change from placebo (SE) | -6.48 (2.770) | -3.36 (2.715) | -5.85 (2.734) | -2.99 (2.685) | - |
| MMRM p-value vs placebo | 0.020 | 0.216 | 0.033 | 0.265 | - |
| Month 6 (n) | 116 | 130 | 118 | 126 | 104 |
| Mean change from Baseline (SD) | -23.5 (25.99) | -21.3 (24.42) | -26.9 (26.91) | -22.4 (26.76) | -15.4 (27.57) |
| LS Mean change from placebo (SE) | -7.54 (2.854) | -6.72 (2.781) | -8.69 (2.847) | -6.18 (2.800) | - |
| MMRM p-value vs placebo | 0.008 | 0.016 | 0.002 | 0.028 | - |
| Month 12 (n) | 97 | 118 | 104 | 104 | 93 |
| Mean change from Baseline (SD) | -20.0 (28.25) | -22.3 (24.84) | -26.1 (27.21) | -21.6 (25.32) | -14.1 (28.67) |
| LS Mean change from placebo (SE) | -6.56 (3.180) | -10.02 (3.040) | -9.96 (3.129) | -6.85 (3.127) | - |
| MMRM p-value vs placebo | 0.040 | 0.001 | 0.002 | 0.029 | - |
| Sleep adequacy | | | | | |
| Baseline (n) | 140 | 149 | 147 | 154 | 135 |
| Mean (SD) | 37.9 (24.43) | 42.3 (24.89) | 38.4 (24.74) | 41.0 (24.86) | 36.6 (23.38) |
| Week 12 (n) | 124 | 135 | 132 | 142 | 116 |
| Mean change from Baseline (SD) | 12.8 (28.30) | 11.0 (26.57) | 17.3 (30.06) | 10.7 (28.33) | 11.3 (26.09) |
| LS Mean change from placebo (SE) | 1.81 (3.081) | 3.61 (3.031) | 6.16 (3.036) | 2.07 (2.990) | - |
| MMRM p-value vs placebo | 0.558 | 0.233 | 0.043 | 0.488 | - |
| Month 6 (n) | 116 | 130 | 118 | 126 | 104 |
| Mean change from Baseline (SD) | 13.2 (28.61) | 15.2 (26.89) | 14.7 (30.12) | 15.2 (28.75) | 9.5 (27.36) |
| LS Mean change from placebo (SE) | 4.07 (3.206) | 9.58 (3.133) | 5.04 (3.193) | 8.94 (3.152) | - |
| MMRM p-value vs placebo | 0.205 | 0.002 | 0.115 | 0.005 | - |
| Month 12 (n) | 97 | 118 | 104 | 104 | 93 |
| Mean change from Baseline (SD) | 10.4 (28.43) | 10.5 (31.07) | 17.6 (31.61) | 13.7 (27.70) | 10.0 (31.76) |
| LS Mean change from placebo (SE) | 0.96 (3.719) | 5.14 (3.568) | 8.58 (3.657) | 7.51 (3.667) | - |
| MMRM p-value vs placebo | 0.796 | 0.150 | 0.019 | 0.041 | - |
| Sleep somnolence | | | | | |
| Baseline (n) | 140 | 149 | 147 | 154 | 135 |
| Mean (SD) | 32.0 (20.66) | 32.3 (22.39) | 35.7 (22.30) | 33.6 (22.29) | 33.7 (20.99) |
| Week 12 (n) | 124 | 135 | 132 | 142 | 116 |
| Mean change from Baseline (SD) | -11.3 (20.70) | -9.2 (17.82) | -10.4 (22.61) | -10.8(21.78) | -8.7 (20.62) |
| LS Mean change from placebo (SE) | -2.69 (2.197) | -1.42 (2.154) | 0.66 (2.168) | -2.07 (2.128) | - |
| MMRM p-value vs placebo | 0.221 | 0.511 | 0.761 | 0.332 | - |
| Month 6 (n) | 116 | 130 | 118 | 126 | 104 |
| Mean change from Baseline (SD) | -10.1 (21.60) | -10.8 (21.46) | -12.4 (22.98) | -10.5 (23.69) | -9.6 (21.64) |
| LS Mean change from placebo (SE) | -1.00 (2.474) | -2.15 (2.411) | -0.91 (2.466) | -0.90 (2.427) | - |
| MMRM p-value vs placebo | 0.687 | 0.373 | 0.714 | 0.711 | - |
| Month 12 (n) | 97 | 118 | 104 | 104 | 93 |
| Mean change from Baseline (SD) | -8.0 (22.93) | -11.1 (21.51) | -13.1 (20.03) | -13.4 (23.82) | -6.7 (25.95) |
| LS Mean change from placebo (SE) | -2.19 (2.682) | -6.01 (2.564) | -4.72 (2.639) | -5.75 (2.637) | - |
| MMRM p-value vs placebo | 0.415 | 0.019 | 0.074 | 0.030 | - |
| Sleep Problems Index I | | | | | |
| Baseline (n) | 140 | 149 | 147 | 154 | 135 |
| Mean (SD) | 47.0 (18.85) | 42.8 (17.51) | 48.3 (19.98) | 45.6 (18.93) | 45.2 (18.36) |
| Week 12 (n) | 124 | 135 | 132 | 142 | 116 |
| Mean change from Baseline (SD) | -15.2 (17.45) | -11.3 (17.04) | -17.7 (19.47) | -13.5 (19.65) | -9.9 (20.50) |
| LS Mean change from placebo (SE) | -3.99 (2.109) | -2.57 (2.067) | -5.10 (2.084) | -3.16 (2.042) | - |
| MMRM p-value vs placebo | 0.059 | 0.215 | 0.015 | 0.122 | - |
| Month 6 (n) | 116 | 130 | 118 | 126 | 104 |
| Mean change from Baseline (SD) | -16.1 (16.64) | -14.3 (16.56) | -18.0 (20.71) | -15.7 (19.34) | -9.8 (20.37) |
| LS Mean change from placebo (SE) | -5.32 (2.135) | -5.76 (2.080) | -5.59 (2.132) | -5.68 (2.093) | - |
| MMRM p-value vs placebo | 0.013 | 0.006 | 0.009 | 0.007 | - |
| Month 12 (n) | 97 | 118 | 104 | 104 | 93 |
| Mean change from Baseline (SD) | -13.3 (20.52) | -13.5 (18.04) | -18.9 (21.49) | -16.1 (20.01) | -9.1 (22.62) |
| LS Mean change from placebo (SE) | -3.39 (2.470) | -6.49 (2.364) | -7.39 (2.434) | -6.69 (2.429) | - |
| MMRM p-value vs placebo | 0.171 | 0.006 | 0.003 | 0.006 | - |
| Sleep Problems Index II | | | | | |
| Baseline (n) | 140 | 149 | 147 | 154 | 135 |
| Mean (SD) | 48.0 (19.08) | 44.8 (17.39) | 49.9 (20.39) | 47.5 (19.13) | 47.3 (18.80) |
| Week 12 (n) | 124 | 135 | 132 | 142 | 116 |
| Mean change from Baseline (SD) | -16.8 (17.14) | -13.1 (16.16) | -18.5 (19.34) | -14.4 (19.19) | -11.8(19.56) |
| LS Mean change from placebo (SE) | -4.21 (2.037) | -2.39 (1.997) | -4.37 (2.012) | -2.22 (1.973) | - |
| MMRM p-value vs placebo | 0.039 | 0.232 | 0.030 | 0.262 | - |
| Month 6 (n) | 116 | 130 | 118 | 126 | 104 |
| Mean change from Baseline (SD) | -17.5 (17.40) | -16.0(16.69) | -19.8 (21.18) | -17.0 (19.02) | -11.6 (19.31) |
| LS Mean change from placebo (SE) | -5.41 (2.119) | -5.54 (2.065) | -5.74 (2.115) | -5.32 (2.078) | - |
| MMRM p-value vs placebo | 0.011 | 0.008 | 0.007 | 0.011 | - |
| Month 12 (n) | 97 | 118 | 104 | 104 | 93 |
| Mean change from Baseline (SD) | -14.7 (21.13) | -15.7 (17.65) | -20.5 (21.49) | -17.4 (19.48) | -10.5 (21.71) |
| LS Mean change from placebo (SE) | -4.21 (2.421) | -7.36 (2.317) | -7.92 (2.384) | -6.78 (2.381) | - |
| MMRM p-value vs placebo | 0.083 | 0.002 | < 0.001 | 0.005 | - |
| Optimal sleep | | | | | |
| Baseline (n) | 139 | 148 | 147 | 154 | 135 |
| Mean (SD) | 0.3 (0.45) | 0.3 (0.47) | 0.3 (0.46) | 0.3 (0.46) | 0.2 (0.43) |
| Week 12 (n) | 123 | 134 | 132 | 142 | 115 |
| Mean change from Baseline (SD) | 0.2 (0.55) | 0.2 (0.54) | 0.2 (0.45) | 0.2 (0.53) | 0.2 (0.58) |
| LS Mean change from placebo (SE) | -0.00 (0.060) | 0.02 (0.059) | -0.03 (0.059) | 0.05 (0.058) | - |
| MMRM p-value vs placebo | 0.938 | 0.779 | 0.646 | 0.421 | - |
| Month 6 (n) | 115 | 129 | 118 | 125 | 104 |
| Mean change from Baseline (SD) | 0.3 (0.52) | 0.2 (0.52) | 0.2 (0.57) | 0.1 (0.57) | 0.2 (0.52) |
| LS Mean change from placebo (SE) | 0.11 (0.063) | 0.11 (0.062) | 0.06 (0.063) | 0.01 (0.062) | - |
| MMRM p-value vs placebo | 0.075 | 0.074 | 0.370 | 0.851 | - |
| Month 12 (n) | 96 | 117 | 104 | 104 | 93 |
| Mean change from Baseline (SD) | 0.2 (0.47) | 0.3 (0.58) | 0.1 (0.57) | 0.1 (0.51) | 0.2 (0.47) |
| LS Mean change from placebo (SE) | -0.05 (0.067) | 0.07 (0.064) | -0.06 (0.065) | -0.04 (0.065) | - |
| MMRM p-value vs placebo | 0.452 | 0.244 | 0.354 | 0.527 | - |

Abbreviations: LS - least square: MOS - medical outcomes study; MITT-VMS - modified intent to treat - vasomotor symptom; E2 - 17β-estradiol; P - progesterone; SD - standard deviation; SE - standard error; MMRM - mixed model repeated measures

### Efficacy Conclusions

TX-001HR demonstrated statistically and clinically significant improvements in the frequency and severity of vasomotor symptoms when compared to placebo. Treatment groups were balanced with regards to age, race, and BMI. The overall mean age for the efficacy population was 54.6 years with a mean BMI of 26.6 kg/m². The mean compliance was 92.5% at Week 12 and 89.1% of subjects completed the 12-week VMS Substudy.
1) At Week 4, a statistically significant reduction in the number of moderate and severe VMS compared to placebo was observed in all active treatment arms, except for 0.5 mg E2/50 mg P (p = 0.141). By Week 6, all active treatment arms demonstrated statistically significant differences from placebo. This was sustained through Week 12.
2) By Week 12, all doses were statistically significantly different from placebo in reducing the number of moderate to severe VMS (p < 0.002). The mean change from Baseline for the active treatment groups ranged from -55.1 (1 mg E2/100 mg P) to -50.2 (0.5 mg E2/50 mg P) compared to -40.2 for placebo.
3) The mean reduction in severity of moderate to severe VMS was statistically significantly different from placebo by Week 3 for the two highest doses, 1 mg E2/100 mg P and 0.5 mg E2/100 mg P. For the 0.5 mg E2/50 mg P group, statistically significant differences were noted at Week 7 and Weeks 9 to 12 and for the 0.25 mg E2/50 mg P dose at Weeks 6, 7, and 9.
4) A CGI based anchor was utilized to determine a clinical responder threshold and provided evidence of clinically significant reductions in moderate to severe VMS in those treated with all doses of TX-001HR.

Secondary efficacy endpoints support the consistency of effect of TX-001HR.
1) Statistically significant reductions from placebo in the number of mild, moderate, and severe VMS were observed by Week 3 for the 1 mg E2/100 mg P and 0.25 mg E2/50 mg P groups, by Week 4 for the 0.5 mg E2/100 mg P group, and by Week 6 for the 0.5 mg E2/50 mg P group. All doses demonstrated a statistically significant reduction in the number of mild, moderate, and severe VMS at Week 12.
1) Statistically significant reductions from placebo in the severity of mild, moderate, and severe VMS were observed by Week 3 for the 1 mg E2/100 mg P group. For the remaining groups, statistically significant reductions in severity were noted at various timepoints; all groups were statistically significantly better than placebo by Week 12 except for the 0.25 mg E2/50 mg P group.
2) There were significantly more subjects (responders) in the active treatment arms who had greater than 50% and 75% reductions in the numbers of their moderate to severe VMS at both Week 4 and Week 12.
3) At Week 12, statistically significant improvements in the MENQOL Total Score were observed for all active treatment groups compared to placebo. The vasomotor domain demonstrated statistically significant improvements for all treatment groups compared to placebo (p < 0.008) at all timepoints measured, this effect was observed throughout the trial indicating sustained efficacy.
4) The MOS measurements of sleep indicated improvements in the active treatment arms compared with placebo in those sleep indices typically associated with postmenopausal sleep difficulties (ie, sleep disturbance and sleep adequacy).
5) Subgroup analyses demonstrated an overall consistency of effect of TX-001HR.
   One exception was noted when data were analyzed by race. The placebo response rate in this study was high in the overall population (55%). A statistically significant difference was observed (p = 0.049) in the placebo response rate between White (51%) and Black/African American subjects (65%) at 12 weeks in the MITT population.

Overall, despite the very high placebo response rate, the two highest doses of TX-001HR (1 mg E2/100 mg P and 0.5 mg E2/100 mg P) demonstrated clear and consistent efficacy with a clinically meaningful and statistically significant improvement in the frequency of VMS and statistically significant reduction in the severity of moderate to severe VMS at both 4 and 12 weeks compared to Baseline and placebo.

TX-001HR 0.5 mg E2/50 mg P demonstrated clinically meaningful and statistically significant reductions in frequency and severity of VMS at Week 12 (statistically significant reductions in frequency were noted by Week 6 and continued through Week 12 and statistically significant reductions in severity were observed at Week 7 and Weeks 9 to 12).

TX-001HR 0.25 mg E2/50 mg P demonstrated statistically significant reductions in the frequency of VMS at Weeks 4 and 12 (as early as Week 3) but did not significantly reduce the severity of VMS at Weeks 4 and 12, only at Weeks 6, 7, and 9, suggesting the lowest dose of estradiol was a no effect dose for the co-primary endpoints.

### J. PK Steady State

### 1. Estradiol Concentration

A summary of the mean serum concentration of estradiol for each time point and by treatment group for the Safety population is shown in Table 48. The overall mean estradiol concentration at Screening was 6.1 pg/mL and the median estradiol concentration was 4.4 pg/mL, consistent with postmenopausal status. The lower limit of quantification for estradiol was 2.00 pg /mL. A dose response was observed for serum concentrations of estradiol and estradiol levels remained consistent over time for each respective treatment arm.

TABLE 48

Serum Concentration of Estradiol

| Estradiol (pg/mL) | 1 mg E2/ 100 mg P (N=415) | 0.5 mg E2/ 100 mg P (N=424) | 0.5 mg E2/ 50 mg P (N=421) | 0.25 mg E2/ 50 mg P (N=424) | Placebo (N=151) |
|---|---|---|---|---|---|
| Screening | 415 | 423 | 421 | 421 | 150 |
| Mean (SD) | 6.28 (6.623) | 6.45 (7.235) | 5.75 (6.060) | 6.29 (6.247) | 5.63 (4.320) |
| Min, Max | 2.0, 67.2 | 2.0, 55.3 | 2.0, 67.3 | 2.0, 56.3 | 2.0, 25.2 |
| Median | 4.6 | 4.5 | 4.2 | 4.5 | 4.0 |
| Week 4 | 382 | 394 | 405 | 402 | 130 |
| Mean (SD) | 42.49 (36.506) | 23.03 (23.906) | 24.88 (25.429) | 18.50 (33.337) | 8.27 (22.495) |
| Min, Max | 2.0, 245.0 | 2.0, 268.0 | 2.0, 241.0 | 2.0, 403.0 | 2.0, 200.0 |
| Median | 34.5 | 18.6 | 18.7 | 12.2 | 3.8 |
| Week 12 | 352 | 365 | 374 | 371 | 117 |
| Mean (SD) | 44.46 (39.110) | 26.52 (27.316) | 26.75 (31.046) | 16.59 (19.258) | 8.54 (23.224) |
| Min, Max | 2.0, 376.0 | 2.0, 269.0 | 2.0, 292.0 | 2.0, 213.0 | 2.0, 238.0 |
| Median | 36.0 | 20.4 | 20.1 | 12.3 | 3.9 |
| Month 6 | 315 | 333 | 338 | 323 | 102 |
| Mean (SD) | 45.58 (49.032) | 24.23 (22.052) | 24.16 (16.533) | 16.61 (16.962) | 5.35 (4.677) |
| Min, Max | 2.0, 478.0 | 2.0, 230.0 | 2.0, 110.0 | 2.0, 164.0 | 2.0, 25.8 |
| Median | 35.9 | 20.2 | 20.9 | 13.2 | 4.2 |
| Month 9 | 292 | 318 | 320 | 296 | 95 |
| Mean (SD) | 44.46 (35.665) | 27.37 (35.265) | 24.56 (20.397) | 15.06 (13.547) | 7.99 (15.176) |
| Min, Max | 2.3. 248.0 | 2.0, 406.0 | 2.0, 182.0 | 2.0, 167.0 | 2.0, 97.5 |
| Median | 36.0 | 20.5 | 20.3 | 12.6 | 4.1 |
| Month 12 | 282 | 301 | 311 | 280 | 91 |
| Mean (SD) | 42.29 (41.206) | 24.60 (26.442) | 23.66 (18.646) | 15.23 (20.076) | 5.73 (7.278) |
| Min, Max | 2.0, 483.0 | 2.0, 295.0 | 2.0, 146.0 | 2.0, 243.0 | 2.0, 63.2 |
| Median | 35.2 | 20.4 | 20.1 | 12.2 | 4.1 |

Abbreviations: E2 - 17β-estradiol; P - progesterone; SD - standard deviation

### 2. Estrone Concentration

A summary of the mean serum concentration of estrone for each time point and by treatment group for the Safety population is shown in Table 49.

The overall mean estrone concentration at Screening was 23.3 pg/mL and the median concentration was 20.8 pg/mL, consistent with postmenopausal ranges. The lower limit of quantification for estrone was 5.00 pg/mL. Generally, estrone levels were related in a dose dependent manner to the estradiol dose given; levels remained consistent over time for each respective treatment arm.

TABLE 49

Serum Concentration of Estrone

| Estrone (pg/mL) | 1 mg E2/ 100 mg P (N=415) | 0.5 mg E2/ 100 mg P (N=424) | 0.5 mg E2/ 50 mg P (N=421) | 0.25 mg E2/ 50 mg P (N=424) | Placebo (N=151) |
|---|---|---|---|---|---|
| Screening | 415 | 422 | 421 | 421 | 150 |
| Mean (SD) | 23.32 (12.590) | 23.31 (12.052) | 22.75 (12.958) | 23.60 (11.018) | 23.38 (11.148) |
| Min, Max | 5.0, 139.0 | 5.0, 98.9 | 5.0, 128.0 | 5.0, 60.7 | 6.6, 71.7 |
| Median | 21.2 | 20.6 | 20.3 | 21.3 | 20.8 |
| Week 4 | 382 | 394 | 405 | 402 | 130 |
| Mean (SD) | 213.79 (158.984) | 113.59 (72.335) | 119.50 (83.787) | 69.02 (39.823) | 23.78 (18.475) |
| Min, Max | 7.8, 1430.0 | 5.0, 485.0 | 5.4, 951.0 | 5.0, 274.0 | 5.0, 159.0 |
| Median | 188.5 | 103.0 | 111.0 | 62.8 | 19.1 |
| Week 12 | 352 | 365 | 373 | 373 | 117 |
| Mean (SD) | 227.31 (168.122) | 127.93 (81.185) | 125.56 (94.267) | 69.75 (38.198) | 25.59 (17.174) |
| Min, Max | 8.9, 1820.0 | 5.0, 582.0 | 5.6, 981.0 | 5.0, 213.0 | 5.0, 115.0 |
| Median | 211.0 | 114.0 | 108.0 | 63.9 | 21.3 |
| Month 6 | 315 | 334 | 338 | 323 | 103 |
| Mean (SD) | 235.03 (176.290) | 128.56 (93.074) | 128.26 (77.033) | 73.43 (43.900) | 24.15 (14.125) |
| Min, Max | 10.4, 1440.0 | 5.0, 876.0 | 9.3, 439.0 | 5.0, 360.0 | 5.0, 124.0 |
| Median | 209.0 | 116.5 | 120.0 | 67.2 | 22.3 |
| Month 9 | 293 | 318 | 320 | 296 | 95 |
| Mean (SD) | 241.57 (185.724) | 126.05 (88.286) | 132.08 (83.538) | 72.92 (41.457) | 29.84 (44.676) |
| Min, Max | 9.3, 1850.0 | 6.0, 800.0 | 5.0, 490.0 | 6.5, 354.0 | 5.0, 424.0 |
| Median | 212.0 | 115.5 | 122.5 | 68.7 | 22.0 |
| Month 12 | 283 | 302 | 311 | 280 | 90 |
| Mean (SD) | 227.80 (188.091) | 119.59 (78.141) | 127.60 (93.805) | 72.48 (46.474) | 28.32 (34.808) |
| Min, Max | 5.0, 1360.0 | 5.0, 450.0 | 6.1, 586.0 | 8.4, 352.0 | 6.8, 322.0 |
| Median | 195.0 | 114.5 | 112.0 | 66.2 | 22.0 |

Abbreviations: E2 - 17β-estradiol; P - progesterone; SD - standard deviation

### 3. Progesterone Concentration

A summary of the mean serum concentration of progesterone for each time point and by treatment group for the Safety population is shown in XXII. The overall mean progesterone concentration at Screening was 57.6 pg/mL and the median concentration was 50 pg/mL. The lower limit of quantification for progesterone was 50.0 pg/mL. A dose response was observed for serum concentrations of progesterone and progesterone levels remained consistent over time for each respective treatment arm.

TABLE 50

Serum Concentration of Progesterone

| Progesterone (pg/mL) | 1 mg E2/ 100 mg P (N=415) | 0.5 mg E2/ 100 mg P (N=424) | 0.5 mg E2/ 50 mg P (N=421) | 0.25 mg E2/50 mg P (N=424) | Placebo (N=151) |
|---|---|---|---|---|---|
| Screening | 415 | 422 | 420 | 419 | 150 |
| Mean (SD) | 55.86 (24.393) | 65.35 (150.258) | 57.43 (52.544) | 55.99 (22.564) | 53.38 (11.325) |
| Min, Max | 50.0, 287.0 | 50.0, 3050.0 | 50.0, 1050.0 | 50.0, 264.0 | 50.0, 142.0 |
| Median | 50.0 | 50.0 | 50.0 | 50.0 | 50.0 |
| Week 12 | 351 | 366 | 374 | 373 | 117 |
| Mean (SD) | 451.68 (621.921) | 547.83 (1884.845) | 228.52 (618.660) | 247.17 (441.289) | 57.32 (30.769) |
| Min, Max | 50.0, 6770.0 | 50.0, 29000.0 | 50.0, 10200.0 | 50.0, 4730.0 | 50.0, 292.0 |
| Median | 284.0 | 250.0 | 123.5 | 132.0 | 50.0 |
| Month 12 | 283 | 301 | 311 | 280 | 91 |
| Mean (SD) | 534.41 (1375.196) | 386.53 (781.045) | 181.41 (242.823) | 219.11 (678.268) | 56.26 (20.231) |
| Min, Max | 50.0, 18800.0 | 50.0, 10500.0 | 50.0, 2240.0 | 50.0, 10300.0 | 50.0, 183.0 |
| Median | 263.0 | 232.0 | 119.0 | 115.0 | 50.0 |

Abbreviations: E2 - 17β-estradiol; P - progesterone; SD - standard deviation

### 4. Safety Conclusions

There were 1835 enrolled subjects who were eligible for inclusion in the Safety population and 1255 subjects met eligibility for the ES population.

The primary safety endpoint was the incidence of endometrial hyperplasia that was assessed in the ES population:
1) no cases of endometrial hyperplasia, as defined a priori by the Pathology Charter, were observed during the trial; the one-sided upper 95% confidence limit was less than 4% for all groups
2) all subjects had a final diagnosis per the Pathology Charter of Category 1 (non-endometrial malignancy/non-hyperplasia)

Secondary safety endpoints included cumulative amenorrhea and bleeding/spotting:
1) cumulative amenorrhea from Cycle 1 to 13 (ie, no bleeding or spotting for the study) was: 79% for placebo; for active treatment groups, subjects in the 0.25 mg E2/50 mg P group had the highest rate of 73.1%, followed by similar percentage for 0.5 mg E2/100 mg P and 0.5 mg E2/50 mg P (~68%) and lower for the 1 mg E2/100 mg P group (56.1%)
2) at the end of the study, cumulative amenorrhea rates were similar for the active treatment groups and placebo except for the 1 mg E2/100 mg group compared to placebo (90.2% vs 97.8%, respectively; p = 0.023)
3) the percentage of subjects reporting spotting during the first trimester ranged from 18.9% to 28.8% in the active groups compared to 9.7% in placebo during the fourth trimester, the percentage of subjects reporting spotting decreased for all groups; for active treatment groups, the range was 6.5% to 16.7% and for placebo 4.3%
4) bleeding during the first trimester ranged from 7.5% to 15.4% of active treatment groups compared to 3.9% in placebo
   during the fourth trimester, the percentage of subjects with bleeding decreased across all groups and was similar between placebo and the two lowest doses of TX-001HR with the two higher doses having a greater percentage of subjects with reported bleeding
5) though bleeding/spotting was reported during the study, the mean number of days that subjects experienced bleeding/spotting was less than one day
   for the three higher doses of TX-001 HR, the number of days ranged up to 28 days while the lowest dose of TX-001HR and placebo ranged up to seven (7) days (five days for 0.25 mg E2/50 mg P and seven days for placebo)

Additional safety data for the 1835 subjects in the Safety population are summarized below:
1) 1270 (69.2%) subjects experienced at least one AE and 1258 (68.6%) reported at least one TEAE during the study
2) TEAEs across active treatment groups were similar (67.9% to 71.6%) compared to 51.7% for the placebo group
3) most frequently occurring TEAEs (occurring in ≥ 3% in any treatment group) and more commonly than placebo were:
   headache, nasopharyngitis, breast tenderness, upper respiratory tract infection, nausea, back pain, abdominal pain, sinusitis, dizziness, pelvic pain, diarrhea, vulvovaginal mycotic infection, abdominal distension, vaginal discharge, hypertension, influenza, and vaginal bleeding
4) most TEAEs were considered mild or moderate in severity and the majority were classified as not related to study drug
5) most frequently occurring drug related TEAEs (occurring in ≥ 3% in any treatment group) and more commonly than placebo were:
   breast tenderness, headache, nausea, pelvic pain, vaginal bleeding, and vaginal discharge
6) 40 subjects reported 47 TESAEs during the study; the percent of subjects with TESAEs in the active treatment groups ranged from 1.9% to 3.1% compared to 1.3% in placebo
   seven TESAEs were assessed as possibly or probably related to study drug; there were no differences across treatment arms: acute pancreatitis, three cases of breast cancer, invasive ductal breast carcinoma, DVT, and infective cholecystitis
7) two additional subjects had related SAEs (occurred more than 15 days after last dose of IP); both subjects were in the 0.25 mg E2/50 mg P group (invasive ductal breast carcinoma and chronic obstructive pulmonary disease)
8) discontinuations due to TEAEs for the active treatment groups ranged from 7.3% to 10.8% compared to 6.6% in placebo
9) there was one death during the study (subject diagnosed with non-small cell lung cancer Stage IV with a pleural effusion on Study Day 60)
10) one case of VTE was reported in a subject with a history of prior left femoral popliteal bypass surgery and a family history of DVT (son)
11) malignancies occurred infrequently
   two subjects were diagnosed with lung cancer during the trial; neither were assessed by the PI as related to study medication given the short duration of study drug exposure and prior medical history (diagnosis was made 60 days and 102 days after initiating study drug)
   six subjects were diagnosed with invasive breast cancer at the end of the study that were assessed as possibly or probably related to IP by the PI and Medical Monitor
12) laboratory evaluations were predominantly within normal limits
   triglycerides increased over the trial and were higher for the active treatment groups as compared to placebo
   there were no differences across groups in total cholesterol, HDL cholesterol, or LDL cholesterol
13) the mean hormone concentrations for estradiol, estrone, and progesterone at Screening were all consistent with postmenopausal status
14) throughout the study, a dose response was observed for serum concentrations of estradiol and for estrone related to the dose dependent manner of the estradiol dose given
   estradiol and estrone levels remained consistent over time for each respective treatment arm, and were similar in the 0.5 mg E/100 mg P and 0.5 mg E2/50 mg P groups
15) a dose response was observed for serum concentrations of progesterone and progesterone levels remained consistent over time for each respective arm

In summary, during the one year trial, there were no cases of endometrial hyperplasia or malignancy. Overall, the incidence of AEs and TEAEs were more common in the active treatment groups than placebo and were generally mild to moderate in severity. Generally, the percentages of AEs of special interest were low and did not occur with greater frequency in the active arms than placebo. Events of special interest such as cardiovascular disease, VTE, and cerebrovascular AEs were within rates of that of a background population of postmenopausal women. Physical examination, ECGs, and laboratory evaluations were predominantly within normal limits. Overall, the incidence and nature of the adverse events reported in this study are consistent with that expected for this population and with estradiol and progesterone treatment.

### K. Discussion and Overall Conclusions

This was a large Phase 3, randomized, double-blind, placebo-controlled, multi-center trial comparing four combinations of 17β-estradiol and progesterone (1 mg E2/100 mg P, 0.5 mg E2/100 mg P, 0.5 mg E2/50 mg P, and 0.25 mg E2/50 mg P) with placebo for the treatment of moderate to severe vasomotor symptoms in women with an intact uterus.

The primary efficacy and safety endpoints were:
1) Primary efficacy: change from Baseline to Weeks 4 and 12 in the frequency and severity of moderate to severe VMS
2) Primary safety: the incidence of endometrial hyperplasia at 12 months (to demonstrate a hyperplasia proportion that was ≤ 1% with an upper bound of the one-sided 95% CI for that rate that does not exceed 4%)

### Efficacy

The pre-specified primary efficacy endpoint was evaluated in the modified intent to treat population from the VMS Substudy and included 141 subjects in the 1 mg E2/100 mg P group, 149 subjects in the 0.5 mg E2/100 mg P group, 147 subjects in the 0.5 mg E2/50 mg P group, 154 subjects in the 0.25 mg E2/50 mg P group, and 135 subjects in the placebo group. Treatment groups were balanced with regard to age, race, and BMI. The overall mean age for the efficacy population was 54.6 years with a mean BMI of 26.6 kg/m². The mean compliance was 92.5% at Week 12 and 89.1% of subjects completed the 12-week VMS Substudy.

At Baseline, subjects in this trial reported, on average, approximately 74 moderate to severe VMS per week. At Week 4, statistically significant reductions in the frequency of moderate to severe VMS were achieved with all TX-001HR doses compared to placebo (p < 0.013), except for the 0.5 mg E/50 mg P dose (mean change from Baseline ranged from -33.6 to -40.6 for active treatment groups compared to -26.4 for placebo). By Week 6 and continuing through Week 12, statistically significant reductions in the frequency of moderate to severe VMS were observed for all TX-001HR doses compared to placebo (p < 0.002) with a mean change from Baseline ranging from -50.2 to -55.1 for active groups compared to -40.2 for placebo, achieving efficacy despite a very high placebo response rate (55% decrease from Baseline).

To assess the clinical meaningfulness of the observed reduction in the frequency of moderate to severe VMS, responder thresholds were determined in order to provide for a definition of clinically meaningful and important changes in the frequency of moderate to severe VMS. An anchor-based method was used to determine the clinically meaningful responder definition for changes in the frequency of weekly moderate to severe VMS (Revicki DA, et al. J Clin Epidemiol, 2008. 61(2):102-109); Gerlinger C, et al., Menopause, 2012. 19(7):799-803). A single global anchor scale, based on the CGI for change in the subject’s overall condition (Gerlinger et al. 2012, above), was used for developing the definition of responders for frequency of moderate to severe VMS. Based on this analysis, which identified a reduction of ≥ 36 VMS at Week 4 and ≥ 39 VMS at Week 12 as the definition of a responder, all doses of TX-001HR were statistically significantly different from placebo at Weeks 4 and 12 (p < 0.05) and provided clinically meaningful effectiveness. A consistency of effect was also supported by discontinuation rates due to lack of efficacy. Discontinuations from the study occurred in only 1.2% of subjects in the combined active treatment groups due to lack of efficacy whereas 8.9% of subjects in the placebo group discontinued for this reason.

The mean severity at Baseline was approximately 2.5, consistent with the inclusion of a moderate to severe VMS population. Beginning at Week 3 and through Week 12, statistically significant improvements in severity compared to placebo were observed with the two higher doses of TX-001HR (1 mg E2/100 mg P and 0.5 mg E2/100 mg P), and at Week 12 with 0.5 mg E2/50 mg P dose.

Additional consistency of the efficacy of TX-001HR for the treatment of moderate to severe VMS, is noted by the statistically significant improvements in the Vasomotor Domain from MENQOL which were observed at the measured timepoints: Week 12, Month 6, and Month 12 for all TX-001HR groups compared to placebo (p ≤ 0.008). These data support not only the efficacy observed at Week 12 in reducing the frequency of VMS but also sustained improvements through Month 12.

Additional supporting evidence for efficacy include:
1) All doses demonstrated a statistically significant reduction in the frequency of mild, moderate, and severe VMS by Week 6 and continued through Week 12
2) Statistically significant reductions from placebo in the severity of mild, moderate, and severe VMS were observed by Week 3 for the 1 mg E2/100 mg P group and by Week 12 for all doses except for the 0.25 mg E2/50 mg P group
3) There were significantly more subjects (responders) in the active treatment arms who had greater than 50% and 75% reductions in the numbers of their moderate to severe VMS at both Week 4 and Week 12
4) At Week 12, statistically significant improvements in the MENQOL Total Score were observed for all active treatment groups compared to placebo
5) The MOS measurements of sleep indicated improvements in the active treatment arms compared with placebo in those sleep indices typically associated with postmenopausal sleep difficulties (ie, sleep disturbance and sleep adequacy)

Subgroup analyses also demonstrated an overall consistency of effect of TX-001HR. One exception was noted when data were analyzed by race. The placebo response rate in this study was high in the overall population (55%). A statistically significant difference was observed (p = 0.049) in the placebo response rate between White (51%) and Black/African American subjects (65%) at 12 weeks in the MITT population.

### Safety

The primary safety endpoint, the incidence of endometrial hyperplasia, was assessed in the ES population (n=1255). No cases of endometrial hyperplasia, as defined a priori by the Pathology Charter, were observed during the trial and the one-sided upper 95% confidence limit was less than 4% for all groups (1.06% for 1 mg E2/100 mg P; 0.98% for 0.5 mg E2/100 mg P; 0.97% for 0.5 mg E2/50 mg P; 1.09% for 0.25 mg E2/50 mg P; and 3.20% for the placebo group). All subjects had a final diagnosis per the Pathology Charter of Category 1 (non-endometrial malignancy/non-hyperplasia), supporting endometrial protection with progesterone in all combined doses of TX-001 HR.

Secondary safety endpoints included cumulative amenorrhea and bleeding/spotting. Cumulative amenorrhea rates from Cycle 1 to 13 were higher for the 0.5 mg E2 treatment groups (~ 68%) compared to the 1 mg E2 group (56.1%); all groups continued to increase over time, including the highest dose of estradiol (1 mg E2/100 mg P). During the first three months of treatment, ≥ 70% of subjects experienced no bleeding. By Cycle 13, amenorrhea rates for all TX-001HR doses were similar to placebo, except for the 1 mg E2/100 mg group compared to placebo though still > 90% (90.2% vs 97.8%, respectively; p = 0.023). At Cycle 13, < 3% of subjects across all groups, with no difference between active treatment groups and placebo. The mean number of days that subjects experienced bleeding/spotting was ≤ 1.5 days for any cycle across the study. By Cycle 13, mean was < 1 day for all groups with the maximum number of days being 28 days for the highest dose of TX-001HR and 5-7 days for the lowest dose of TX-001HR and placebo. To put this into perspective, another combination estrogen/progestin product (Prempro®) had a reported cumulative amenorrhea rate ranging from 22.1% (CEE 0.625/MPA 2.5) to 45.2% (CEE 0.3/MPA1.5) and there was more bleeding reported that required sanitary protection (Archer DF, et al., Fertil Steril, 2001. 75:1080-1087).

Additional safety evaluations were performed for the 1835 enrolled subjects who were eligible for inclusion in the Safety population: 415 subjects in the 1 mg E2/100 mg P group, 424 in the 0.5 mg E2/100 mg P group, 421 in the 0.5 mg E2/50 mg P group, 424 in the 0.25 mg E2/50 mg group, and 151 subjects in placebo.

TEAEs occurred more frequently in the active treatment groups than in the placebo group (67.9% to 71.6% vs 51.7%, respectively). The majority of AEs were considered mild or moderate in severity and were classified as not related to study drug. Related TEAEs occurred more frequently in the active treatment groups (combined 34.4%) as compared to placebo (17.9%); the most frequently occurring drug related TEAEs (occurring in ≥ 3% in any treatment group) and more commonly than placebo were: breast tenderness, headache, nausea, pelvic pain, vaginal bleeding, and vaginal discharge. Discontinuations due to TEAEs were < 11% for all groups. The number of subjects who reported SAEs was similar across groups (1.9% to 3.1% for active treatment groups vs 1.3% for the placebo group). Seven TESAEs were assessed as related to study drug (six for the active treatment groups and one for placebo); the only TESAE reported more than once was breast cancer. Two additional subjects had related SAEs (occurred more than 15 days after last dose of IP); invasive ductal breast carcinoma and chronic obstructive pulmonary disease.

There was one death during the study (subject diagnosed with non-small cell lung cancer Stage IV with a pleural effusion on Study Day 60, which in retrospect was present at Randomization).

AEs of special interest included: VTEs; superficial thrombosis/phlebitis; cardiac AEs of interest; ECG reported AEs; cerebrovascular AEs of interest; chest pain AEs; syncope; breast cancer AEs; other breast AEs of interest; cervical AEs; AEs related to the endometrium; and malignancies. Overall, malignancies diagnosed during the study were infrequent; two subjects were diagnosed with lung cancer (reported on Study Day 60 and the second on Study Day 102) and six subjects (0.36% active treatment arms) were diagnosed with invasive breast cancer. The incidence rates of invasive breast cancer at one year in this trial are consistent with the background rates as observed in the PEPI trial (0.73%; PEPI 1995) and from the SEER database (Howlader N, Noone AM, Krapcho M, Miller D, Bishop K, Kosary CL, Yu M, Ruhl J, Tatalovich Z, Mariotto A, Lewis DR, Chen HS, Feuer EJ, Cronin KA (eds). SEER Cancer Statistics Review, 1975-2014, National Cancer Institute. Bethesda, MD, https://seer.cancer.gov/csr/1975_2014/, based on November 2016 SEER data submission, posted to the SEER web site, April 2017). Events of special interest such as cardiovascular disease, VTE, and cerebrovascular AEs were within rates of that of a background population of postmenopausal women.

Triglycerides increased over the trial and at Month 12 were higher for all treatment groups compared to placebo, though lower than reported in a previous trial of combination estrogen and progestin (PEPI, 1995). Effects on total cholesterol, HDL, and LDL were neutral as the active treatment groups were similar to placebo.

### Overall Conclusions

The two highest doses of TX-001HR (1 mg E2/100 mg P and 0.5 mg E2/100 mg P) demonstrated clear and consistent efficacy in the reduction of the frequency and severity of moderate to severe VMS at both 4 and 12 weeks compared to Baseline and placebo. TX-001HR 0.5 mg E2/50 mg P and 0.25 mg E2/50 mg P doses demonstrated statistically significant reductions in the of frequency of VMS by 12 weeks; the 0.25 mg E2/50 mg P group was statistically significantly different from placebo by Week 3 and continued to show improvement through Week 12 and the 0.5 mg E2/50 mg P group by Week 6 through Week 12. TX-001HR 0.5 mg E2/50 mg P demonstrated improvement in severity of VMS by Week 12. Of note, in this study there was a very high placebo response rate (55%), yet despite this both statistical and clinically meaningful benefits were observed in the treatment groups versus the placebo group. Additional analyses demonstrated the clinical meaningful effectiveness of all doses. The consistency of effect was observed across co-primary and multiple secondary endpoints focused on patient reported outcomes, including responder rates, CGI, MENQOL, and MOS-Sleep.

The incidence of endometrial hyperplasia at 12 months was the primary safety endpoint. There were no cases of endometrial hyperplasia or malignancy. TEAEs and related TEAEs occurred more frequently in the active treatment groups compared to placebo; however, SAEs and AEs of special did not occur with greater frequency in the active arms than placebo. Physical examination, ECGs, and laboratory evaluations were predominantly within normal limits and not different among active and placebo subjects. Overall, the incidence and nature of the adverse events reported in this study are consistent with that expected for this population and with estradiol and progesterone treatment.

In conclusion, the totality of the data from this double-blind, randomized, placebo-controlled trial demonstrated that TX-00 1HR exhibited consistent efficacy for the treatment of moderate to severe VMS in women with an intact uterus, and that the fixed estradiol and progesterone combinations protected the endometrium from hyperplasia and malignancy. TX-001HR was well tolerated with no clinically significant differences in AEs compared with placebo.

### Example 8

### 1. Introduction

A decrease in estrogen production at the time of menopause is associated with vasomotor instability (hot flushes and sweating), agitation, sleep disturbances, nervousness, mood changes, and urogenital atrophy. The predominant and most bioactive estrogen is 17β-estradiol that is produced by the ovaries. Estrogen therapy has been used for several decades for the management of menopausal symptoms, including vasomotor and vulvar and vaginal atrophy symptoms.

The prolonged use of unopposed estrogens increases the risk of endometrial hyperplasia which is a possible precursor to endometrial adenocarcinoma in women after menopause (Thom MH, et al., Br. J. Hosp. Med. 1980 May; 23 (5): 506, 508-9, 511-3). Progestogens are intended for use in women with an intact uterus as an adjunct to estrogen replacement therapy in order to protect the uterus from hyperplasia and cancer (Graham JD, et al., Endocr. Rev. 1997 Aug; 18 (4): 502-19).

Numerous fixed-dose combination estrogen and progestin products are commercially available in various dosages and formulations, including oral tablets and transdermal products. Combination menopausal hormone therapy has been available for more than two decades beginning with the Food and Drug Administration approval of Prempro® in 1994. Combining an estrogen and a progestin in a formulation not only aids in compliance and convenience to the patient but also when delivered continuously as a combined therapy, reduces the incidence of withdrawal bleeding (Ellerington MC, et al. Br. Med. Bull. 1992 Apr; 48 (2): 401-25).

Currently marketed menopausal hormone therapy combinations incorporate conjugated estrogens, semisynthetic estrogens such as ethinyl estradiol or 17β-estradiol, and synthetic progestins such as norethindrone acetate, drospirenone, norgestimate, and medroxyprogesterone acetate.

The investigational drug TX-001HR (solubilized estradiol and micronized progesterone capsules) is a combination liquid-filled softgel capsule intended for the treatment of moderate to severe vasomotor symptoms associated with the menopause in women with a uterus. TX-001HR contains 17β-estradiol and progesterone, which are naturally occurring steroid hormones that are chemically identical to those of endogenous origin. TX-001HR is intended to provide a continuous combined hormone therapy regimen for the treatment of moderate to severe vasomotor symptoms with endometrial protection in menopausal women with an intact uterus.

Prior to menopause, the primary source of estrogen in normally cycling adult women is the ovarian follicle, which secretes 70 to 500 µg of estradiol daily, depending on the phase of the menstrual cycle. Following menopause, the majority of endogenous estrogen is produced by conversion of androstenedione, secreted by the adrenal cortex, to estrone in the peripheral tissues.

Progesterone is produced in high amounts in the ovaries (by the corpus luteum) from the onset of puberty and ceases at menopause. It is produced in smaller amounts by the adrenal glands after the onset of adrenarche in both males and females.

This Phase 1 study was designed to evaluate the effect of food on the bioavailability of estradiol and progesterone following a single dose of TX-001HR at the highest fixed-dose combination that was demonstrated in a large Phase 3 clinical trial to provide efficacy with an acceptable safety profile in healthy postmenopausal women with no contraindications to estrogen or progesterone therapy. Plasma levels of estrone were also measured as part of this study as estrone is a metabolite of estradiol.

The effect of food on the bioavailability of estradiol and progesterone was assessed previously through a cross-study comparison of the results from Studies EPROG-1K-351-12 and EPROG-1K-352-12, which were performed at the same clinical site during the same timeframe. These studies were open-label, balanced, randomized, two-treatment, two-period, two-sequence, single-dose, crossover oral comparative bioavailability studies of TX-001HR (2 mg estradiol/200 mg progesterone) versus the reference products consisting of 2 mgEstrace® (estradiol tablets USP) coadministered with 200 mgPrometrium® (progesterone USP) softgel capsules in normal healthy, adult postmenopausal female subjects under fasting and high-fat fed conditions, respectively. Plasma concentrations of progesterone were greatly affected by dosing with food, with peak plasma concentrations (Cₘₐₓ) approximately 21-fold higher. Furthermore, area under the concentration-time curve from 0 to the last measurable concentration (AUC₀₋ₜ) increased 13-fold and area under the concentration-time curve from 0 to infinity (AUC_{0-∞}) increased 9-fold. In contrast, food had little to no effect on the plasma concentrations of estradiol. Cₘₐₓ for unadjusted estradiol decreased approximately 33% for TX-001HR with food, while AUC₀₋ₜ increased by 1.2-fold and AUC_{0-∞} increased by 2.0-fold. The effects of food on exposures of the metabolites, unconjugated and total estrone, paralleled the effects on parent estradiol.

### 2. Study Objectives and Endpoints

### 2.1. Objectives

The primary objectives of this study were as follows:
To characterize the effect of food on the bioavailability of estradiol and progesterone following a single dose of TX-001HR 1 mg estradiol/100 mg progesterone under fasting and fed (high-fat meal) conditions
To characterize the plasma levels of estradiol metabolite, estrone

The secondary objective of this study was as follows:
To assess the safety and tolerability of TX-001HR after a single dose under fasted and fed (high-fat meal) conditions

### 3. Investigational Plan

### 3.1. Description of Overall Study Design and Overview of Study Procedures Performed

This was a Phase 1, open-label, randomized, balanced, single-dose, two-treatment (fed and fasting), crossover, single-center study to assess the effect of food on the bioavailability of TX-001HR (estradiol and micronized progesterone capsules) in healthy postmenopausal female subjects meeting the eligibility criteria. In addition to the Screening Visit (Visit 1), eligible subjects attended two in-patient research facility visits, one during each period. The first was an overnight visit occurring on Period 1, Day -1 through Day 4 (Visit 2). Similarly, during Period 2, there was an overnight visit in the research facility occurring Day -1 through Day 4 (Visit 3).

A total of 24 eligible subjects were randomized to receive a single oral dose of TX-001HR 1 mg estradiol/100 mg progesterone under either fasting or fed conditions, in a 1:1 ratio, in the morning of Period 1, Day 1. The assigned meal condition was specified from a randomization table. Before 8:00 PM in the evening of Day -1, subjects were admitted to the research facility and served a moderate-fat meal. Regardless of treatment, subjects fasted overnight, for at least 10 hours. For treatment under fasting conditions, a single dose of study drug was administered by the Investigator (or a staff member as delegated by the Investigator) along with 240 mL of ambient-temperature water within 5 minutes after the 0-hour pharmacokinetic (PK) blood draw. Subjects continued fasting until following the 4-hour blood draw when the subjects were served the moderate-fat meal. For treatment under fed conditions, a single dose of study drug was administered by the Investigator (or a staff member as delegated by the Investigator), along with 240 mL of ambient-temperature water, approximately 30 minutes after beginning a standardized high-fat meal, such as would be known to those of skill in the art, and within 5 minutes after the 0-hour PK blood draw on Day 1. Subjects then fasted until after the 4-hour blood draw when the subjects were served the moderate-fat meal. Venous blood samples of approximately 10 mL each were obtained at the following times with respect to the Day 1 dose to assess estradiol, progesterone, and estrone concentrations: -60, -30, and 0 minutes (the average of which represented baseline) and then 20, 40, 60, and 90 minutes (±5 minutes), and 2, 3, 4, 6, 8, 12, 18, 24, 36, 48 (±10 minutes), and 72 hours (±2 hours) after study drug administration. Following the collection of the 72-hour PK sample, the subjects were discharged from the research facility.

Following a 14-day washout period between treatments, the subjects were admitted to the research facility on Period 2, Day -1 for the alternate meal condition per the randomization schedule. Regardless of treatment, subjects fasted overnight for at least 10 hours. For treatment under fasting conditions, a single dose of study drug was administered by the Investigator (or a staff member as delegated by the Investigator) along with 240 mL of ambient-temperature water within 5 minutes after the 0-hour PK blood draw on Day 1. Subjects continued fasting until following the 4-hour blood draw when the subjects were fed the moderate fat meal. For treatment under fed conditions, a single dose of study drug was administered by the Investigator (or a staff member as delegated by the Investigator) along with 240 mL of ambient-temperature water, 30 minutes after beginning the standardized high-fat meal and within 5 minutes after the 0-hour PK blood draw on Day 1. Subjects then fasted until after the 4-hour blood draw when the subjects were served the moderate-fat meal. Venous blood samples of approximately 10 mL each were obtained at the following times with respect to the Day 1 dose to assess estradiol, progesterone, and estrone concentrations: -60, -30, and 0 minutes (the average of which represented baseline) and then 20, 40, 60, and 90 minutes (±5 minutes), and 2, 3, 4, 6, 8, 12, 18, 24, 36, 48 (±10 minutes), and 72 hours (±2 hours) after study drug administration.

Following the collection of the 72-hour PK sample, the subjects were discharged from the study.

Unscheduled visits were allowed at Investigator discretion for safety reasons, administrative reasons, or to address subject concerns or questions about the study. Unscheduled visits could have also occurred in order to fulfill protocol requirements (e.g., laboratory re-draws).

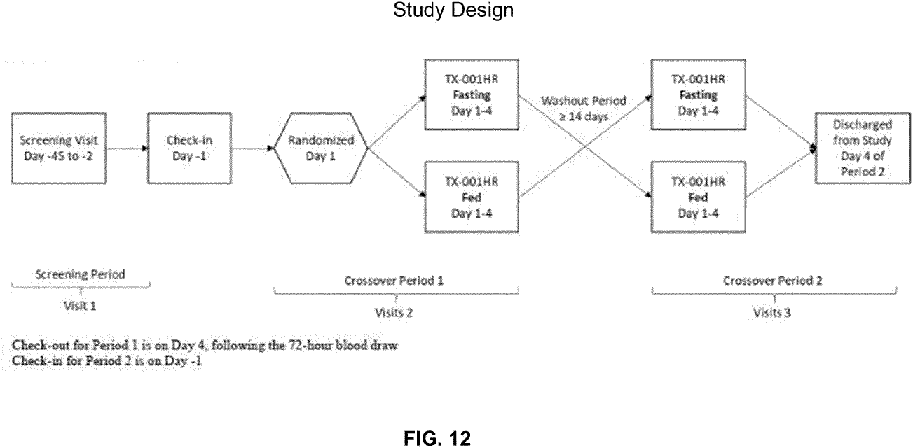

The schematic design of the study is presented in FIG. 12.

### 3.2. Discussion of the Study Design

This was a Phase 1, open-label, randomized, balanced, single-dose, two-treatment, crossover study to assess the effect of food on the bioavailability of TX-001HR in healthy postmenopausal female subjects. Eligible subjects were randomized in a 1:1 ratio to receive a single oral dose of 1 mg estradiol/100 mg progesterone under either fasting or fed conditions. TX-001HR is an estradiol-progesterone combination oral product intended for the treatment of moderate to severe vasomotor symptoms associated with menopause in women with an intact uterus and this study was conducted in postmenopausal females. The high-fat meal was a standardized meal defined and described in the protocol, including a suggested meal with a breakdown of calories from fat, protein, and carbohydrates. The moderate-fat meal was described in terms of calorie content with a breakdown of calories from fat, protein, and carbohydrates.

### 3.3. Selection of Study Population

### 3.3.1. Inclusion Criteria

Volunteers who met all of the following criteria were considered for enrollment in the study:
1. Postmenopausal female subjects between 40 and 65 years old, inclusive (at the time of randomization) with at least:
   a. 12 consecutive months of spontaneous amenorrhea without an alternative medical cause; OR
   b. Women < 55 years of age who were not at least 6 weeks postsurgical bilateral oophorectomy must have had follicle stimulating hormone (FSH) levels > 40 mIU/mL; OR
   c. 6 months of spontaneous amenorrhea with FSH levels > 40 mIU/mL; OR
   d. 6 weeks postsurgical bilateral oophorectomy
2. No underlying disease which, in the opinion of the Principal Investigator or Medical Sub-Investigator, would prevent the subject from safely participating in the study or complying with protocol requirements
3. A body mass index (BMI) between 18 and 30 kg/m². BMI values were rounded to the nearest integer (eg, 30.4 rounded down to 30, while 26.5 rounded up to 27).
4. Screening laboratory values within normal limits or considered by the physician or Principal/Clinical Investigator to be of no clinical significance
5. In the opinion of the Investigator, the subject would comply with the protocol and had a high probability of completing the study
6. Willingness to consume a non-vegetarian diet and a high-fat meal

### 3.3.2. Exclusion Criteria

Volunteers with a history or significant presence of any of the following criteria were excluded from enrollment into the study:
1. Any contraindication to estrogen and/or progestin therapy or allergy to the use of estradiol and/or progesterone or any components of the investigational drug
2. Use of any of the following:
   a. Oral estrogen-, progestin-, androgen (including prasterone)-, or selective estrogen receptor modulator- (SERM-) containing drug products within 8 weeks before Screening Visit
   b. Transdermal hormone products within 4 weeks before Screening Visit
   c. Vaginal hormone products (rings, creams, gels) within 4 weeks before Screening Visit
   d. Intrauterine progestins within 8 weeks before Screening Visit
   e. Progestin implants/injectables or estrogen pellets/injectables within 6 months before Screening Visit
   f. Drugs that are moderate-to-potent inhibitors or inducers of cytochrome P450 3A4 (CYP3A4). A washout of at least 2 weeks prior to randomization (Day 1) was required for eligibility
   g. Any medications, including over-the-counter (OTC) products, herbal products, or nutritional supplements (eg, soy products, injectable corticosteroids, testosterone, or dehydroepiandrosterone), known or suspected to affect estrogen/progestin drug metabolism. A washout of at least 4 weeks prior to randomization (Day 1) was required for eligibility
3. A history or active presence of clinically important medical disease that might confound the study or be detrimental to the subject, including but not limited to:
   a. Hypersensitivity to estrogens or progesterone or other related drugs
   b. Endometrial hyperplasia
   c. Undiagnosed vaginal bleeding
   d. Had a history of a chronic liver or kidney dysfunction/disorder (eg, Hepatitis C or chronic renal failure)
   e. Thrombophlebitis, thrombosis, or thromboembolic disorders
   f. Cerebrovascular accident, stroke, or transient ischemic attack
   g. Myocardial infarction or ischemic heart disease
   h. Malignancy or treatment for malignancy, within the previous 5 years, except for basal cell carcinoma of the skin or squamous cell carcinoma of the skin
   i. A history of estrogen dependent neoplasia, breast cancer, melanoma, or any gynecologic cancer, at any time
   j. Endocrine disease (except for controlled hypothyroidism or controlled non-insulin dependent diabetes mellitus)
4. History of known alcohol or drug abuse within 1 year of Screening Visit
5. Positive urine drug or alcohol screen at Screening Visit or Period 1, Day -1 check-in. Subjects with a positive urine drug screen were eligible if, in the opinion of the Principal Investigator or Medical Sub-Investigator, there was no documented evidence of drug abuse and the reason for the positive drug screen was because of a medically prescribed medication
6. Current cigarette smoker or current use of any tobacco-containing product including electronic cigarettes
7. Current marijuana use
8. Use of an investigational drug or biologic within 60 days before Screening Visit
9. Any clinically important abnormalities on Screening physical exam, assessments, electrocardiogram (ECG), or laboratory tests, including but not limited to:
   a. Unresolved cervical cytologic smear report of atypical glandular cells of undetermined significance (AGUS) or atypical squamous cells of undetermined significance (ASCUS). Cervical cytologic smear report of low-grade squamous intraepithelial lesion (SIL) or greater, cervical intraepithelial neoplasia, grade 1 or greater, or any reported dysplasia; Subjects with ASCUS were eligible only if high risk human papilloma virus (HPV) result was negative.
   b. Unresolved findings suspicious for malignancy on the breast exam; incomplete mammogram result (breast imaging-reporting and data system [BI-RADS] 0) or unresolved findings suggestive of malignant changes or findings requiring short interval follow-up on the pre-study mammogram (subjects must have had mammography result of BI-RADS 1 or 2 to enroll). Mammogram performed within 9 months prior to the Screening Visit with documentation available. If the subject had not had a mammogram performed within 9 months of the Screening Visit, a mammogram was to be performed during the screening period. (The site must have obtained a copy of the official report for the subject’s study file, and it must have been verified that the mammogram itself was available if needed for additional assessment.)
   c. Hematocrit less than 35% or greater than 45%
   d. Serum creatinine greater than 15% of the upper limit of normal for the laboratory used
   e. Serum alanine aminotransferase (ALT) or serum aspartate aminotransferase (AST) greater than 1.5 times the upper limit of normal for the laboratory used
   f. Fasting total cholesterol greater than 300 mg/dL (7.77 mmol/L) or triglycerides greater than 300 mg/dL (3.39 mmol/L)
   g. Positive laboratory finding for Factor V Leiden mutation
   h. Fasting glucose > 125 mg/dL
   i. Uncontrolled hypertension at Screening or Period 1, Day -1; subjects with elevated sitting blood pressure, greater than 140 mm Hg systolic or greater than 90 mm Hg diastolic and not taking more than two antihypertensive medications for the treatment of hypertension
   j. Uncontrolled hypotension at Screening or Day -1; subjects with sitting blood pressure lower than 95 mm Hg systolic or lower than 65 mm Hg diastolic
   k. A clinically significant abnormal 12-lead ECG (such as myocardial infarction or other findings suggestive of ischemia)
10. Pregnancy or a positive urine pregnancy test. (Note: A pregnancy test was not required for subjects who had had bilateral tubal ligation, bilateral oophorectomy, hysterectomy, or were 55 years old or greater and had experienced cessation of menses for at least one year.)
11. Donation of whole blood within 56 days prior to randomization or platelets within 1 month prior to administration of study drug
12. Consumption of grapefruit and/or its juice, starfruit, Seville orange juice or Seville oranges, or poppy seed-containing foods within 48 hours prior to Randomization and throughout the entire study
13. History of difficulty in vein access

### 3.3.3. Removal of Patients From Therapy or Assessment

Participation in the study was strictly voluntary. Every subject had the right to discontinue participation in the study at any time for any reason. Subjects could have discontinued or could have been withdrawn from the study for any of the following reasons:
Adverse event (AE) which warranted withdrawal of subject
Willful withholding of information by subjects that would have excluded the subject from study participation at Screening
Subject became pregnant. If a subject pregnancy had been reported during study participation, the pregnancy would have been followed as medically appropriate
Undue difficulty in obtaining blood sample (i.e., failure to establish an indwelling cannula
Non-compliance with procedures
Premature termination of study
It was in the best interest of the study participant that she be withdrawn
Withdrawal of consent for participation in the study by the subject
Significant protocol deviation/violation or a trend in deviations/violations (defined as a deviation/violation that affected the subjects’ rights, safety, or the integrity of the study data)
Prohibited concomitant therapy was reported or required

If a randomized subject discontinued from the study prior to Period 2, Day 4, the Investigator was to attempt to complete the Early Termination Visit assessment. Further, Investigators were to follow any ongoing AEs until they were resolved or until the subject was clinically stable. In this study, no subjects discontinued. All randomized subjects completed the study.

### 3.4. Treatments

### 3.4.1. Treatments Administered

Treatment consisted of one dose of TX-001HR 1 mg estradiol/100 mg progesterone administered under a fed condition and one dose of TX-001HR 1 mg estradiol/100 mg progesterone administered under a fasting condition. Each treatment was administered by study staff in the research facility. The washout period between treatments was 14 days.

3.4.2. Identity of Investigational Product

TABLE 51

Investigational Product

| Product Name: | TX-001HR |
|---|---|
| Dosage Form: | Soft gelatin capsule |
| Unit Dose: | 1 mg estradiol/100 mg progesterone |
| Route of Administration: | Oral |
| Physical Description: | Oval, opaque, bi-color pink, soft gelatin capsule |
| Manufacturer: | Catalent Pharma Solutions St. Petersburg, FL |
| Batch number: | 3012843 |

### 3.4.3. Method of Assigning Patients to Treatment Groups

Subjects were randomized in a 1:1 ratio to receive TX-001HR at 1 mg estradiol/100 mg progesterone in the sequence of fasting in Period 1 and fed in Period 2 (Sequence A) or fed in Period 1 and fasting in Period 2 (Sequence B). Randomization occurred on Period 1, Day 1. The sequence of treatments was specified from a randomization table. The randomization schedule was generated with the SAS® software, Version 9.2 or higher, SAS Institute Inc, USA.

### 3.4.4. Selection of Doses in the Study

The dose of the study drug TX-001HR used in this study (1 mg estradiol/100 mg progesterone) was selected for evaluation as it is the highest dose for which marketing approval will be sought.

### 3.4.5. Selection and Timing of Dose for Each Patient

On Day 1 of each period, a single dose of study drug was administered in the research facility by the Investigator (or a staff member as delegated by the Investigator) in the morning, either under fasting conditions or approximately 30 minutes after beginning a standardized high-fat meal, based on the randomization schedule. Each dose was administered with 240 mL of ambient-temperature water.

### 3.4.6. Blinding

This study was an open-label and no blinding procedures were necessary.

### 3.4.7. Prior and Concomitant Therapy

All treatments (including prescription or OTC treatments and dietary supplements) taken by the subject within 30 days prior to the first dose of study drug were considered to be prior treatments and were recorded as such in the electronic case report form (eCRF). Any treatments taken from the start of study drug were considered concomitant treatments and were recorded as such in the eCRF. When known, the start date and stop dates, dose, frequency, and indication were recorded.

The following concomitant medications, therapies, and products were prohibited:
Oral estrogen-, progestin-, androgen (including prasterone)-, or SERM-containing drug products were prohibited from 8 weeks before the Screening Visit until the end of the study
Transdermal hormone products were prohibited from 4 weeks before the Screening Visit until the end of the study
Vaginal hormone products (rings, creams, gels) were prohibited from 4 weeks before the Screening Visit until the end of the study
Intrauterine progestins were prohibited from 8 weeks before the Screening Visit until the end of the study
Progestin implants/injectables or estrogen pellets/injectables were prohibited from 6 months before the Screening Visit until the end of the study
Any medication, herbal product, or nutritional supplement known or suspected to interact with estradiol or progesterone therapy was prohibited from 4 weeks prior to the Screening Visit until the end of the study
Moderate to potent inhibitors or inducers of cytochrome P450 3A4 (CYP3A4) were prohibited from at least 2 weeks prior to randomization (Period 1, Day 1) until the end of the study
Any medications, including OTC products, herbal products or nutritional supplements (eg, soy products, injectable corticosteroids, testosterone, or dehydroepiandrosterone), known or suspected to affect estrogen/progestin drug metabolism were prohibited from at least 4 weeks prior to randomization (Period 1, Day 1) until the end of the study
Any other investigational drug or biologic was prohibited from 60 days before the Screening Visit until the end of the study
Subjects were encouraged to refrain from consumption of alcohol from the Screening Visit through the end of the study. Subjects with positive alcohol urine screens at Screening or Period 1, Day -1 were excluded from the study
Subjects who smoked, used other tobacco-containing products, or used marijuana were not eligible for the study

### 3.4.8. Treatment Compliance

Compliance on Period 1, Day 1 and Period 2, Day 1 was assured by a staff member as the study drug was administered under the supervision of the Investigator or a staff member as delegated by the Investigator.

### 3.5. Pharmacokinetic and Safety Variables

### 3.5.1. Pharmacokinetics and Safety Parameters Assessed and Flow Chart

The schedule of assessments is presented in Table 52.

TABLE 52

Schedule of Assessments

| Assessment | Screening Visit* Visit 1 | Periods 1 &2 | | | | | Early Termination Visit |
|---|---|---|---|---|---|---|---|
| | | In-clinic Overnightvisits 2 &3 | | | | | |
| Study Day | -45 to -2 | -1 | 1 | 2 | 3 | 4 | |
| Informed consent | X | | | | | | |
| Review of Inclusion/Exclusion Criteria | X | | | | | | |
| Confirmation of eligibility | | Xⁱ | | | | | |
| Demographics/medical and gynecological history | X | | | | | | |
| Interim medical history | | Xⁱ | | | | | |
| Physical examination (including breast exam, height, weight and BMI calculation) | X | | | | | | |
| Pelvic examination including bimanual examination and Pap smear | X | | | | | | |
| Mammography | X^{a} | | | | | | |
| Prior and concomitant medications and non-pharmacological treatments | X | X | | | | X | X |
| Vital signs (BP, HR, RR, and temperature) | X^{b} | x^{b} | X | X | X | X | X |
| 12-lead ECG | X^{c} | | | | | | |
| Study Day | -45 to -2 | -1 | 1 | 2 | 3 | 4 | |
| Hematology laboratory tests | X | | | | | | |
| Chemistry laboratory tests | X^{d} | | | | | | |
| Factor V Leiden | X | | | | | | |
| Thyroid function (if applicable) | X^{e} | | | | | | |
| FSH (if applicable) | X^{f} | | | | | | |
| Urinalysis | X^{g} | | | | | | |
| Urine pregnancy test (if applicable) | X^{h} | X^{hi} | | | | | |
| Urine drug and alcohol screen | X | Xⁱ | | | | | |
| Randomization | | | X^{j} | | | | |
| Administration of study drug | | | X^{k} | | | | |
| High-fat meal | | | X¹ | | | | |
| Moderate-fat meal | | X^{m} | Xⁿ | Xⁿ | Xⁿ | Xⁿ | |
| Pharmacokinetic blood samples | | | X^{o} | X^{o} | X^{o} | X^{o} | |
| Adverse events | X | X | X | X | X | X | X |
| Research facility check-in | | X^{p} | | | | | |
| Research facility discharge | | | | | | X | |
| Discharge from the study | | | | | | X^{q} | |

Abbreviations: BP = blood pressure; ECG = electrocardiogram; FSH = follicle-stimulating hormone: HR = heart rate; RR = respiratory rate
* Screening must occur within 45 days prior to check-in at the research facility.
^{a} Was performed within 9 months prior to screening visit with documentation available or if a mammogram is needed, it was completed within the screening time frame of -45 days prior to dosing.
^{b} Measured after subject has been sitting for at least 5 minutes. Screening and Day -1 blood pressure results outside eligible range per protocol could have been repeated up to 2 times before declaring the subject a screen failure. On Day 1 of both periods, vital signs were to be measured prior to the -60-minutes blood draw and again 1 to 3 hours after dosing. On Days 2 to 4, vital signs were collected prior to the first PK sample draw that day.
^{c} Obtained after subject had been in a recumbent or semi-recumbent position for at least 10 minutes.
^{d} Glucose and triglyceride assessments required that subject be fasting for a minimum of 8 hours.
^{e} If thyroid stimulating hormone (TSH) was abnormal as per lab range, reflex testing of free triiodothyronine (T3) and free thyroxine (T4) was performed.
^{f} Serum FSH is required for subjects < 55 years of age who were not at least 6 weeks postsurgical bilateral oophorectomy OR subjects with 6 to 12 months of spontaneous amenorrhea
^{g} Dipstick with reflex microscopic examination for any positive findings for blood, white blood cells, nitrates, and protein (≥ +1 only).
^{h} Not required for subjects who had had bilateral tubal ligation, bilateral oophorectomy, hysterectomy, or for subjects who are 55 years old or greater and have experienced cessation of menses for at least 1 year.
ⁱ On Day -1 of Period 1 only.
^{j} On Day 1 of Period 1 only.
^{k} On Day 1 of Periods 1 and 2 study drug was administered with 240 mL of ambient-temperature water by the Investigator (or a staff member as delegated by the Investigator) following and overnight fast of at least 10 hours and either under fasting conditions or 30 minutes after beginning a high-fat meal per randomization table.
^{l} Only for subjects who were treated under fed conditions, a standardized high-fat meal will be served approximately 30 minutes prior to dosing on Day 1.
^{m} Before 8PM in the evening on Day -1, subjects were admitted to the research facility and served a moderate-fat meal.
ⁿ All subjects were served a moderate-fat meal after the 4-hour blood draw on Day 1 and regularly scheduled, standardized, moderate-fat meals continued through Day 4.
^{o} Samples were taken -60, -30 (± 5 minutes), and 0 minutes (-5 minutes) before dosing, and then 20, 40, 60, and 90 minutes (±5 minutes), and 2, 3, 4, 6, 8, 12, 18, 24, 36, 48 hours (±10 minutes), and 72 hours (±2 hours) after the Day 1 dose of study drug.
^{p} Research facility check-in occurred prior to 8:00 PM on Day -1 of Periods 1 and 2.
^{q} On Day 4 of Period 2 only

### 3.5.2. Drug Concentration Measurements

Venous blood samples of 10 mL each were collected with K₃EDTA anticoagulant for assessment of plasma estradiol, estrone, and progesterone at the times indicated below in Table 53. The actual time of collection of each blood sample (to the nearest minute) was recorded on the appropriate source documentation and in the eCRF. The acceptable windows for blood draw from the scheduled time are noted in Table 53. The baseline (0-hour) time point was collected within 5 minutes prior to dosing. Differences outside this range were not considered protocol deviations. However, only actual times were used for the PK analyses.

Estradiol/estrone bioanalysis was performed using a validated liquid chromatographic-tandem mass spectroscopy (LC-MS/MS) assay with a lower limit of quantification (LLOQ) of 5.00 pg/mL for both analytes. Progesterone bioanalysis was performed using a validated LC-MS/MS assay with an LLOQ of 0.1 ng/mL.

TABLE 53

Pharmacokinetic Sample Times

| Period 1, Day 1 | Period 2, Day 1 | Blood draw window (minutes) |
|---|---|---|
| Time Points (hr:min) | Time Points (hr:min) | |
| -00:60 | -00:60 | ±5 |
| -00:30 | -00:30 | ±5 |
| 00:00 | 00:00 | -5 |
| 00:20 | 00:20 | ±5 |
| 00:40 | 00:40 | ±5 |
| 1:00 | 1:00 | ±5 |
| 1:30 | 1:30 | ±5 |
| 2:00 | 2:00 | ±10 |
| 3:00 | 3:00 | ±10 |
| 4:00 | 4:00 | ±10 |
| 6:00 | 6:00 | ±10 |
| 8:00 | 8:00 | ±10 |
| 12:00 | 12:00 | ±10 |
| 18:00 | 18:00 | ±10 |
| 24:00 | 24:00 | ±10 |
| 36:00 | 36:00 | ±10 |
| 48:00 | 48:00 | ±10 |
| 72:00 | 72:00 | ±120 |

Abbreviations: hr = hour; min = minute

### 3.5.3. Pharmacokinetic Parameters Assessed

The following PK parameters were assessed for the Day 1 of Periods 1 and 2, for baseline-adjusted and baseline-unadjusted results, for estradiol, estrone, and progesterone:
Area under the concentration-time curve from zero to the last measurable concentration (AUC₀₋ₜ)
Area under the concentration-time curve extrapolated to infinity (AUC_{0-∞})
Peak (maximum) plasma concentration of the drug, (Cₘₐₓ)
Time to peak (maximum) plasma concentration, (tₘₐₓ)
Terminal elimination rate constant, (λ_{z})
Elimination half-life (t_{½})

### 3.5.4. Safety Parameters Assessed

The safety parameters below were assessed in the Safety Population.

### 3.5.4.1. Vital Signs

Vital signs including blood pressure, heart rate, respiratory rate, and temperature were taken at the Screening Visit and Day -1 of both Periods 1 and 2 (at the time of check-in), on Day 1 prior to the -60-minutes PK blood draw, within 1 to 3 hours post-dose, and on Days 2 thru 4 prior to the first PK blood draw that day of both Periods 1 and 2. Vital signs were to be taken at the Early Termination Visit, as needed. Measurements were obtained after the subject had been sitting for at least 5 minutes.

At Screening and at Day -1, subjects with systolic blood pressure greater than 140 mm Hg or lower than 95 mm Hg and subjects with diastolic blood pressure greater than 90 mm Hg or lower than 65 mm Hg, were excluded from the study. Results outside this range were repeated up to 2 times before declaring the subject a screen failure based on exclusionary blood pressure measurements.

### 3.5.4.2. Screening Physical Examination Including Breast Exam, Height, Weight, and Body Mass Index Calculation

A complete physical examination was performed at the Screening Visit. The physical examination included, at a minimum, examination of the subject’s general appearance, head, eyes, ears, nose, throat, heart, lungs, musculoskeletal system, gastrointestinal system, neurological system, lymph nodes, abdomen, and extremities.

Subjects had a breast examination performed during the Screening Visit.

The subject’s body weight (while lightly clothed) and height (without shoes) was recorded, and the BMI was calculated at the Screening Visit.

### 3.5.4.3. Screening Electrocardiogram

A standard 12-lead ECG was obtained at the Screening Visit after the subject had been in a recumbent or semi-recumbent position for at least 10 minutes. Results must have been normal or deemed not to be of clinical significance by the Investigator and the Medical Monitor. The site obtained a copy of the official report for the subject’s study file, and it was verified that the ECG itself was available if needed for additional assessment.

### 3.5.4.4. Screening Pelvic Examination and Papanicolaou Smear

Subjects were required to have a bimanual pelvic examination and Papanicolaou (Pap) smear performed during the Screening Visit. The Pap smear was required for all subjects with or without an intact uterus and cervix. For subjects without an intact cervix the Pap smear was obtained by sampling the apex of the vaginal cuff. All subjects must have had a Pap smear done during Screening, regardless of any recent prior assessment. Pap smear results reported as ASCUS had reflex HPV analysis to determine eligibility.

### 3.5.4.5. Screening Mammogram

Subjects must have had a mammogram completed within 9 months prior to the Screening Visit or must have undergone mammography during the Screening period. The site obtained a copy of the official report for the subject’s study file, and it verified that the mammogram itself was available if needed for additional assessment

### 3.5.4.6. Assessment of Laboratory Parameters

Blood samples for blood chemistry, hematology, and hormone levels and urine samples for urinalysis were collected at the Screening Visit only.

### Hematology and Coagulation

The following hematology laboratory parameters were assessed at the Screening Visit: hemoglobin, hematocrit, mean corpuscular hemoglobin, mean corpuscular hemoglobin concentration, mean corpuscular volume, platelet count, red blood cell count, white blood cell (WBC) count, and WBC differential. Factor V Leiden was assessed at the Screening Visit only (to determine eligibility).

### Blood Chemistry

The following chemistry laboratory parameters were assessed at the Screening Visit: glucose, sodium, chloride, potassium, bicarbonate, calcium, albumin, total protein, AST, ALT, alkaline phosphatase, total bilirubin, total cholesterol, triglycerides, creatinine, blood urea nitrogen, and uric acid. Glucose and triglyceride assessments required that the subject be fasting for a minimum of 8 hours.

### Thyroid Function

Thyroid stimulating hormone (TSH) was assessed at the Screening Visit only (to determine eligibility). If TSH was abnormal as per lab range, reflex testing of free triiodothyronine and free thyroxine was performed.

### Urinalysis

At the Screening Visit, urine was collected for urinalysis with reflex microscopic examination for any positive findings on dipstick for blood, WBC esterase (leukocyte esterase), nitrates, and protein (≥ +1 only). The following urinalysis laboratory parameters were assessed: pH, protein, specific gravity, glucose, ketones, blood, WBC esterase, nitrites, urobilinogen, and myoglobin.

### Urine Drug and Alcohol Screen

Urine was collected at the Screening Visit and upon admission to the clinic on Period 1, Day 1 for the following drugs: cocaine, tetrahydrocannabinol, phencyclidine, amphetamines (including methamphetamines), opiates (including heroin and codeine), benzodiazepines, barbiturates, methadone, tricyclic antidepressants, oxycodone, buprenorphine, and alcohol.

### Pregnancy Screen

A urine pregnancy test was performed at the Screening Visit and on Period 1, Day 1. Note: A pregnancy test was not required for subjects who had had a hysterectomy, bilateral tubal ligation, or bilateral oophorectomy or for subjects who were 55 years of age or greater and had experienced cessation of menses for at least 1 year.

### Follicle-Stimulating Hormone

In this study, postmenopausal was defined as ≥ 12 months of spontaneous amenorrhea without an alternate medical cause; women < 55 years of age were not at least 6 weeks postsurgical bilateral oophorectomy must have had a FSH level > 40 mIU/mL); ≥ 6 months of amenorrhea and a serum FSH > 40 mIU/mL; or ≥ 6 weeks postsurgical bilateral oophorectomy. If needed to confirm postmenopausal status, a blood sample was taken at the Screening Visit to assess FSH.

### 3.5.4.7. Assessment of Adverse Events

An AE in this study included an undesirable medical condition occurring at any time, including the Screening period, even if no study treatment had been administered. All AEs that occurred after any subject had enrolled, before treatment, during treatment, or through the 72-hour blood draw on Day 4 of Period 2, whether or not they were related to the study, were recorded in source documentation on the eCRF for randomized subjects.

A treatment-emergent adverse event (TEAE) was the development of an undesirable medical condition or the deterioration of a preexisting medical condition following or during exposure to a pharmaceutical product, whether or not considered casually related to the product. A TEAE could occur from overdose of study drug. Any medical condition that occurred during the Screening period (eg, intercurrent infection), was not considered a TEAE (it was considered an AE).

An AE that occurred during any study phase (screening, treatment, or follow-up) and at any dose of the study drug that fulfilled one or more of the following criteria below was considered a serious adverse event (SAE).
Resulted in death
Was life-threatening. The subject was at immediate risk of death from the AE as it occurred
Required in-subject hospitalization or prolongation of existing hospitalization. Hospitalization itself was not considered an SAE
Resulted in persistent or significant disability or incapacity. Disability was defined as a substantial disruption in a person’s ability to conduct daily functions
Resulted in a congenital abnormality or birth defect
Was an important medical event that may have jeopardized the subject or required medical intervention to prevent one of the outcomes listed above. An important medical event that did not result in death, was not life-threatening, or did not require hospitalization may have been considered an SAE when, based upon appropriate medical judgment, the event may have jeopardized the subject and required medical or surgical intervention to prevent one of the outcomes listed in this definition. Examples of such events are intensive treatments in an emergency room or at home for allergic bronchospasm, blood dyscrasias, or convulsions that did not result in hospitalization, or development of drug dependency or drug abuse. Adverse events resulting in hospitalization were considered serious.

All SAEs that occurred after the signing of the ICF up through discharge on Day 4 of Period 2 must have been reported whether or not they were related to the study drug. Any SAEs that were considered at least possibly related to the study drug were to be reported at any time after the study.

The Investigator was required to assess the severity of each AE (mild, moderate, or severe) and relationship to study drug (not related, possibly related, or probably related).

### 3.5.5. Appropriateness of Measurements

The PK and safety assessments used in this study were standard measurements and considered appropriate to meet the objectives of the study.

### 3.6. Data Quality Assurance

This clinical study was performed in compliance with the Sponsor or designee’s standard operating procedures as well as regulations set forth by ICH GCP guidelines and other relevant regulatory authorities.

The Investigator or designee entered study data required by the protocol into an Electronic Data Capture (EDC) system. The clinical research associates visited the clinical site to review eCRFs for completeness and accuracy. Any discrepancies found between source documents and completed eCRFs were entered as a discrepancy in the EDC system by the clinical research associate. Appropriate clinical site personnel then addressed those discrepancies in the EDC system. Uniform procedures for eCRF correction (queries) were discussed at the eCRF completion training, and were documented in supplemental study-specific guides and instructional manuals. Quality control, monitoring and data validation procedures were applied to ensure the validity and accuracy of the clinical database.

Computerized and manual procedures were used to review and check data from eCRFs and data from external sources for omissions, apparent errors, and values that required further clarification. Data queries requiring clarification were documented and requested of the clinical site for review and resolution. Only authorized personnel could make corrections to the clinical database and all corrections were documented in an audit trail.

### 3.7. Statistical Methods Planned in the Protocol and Determination of Sample Size

### 3.7.1. Populations

The statistical analyses were to be performed on the following planned subject populations:
1. Safety Population: This population consisted of all subjects who were randomized to the study and received at least one dose of study drug. The demographic and safety summaries were based on the Safety Population and equivalent to the ITT Population.
2. PK Population: This population consisted of subjects who completed both periods and had sufficient data to calculate Cₘₐₓ, AUC₀₋ₜ, and AUC_{0-∞}. The PK summaries were based on the PK Population.
3. Completers: This population consisted of all subjects in the PK population that were discharged from the clinic following the 72-hour blood draw in Period 2.

### 3.7.2. Subject Disposition

All subjects randomized were accounted for. The number of subjects randomized into the study was summarized by treatment sequence. All randomized subjects completed the study. No subjects discontinued from the study.

### 3.7.3. Demographic and Baseline Characteristics

Categorical data were summarized using numbers and percentages. The percentages were based on the total number of subjects with a corresponding assessment. Continuous data were presented using the number of subjects (N), mean, standard deviation (SD), median, minimum, and maximum. Summaries were conducted for the Safety Population by treatment sequence. All baseline characteristics were also listed. Missing values for demographics and baseline characteristics were not imputed.

Medical history results were coded using the Medical Dictionary for Regulatory Activities (MedDRA) version 18.0 and summarized by system organ class (SOC) and preferred term (PT).

### 3.7.4. Prior and Concomitant Treatments

Prior and concomitant medications were listed by subject, coded using the World Health Organization (WHO) Drug Dictionary version 01MAR2014, and summarized.

Prior medications were defined as any medications started prior to the day of the first dose of study drug. Prior medication was summarized by treatment sequence.

Concomitant medications were defined as any medications taken on the day of or after the first dose of study drug. Concomitant medications were summarized and presented for all subjects.

Medications were classified according to primary 4^{th} level Anatomical Therapeutic Chemical codes and WHO Drug PTs in the listing. Subjects may have had more than one concomitant treatment per drug class and per PT. Summary tables were generated for both prior and concomitant treatments, and in the tabular summaries, a subject was counted only once for a given prior or concomitant treatment. Prior and concomitant non-medicinal therapies (non-drug treatments) were coded using MedDRA and then summarized in a similar manner as prior and concomitant medications. If the timing of the dose of a concomitant medication could not be established in relation to the administration of study drug, it was considered as concomitant medication.

### 3.7.5. Protocol Deviations

Protocol deviations are summarized by deviation category.

### 3.7.6. Compliance

The number of subjects who received study drug under fed and fasting conditions is summarized by treatment sequence for the Safety Population.

### 3.7.7. Pharmacokinetic Analyses

A number of different PK parameters were evaluated for the PK Population.

PK parameters were calculated using the SAS® program which followed the same algorithm as that implemented in WinNonlin® Professional (Version 7.0, Pharsight Corporation, A Certara Company, St. Louis, MO). In addition, the SAS results were validated using the parameters generated from WinNonlin 7.0. The baseline-adjusted and baseline-unadjusted PK parameters were calculated for each subject for estradiol, estrone, and progesterone, respectively. If any concentration was missing, the reason of the missing (eg, lost sample; sample not collected) was identified.

Plasma concentrations were tabulated and listed by nominal sample time and treatment (fed and fasting). Subjects excluded from the PK Population were presented in the concentration data listings, but were excluded from the summary statistics and noted as such in the tables. All values below the limit of quantification (BLQ) were presented as “BLQ” in the individual concentration data listing and footnoted accordingly and treated as “0” in the descriptive summary statistics of concentration data. Leading BLQ values were treated as 0 when calculating the PK parameters; embedded and trailing BLQ values were treated as “missing” when calculating PK parameters (eg, AUC₀₋ₜ, λ_{z}).

Baseline concentrations were defined as the average of the -60, -30, and 0 minutes (just before dosing) samples on Day 1 for each period. The baseline concentrations were subtracted from each concentration after dosing for the baseline-adjusted concentrations and the subsequent PK parameters were estimated based on these adjusted values for each period. If the baseline-adjustment resulted in a negative plasma concentration value, the value was set equal to 0 before calculating the baseline-adjusted PK parameters. If the baseline concentration and post-baseline concentration were both BLQ, the baseline-adjusted concentration value was set equal to 0 as well before calculating the baseline-adjusted PK parameters.

Figures were created to display mean and individual concentration-time curves using both raw and log-transformed data for the baseline-adjusted and unadjusted concentration data. Mean and individual concentration-time profiles of estradiol, estrone, and progesterone concentration data are presented on linear and semi-log scales. Figures were presented with their SD for linear mean plots and without their SD for semi-log plots.

Plasma concentrations of estradiol, estrone, and progesterone were summarized by treatment (fed or fasting) descriptively: number of observations, arithmetic mean, SD, coefficient of variation (CV), geometric mean, geometric CV, median, minimum, and maximum.

For each analyte, the baseline-adjusted and baseline-unadjusted PK parameters including Cₘₐₓ, tₘₐₓ, AUC₀₋ₜ, AUC_{0-∞}, λ_{z} and t_{½} were summarized descriptively. For tₘₐₓ and t_{½}, the geometric mean and the geometric CV% were omitted.

An equivalence approach was taken to evaluate a food-effect on the log-transformation AUC₀₋ₜ, AUC_{0-∞}, and Cₘₐₓ for both baseline-adjusted and baseline-unadjusted concentrations of estradiol, estrone, and progesterone. The fasted condition served as the reference. Baseline-adjusted and baseline-unadjusted AUC₀₋ₜ, AUC_{0-∞}, and Cₘₐₓ endpoints were analyzed using a linear mixed-effects model using the PK Population. The model was fitted after taking the natural logarithm of the values. The fitted model (log-scale) for each parameter included the fixed effects period, sequence and treatment condition (fed and fasting), and subject as random effect. Each fitted model was used to derive point estimates and associated 90% confidence intervals (CIs) for the adjusted mean difference (log scale) between the two treatments. These CIs were finally exponentiated to obtain the adjusted geometric mean ratio (GMR) point estimates and associated 90% CIs. If the 90% CI of the adjusted GMR of fed versus fasting was within the interval of 80 to 125%, it was to be concluded that there was no food effect.

In addition to the adjusted GM and 90% CI for the GMRs, the intra-subject variation is presented. The intra-subject variation was measured by the geometric CV and was derived as 100*sqrt(exp(S²)-1) where S² was the residual variation from the log transformed linear mixed-effect model.

### 3.7.8. Safety Analyses

Safety parameters were evaluated using the Safety Population. Missing values were treated as missing, except for causality, intensity, seriousness, and outcome of AEs. In such cases, a “worst case” approach was used, namely, for causality it was assumed to be related to study drug, for intensity the maximum severity was assumed, for seriousness it was assumed to be serious, and for outcome it was assumed to be ongoing. Data are presented in summary tables and listings. Categorical data were summarized by treatment using the number and percentage of subjects in each category. For calculation of percentages, the denominator was the total number of subjects on that respective treatment in the Safety Population. Continuous data were summarized by treatment using number, mean, SD, median, minimum, and maximum.

Adverse events were coded using the MedDRA version 18.0. All AEs, including those that were not treatment-emergent were listed. A TEAE was defined as an AE that was new or worsened in severity after the first dose of study drug. All TEAEs were summarized by SOC, PT, and treatment. Summaries were prepared for all AEs, TEAEs, and SAEs. A summary of TEAEs by severity is also presented.

For summary tables, AEs coded to the same PT were counted only once within a given subject. If an AE was recorded on multiple occasions in the same treatment period, only the highest severity and the highest degree of relationship to the study drug was presented. If two or more clinical events were reported within a single AE entry, then the corresponding individual PTs were coded separately.

Deaths, SAEs, and AEs leading to discontinuation were to be listed by subject.

The period baseline for all vital sign analyses was the last assessment prior to the period dose. For each treatment period, the vital sign assessments from Day -1 to Day 4 were summarized by treatment condition for each assessment time by treatment condition (fed and fasting). Furthermore, for each period, change from period baseline was derived for Day 1 to Day 4 vital signs assessments. These changes from baseline values were also summarized for each assessment time by treatment condition. The statistical summaries included the number of subjects with data, mean (SD), median, minimum, and maximum values. Results will also be listed by subject.

Screening ECG, physical examination, hematology, chemistry, and urinalysis results were summarized descriptively and listed by subject. Other screening safety assessments (pelvic examination, Pap smear, mammogram, urine drug and alcohol screen results, urine pregnancy tests) were listed by subject.

### 3.7.9. Determination of Sample Size

Based on what is known in the literature about the effect of food on oral progesterone bioavailability and TherapeuticsMD’s previous experience in studies EPROG-1K-351-12, EPROG-1K-352-12, and EPROG-1K-459-12, 24 subjects were planned to be enrolled with 20 subjects planned to complete in order to accurately characterize food effect on TX-001HR.

### 3.7.10. Interim Analyses

No interim analyses were planned or conducted.

### 3.8. Changes in the Conduct of the Study or Planned Analyses

### 3.8.1. Changes to the Protocol

There was one amendment made to the original protocol: Amendment 1 (11Aug2017) to the protocol, completed prior to screening, included changes in the document for clarification and consistency of information. The most substantial revisions included the following:
Clarification of inclusion criterion 1a to further define postmenopausal as 12-consecutive months of spontaneous amenorrhea without an alternative medical cause.
Modification of inclusion criterion 1b to require that all women < 55 years of age who are not at least 6 weeks postsurgical bilateral oophorectomy must have had follicle stimulating hormone levels > 40 mIU/mL.
Clarification in the protocol to indicate that all women who are < 55 years of age and not at least 6 weeks postsurgical bilateral oophorectomy must have an FSH performed.
Addition of a requirement to have medical history based on subject interview and that medical records be made available to assess eligibility.
Clarification in the Schedule of Assessments and List of Assessments that urine pregnancy test was to be performed on Day -1 of Period 1 and not required on Day -1 of Period 2.

### 3.8.2. Changes to the Planned Analyses

Subject 01-109 was found to have baseline levels of estradiol higher than the upper limit of the range generally accepted for postmenopausal women in Period 2 (108 to 115 pg/mL). The subject had previously undergone a partial hysterectomy, without oophorectomy, in 1997 at the age of 36 years and was reported postmenopausal since 1999. Since the subject was 56 years old at the time of screening, she did not undergo FSH testing at that time. Given the impact of these unexpectedly high baseline levels of estradiol on unadjusted concentrations, it was decided that PK analyses would be performed with and without this subject. The PK analysis results presented within this clinical study report are based on sensitivity analyses excluding this subject; however, other PK analyses were performed with this subject included and are presented in post-text figures and tables.

### 4. Study Subjects

### 4.1. Disposition of Subjects

Subject enrollment and disposition are presented in Table 54. Twenty-four subjects were randomized to the sequence of fasting-fed or fed-fasting conditions, with 12 subjects in each group. All of the randomized subjects were included in the Safety Population and PK Population. No subjects discontinued prematurely; all subjects completed the study.

TABLE 54

Subject Disposition (All Subjects)

| Disposition | Fasting-Fed N=12 n (%) | Fed-Fasting N=12 n (%) | Total N=24 n (%) |
|---|---|---|---|
| Randomized Subjects | 12 (100.0) | 12 (100.0) | 24 (100.0) |
| Safety Population ^{a} | 12 (100.0) | 12 (100.0) | 24 (100.0) |
| PK Population ^{b} | 12 (100.0) | 12 (100.0) | 24 (100.0) |
| Completed | 12 (100.0) | 12 (100.0) | 24 (100.0) |

Abbreviations: PK = Pharmacokinetic
^{a} All subjects who received at least one dose of study drug were included.
^{b} Subjects who completed both periods and had sufficient data to calculate Cₘₐₓ, AUC₀₋ₜ, and AUC_{o-∞} were included.

### 4.2. Protocol Deviations

Protocol deviations are summarized by category in Table 55. All subjects had at least 1 minor protocol violation. The Period 1 Day 1 post-dose vital sign assessments were performed outside the time window of 1 to 3 hours post-dose for all subjects. Ten subjects (41.7%) had protocol deviations categorized as “other.” These included pregnancy test or FSH being done in error, screening diastolic blood pressure being out-of-range, Day 1 dose administered 4 minutes outside the window, and pre-dose PK blood draw being outside the time window. A missed assessment/procedure was reported in 3 subjects (12.5%). These findings were considered to be minor and had little or no impact on the study results.

TABLE 55

Protocol Deviations

| Deviation | Fasting-Fed N=12 n (%) | Fed-Fasting N=12 n (%) | Total N=24 n (%) |
|---|---|---|---|
| Any minor deviation | 12 (100.0) | 12 (100.0) | 24 (100.0) |
| Study drug administration | 0 | 0 | 0 |
| Study drug compliance | 0 | 0 | 0 |
| Missed assessment/procedure | 2 (16.7) | 1 (8.3) | 3 (12.5) |
| Missed visit | 0 | 0 | 0 |
| Other | 5 (41.7) | 5 (41.7) | 10(41.7) |
| Vital signs assessment out of window^{a} | 12 (100.0) | 12 (100.0) | 24 (100.0) |

^{a} Per protocol, vital signs were to be taken within 1 to 3 hours post-dose on Day 1. Post-dose vital signs were completed out of window on Period 1 Day 1.
Note: Some subjects may have had more than one protocol deviation.

### 5. Pharmacokinetics Evaluation

### 5.1. Data Sets Analyzed

The statistical analysis was performed on the following subject population: PK Population: This population consisted of subjects who completed both periods and had sufficient data to calculate Cₘₐₓ, AUC₀₋ₜ, and AUC_{0-∞}. The PK summaries were based on the PK Population.

In this study, the PK Population and the Safety Population consisted of the same subjects.

### 5.2. Demographic and Other Baseline Characteristics

### 5.2.1. Subject Demographics

Demographic characteristics are summarized for the Safety Population in Table 56.

All participants in the study were female (24/24 [100.0%]). The mean age was 57.5 years. The majority of subjects were white (17/24 [70.8%]) and not Hispanic or Latino (20/24 [83.3%]). The mean weight, height, and BMI were 71.05 kg, 163.11 cm, and 26.9 kg/m², respectively.

TABLE 56

Demographics (Safety Population)

| Characteristic | Category/Statistics | Fasting-Fed N=12 n (%) | Fed-Fasting N=12 n (%) | Total N=24 n (%) |
|---|---|---|---|---|
| Age (years) | | | | |
| | n | 12 | 12 | 24 |
| | Mean (SD) | 55.9 (5.09) | 59.0 (3.88) | 57.5 (4.70) |
| | Median | 56.0 | 60.0 | 57.0 |
| | Min, Max | 44, 65 | 52, 65 | 44, 65 |
| Sex - n (%) | | | | |
| | Female | 12 (100.0) | 12 (100.0) | 24 (100.0) |
| Race - n (%) | | | | |
| | American Indian or Alaska Native | 0 | 0 | 0 |
| | Asian | 1 (8.3) | 0 | 1 (4.2) |
| | Black or African American | 2 (16.7) | 4 (33.3) | 6 (25.0) |
| | Native Hawaiian or Other Pacific Islander | 0 | 0 | 0 |
| | White | 9 (75.0) | 8 (66.7) | 17 (70.8) |
| Ethnicity - n (%) | | | | |
| | Hispanic or Latino | 2 (16.7) | 2 (16.7) | 4 (16.7) |
| | Not Hispanic or Latino | 10 (83.3) | 10 (83.3) | 20 (83.3) |
| Weight (kg) | | | | |
| | n | 12 | 12 | 24 |
| | Mean (SD) | 73.10 (4.641) | 68.99 (8.479) | 71.05 (7.007) |
| | Median | 71.00 | 67.90 | 70.40 |
| | Min, Max | 68, 83.5 | 52, 84 | 52, 84 |
| Height (cm) | | | | |
| | n | 12 | 12 | 24 |
| | Mean (SD) | 163.48 (5.862) | 162.75 (6.757) | 163.11 (6.198) |
| | Median | 163.40 | 162.50 | 163.40 |
| | Min, Max | 154.5, 171 | 153, 176 | 153, 176 |
| BMI (kg/m²)^{a} | | | | |
| | n | 12 | 12 | 24 |
| | Mean (SD) | 27.4 (2.27) | 26.3 (3.08) | 26.9 (2.71) |
| | Median | 27.5 | 28.0 | 28.0 |
| | Min, Max | 24, 30 | 21, 30 | 21, 30 |

Note: BMI = body mass index; SD = standard deviation
^{a} BMI = weight (kg)/(height (m))², rounded to one decimal

### 5.2.2. Medical History

Medical history findings for the Safety Population was summarized. All subjects had a medical history of being postmenopausal. Other common findings were eye disorders (87.5%), surgical and medical procedures (87.5%), infections and infestations (83.3%), immune system disorders (41.7%), and musculoskeletal and connective tissue disorders (37.5%).

Gynecologic history findings, including type of menopause and history of hysterectomy or bilateral oophorectomy were also noted.

### 5.2.3. Prior Treatments

Prior medications are summarized in post-text. Ten subjects (41.7%) had taken at least one prior medication.

Prior non-drug therapies and procedures were noted in post-text. One subject (4.2%) reported using a prior non-drug therapy.

### 5.2.4. Screening Physical Examination

Screening physical examination results are summarized below in Table 58. At the time of screening, the majority of subjects in the Safety Population had normal evaluations during the physical evaluation. No abnormal, clinically significant findings were identified in subjects during the evaluation.

TABLE 58

Physical Examination (Safety Population)

| Category | Evaluation | Fasting-Fed N=12 n (%) | Fed-Fasting N=12 n (%) | Total N=24 n (%) |
|---|---|---|---|---|
| General appearance | Normal | 10 (83.3) | 12(100.0) | 22 (91.7) |
| | Abnormal, not clinically significant | 2 (16.7) | 0 | 2 (8.3) |
| Breast | Normal | 11 (91.7) | 12(100.0) | 23 (95.8) |
| | Abnormal, not clinically significant | 1 (8.3) | 0 | 1 (4.2) |
| HEENT | Normal | 12(100.0) | 12(100.0) | 24 (100.0) |
| | Abnormal, not clinically significant | 0 | 0 | 0 |
| Heart | Normal | 11 (91.7) | 12(100.0) | 23 (95.8) |
| | Abnormal, not clinically significant | 1 (8.3) | 0 | 1 (4.2) |
| Lungs | Normal | 12(100.0) | 12(100.0) | 24 (100.0) |
| | Abnormal, not clinically significant | 0 | 0 | 0 |
| Musculoskeletal system | Normal | 12(100.0) | 11 (91.7) | 23 (95.8) |
| | Abnormal, not clinically significant | 0 | 1 (8.3) | 1 (4.2) |
| Gastrointestinal | Normal | 12(100.0) | 12(100.0) | 24 (100.0) |
| | Abnormal, not clinically significant | 0 | 0 | 0 |
| Neurological system | Normal | 12 (100.0) | 12 (100.0) | 24 (100.0) |
| | Abnormal, not clinically significant | 0 | 0 | 0 |
| Lymph nodes | Normal | 12(100.0) | 12(100.0) | 24 (100.0) |
| | Abnormal, not clinically significant | 0 | 0 | 0 |
| Abdomen | Normal | 12(100.0) | 12(100.0) | 24 (100.0) |
| | Abnormal, not clinically significant | 0 | 0 | 0 |
| Extremities | Normal | 12(100.0) | 12(100.0) | 24 (100.0) |
| | Abnormal, not clinically significant | 0 | 0 | 0 |

Abbreviations: HEENT = the physical examination that concerns the head, eyes, ears, nose, and throat
Source: Table 14.1.8

### 5.2.5. Screening Pelvic Examination

Pelvic examination and Pap smear results are listed by subject in Listing 16.2.4.6. The majority of subjects had a normal pelvic examination result (23/24 [95.8%]). One subject had a non-clinically significant abnormal result. All subjects had normal pap smear results.

### 5.2.6. Screening Electrocardiogram

The overall interpretation of results from the screening 12-lead ECGs is summarized below in Table 59. The majority of subjects had normal results (13/24 [54.2%]). No abnormal clinically significant results were reported.

TABLE 59

Overall Interpretation of 12-Lead Electrocardiogram at Screening (Safety Population)

| Overall Interpretation | Fasting-Fed N=12 n (%) | Fed-Fasting N=12 n (%) | Total N=24 n (%) |
|---|---|---|---|
| Normal | 6 (50.0) | 7 (58.3) | 13 (54.2) |
| Abnormal, not clinically significant | 6 (50.0) | 5 (41.7) | 11 (45.8) |
| Abnormal, clinically significant | 0 | 0 | 0 |

### 5.2.7. Screening Mammogram

Screening mammography results were noted. All results were benign; 22 subjects (91.7%) had results of BI-RADS 2 and two subjects (8.3%) had a result of BI-RADS 1.

### 5.3. Measurements of Treatment Compliance

Drug exposure and treatment compliance are summarized below in Table 60. All subjects received both doses.

TABLE 60

Drug Exposure and Treatment Compliance (Safety Population)

| 1 mg estradiol/100 mg progesterone | Treatment Sequence | | Total N=24 n (%) |
|---|---|---|---|
| | Fasting-Fed N=12 n (%) | Fed-Fasting N=12 n (%) | |
| Received dose under fed condition | 12 (100.0) | 12 (100.0) | 24 (100.0) |
| Received dose under fasting condition | 12 (100.0) | 12 (100.0) | 24 (100.0) |

### 5.4. Pharmacokinetics Results and Tabulations of Individual Patient Data

### 5.4.1. Analysis of Pharmacokinetics

Pharmacokinetic parameters were determined using baseline-adjusted and unadjusted plasma estradiol, estrone, and progesterone concentrations.

Subject 01-109 was found to have baseline levels of estradiol higher than the upper limit of the range consistent with postmenopausal status in Period 2 (108 to 115 pg/mL). This subject was 56 years old at the time of screening and had had a partial hysterectomy, without oophorectomy, in 1997 and was postmenopausal since 1999. The PK analysis results presented within this section of the clinical study report are based on sensitivity analyses which exclude this subject. However, other PK analyses were also performed with this subject included.

### 5.4.1.1. Plasma Concentrations

### 5.4.1.1.1. Estradiol

The plasma estradiol concentrations (baseline-adjusted and unadjusted) were summarized by time point in post-text.

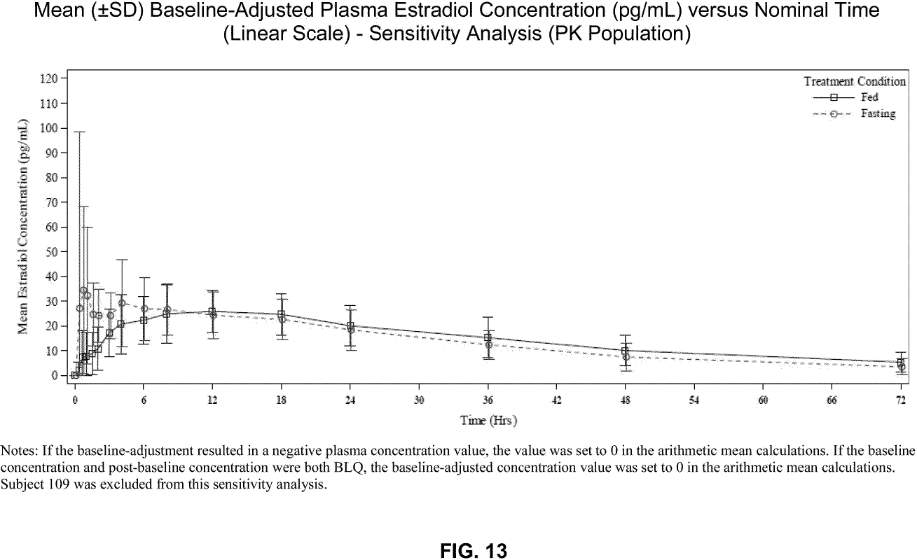

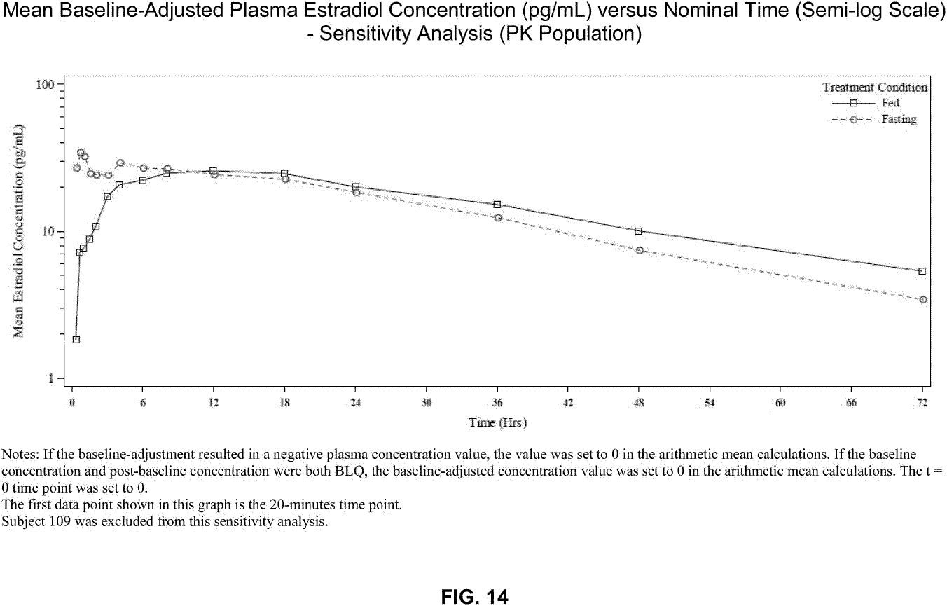

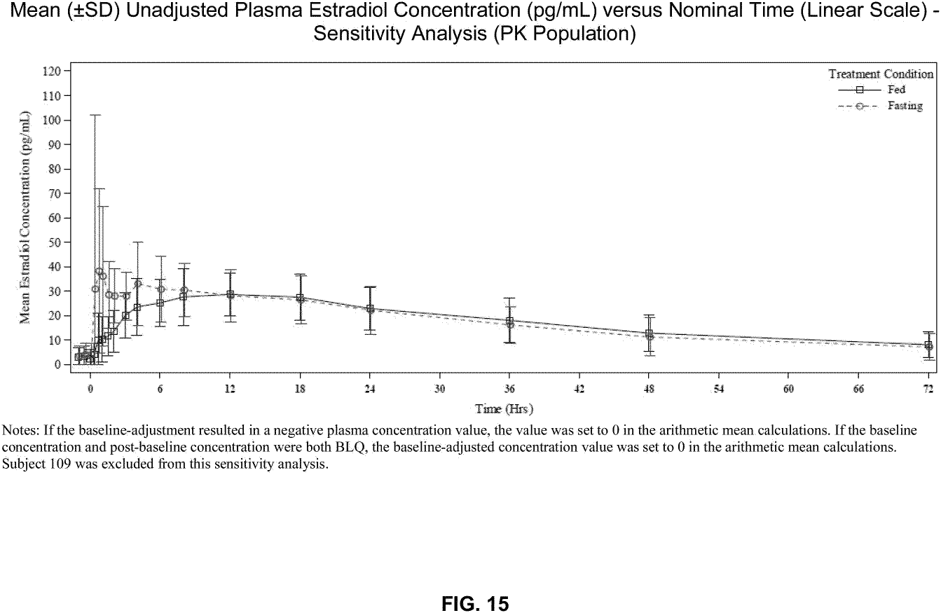

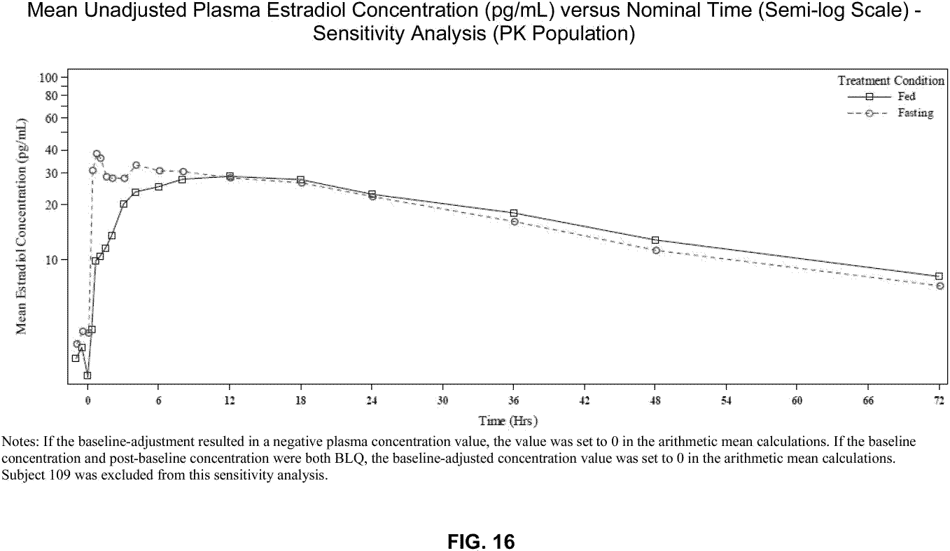

FIG. 13 and FIG. 14 show fed and fasting mean baseline-adjusted estradiol concentration versus nominal time on a linear scale and semi-log scale, respectively. FIG. 15 and FIG. 16 show mean baseline-unadjusted estradiol concentration versus nominal time on a linear scale and semi-log scale, respectively. The LLOQ of the LC-MS/MS assay for estradiol is 5.00 pg/mL.

Spaghetti plots of baseline-adjusted estradiol concentration versus actual time are presented for the fed condition on a linear scale elsewhere and on a semi-log scale in post-text elsewhere. Spaghetti plots of baseline-adjusted estradiol concentration versus actual time are presented for the fasting condition on a linear scale in post-text elsewhere and on a semi-log scale in post-text elsewhere.

### 5.4.1.1.2. Estrone

The plasma estrone (metabolite of estradiol) concentrations (baseline-adjusted and unadjusted) are summarized by time point in post-text elsewhere.

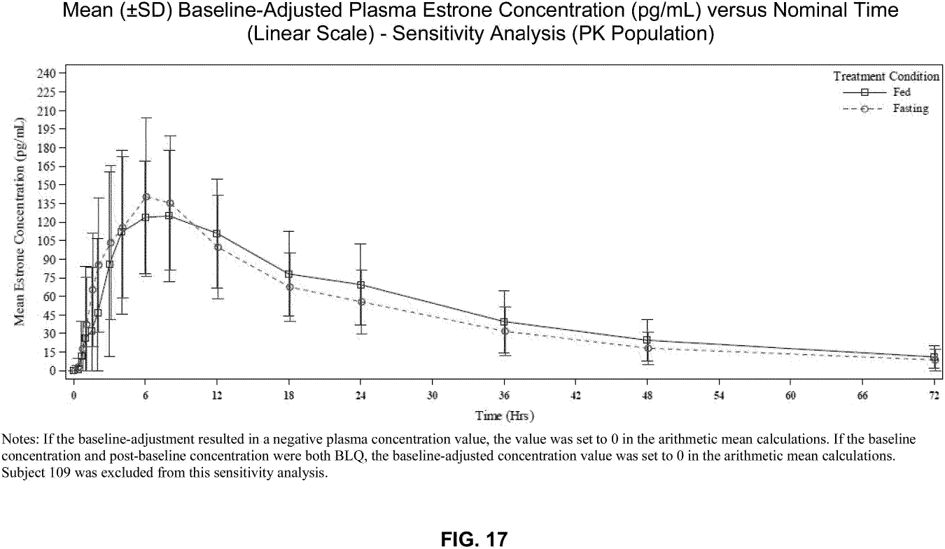

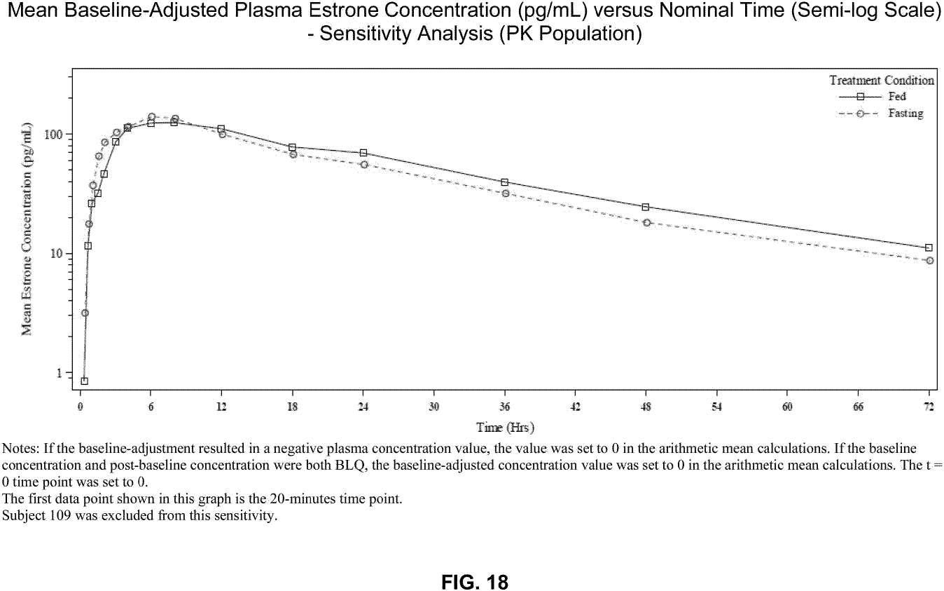

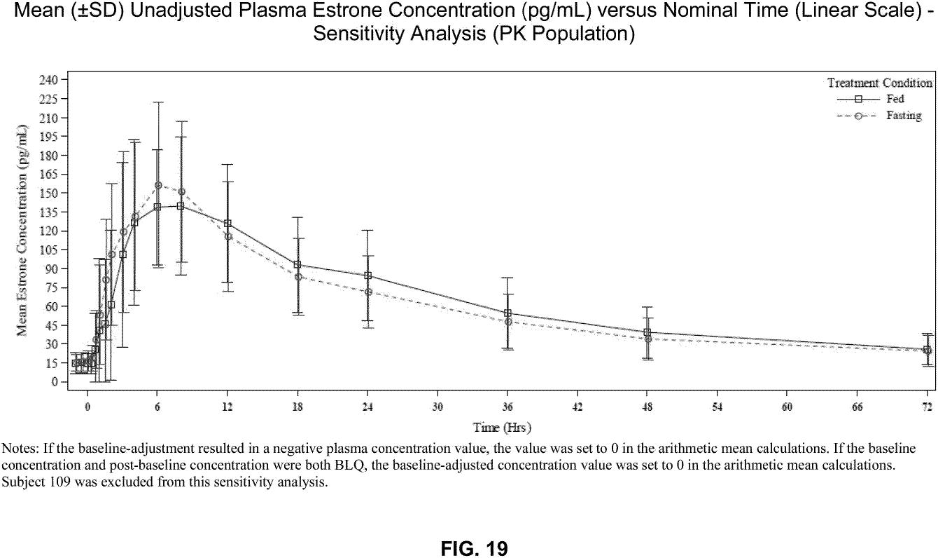

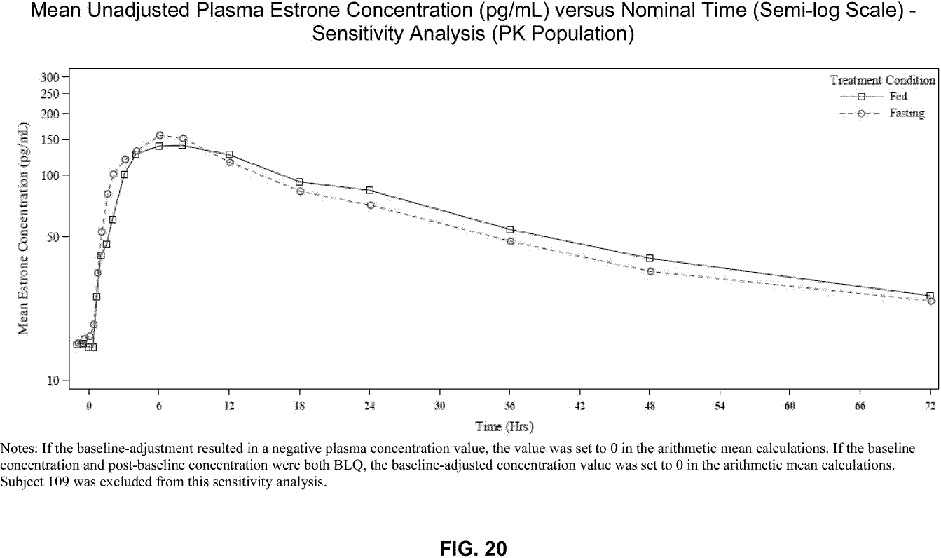

FIG. 17 and FIG. 18 show fed and fasting mean baseline-adjusted estrone concentration versus nominal time on a linear and semi-log scale, respectively. FIG. 19 and FIG. 20 show mean baseline-unadjusted estrone concentration versus nominal time on a linear and semi-log scale, respectively. The LLOQ of the LC-MS/MS assay for estrone is 5.00 pg/mL.

Spaghetti plots of baseline-adjusted estrone concentration versus actual time are presented for the fed condition on a linear scale elsewhere and on a semi-log scale in post-text elsewhere. Spaghetti plots of baseline-adjusted estrone concentration versus actual time are presented for the fasting condition on a linear scale in post-text elsewhere and on a semi-log scale in post-text elsewhere.

### 5.4.1.1.3. Progesterone

The plasma progesterone concentrations (baseline-adjusted and unadjusted) are summarized by time point.

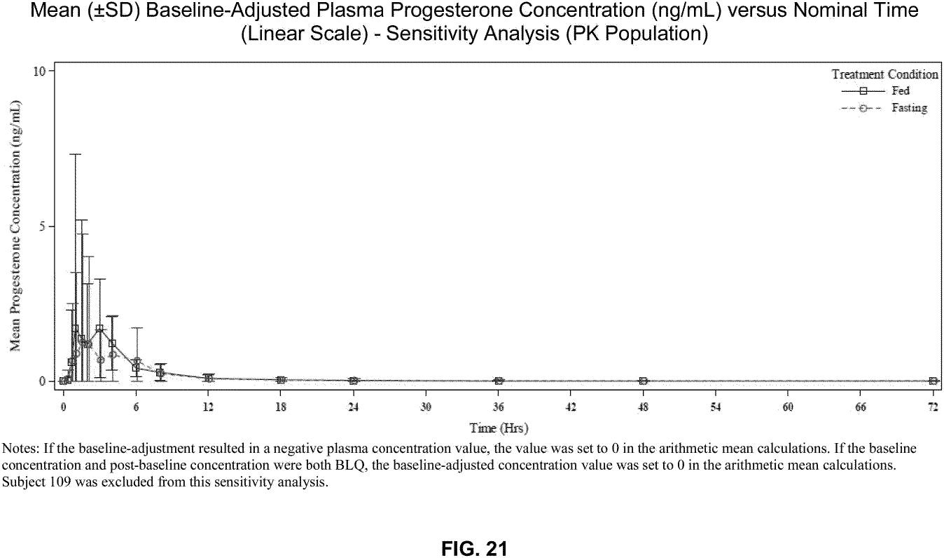

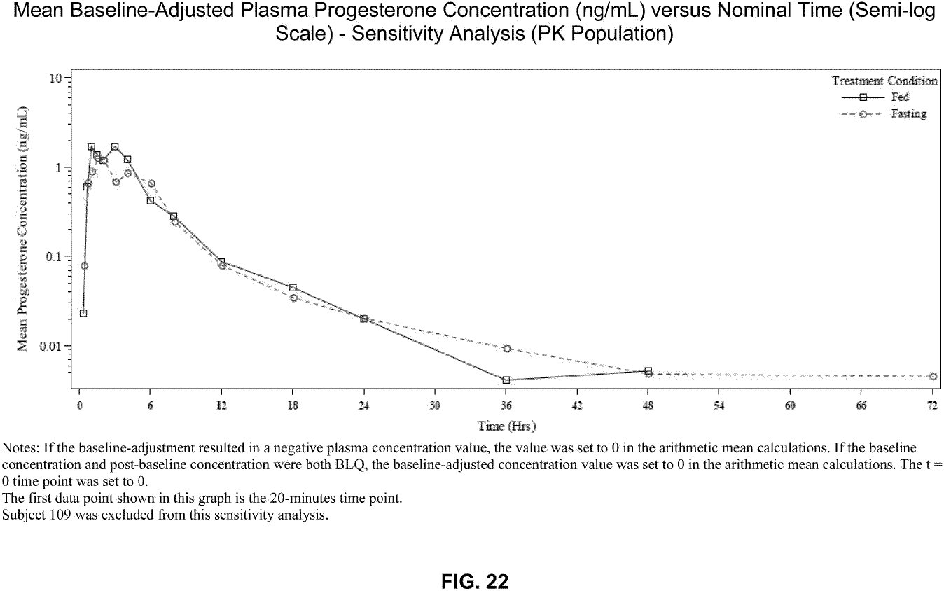

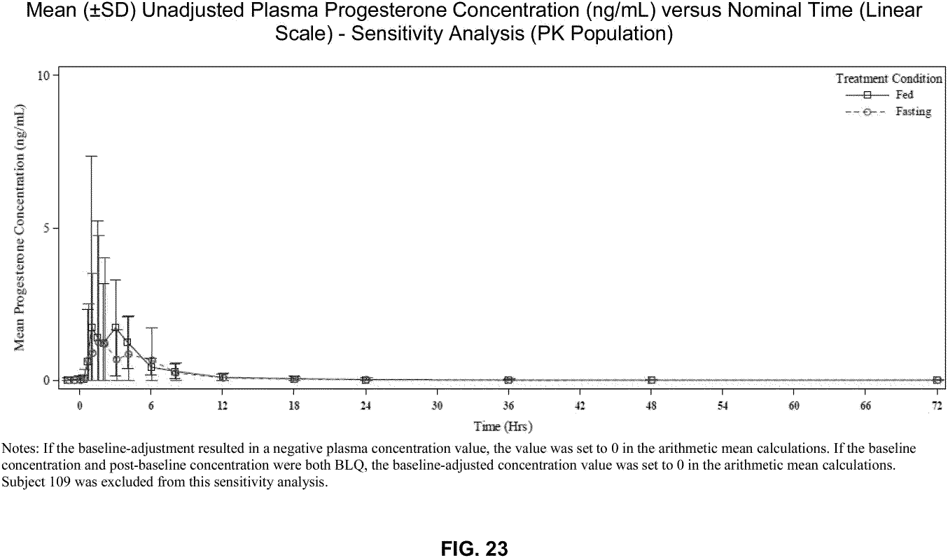

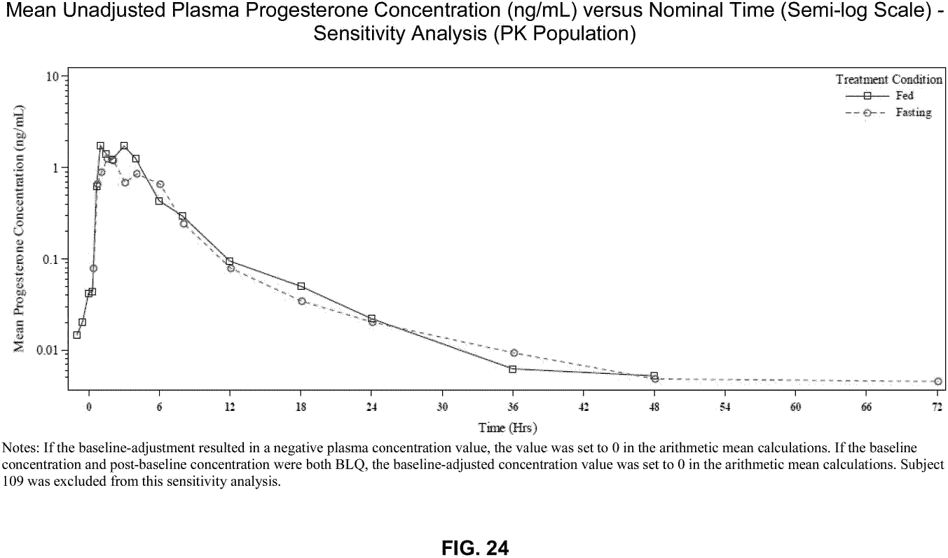

FIG. 21 and FIG. 22 show fed and fasting mean baseline-adjusted progesterone concentration versus nominal time on a linear and semi-log scale, respectively. FIG. 23 and FIG. 24 show mean baseline-unadjusted progesterone concentration versus nominal time on a linear scale and semi-log scale, respectively. The LLOQ of the LC-MS/MS assay for progesterone is 0.10 ng/mL.

Spaghetti plots of baseline-adjusted progesterone concentration versus actual time are presented for the fed condition on a linear scale in post-text elsewhere and on a semi-log scale in post-text elsewhere. Spaghetti plots of baseline-adjusted progesterone concentration versus actual time are presented for the fasting condition on a linear scale in post-text elsewhere and on a semi-log scale in post-text elsewhere.

### 5.4.1.2. Pharmacokinetics Parameters

### 5.4.1.2.1. Estradiol

The PK parameters for plasma estradiol (baseline-adjusted and unadjusted), fed and fasting, are summarized below in Table 61.

Mean AUC_{0-t,} AUC_{0-∞}, and λ_{z} were not different under fed and fasting conditions in both baseline-adjusted and unadjusted analyses. Mean baseline-adjusted Cₘₐₓ was greater under fasting conditions (74.68 pg/mL) than fed conditions (29.55 pg/mL). Similar findings were observed for unadjusted estradiol. In baseline-adjusted and unadjusted analyses, mean tₘₐₓ was greater under fed conditions (11.57 hours) than fasting conditions (2.58 hours). The observed mean t½ value was not different for baseline-adjusted estradiol under fed and fasting conditions (21.77 hours and 25.49 hours, respectively).

TABLE 61

Summary of PK Parameters for Baseline-Adjusted and Unadjusted Plasma Estradiol Concentration - Sensitivity Analysis (PK Population)

| | | Adjusted Concentration | | Unadjusted Concentration | |
|---|---|---|---|---|---|
| Parameter | Statistics | Fed N=23 | Fasting N=23 | Fed N=23 | Fasting N=23 |
| AUC₀₋ₜ (pg·h/mL) | n | 23 | 23 | 23 | 23 |
| | Mean (SD) | 1063 (441.3) | 1002 (386.2) | 1263 (510.0) | 1276 (519.9) |
| AUC_{0-∞} (pg·h/mL) | n | 20 | 22 | 19 | 23 |
| | Mean (SD) | 1286 (571.1) | 1249 (454.5) | 1723 (818.9) | 1874 (1004) |
| Cmax (pg/mL) | n | 23 | 23 | 23 | 23 |
| | Mean (SD) | 29.55 (11.30) | 74.68 (60.46) | 32.36 (11.40) | 78.48 (59.76) |
| tₘₐₓ (h) | n | 23 | 23 | 23 | 23 |
| | Mean (SD) | 11.57 (5.846) | 2.58 (5.006) | 11.57 (5.846) | 2.58 (5.006) |
| λ_{z}, (/h) | n | 20 | 22 | 19 | 23 |
| | Mean (SD) | 0.04 (0.042) | 0.04 (0.026) | 0.03 (0.006) | 0.02 (0.009) |
| t_{½} (h) | n | 20 | 22 | 19 | 23 |
| | Mean (SD) | 21.77 (6.910) | 25.49 (15.07) | 28.66 (7.202) | 47.01 (71.41) |

Abbreviations; SD = standard deviation
Notes: PK parameters were derived based on the concentrations collected from 0 to 72 hours post-dose.
Subject 109 was excluded from this sensitivity analysis.
Please note data was rounded using four significance figures, when possible.

### 5.4.1.2.2. Estrone

The PK parameters for plasma estrone (baseline-adjusted and unadjusted), fed and fasting, are summarized below in Table 62.

Mean AUC₀₋ₜ AUC_{0-∞} Cₘₐₓ, tₘₐₓ, and λ_{z} were generally not different under fed and fasting conditions in both baseline-adjusted and unadjusted analyses. Mean t_{½} was also not different under fed and fasting conditions, but was generally greater in the unadjusted estrone analysis compared to the baseline-adjusted estrone.

TABLE 62

Summary of PK Parameters for Baseline-Adjusted and Unadjusted Plasma Estrone Concentration - Sensitivity Analysis (PK Population)

| | | Adjusted Concentration | | Unadjusted Concentration | |
|---|---|---|---|---|---|
| Parameter | Statistics | Fed N=23 | Fasting N=23 | Fed N=23 | Fasting N=23 |
| AUC₀₋ₜ (pg·h/mL) | N | 23 | 23 | 23 | 23 |
| | Mean (SD) | 3640 (1541) | 3289 (1385) | 4707 (1768) | 4435 (1602) |
| AUC_{0-∞} (pg·h/mL) | N | 19 | 22 | 19 | 21 |
| | Mean (SD) | 4173 (1734) | 3729 (1734) | 6155 (2369) | 6065 (2715) |
| Cₘₐₓ (pg/mL) | N | 23 | 23 | 23 | 23 |
| | Mean (SD) | 145.6 (62.03) | 154.9 (62.36) | 160.5 (62.85) | 170.8 (64.24) |
| tₘₐₓ (h) | N | 23 | 23 | 23 | 23 |
| | Mean (SD) | 7.79 (4.675) | 7.26 (4.244) | 7.79 (4.675) | 7.26 (4.244) |
| λ_{z} (/h) | N | 19 | 22 | 19 | 21 |
| | Mean (SD) | 0.05 (0.016) | 0.05 (0.023) | 0.03 (0.008) | 0.02 (0.009) |
| t_{½} (h) | N | 19 | 22 | 19 | 21 |
| | Mean (SD) | 16.72 (5.286) | 17.55 (12.37) | 30.18 (12.87) | 34.35 (18.54) |

Abbreviations: SD = standard deviation
Notes: PK parameters were derived based on the concentrations collected from 0 to 72 hours post-dose.
Subject 109 was excluded from this sensitivity analysis.
Please note data was rounded using four significance figures, when possible.

### 5.4.1.2.3. Progesterone

The PK parameters for plasma progesterone (baseline-adjusted and unadjusted), fed and fasting, are summarized below in Table 63.

Mean AUC_{0-t,} AUC_{0-∞}, and Cₘₐₓ were generally greater under fed conditions as compared to fasting conditions in both baseline-adjusted and unadjusted analyses. Mean tₘₐₓ, λ_{z} and t_{½} for progesterone were not different between fasting and fed conditions in both baseline-adjusted and unadjusted analyses.

TABLE 63

Summary of PK Parameters for Baseline-Adjusted and Unadjusted Plasma Progesterone Concentration - Sensitivity Analysis (PK Population)

| | | Adjusted Concentration | | Unadjusted C Concentration | |
|---|---|---|---|---|---|
| Parameter | Statistics | Fed N=23 | Fasting N=23 | Fed N=23 | Fasting N=23 |
| AUC₀₋ₜ (ng·h/mL) | N | 23 | 23 | 23 | 23 |
| | Mean (SD) | 8.48 (9.390) | 7.14 (10.78) | 8.72 (9.655) | 7.14 (10.78) |
| AUC_{0-∞} (ng·h/mL) | N | 13 | 8 | 13 | 8 |
| | Mean (SD) | 9.80 (12.44) | 9.71 (15.15) | 10.10 (13.24) | 9.71 (15.15) |
| Cₘₐₓ (ng/mL) | N | 23 | 23 | 23 | 23 |
| | Mean (SD) | 3.60 (5.312) | 1.82 (3.532) | 3.63 (5.315) | 1.82 (3.532) |
| tₘₐₓ (h) | N | 23 | 23 | 23 | 23 |
| | Mean (SD) | 2.48 (1.071) | 2.64 (1.768) | 2.48 (1.071) | 2.64 (1.768) |
| λ_{z} (/h) | N | 13 | 8 | 13 | 8 |
| | Mean (SD) | 0.26 (0.126) | 0.21 (0.128) | 0.25 (0.129) | 0.21 (0.128) |
| t_{½} (h) | N | 13 | 8 | 13 | 8 |
| | Mean (SD) | 3.73 (2.881) | 7.48 (9.537) | 3.99 (3.429) | 7.48 (9.537) |

Abbreviations: SD = standard deviation
Notes: PK parameters were derived based on the concentrations collected from 0 to 72 hours post-dose.
Subject 109 was excluded from this sensitivity analysis.
Please note data was rounded using four significance figures, when possible.

### 5.4.1.3. Statistical Analysis of Pharmacokinetic Parameters for Food Effect

### 5.4.1.3.1. Estradiol

The statistical analyses of baseline-adjusted and unadjusted PK parameters for plasma estradiol fed versus fasting are presented below in Table 64 and Table 65, respectively.

No food effect was to be declared if the 90% CIs for the GMR of fed versus fasting PK parameters were within the 80% to 125% interval. The 90% CIs for GMRs of AUC₀₋ₜ and AUC_{0-∞}representing extent of absorption, were each within this interval in baseline-adjusted and unadjusted analyses. However, in an analysis of baseline-adjusted and unadjusted Cₘₐₓ, the 90% CIs for the adjusted GMRs were found to be below 80%. This is not unexpected given the more rapid absorption in the fasted state, as indicated by a much earlier tₘₐₓ. Also, the intra-subject variability for Cₘₐₓ was greater than 30% (46.7, baseline-adjusted and 41.7, unadjusted).

TABLE 64

Statistical Analysis of Baseline-Adjusted PK Parameters for Plasma Estradiol - Sensitivity Analysis (PK Population)

| Parameter | Adjusted Mean^{a} Geometric | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (pg·h/mL) | 959.32 | 916.99 | 104.6 | (95.5, 114.6) | 18.1 |
| AUC_{0-∞} (pg·h/mL) | 1144.57 | 1123.41 | 101.9 | (90.8, 114.3) | 20.7 |
| Cₘₐₓ (pg/mL) | 27.71 | 60.37 | 45.9 | (36.6, 57.5) | 46.7 |

Abbreviations: CI = confidence interval; GMR = geometric mean ratio
Note: The fitted model (log-scale) for each parameter included the fixed effects period, sequence, and treatment condition
(fed and fasting), and subject as random effect.
^{a} Exponentiated value of the least square means from the mixed-effects model of the log-transformed data.
^{b} The intra-subject variability is measured by the geometric coefficient of variation and derived as 100*sqrt {exp(S2)-1) where S2 is the residual variation from the log-transformed linear mixed model.
Subject 109 was excluded from this sensitivity analysis.

TABLE 65

Statistical Analysis of Unadjusted PK Parameters for Plasma Estradiol -Sensitivity Analysis (PK Population)

| Parameter | Adjusted Geometric Mean^{a} | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (pg·h/mL) | 1166.12 | 1172.30 | 99.5 | (92.4, 107.0) | 14.5 |
| AUC_{0-∞} (pg·h/mL) | 1670.21 | 1660.47 | 100.6 | (92.6, 109.2) | 14.7 |
| Cₘₐₓ (pg/mL) | 30.82 | 64.65 | 47.7 | (38.9, 58.4) | 41.7 |

Abbreviations: CI = confidence interval; GMR = geometric mean ratio
Note: The fitted model (log-scale) for each parameter included the fixed effects period, sequence, and treatment condition
(fed and fasting), and subject as random effect.
^{a} Exponentiated value of the least square means from the mixed-effects model of the log-transformed data.
^{b} The intra-subject variability is measured by the geometric coefficient of variation and derived as 100*sqrt (exp(S2)-1) where S2 is the residual variation from the log-transformed linear mixed model.
Subject 109 was excluded from this sensitivity analysis.

### 5.4.1.3.2. Estrone

The statistical analyses of baseline-adjusted and unadjusted PK parameters for plasma estrone fed versus fasting are presented below in Table 66 and Table 67, respectively. No food effect was to be declared if the 90% CIs for the GMRs of fed versus fasting PK parameters were within the 80% to 125% interval.

No food effect on the plasma levels of estrone was observed in the analyses of baseline-adjusted and unadjusted geometric mean AUC_{0-t,} AUC_{0-∞}, and Cₘₐₓ. All CIs were within the 80 to 125% interval with the intra-subject variability less than 30%.

TABLE 66

Statistical Analysis of Baseline-Adjusted PK Parameters for Plasma Estrone - Sensitivity Analysis (PK Population)

| Parameter | Adjusted Mean^{a} Geometric | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (pg·h/mL) | 3320.51 | 2983.92 | 111.3 | (102.6, 120.7) | 16.0 |
| AUC_{0-∞}(pg·h/mL) | 3691.06 | 3227.13 | 114.4 | (106.2, 123.2) | 12.8 |
| Cₘₐₓ (pg/mL) | 135.25 | 143.06 | 94.5 | (86.8, 103.0) | 17.0 |

Abbreviations: CI = confidence interval: GMR = geometric mean ratio
Note: The fitted model (log-scale) for each parameter included the fixed effects period, sequence, and treatment condition
(fed and fasting), and subject as random effect.
^{a} Exponentiated value of the least square means from the mixed-effects model of the log-transformed data.
^{b} The intra-subject variability is measured by the geometric coefficient of variation and derived as 100*sqrt(exp(S2)-1) where S2 is the residual variation from the log-transformed linear mixed model.
Subject 109 was excluded from this sensitivity analysis.

TABLE 67

Statistical Analysis of Unadjusted PK Parameters for Plasma Estrone -Sensitivity Analysis (PK Population)

| Parameter | Adjusted Geometric Mean^{a} | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (pg·h/mL) | 4410.60 | 4164.68 | 105.9 | (100.0, 112.2) | 11.4 |
| AUC_{0-∞}(pg·h/mL) | 5911.95 | 5463.30 | 108.2 | (101.2, 115.7) | 11.5 |
| Cₘₐₓ (pg/mL) | 150.94 | 159.45 | 94.7 | (87.7, 102.2) | 15.1 |

Abbreviations: CI = confidence interval; GMR = geometric mean ratio
Note: The fitted model (log-scale) for each parameter included the fixed effects period, sequence, and treatment condition (fed and fasting), and subject as random effect.
^{a} Exponentiated value of the least square means from the mixed-effects model of the log-transformed data.
^{b} The intra-subject variability is measured by the geometric coefficient of variation and derived as 100*sqrt(exp(S2)-1) where S2 is the residual variation from the log-transformed linear mixed model.
Subject 109 was excluded from this sensitivity analysis.

### 5.4.1.3.3. Progesterone

The statistical analyses of baseline-adjusted and unadjusted PK parameters for plasma progesterone fed versus fasting are presented below in Table 68 and Table 69, respectively.

No food effect was to be declared if the 90% CIs for the GMRs of fed versus fasting PK parameters were within the 80% to 125% interval. A food effect was observed as the CIs for the adjusted GMRs of AUC_{0-t,} AUC_{0-∞}, and Cₘₐₓ were outside the 80 to 125% interval, with greater exposure in the fed conditions. In addition, the intra-subject variability was greater than 30% for all PK parameters creating wide CIs.

TABLE 68

Statistical Analysis of Baseline-Adjusted PK Parameters for Plasma Progesterone - Sensitivity Analysis (PK Population)

| Parameter | Adjusted Geometric Mean^{a} | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (ng·h/mL) | 6.45 | 3.54 | 182.2 | (131.7, 251.9) | 70.9 |
| AUC_{0-∞} (ng·h/mL) | 6.72 | 5.26 | 127.8 | (49.6, 329.6) | 57.7 |
| Cₘₐₓ (ng/mL) | 2.50 | 0.92 | 270.9 | (188.2, 389.9) | 82.0 |

Abbreviations: CI = confidence interval; GMR = geometric mean ratio
Note: The fitted model (log-scale) for each parameter included the fixed effects period, sequence, and treatment condition (fed and fasting), and subject as random effect.
^{a} Exponentiated value of the least square means from the mixed-effects model of the log-transformed data.
^{b} The intra-subject variability is measured by the geometric coefficient of variation and derived as 100*sqrt(exp(S2)-1) where S2 is the residual variation from the log-transformed linear mixed model.
Subject 109 was excluded from this sensitivity analysis.

TABLE 69

Statistical Analysis of Unadjusted PK Parameters for Plasma Progesterone -Sensitivity Analysis (PK Population)

| Parameter | Adjusted Geometric Mean^{a} | | Adjusted GMR Fed/Fasting (%) | 90% CI for Adjusted GMR (%) | Intra-Subject Variability^{b} |
|---|---|---|---|---|---|
| | Fed | Fasting | | | |
| AUC₀₋ₜ (ng·h/mL) | 6.79 | 3.54 | 191.6 | (136.9, 268.1) | 74.1 |
| AUC_{0-∞} (ng·h/mL) | 6.79 | 5.30 | 128.2 | (49.8, 330.1) | 57.3 |
| Cₘₐₓ (ng/mL) | 2.53 | 0.92 | 274.0 | (189.7, 395.8) | 83.0 |

Abbreviations: CI = confidence interval; GMR = geometric mean ratio
Note: The fitted model (log-scale) for each parameter included the fixed effects period, sequence, and treatment condition (fed and fasting), and subject as random effect.
^{a} Exponentiated value of the least square means from the mixed-effects model of the log-transformed data.
^{b} The intra-subject variability is measured by the geometric coefficient of variation and derived as 100*sqrt(exp(S2)-1) where S2 is the residual variation from the log-transformed linear mixed model.
Subject 109 was excluded from this sensitivity analysis.

### 5.4.2. Statistical/Analytical Issues

### 5.4.2.1. Adjustments for Covariates

Not applicable.

### 5.4.2.2. Handling of Dropouts or Missing Data

There was no imputation of missing data in PK analyses.

### 5.4.3. Tabulation of Individual Response Data

Individual plasma concentrations of estradiol, estrone, and progesterone are listed by subject elsewhere. Individual PK parameters based on plasma estradiol, estrone, and progesterone are listed by subject elsewhere.

Individual spaghetti plots of baseline-adjusted estradiol concentration versus actual time are presented on a linear scale elsewhere and on a semi-log scale elsewhere.

Individual spaghetti plots of baseline-adjusted estrone concentration versus actual time are presented on a linear scale elsewhere and on a semi-log scale in post-text elsewhere.

Individual spaghetti plots of baseline-adjusted progesterone concentration versus actual time are presented on a linear scale in post-text elsewhere and on a semi-log scale in post-text elsewhere.

### 5.4.4. Discussion of Pharmacokinetic Results

Pharmacokinetic parameters were assessed using baseline-adjusted and unadjusted plasma estradiol, estrone, and progesterone concentrations. The overall conclusions are presented. An equivalence approach was taken to evaluate whether there was a food-effect on the log-transformed PK parameters. No food effect was to be declared if the 90% CIs for the GMRs of fed versus fasting PK parameters were within the 80% to 125% interval.

Subject 01-109, a 56-year-old female, presented with an unexpectedly high level of estradiol at baseline (108 to 115 pg/mL) in Period 2; unknown until sample analysis was completed. Therefore, all of the PK analyses performed for this study were repeated without this subject and labeled as sensitivity analyses.

The baseline-adjusted estradiol AUC₀₋ₜ and Cₘₐₓ for the fed and fasting conditions were not different with Subject 01-109 included or excluded. The baseline-unadjusted Cₘₐₓ, under both the fed and fasting conditions, was also not different with Subject 01-109 included or excluded. The baseline-unadjusted estradiol AUC₀₋ₜ under the fed condition was not different; however, under fasting conditions, AUC₀₋ₜ was higher with Subject 01-109 included than when that subject was excluded (1525 vs 1275 pg·h/mL, respectively).

The baseline-adjusted and unadjusted estrone AUC₀₋ₜ and Cₘₐₓ for both the fed and fasting conditions were no different with Subject 01-109 included or excluded.

The baseline-adjusted and unadjusted progesterone AUC₀₋ₜ and Cₘₐₓ for both the fed and fasting conditions were modestly lower with Subject 01-109 excluded compared to when this subject was included.

The overall PK conclusions were not altered when this subject was included in the analyses. The tables and figures presented in the body of this clinical study report exclude Subject 01-109. For completeness and comparison, PK analyses with and without Subject 01-109 are included herein.

Pharmacokinetic Conclusions
Mean estradiol AUC₀₋ₜ and AUC_{0-∞} were not different under fasting and fed conditions based on both baseline-adjusted and unadjusted estradiol concentrations. Mean Cₘₐₓ was greater and mean tₘₐₓ was earlier under fasting conditions based on both baseline-adjusted and unadjusted concentrations. Statistical analysis of adjusted GMRs of estradiol AUC₀₋ₜ and AUC_{0-∞} indicate that food had no effect on these estradiol parameters; however, Cₘₐₓ was significantly greater under the fasting condition than the fed condition.
Mean PK parameters for estrone were not different under fed and fasting conditions based on both baseline-adjusted and unadjusted concentrations. Statistical analysis of adjusted GMRs of estrone AUC_{0-t,} AUC_{0-∞}, and Cₘₐₓ indicate that food had no effect on these estrone PK parameters.
Mean progesterone AUC_{0-t,} AUC_{0-∞}, and Cₘₐₓ were greater under fed conditions as compared to fasting conditions based on both baseline-adjusted and unadjusted concentrations. Statistical analysis of adjusted GMRs of progesterone AUC_{0-t,} AUC_{0-∞}, and Cₘₐₓ indicate that food increases exposure to progesterone following administration of TX-001HR 1 mg estradiol/100 mg progesterone.
The overall PK conclusions were not altered whether Subject 01-109 was included in the analyses or excluded.

### 6. Safety Evaluation

### 6.1. Extent of Study Drug Exposure

Study drug administration and exposure details are listed by subjects elsewhere. All of the 24 subjects received both doses of study drug.

### 6.2. Adverse Events

### 6.2.1. Brief Summary of Adverse Events

An overview of AEs is presented below in Table 70. Nine subjects (37.5%) experienced a TEAE during the study and four subjects (16.7%) experienced a study drug-related TEAE. No deaths, SAEs, or AEs leading to study drug withdrawal were reported.

TABLE 70

Overview of Adverse Events (Safety Population)

| Categories | Total N=24 n (%) |
|---|---|
| Any AE | 10 (41.7) |
| Any serious AE | 0 |
| Any TEAE | 9 (37.5) |
| Any serious TEAE | 0 |
| Any study drug related TEAE | 4 (16.7) |
| Any AE leading to drug withdrawal | 0 |
| Any AE leading to death | 0 |

AE = adverse event, TEAE = treatment-emergent adverse event
A TEAE is defined as an AE that is new or worsened in severity after the first dose of study drug.
Subjects who reported more than one event within a category were counted only once.
A “study drug related TEAE” was defined as a TEAE with reported relationship to study drug as possible, probable or missing.

### 6.2.2. Display of Adverse Events

The TEAEs are summarized by SOC and PT for the Safety Population below in Table 71. Six subjects (25.0%) experienced headache, but no other TEAEs were reported by more than one subject.

Adverse events by SOC and PT are summarized elsewhere.

TABLE 71

Treatment-Emergent Adverse Event by System Organ Class and Preferred Term (Safety Population)

| System Organ Class Preferred Term | Total N=24 n (%) |
|---|---|
| Any TEAEs | 9 (37.5) |
| Gastrointestinal disorders | 1 (4.2) |
| Abdominal pain upper | 1 (4.2) |
| Constipation | 1 (4.2) |
| General disorders and administration site conditions | 1 (4.2) |
| Energy increased | 1 (4.2) |
| Injury, poisoning and procedural complications | 1 (4.2) |
| Contusion | 1 (4.2) |
| Musculoskeletal and connective tissue disorders | 1 (4.2) |
| Joint swelling | 1 (4.2) |
| Nervous system disorders | 6 (25.0) |
| Headache | 6 (25.0) |
| Post-traumatic headache | 1 (4.2) |
| Psychiatric disorders | 1 (4.2) |
| Libido increased | 1 (4.2) |
| Skin and subcutaneous tissue disorders | 1 (4.2) |
| Night sweats | 1 (4.2) |
| Vascular disorders | 1 (4.2) |
| Hot flush | 1 (4.2) |

TEAE = treatment-emergent adverse event
Adverse events (AEs) were coded using the Medical Dictionary for Regulatory Activities (MedDRA version 18.0). A TEAE was defined as an AE that was new or worsened in severity after the first dose of study drug.
Subjects were counted only once within the same system organ class (SOC) or preferred term (PT) if they reported more than once.
SOCs were sorted alphabetically and PTs were sorted by frequency of subjects from high to low.

TEAEs are summarized by severity, SOC, and PT below in Table 72. The majority of TEAEs were mild. One subject had moderate post-traumatic headache and four subjects had moderate headache. The incidence of headache was not different between the fed or fasting condition. No severe TEAEs were reported.

TABLE 72

Treatment-Emergent Adverse Event by System Organ Class and Preferred Term and by Severity (Safety Population)

| System Organ Class Preferred Term | Severity | Total N=24 n (%) |
|---|---|---|
| Any TEAEs | Mild | 5 (20.8) |
| | Moderate | 4 (16.7) |
| Gastrointestinal disorders | Mild | 1 (4.2) |
| Abdominal pain upper | Mild | 1 (4.2) |
| Constipation | Mild | 1 (4.2) |
| General disorders and administration site conditions | Mild | 1 (4.2) |
| Energy increased | Mild | 1 (4.2) |
| Injury, poisoning and procedural complications | Mild | 1 (4.2) |
| Contusion | Mild | 1 (4.2) |
| Musculoskeletal and connective tissue disorders | Mild | 1 (4.2) |
| Joint swelling | Mild | 1 (4.2) |
| Nervous system disorders | Mild | 2 (8.3) |
| | Moderate | 4 (16.7) |
| Headache | Mild | 2 (8.3) |
| | Moderate | 4 (16.7) |
| Post-traumatic headache | Moderate | 1 (4.2) |
| Psychiatric disorders | Mild | 1 (4.2) |
| Libido increased | Mild | 1 (4.2) |
| Skin and subcutaneous tissue disorders | Mild | 1 (4.2) |
| Night sweats | Mild | 1 (4.2) |
| Vascular disorders | Mild | 1 (4.2) |
| Hot flush | Mild | 1 (4.2) |

TEAE = treatment-emergent adverse event
Adverse events (AEs) were coded using the Medical Dictionary for Regulatory Activities (MedDRA version 18.0). A TEAE was defined as an AE that is new or worsened in severity after the first dose of study drug.
Subjects were counted only once within the same system organ class (SOC) or within the same preferred term (PT) and severity if they reported more than once.
SOCs were sorted alphabetically and PTs were sorted by frequency of subjects from high to low.

### 6.2.3. Analysis of Adverse Events

Nine subjects (37.5%) experienced a TEAE during the study. No deaths, other SAEs, or AEs leading to study drug withdrawal were reported. The most frequent TEAE was headache, which was reported by six subjects (25.0%). No other TEAEs were reported by more than one subject. Four subjects (16.7%) had TEAEs that were considered related to study drug, which included headache in one subject, libido increased and energy increased in one subject, night sweats in one subject, and hot flush in one subject. The majority of TEAEs were mild. Four subjects had moderate headache and one subject had moderate post-traumatic headache. There were no severe TEAEs.

### 6.2.4. Listing of Adverse Events by Subject

Adverse events are listed by subject elsewhere.

### 6.3. Deaths, Other Serious Adverse Events, and Other Significant Adverse Events

Serious adverse events were to be summarized elsewhere, and listings of SAEs, AEs leading to death, and AEs leading to withdrawal were to be presented elsewhere, respectively. However, there were no SAEs, AEs leading to death, and no AEs leading to withdrawal of study drug reported.

### 6.4. Clinical Laboratory Evaluation

All laboratory tests were performed at screening, and therefore, no post-treatment evaluations were performed.

Results of screening tests for FSH, Factor V Leiden mutation, and thyroid function are listed by subject elsewhere.

Urine drug and alcohol tests are listed by subject elsewhere, and urine pregnancy test results are listed by subject elsewhere.

Results of screening hematology tests, chemistry tests, and urinalyses are listed by subject elsewhere. Hematology and chemistry laboratory parameters assessed at screening are summarized elsewhere. Urinalysis parameters assessed at screening are presented elsewhere.

### 6.5. Vital Signs, Physical Findings, and Other Observations Related to Safety

### 6.5.1. Vital Signs

The change from period baseline in vital signs is summarized elsewhereand vital signs are listed by subject in the Safety Population elsewhere. Very small decreases in mean systolic and diastolic blood pressure were observed under both fed and fasting conditions.

The overall interpretations of vital signs are summarized elsewhere. There were no abnormal, clinically significant results in systolic blood pressure, diastolic blood pressure, heart rate, respiratory rate, or temperature observed during the study.

### 6.5.2. Concomitant Therapies

Concomitant medications are summarized elsewhere and listed by subject elsewhere. Eleven subjects (45.8%) took at least one concomitant medication during the study. The most common concomitant medication was ibuprofen, which was taken by 5 subjects (20.8%).

Concomitant non-drug therapies and procedures are summarized elsewhere and listed by subject elsewhere. One subject (4.2%) used a concomitant non-drug therapy: transcutaneous electrical nerve stimulation. Subject 01-106 had used this treatment for chronic lower back pain since 2009 and continued to use it during the study.

### 6.6. Safety Conclusions

Overall, TX-001HR 1 mg estradiol/100 mg progesterone was safe and well-tolerated.
Nine subjects (37.5%) experienced a TEAE during the study.
The most frequent TEAE was headache, which was reported by six subjects (25.0%). No other TEAEs were reported by more than one subject.
Four subjects (16.7%) had TEAEs that were considered related to study drug, which included headache in one subject, libido increased and energy increased in one subject, night sweats in one subject, and hot flush in one subject.
The majority of TEAEs were mild. There were no severe TEAEs. One subject had moderate post-traumatic headache and four subjects had moderate headache.
No deaths, other SAEs, or TEAEs leading to study drug withdrawal were reported.
Very small decreases in mean systolic and diastolic blood pressure were observed under both fasting and fed conditions, but no abnormal, clinically significant vital sign results were observed in any subject during the study.

### 7. Discussion and Overall Conclusions

This was a Phase 1, open-label, randomized, balanced, single-dose, two-treatment (fed and fasting), crossover, single-center study to assess the effect of food on the bioavailability of TX-001HR (estradiol and micronized progesterone capsules) in healthy postmenopausal female subjects. Twenty-four subjects were enrolled and randomized in a 1:1 ratio to the sequence of fasting or fed conditions. Each subject received a single oral dose of study drug under each condition, separated by a 14-day washout.

The PK parameters were estimated using baseline-adjusted and unadjusted plasma estradiol, estrone, and progesterone concentrations. The PK analyses were performed with and without Subject 01-109 due to her baseline concentrations of estradiol in Period 2 being higher than anticipated for postmenopausal women. The overall PK conclusions were not altered whether this subject was included in the analyses or excluded.

The GMRs of AUC₀₋ₜ and AUC_{0-∞} for estradiol were not different under fed and fasting conditions based on both baseline-adjusted and unadjusted concentrations indicating that food had no effect on these PK parameters and that the estradiol component of TX-001HR was bioequivalent under fasting and fed conditions. However, mean estradiol Cₘₐₓ (baseline-adjusted and unadjusted) was generally 2-fold greater and the tₘₐₓ earlier under fasting conditions compared to fed conditions.

The GMRs of AUC_{0-t,} AUC_{0-∞}, and Cₘₐₓ for estrone were similar under fed and fasting conditions based on both baseline-adjusted and unadjusted estrone concentrations indicating that food had no effect on estrone PK parameters and that estrone levels derived from the estradiol component of TX-001HR were bioequivalent under fasting and fed conditions.

Mean progesterone AUC_{0-t,} AUC_{0-∞}, and Cₘₐₓ were generally greater under fed conditions as compared to fasting conditions based on both baseline-adjusted and unadjusted progesterone concentrations indicating that food increases the bioavailability of the progesterone component of TX-001HR following administration.

Overall, TX-001HR was safe and well-tolerated. The most frequent AE was headache, and the majority of events were mild. No severe or serious TEAEs were reported and no subjects discontinued treatment due to TEAEs. Food had no effect on estradiol bioavailability based on specified statistical analyses of the extent of absorption (AUC). Food increased progesterone absorption following a single dose of TX-001HR.

It will be apparent to those skilled in the art that various modifications and variations can be made in the present disclosure without departing from the spirit or scope of the disclosure. Thus, it is intended that the present disclosure cover the modifications and variations of this disclosure provided they come within the scope of the appended claims and their equivalents.

Likewise, numerous characteristics and advantages have been set forth in the preceding description, including various alternatives together with details of the structure and function of the devices or methods. This disclosure is intended as illustrative only and as such is not intended to be exhaustive. It will be evident to those skilled in the art that various modifications may be made, especially in matters of structure, materials, elements, components, shape, size and arrangement of parts including combinations within the principles of the disclosure, to the full extent indicated by the broad general meaning of the terms in which the appended claims are expressed. To the extent that these various modifications do not depart from the spirit and scope of the appended claims, they are intended to be encompassed therein.

What is claimed is:

1. A method of treating a moderate to severe vasomotor symptom associated with estrogen deficiency in a menopausal woman, the method comprising:
administering to the menopausal woman a pharmaceutical composition comprising 1 mg of estradiol and 100 mg of progesterone,
wherein the pharmaceutical composition is orally administered to the menopausal woman once daily with food,
wherein the moderate to severe vasomotor symptom is hot flushes, and the menopausal woman has ≥ 50 moderate to severe hot flushes per week before treatment; and
wherein oral, daily administration of the composition with food for at least 4 weeks to a treatment group of menopausal women provides a reduction in the frequency or the severity of the moderate to severe vasomotor symptom as compared to a placebo group.

2. The method of claim 1, wherein the menopausal women in the treatment group have a baseline mean of 72.1 ± 27.80 hot flushes per week before treatment, and the menopausal women in the placebo group have a baseline mean of 72.3 ± 23.44 hot flushes per week before treatment.

3. The method of claim 2, wherein oral, daily administration of the composition with food for at least 4 weeks to menopausal women in the treatment group provides a mean weekly change from baseline in the frequency of the moderate to severe vasomotor symptom of -40.6 ± 30.59 hot flushes per week.

4. The method of claim 2, wherein oral, daily administration of the composition with food for at least 12 weeks to menopausal women in the treatment group provides a mean weekly change from baseline in the frequency of the moderate to severe vasomotor symptom of -55.1 ± 31.36 hot flushes per week.

5. The method of claim 1, wherein oral, daily administration of the composition with food for at least 12 weeks to menopausal women in the treatment group provides a mean weekly change from baseline in the severity of the moderate to severe vasomotor symptom of -1.12 ± 0.963 hot flushes per week.

6. The method of claim 2, wherein oral, daily administration of the composition with food for at least 4 weeks to menopausal women in the treatment group provides a mean weekly difference from the placebo group in the frequency of the moderate to severe vasomotor symptom of -12.81 ± 3.30 hot flushes per week.

7. The method of claim 2, wherein oral, daily administration of the composition with food for at least 12 weeks to menopausal women in the treatment group provides a mean weekly difference from the placebo group in the frequency of the moderate to severe vasomotor symptom of -16.58 ± 3.44 hot flushes per week.

8. The method of claim 2, wherein the menopausal women in the treatment group have a baseline mean severity of moderate to severe vasomotor symptoms of 2.54 ± 0.325 before treatment, and the menopausal women in the placebo group have a baseline mean severity of moderate to severe vasomotor symptoms of 2.52 ± 0.246 before treatment.

9. The method of claim 8, wherein oral, daily administration of the composition with food for at least 4 weeks to menopausal women in the treatment group provides a mean weekly difference from the placebo group in the severity of the moderate to severe vasomotor symptom of -0.48 ± 0.547 hot flushes per week.

10. The method of claim 8, wherein oral, daily administration of the composition with food for at least 4 weeks to menopausal women in the treatment group provides a mean weekly difference from the placebo group in the severity of the moderate to severe vasomotor symptom of -0.13 ± 0.061 hot flushes per week.

11. The method of claim 8, wherein oral, daily administration of the composition with food for at least 12 weeks to menopausal women in the treatment group provides a reduction in both the frequency and the severity of the moderate to severe vasomotor symptom compared to the placebo group.

12. The method of claim 1, wherein the pharmaceutical composition further comprises a solubilizing agent comprising a C6-C12 oil.

13. The method of claim 12, wherein the C6-C12 oil comprises predominantly C6-C12 fatty acid esters of glycerol, polyethylene glycol, propylene glycol, or a combination thereof.

14. The method of claim 12, wherein the C6-C12 oil comprises predominantly C6-C12 monoglycerides, diglycerides, triglycerides, or a combination thereof.

15. The method of claim 1, wherein the pharmaceutical composition further comprises a surfactant.

16. The method of claim 15, wherein the surfactant comprises lauroyl macrogol-32 glycerides, lauroyl polyoxyl-32 glycerides, lauroyl polyoxyglycerides, or a combination thereof.

17. The method of claim 1, wherein at least 80% of the estradiol in the composition is solubilized.

18. The method of claim 1, wherein at least 90% of the estradiol in the composition is solubilized.

19. The method of claim 1, wherein the composition is provided in a capsule.

20. The method of claim 1, wherein the method is effective at achieving a ≤ 1% incidence rate of endometrial hyperplasia following 12 months of treatment.

21. The method of claim 1, wherein the administration with food is at bedtime.

22. The method of claim 1, wherein the wherein the method results in a lower incidence of somnolence compared to the reference listed drug.

23. The method of claim 22, wherein the reference listed drug is progesterone in peanut oil.

24. A method of treating a moderate to severe vasomotor symptom associated with estrogen deficiency in a female subject, the method comprising:
administering to the subject a pharmaceutical composition comprising 1 mg of estradiol and 100 mg of progesterone,
wherein the pharmaceutical composition is orally administered to the subject once daily with food,
wherein the moderate to severe vasomotor symptom is hot flushes, and the subject has ≥ 50 moderate to severe hot flushes per week before treatment; and
wherein the pharmaceutical composition is effective at achieving a ≤1% incidence rate of endometrial hyperplasia following 12 months of therapy.
